(19) United States
(12) Patent Application Publication
(10) Pub. No.: US 2020/0347461 A1
(43) Pub. Date: Nov. 5, 2020

(54) PHENOTYPIC AGE AND DNA METHYLATION BASED BIOMARKERS FOR LIFE EXPECTANCY AND MORBIDITY

(71) Applicants: The Regents of the University of California, Oakland, CA (US); National Institutes of Health, Bethesda, MD (US)

(72) Inventors: Stefan Horvath, Los Angeles, CA (US); Morgan E. Levine, Bethany, CT (US); Luigi Ferrucci, Baltimore, MD (US)

(73) Assignees: The Regents of the University of California, Oakland, CA (US); National Institutes of Health, Bethesda, MD (US)

(21) Appl. No.: 16/963,065

(22) PCT Filed: Jan. 17, 2019
(86) PCT No.: PCT/US2019/014053

§ 371 Date: Jul. 17, 2020

Related U.S. Application Data
(60) Provisional application No. 62/618,422, filed on Jan. 17, 2018.

(51) Int. Cl. C12Q 1/6886; G16H 50/20

(52) U.S. Cl. CPC C12Q 1/6886; G16H 50/20; C12Q 2523/125; C12Q 2600/118; C12Q 2600/154

(57) ABSTRACT

Identifying reliable biomarkers of aging is a major goal in geroscience. While the first generation of epigenetic biomarkers of aging were developed using chronological age as a surrogate for biological age, we hypothesized that composite clinical measures of “phenotypic age”, may facilitate the development of a more powerful epigenetic biomarker of aging. Here we show that our newly developed epigenetic biomarker of aging “DNAm PhenoAge” strongly outperforms previous measure in regards to predictions for a variety of aging outcomes, including all-cause mortality, cancers, physical functioning, and, age-related dementia. It is also associated with Down syndrome, HIV infection, socioeconomic status, and various life style factors such as diet, exercise, and smoking. Overall, this single epigenetic biomarker of aging is able to capture risks for an array of diverse outcomes across multiple tissues and cells, and in moving forward, will facilitate the development of anti-aging interventions.

### CROSS-REFERENCE TO RELATED APPLICATIONS

This application claims priority under Section 119(e) from U.S. Provisional Application Ser. No. 62/618,422, filed Jan. 17, 2018, entitled “PHENOTYPIC AGE AND DNA METHYLATION BASED BIOMARKERS FOR LIFE EXPECTANCY AND MORBIDITY” the contents of each which are incorporated herein by reference.

### STATEMENT OF GOVERNMENT INTEREST

This invention was made with Government support under Grant Numbers AG051425 and AG052604, awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on Jan. 14, 2019, is named 30435_0341WOU1_SL.txt and is 201,768 bytes in size.

### TECHNICAL FIELD

The invention relates to methods and materials for examining biological aging in individuals.

### BACKGROUND OF THE INVENTION

One of the major goals of geroscience research is to define ‘biomarkers of aging’^{1,2}, which are individual-level measures of aging that can account for differences in the timing of disease onset, functional decline, and death over the life course. While chronological age is arguably the strongest risk factor for aging-related death and disease, it is important to distinguish chronological time from biological aging. Individuals of the same chronological age may exhibit greatly different susceptibilities to age-related diseases and death, which is likely reflective of differences in their underlying biological aging processes. Such biomarkers of aging will be crucial to enable instantaneous evaluation of interventions aimed at slowing the aging process, by providing a measurable outcome other than incidence of death and/or disease, which require extremely long follow-up observation.

One potential biomarker that has gained significant interest in recent years is DNA methylation (DNAm), given that chronological time has been shown to elicit predictable hypo- and hyper-methylation changes at many regions across the genome ³⁻⁷. As a result, the first generation of DNAm based biomarkers of aging were developed to predict chronological age⁸⁻¹⁰. The blood-based algorithm by Hannum⁹ and the multi-tissue algorithm by Horvath¹⁰ produced age estimates (DNAm age) that correlate with chronological age well above r=0.90 for full age range samples. Nevertheless, while the current epigenetic age estimators exhibit statistically significant associations with many age-related diseases and conditions¹¹⁻¹⁷, the effect sizes are typically small to moderate. Further, using chronological age as the reference, by definition, may exclude CpGs whose methylation patterns don't display strong time-dependent changes, but instead signal the departure of biological age from chronological age.

Previous work by us and others have shown that “phenotypic aging measures”, derived from clinical biomarkers¹⁸⁻²², strongly predict differences in the risk of all-cause mortality, cause-specific mortality, physical functioning, cognitive performance measures, and facial aging among same-aged individuals. What's more, in representative population data, some of these measures have been shown to be better indicators of remaining life expectancy than chronological age¹⁸, suggesting that they are approximating individual-level differences in biological aging rates.

Accordingly, there is a need for improved methods of observing phenotypic aging, which is predictive of an earlier age of death (all-cause mortality) that is independent of chronological age and traditional risk factors of mortality.

### SUMMARY OF THE INVENTION

This invention provides methods and materials useful to examine one or more clinical variables and DNA methylation biomarkers. As discussed in detail below, typically these biomarkers are based on variables that lend themselves to predicting life expectancy and risk for age-related diseases. For example, a first biomarker, referred to as “phenotypic age estimator”, is based on clinical variables such as measurements of factors such as Albumin, Creatinine, Glucose, C-reactive Protein, Lymphocyte Percentage, Mean Cell Volume, Red Blood Cell Distribution Width, Alkaline Phosphatase, White Blood Cell Count, and age at the time of assessment. A second biomarker, referred to as “DNA methylation PhenoAge”, is based on DNA methylation measurements at 513 locations across the human DNA molecule. As discussed below, by examining such biomarkers in an individual, it is possible to obtain information that is highly predictive of multiple morbidity and mortality outcomes in that individual.

The idea of using DNA methylation (DNAm) to estimate biological age has recently gained interest following the discovery that many CpGs throughout the genome display hyper- or hypo-methylation patterns as a function of chronological age. While most of the first-generation epigenetic biomarkers of aging capitalized on these age associations to identify CpGs from which to build composite scores, we hypothesized that a more powerful epigenetic biomarker of aging could be generated from DNA methylation data by replacing chronological age with a surrogate measure of “phenotypic aging” that, in and of itself, differentiates morbidity and mortality risk among same-age individuals. Using multiple large epidemiological studies, we demonstrate that our new epigenetic biomarker that is examines the above-noted combination of factors, DNAm PhenoAge, is highly predictive of multiple morbidity and mortality outcomes—including, but not limited to: life expectancy, heart disease, cancer, and age related dementia. Further, it produces reliable age estimates and risk predictions when measured in various tissues. This shows that our single DNAm based biomarker (DNAm PhenoAge) is capable of capturing risk for an array of diverse diseases and conditions across multiple tissues and cells. As such, DNAm PhenoAge will be useful for assessing personalized risk, improving our understanding of the biological aging process and, evaluating promising interventions aimed at slowing aging and preventing disease.

The invention disclosed herein has a number of embodiments. Embodiments of the invention include method of obtaining information on a phenotypic age of an individual, the method comprising observing methylation of genomic DNA obtained from the individual, wherein methylation is observed in at least 10 CpG methylation markers in polynucleotides having SEQ ID NO: 1-SEQ ID NO: 513 so that information on the phenotypic age of the individual is obtained. Typically in these methods, observing methylation of genomic DNA comprises hybridizing genomic DNA from the individual to a methylation array comprising the polynucleotides having sequences of SEQ ID NO: 1-SEQ ID NO: 513 coupled to a matrix; and/or comprises performing a bisulfite conversion process on the genomic DNA so that cytosine residues in the genomic DNA are transformed to uracil, while 5-methylcytosine residues in the genomic DNA are not transformed to uracil. In such embodiments, the method can comprise observing a clinical variable in the individual comprising at least one of: concentrations of albumin in the individual, concentrations of creatine in the individual, concentrations of glucose in the individual, concentrations of c-reactive protein in the individual, concentrations of alkaline phosphatase in the individual lymphocyte percentage in the individual, mean cell volume in the individual, red blood cell distribution width in the individual, white blood cell count in the individual, and age of the individual at the time of assessment. In certain embodiments of the invention, at least 3, 4, 5, 6, 7 or 8 clinical variables are observed.

Embodiments of the invention can include additional steps such as comparing the chronological age of the individual at the time of assessment and the phenotypic age so as to obtain information on life expectancy of the individual. Embodiments of the invention include using information on the phenotypic age obtained by the method to predict an age at which the individual may suffer from one or more age related diseases or conditions. Embodiments of the invention include those that compare the CG locus methylation profile observed in the individual to the CG locus methylation profile of genomic DNA having SEQ ID NO: 1-SEQ ID NO: 513 present in white blood cells or epithelial cells derived from a group of individuals of known ages; and then correlating the CG locus methylation observed in the individual with the CG locus methylation and known ages in the group of individuals. In typical embodiments of the invention, methylation is observed by a process comprising hybridizing genomic DNA obtained from the individual with at least 100, 200, 300, 400 or 500 polynucleotides comprising SEQ ID NO: 1-SEQ ID NO: 513 disposed in an array. In embodiments of the invention, the phenotypic age of the individual can be estimated using a weighted average of methylation markers within the set of 513 methylation markers. Optionally, methylation marker data is further analyzed, for example by a regression analysis. Optionally in these methods, methylation is observed in genomic DNA obtained from leukocytes or epithelial cells obtained from the individual.

A specific embodiment of the invention is a method of observing a phenotypic age of an individual, the method comprising observing methylation of genomic DNA obtained from the individual, wherein methylation is observed in 513 CpG methylation markers in polynucleotides having SEQ ID NO: 1-SEQ ID NO: 513; and the method comprises hybridizing genomic DNA from the individual to a methylation array comprising the polynucleotides of SEQ ID NO: 1-SEQ ID NO: 513 coupled to a matrix, so that the phenotypic age of the individual is observed.

In certain embodiments of the invention, methods include observing a clinical variable in the individual comprising at least one of: concentrations of albumin in the individual, concentrations of creatine in the individual, concentrations of glucose in the individual, concentrations of c-reactive protein in the individual, concentrations of alkaline phosphatase in the individual lymphocyte percentage in the individual, mean cell volume in the individual, red blood cell distribution width in the individual, white blood cell count in the individual, and age of the individual at the time of assessment. In some embodiments of the invention, the method further comprising observing at least one factor selected from individual diet history, individual smoking history and individual exercise history. Optionally, the observed phenotypic age is then used to assess a risk of a cancer mortality in the individual (e.g. to asses a risk of breast cancer, lung cancer or the like, or to assess a risk of dementia or diabetes mortality in the individual).

A related embodiment of the invention is a tangible computer-readable medium comprising computer-readable code that, when executed by a computer, causes the computer to perform operations including: receiving information corresponding to methylation levels of a set of methylation markers in a biological sample, wherein the set of methylation markers comprises 513 methylation markers that are identified in Table 5; determining an epigenetic age by applying a statistical prediction algorithm to methylation data obtained from the set of methylation markers; and then determining an epigenetic age using a weighted average of the methylation levels of the 513 methylation markers. Optionally in this embodiment, the tangible computer-readable medium comprising computer-readable code, when executed by a computer, further causes the computer to perform operations including: receiving information corresponding to methylation levels of a set of clinical variables in a biological sample, information that is then used for determining an epigenetic age.

Both phenotypic age, and in particular DNAm PhenoAge, are useful biomarkers for human anti-aging studies given that these are highly robust, blood based biomarkers that capture organismal age and the functional state of many organ systems and tissues, thus allowing efficacy of interventions to be evaluated based on real-time measures of aging, rather than relying on long-term outcomes, such as morbidity and mortality. Finally, this measure may be another component of the personalized medicine paradigm, as it allows for evaluation of risk based on an individual's personalized DNAm profile.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1. Roadmap for developing DNAm PhenoAge. The roadmap depicts our analytical procedures. In step 1, we developed an estimate of ‘Phenotypic Age’ based on clinical measure. Phenotypic age was developed using the NHANES III as training data, in which we employed a proportional hazard penalized regression model to narrow 42 biomarkers to 9 biomarkers and chronological age. This measure was then validated in NHANES IV and shown to be a strong predictor of both morbidity and mortality risk. In step 2, we developed an epigenetic biomarker of phenotypic age, which we call DNAm PhenoAge, by regressing phenotypic age (from step 1) on blood DNA methylation data, using the InCHIANTI data. This produced an estimate of DNAm PhenoAge based on 513 CpGs. In step 3, we validated our new epigenetic biomarker of aging, DNAm PhenoAge, using multiple cohorts, aging-related outcomes, and tissues/cells. We also performed heritability and functional enrichment analysis.

FIG. 2. Mortality Prediction by DNAm PhenoAge. FIG. 2A: Using four samples from large epidemiological cohorts-two samples from the Women's health Initiative, the Framingham Heart Study, and the Normative Aging Study-we tested whether DNAm PhenoAge was predictive of all-cause mortality. The figure displays a forest plot for fixed-effect meta-analysis, based on Cox proportional hazard models, and adjusting for chronological age. Results suggest that DNAm PhenoAge is predictive of mortality in all samples, and that overall, a one year increase in DNAm PhenoAge is associated with a 4.2% increase in the risk of death (p=1.1E-36). This is in contrast to the first generation of epigenetic aging biomarkers by Hannum and Horvath, for which the Hannum measure predicts mortality, but to a much lesser degree, and the Horvath measure is not significantly associated with mortality. FIG. 2B & C: Using the WHI sample 1, we plotted Kaplan-Meier survival estimates using actual data from WHI sample 1 for the fastest versus the slowest agers (2B), and we used equation from the proportional hazard model to predict remaining life expectancy and plotted predicted survival assuming a chronological age of 50 and a DNAm PhenoAge of either 40 (slow ager), 50 (average ager), or 60 (fast ager) (2C). Median life expectancy was higher for slower agers, such that it was predicted to be approximately 81 years for the fastest agers, 83.5 years for average agers, and 86 years for the slowest agers.

FIG. 3. Chronological age prediction of DNAm PhenoAge in a variety of tissues and cells. Although DNAm PhenoAge was developed using methylation data from whole blood, FIG. 3 suggests that it also tracks chronological age in a wide variety of tissues and cells. For instance, the correlation across all tissues/cells we examined is r=0.71. C\Overall, correlations range from r=0.35 (breast) to r=0.92 (temporal cortex in brain).

FIG. 4. DNAm PhenoAge measured in dorsolateral prefrontal cortex relates to Alzheimer's disease and related neuropathologies. Using postmortem data from the Religious Order Study (ROS) and the Memory and Aging Project (MAP), we find a moderate/high correlation between chronological age and DNAm PhenoAge (FIG. 4A), that is further increased after adjusting for the estimated proportion on neurons in each sample (panel C). We also find that DNAm PhenoAge is significantly higher (p=0.00046) among those with Alzheimer's disease versus controls (panel D), and that it positively correlates with amyloid load (p=0.012, panel E), neuritic plaques (p=0.0032, panel F), diffuse plaques (p=0.036, panel G), and neurofibrillary tangles (p=0.0073, panel H).

FIG. 5. Association between phenotypic age and morbidity. Using NHANES IV as validation data, we tested whether phenotypic age, adjusting for chronological age, was associated with morbidity. Results showed strong dose-effects, such that those with high phenotypic ages tended to have more coexisting morbidities (A) and greater physical functioning problems (B) compared to phenotypically younger persons of the same chronological age.

FIG. 6. Longitudinal comparisons of phenotypic age and DNAm PhenoAge. The top two panels show the distributions of the change in phenotypic age (A) and DNAm PhenoAge (B) over nine years of follow-up in InCHIANTI. The second row depicts the age-adjusted correlations between the two time-points for phenotypic age (C) and DNAm PhenoAge (D). Both variables showed moderate/high correlations, suggesting that, above and beyond the expected increase with chronological time, they remain stable-those who are fast agers, remain fast agers. Finally, panel E shows the correlation between change in phenotypic age and change in DNAm PhenoAge, suggesting that those who experience an acceleration of phenotypic age based on clinical markers also experience age acceleration on an epigenetic level.

FIG. 7. Associations between smoking and DNAm PhenoAge. When comparing DNAm PhenoAge by smoking status, we find that current smokers have significantly high epigenetic ages (A). This is also true when comparing DNAm PhenoAge as a function of pack-years (B). However, no associations with pack-years are found when stratifying by smoking status-former versus current (C & D).

FIG. 8. Fixed effect meta-analysis of the effect of DNAm PhenoAge on the hazard of all cause mortality, stratifying by smoking. In smoking stratified analyses, adjusting for pack-years (in smokers) and chronological age, we find that DNAm PhenoAge significantly predicts mortality even within groups, and despite much smaller sample sizes. The Hannum measure also relates to mortality in both smokers and non-smokers; although to a lesser degree than DNAm PhenoAge.

FIG. 9. Effect of ethnicity on DNAm PhenoAge in the WHI. When comparing DNAm PhenoAge by race/ethnicity, we find that non-Hispanic blacks have the highest ages, whereas non-Hispanic whites have the lowest (A). Despite the fact that DNAm PhenoAge was trained in a mostly non-Hispanic white population, the differences by race/chronological age and ethnicity do not appear to be a reflection of the reliability of the measure within the various strata, given that it shows very consistent age trends across all three groups (B, C, & D).

FIG. 10. Associations with measures of age acceleration in the WHI. FIG. 10A: Correlations (bicor, biweight midcorrelation) between select variables and the three measures of epigenetic age acceleration are colored according to their magnitude with positive correlations in red, negative correlations in blue, and statistical significance (p-values) in green. Blood biomarkers were measured from fasting plasma collected at baseline. Food groups and nutrients are inclusive, including all types and all preparation methods, e.g. folic acid includes synthetic and natural, dairy includes cheese and all types of milk, etc. Variables are adjusted for ethnicity and dataset (BA23 or AS315). FIG. 10B: Multivariate linear regression analysis was also used to examine the associations, adjusting for covariates. Again we find that minority race/ethnicity, lower education, higher BMI, higher CRP, smoking and having metabolic syndrome is associated with higher DNAm PhenoAge. Red meat consumption is also associated positively associated with DNAm PhenoAge in model 2; however the association becomes marginal after adjusting for biomarkers, which may suggest that various biomarkers mediate the association between red meat consumption and DNAm PhenoAge.

FIG. 11. Age adjusted blood cell counts versus phenotypic age acceleration in the Women's Health Initiative (BA23 data). DNAm PhenoAge acceleration (x-axis) versus age adjusted estimates of various measures of abundance of blood cell counts. (A) plasma blasts (activated B cells), (B) percentage of exhausted CD8+ T cells (defined as CD8+CD28-CD45RA−), (C) naïve CD8+ T cell count, (D) naïve CD4+ T cell count, E) proportion of CD+8 T cells, F) proportion of CD4+ helper T cells, G) proportion of natural killer cells, H) proportion of B cells, I) proportion of monocytes, J) proportion of granulocytes (mainly neutrophils). The correlation coefficient and p-value results from the Pearson correlation test. Two software tools were used to estimate the blood cell counts using DNA methylation data. First, Houseman's estimation method 6, which is based on DNA methylation signatures from purified leukocyte samples, was used to estimate the proportions of CDS+ T cells, CD4+T, natural killer, B cells, and granulocytes. Granulocytes are also known as polymorphonuclear leukocytes. Second, the advanced analysis option of the epigenetic clock software 7,s was used to estimate the percentage of exhausted CD8 T cells (defined as CD28-CD45RA−) and the number (count) of naïve CD8+ T cells (defined as (CD45RA+CCR7+). Points are colored by race/ethnicity (blue=Hispanic, green=African Ancestry, grey=non-Hispanic white).

FIG. 12. Fixed effects meta analysis of the effect of DNAm phenotypic age acceleration on the hazard of death after adjusting for blood cell counts. The Cox regression model adjusted for chronological age, race/ethnicity, smoking pack years, and imputed blood cell counts (exhausted CD8+ T cells, naïve CD8+ T cells, CD4T cells, natural killer cells monocytes, granulocytes). The meta analysis p value is colored in red. A significant heterogeneity p value (red font) indicates that the hazard ratios differ significantly across studies.

FIG. 13. Properties of the 513 CpGs that underly DNAmPhenoAge. In our functional enrichment analysis of the chromosomal locations of the 513 CpGs, we distinguished CpGs with positive age correlation from CpGs with negative age correlation. CpGs with positive age correlation exhibited a lower variance but a similar mean methylation level compared to CpGs with negative age correlation (B,C). The 149 CpGs whose age correlation exceeded 0.2 tended to be located in CpG islands (E) and were significantly enriched with polycomb group protein targets (p=8.7E-5, D). A) Each CpGs was correlated with chronological age in whole blood. The histogram shows the correlation coefficients. To carry out a functional annotation analysis, we split the 513 CpGs into 3 groups according to the thresholds visualized as vertical red lines. Group 1 is comprised of 126 CpGs with a negative age correlation(<−0.2). Group 3 is comprised of 149 CpG with a positive age correlation(>0.2). Group 2 is comprised of 238 whose age correlation lies between −0.2 and +0.2. B) Variance of the DNA methylation levels versus the 3 groups. Note that CpGs with positive age correlation (i.e. CpGs in group 3) exhibit the lowest variance. C) Mean methylation levels in blood versus group status. D) Proportion of polycomb group protein targets (y-axis) versus membership in group 3, i.e. the set of clock CpGs that exhibit an age correlation >0.2. To avoid biasing the analysis, the comparison group was comprised of all CpGs that are located on the Illumina 27k array. E) Proportion of CpGs that are located in a CpG island (y-axis) versus membership in group 3. F) Proportion of CpGs that are located in a CpG island (y-axis) versus membership in group 2.

FIG. 14. Partial likelihood versus log(lambda) parameter for elastic net proportional hazard model. Ten-fold cross-validation was employed to select the parameter value, lambda, for the penalized regression. In order to develop a sparse phenotypic age estimator (the fewest biomarker variables needed to produce robust results) we selected a lambda of 0.0192, which represented a one standard deviation increase over the lambda with minimum mean-squared error during cross-validation. Of the forty-two biomarkers included in the penalized Cox regression model, this resulted in ten variables (including chronological age) that were selected for the phenotypic age predictor.

FIG. 15. Partial likelihood versus log(lambda) parameter for elastic net regression. The CpGs used in the elastic net represent those that are found on the Illumina Infinium 450k chip, the EPIC chip, and the Illumina Infinium 27k chip. Lambda was selected using 10-fold cross-validation; however, given that sparseness was not a goal with this model, the lambda with the minimum mean-squared error was selected (lambda=0.35). This lambda, produced a model in which phenotypic age is predicted by DNAm levels at 513 CpGs.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized and structural changes may be made without departing from the scope of the present invention. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

All publications mentioned herein are incorporated herein by reference to disclose and describe aspects, methods and/or materials in connection with the cited publications. For example, Levine et al., Aging, 2018 Apr. 18; 10(4):573-591; U.S. Patent Publication 20150259742, U.S. patent application Ser. No. 15/025,185, titled “METHOD TO ESTIMATE THE AGE OF TISSUES AND CELL TYPES BASED ON EPIGENETIC MARKERS”, filed by Stefan Horvath; U.S. patent application Ser. No. 14/119,145, titled “METHOD TO ESTIMATE AGE OF INDIVIDUAL BASED ON EPIGENETIC MARKERS IN BIOLOGICAL SAMPLE”, filed by Eric Villain et al.; and Hannum et al. “Genome-Wide Methylation Profiles Reveal Quantitative Views Of Human Aging Rates.” Molecular Cell. 2013; 49(2):359-367 and patent US2015/0259742, are incorporated by reference in their entirety herein.

DNA methylation refers to chemical modifications of the DNA molecule. Technological platforms such as the Illumina Infinium microarray or DNA sequencing based methods have been found to lead to highly robust and reproducible measurements of the DNA methylation levels of a person. There are more than 28 million CpG loci in the human genome. Consequently, certain loci are given unique identifiers such as those found in the Illumina CpG loci database (see, e.g. Technical Note: Epigenetics, CpG Loci Identification ILLUMINA Inc. 2010). These CG locus designation identifiers are used herein. In this context, one embodiment of the invention is a method of obtaining information useful to observe biomarkers associated with a phenotypic age of an individual by observing the methylation status of one or more of the 513 methylation marker specific GC loci that are identified in Table 5.

The term “epigenetic” as used herein means relating to, being, or involving a chemical modification of the DNA molecule. Epigenetic factors include the addition or removal of a methyl group which results in changes of the DNA methylation levels. Novel molecular biomarkers of aging that observe methylation patterns in genomic DNA, such as those termed “DNA methylation PhenoAge”, or “phenotypic age” (allow one to prognosticate mortality, are interesting to gerontologists (aging researchers), epidemiologists, medical professionals, and medical underwriters for life insurances. Exclusively clinical biomarkers such as lipid levels, body mass index, blood pressures have a long and successful history in the life insurance industry. By contrast, molecular biomarkers of aging have rarely been used.

The profitability of a life insurance product directly depends on the accurate assessment of mortality risk because the costs of life insurance (to the insurance company) are directly proportional to the number of deaths in a given category. Thus, any improvement in assessing mortality risk and in improving the basic classification will directly translate into cost savings. For the reasons noted above, DNA methylation (DNAm) based biomarkers of aging are useful for predicting mortality. Consequently, they are useful the life insurance industry due to their ability to increase the accuracy of medical underwriting. DNAm measurements can provide a host of complementary information that can inform the medical underwriting process. In this context, the DNAm based biomarkers and associated method disclosed herein can be used both to molecularly estimate complete blood counts and to estimate biological age, as well as to directly predict/prognosticate mortality. Using embodiments of the invention disclosed herein, upon completing a medical exam, an insurer can, for example, look at a combination of the clinical biomarker and DNA methylation test results as well as other factors such as family health history and lifestyle choices to classify the applicant into useful classification categories such as: 1) preferred plus/super preferred/preferred select/preferred elite, 2) preferred, 3) standard plus, 4) standard, 5) preferred smoker, 6) standard smoker, 7) table rate A, 8) table rate B, etc. Each of these categories has a distinct mortality risk and usually directly relates to the pricing of the insurance product. The basic classification is largely determined by well established risk factors of mortality such as sex, smoking status, family history of death, prior history of disease (e.g. diabetes status, cancer), and a host of clinical biomarkers (blood pressure, body mass index, cholesterol, glucose levels, hemoglobin A1C).

The term “nucleic acids” as used herein may include any polymer or oligomer of pyrimidine and purine bases, preferably cytosine, thymine, and uracil, and adenine and guanine, respectively. The present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “methylation marker” as used herein refers to a CpG position that is potentially methylated. Methylation typically occurs in a CpG containing nucleic acid. The CpG containing nucleic acid may be present in, e.g., in a CpG island, a CpG doublet, a promoter, an intron, or an exon of gene. For instance, in the genetic regions provided herein the potential methylation sites encompass the promoter/enhancer regions of the indicated genes. Thus, the regions can begin upstream of a gene promoter and extend downstream into the transcribed region.

The phrase “selectively measuring” as used herein refers to methods wherein only a finite number of methylation marker or genes (comprising methylation markers) are measured rather than assaying essentially all potential methylation marker (or genes) in a genome. For example, in some aspects, “selectively measuring” methylation markers or genes comprising such markers can refer to measuring more than (or not more than) 500, 200, 100, 75, 50, 25, 10 or 5 different methylation markers or genes comprising methylation markers.

The invention described herein provides novel and powerful predictors of life expectancy, mortality, and morbidity based on DNA methylation levels. In this context, it is critical to distinguish clinical from molecular biomarkers of aging. Clinical biomarkers such as lipid levels, blood pressure, blood cell counts have a long and successful history in clinical practice. By contrast, molecular biomarkers of aging are rarely used. However, this is likely to change due to recent breakthroughs in DNA methylation based biomarkers of aging. Since their inception, DNA methylation (DNAm) based biomarkers of aging promise to greatly enhance biomedical research, clinical applications, patient care, and even medical underwriting when it comes to life insurance policies and other financial products. They will also be more useful for clinical trials and intervention assessment that target aging, since they are more proximal to the biological changes that characterize the aging process compared to upstream clinical read outs of health and disease status.

The disclosure presented herein surrounding the prediction of mortality and morbidity show that these combinations of clinical and DNAm based biomarkers are highly robust and informative for a range of applications. DNAm PhenoAge can not only be used to directly predict/prognosticate mortality but also relate to a host of age related conditions such as heart disease risk, cancer risk, dementia status, cardiovascular disease and various measures of frailty.

The invention disclosed herein has a number of embodiments. One embodiment of the invention is a method of observing biomarkers that are associated with a phenotypic age of an individual. In such embodiments, the method comprises observing a biomarker comprising the state of a clinical variable in the individual comprising at least one of: concentrations of albumin in the individual, concentrations of creatine in the individual, concentrations of glucose in the individual, concentrations of c-reactive protein in the individual, concentrations of alkaline phosphatase in the individual lymphocyte percentage in the individual, mean cell volume in the individual, red blood cell distribution width in the individual, white blood cell count in the individual, and age of the individual at the time of assessment; and, in addition, further observing another biomarker comprising the individual's methylation status at at least 10 513 CpG methylation markers that are identified in Table 5 such that biomarkers associated with the phenotypic age of the individual are observed. In some embodiments, methylation is observed by a process comprising hybridizing genomic DNA obtained from the individual with 513 complementary sequences disposed in an array on a substrate. Optionally, methylation is observed by a process comprising treatment of genomic DNA from the population of cells from the individual with bisulfite to transform unmethylated cytosines of CpG dinucleotides in the genomic DNA to uracil.

In typical embodiments of the invention, at least 3, 4, 5, 6, 7 or 8 clinical variables are observed. In some embodiments of the invention, the second DNA methylation biomarker is observed in a population of leukocytes or epithelial cells obtained from the individual. Optionally the method comprises assessing on or more of the biomarkers in a regression analysis. In certain embodiments, the phenotypic age of the individual is estimated using a weighted average of methylation markers within the set of 513 methylation markers. Embodiments of the invention can further comprise examining at least one factor selected from the diet of the individual, whether the individual smokes and the levels that the individual exercises. Embodiments of the invention can compare the age of the individual at the time of assessment and the phenotypic age so as to obtain information on life expectancy of the individual. In certain embodiments of the invention, the method includes using the phenotypic age to predict the age at which the individual may suffer from one or more age related diseases or conditions. Further embodiments and aspects of the invention are discussed below.

### Description of the Phenotypic Age Estimator

Previous work has shown that “phenotypic aging measures”, derived from clinical biomarkers (see, e.g. Levine M E., The Journals of Gerontology Series A: Biological Sciences and Medical Sciences. 2013; 68(6):667-674; Li S et al., Twin Res Hum Genet. 2015; 18(6):720-726; Sebastiani et al., Aging Cell. 2017; and Ferrucci L et al., Public Health Reviews. 2010; 32(2):475-488), strongly predict differences in the risk of all-cause mortality, cause-specific mortality, physical functioning, cognitive performance measures, and facial aging among same-aged individuals. What's more, in representative population data, some of these measures have been shown to be better indicators of remaining life expectancy than chronological age (Levine M E., The Journals of Gerontology Series A: Biological Sciences and Medical Sciences. 2013; 68(6):667-674), suggesting that they are approximating individual-level differences in biological aging rates. We developed a new phenotypic age predictor based on 10 variables total (9 clinical biomarkers and chronological age at the time of the assessment). These variables were selected out of a possible 42 biomarkers, using an elastic net proportion hazards model, and are aggregated into a composite score by forming a weighted average

WeightedAverage=(−Albumin*0.0336+log(Creatinine)*0.0095+Glucose*0.1953+C−reactiveProtein*0.0954−LymphocytePerc*0.0120+MeanCellVolume*0.0268+RedBloodCellDistributionWidth*0.3306+AlkalinePhosphatase*0.0019+WhiteBloodCellCount*0.0554+age*0.0804−19.9067).

Next the weighted average is transformed using a monotonically increasing function to arrive at a phenotypic age estimate (in units of years). Validation data for phenotypic age came from the fourth National Health and Nutrition Examination Survey (NHANES IV), and included up to 17 years of mortality follow-up for n=6,209 national representative US adults. Mortality results show that a one year increase in phenotypic age is associated with a 9% increase in the hazard of all-cause mortality (hazard ratio, HR=1.09, p-value=3.8E-49), a 9% increase in the risk of aging-related mortality(HR=1.09, p=4.5E-34), a 10% increase in the risk of CVD mortality (HR=1.10, p=5.1E-17), a 7% increase in the risk of cancer mortality (HR=1.07, p=7.9E-10), a 20% increase in the risk of diabetes mortality (HR=1.20, p=1.9E-11), and a 9% increase in the risk of lung disease mortality (HR=1.09, p=6.3E-4). Finally, in the proportional hazard model, phenotypic age completely accounted for the effect of chronological age, such that chronological age no longer exhibited a significant positive association with mortality.

Finally, we tested the association between phenotypic age and 1) the number of coexisting morbidities a participant had been diagnosed with, and 2) levels of physical functioning problems. Results showed that after adjusting for chronological age, persons with more coexisting morbidities also display higher phenotypic ages on average (p=3.9E-21). Similarly, those with worse physical functioning tended to have higher phenotypic ages (p=2.1E-10).

### Description of DNAm PhenoAge Estimator

Data from the Invecchiare in Chianti (InCHIANTI) study was used to relate blood DNAm levels to phenotypic age. Elastic net regression produced a model in which phenotypic age is predicted by DNAm levels at 513 CpGs. The linear combination of the weighted 513 CpGs yields a DNAm based estimator of phenotypic age, that we refer to as ‘DNAm PhenoAge’.

To demonstrate the utility of DNAm PhenoAge, we used four independent large-scale samples-two samples from Women's Health Initiative (WHI) (n=2,016; and n=2,191), the Framingham Heart Study (FHS) (n=2,553), and the Normative Aging Study (n=657). In these studies, DNAm PhenoAge correlated with chronological age at r=0.67 in WHI (Sample 1), r=0.69 in WHI (Sample2), r=0.78 in FHS, and r=0.62 in the Normative Aging Study. The four validation samples were then used to assess the effects of DNAm PhenoAge on mortality. DNAm PhenoAge was significantly associated with subsequent mortality risk in all studies (independent of chronological age), such that, a one year increase in DNAm PhenoAge is associated with a 4% increase in the risk of all-cause mortality (Meta(FE)=1.042, Meta p=1.1E-36). We also observe strong associations between DNAm PhenoAge and a variety of other aging outcomes. For instance, independent of chronological age, higher DNAm PhenoAge is associated with an increase in a person's number of coexisting morbidities (Meta P-value=4.56E-15), a decrease in likelihood of being disease-free (Meta P-value=1.06E-7), an increase in physical functioning problems (Meta P-value=2.05E-13), an increase in the risk of coronary heart disease (CHD) risk (Meta P-value=2.43E-10, and an earlier age at menopause (Meta P-value=8.22E-4)—suggesting that women were epigenetically older if they had entered menopause earlier.

Additional replication data was used to test for associations with other aging outcomes. For instance, we find that among the 527 women who were cancer free at age 50, accelerated DNAm PhenoAge predicts incident breast cancer (p=0.033, OR: 1.037). We also find a marginally significant reduction of approximately 2.4 years for the DNAm PhenoAge of semi-super centenarian offspring, relative to controls (p=0.065). Using blood methylation data, we evaluated whether DNAm PhenoAge relates to clinically diagnosed dementia in living individuals. Results suggest that those with presumed Alzheimer's disease (AD, n=154) and/or frontotemporal dementia (FTD, n=116) have significantly higher DNAm PhenoAge compared to non-demented (n=334) individuals (P=2.2E-2), and the strength of the association is further increased (P=9.4E-3) when limiting the sample to those ages 75 and older. We also find that DNAm PhenoAge, relates to Down syndrome in two separate blood methylation datasets (p=0.0046 and p=4.0E-11), and similarly relates to HIV infection in two blood datasets (p=6E-6 and p=8.6E-6). We observe a suggestive relationship between DNAm PhenoAge in blood and Parkinson's disease status (p=0.028) for individuals from European ancestry.

We examined the association between DNAm PhenoAge and smoking and found that DNAm PhenoAge also significantly differs by smoking status (p=0.0033). Next, we re-evaluated the morbidity and mortality associations (fully-adjusted) in our four samples, stratifying by smoking status (smokers vs. non-smokers). We find that DNAm PhenoAge is associated with mortality both among smokers (adjusted for pack-years) (Meta(FE)=1.041, Meta p=2.6E-14), and among persons who have never smoked (Meta(FE)=1.027, Meta p=7.9E-7). Moreover, among never smokers, DNAm PhenoAge relates to the number of coexisting morbidities (Meta P-value=7.83E-6), physical functioning status (Meta P-value=2.63E-3), disease free status (Meta P-value=4.38E-4), and CHD (Meta P-value=1.80E-4), while among current smokers, it relates to the number of coexisting morbidities (Meta P-value=4.61E-5), physical functioning status (Meta P-value=1.01E-4), and disease free status (Meta P-value=0.0048), but only exhibits a suggestive association with CHD (Meta P-value=0.084).

We studied whether DNAm PhenoAge of blood predicts lung cancer risk in the first WHI sample. After adjusting for chronological age, race/ethnicity, pack-years, and smoking status, results showed that a one year increase in DNAm PhenoAge is associated with a 5% increase in lung cancer risk (HR=1.05, p=0.031), and when restricting the model to current smokers only, we find that the effect of DNAm PhenoAge on lung cancer mortality is even stronger (HR=1.10, p=0.014).

We also find evidence of social gradients in DNAm PhenoAge, such that those with higher education (p=6E-9) and higher income (p=9E-5) appear younger. DNAm PhenoAge relates to exercise and dietary habits, such that increased exercise (p=7E-5) and markers of fruit/vegetable consumption (such as carotenoids, p=5E-22) are associated with lower DNAm PhenoAge.

We also evaluated DNAm PhenoAge in other non-blood tissues. Although DNAm PhenoAge was developed from DNAm levels assessed in whole blood, our empirical results show that it strongly correlates with chronological age in a host of different tissues. For instance, when examining all tissue concurrently, the correlation between DNAm PhenoAge and chronological age was 0.71. Age correlations in brain tissue ranged from 0.54 to 0.92. Consistent age correlations were also found in breast (r=0.47), buccal cells (r=0.88), dermal fibroblasts (r=0.87), epidermis (r=0.84), colon (r=0.88), heart (r=0.66), kidney (r=0.64), liver (r=0.80), lung (r=055), and saliva (r=0.81).

### Novelty Surrounding DNAm PhenoAge

DNA methylation (DNAm) data have given rise to highly accurate age estimation methods known as “epigenetic clocks”. These recently developed DNA methylation-based biomarkers allow one to estimate the epigenetic age of an individual (see, e.g. Levine M E., The Journals of Gerontology Series A: Biological Sciences and Medical Sciences. 2013; 68(6):667-674; Li S et al., Twin Res Hum Genet. 2015; 18(6):720-726; Sebastiani et al., Aging Cell. 2017; and Ferrucci L et al., Public Health Reviews. 2010; 32(2):475-488). For example, the “epigenetic clock”, developed by Horvath, which is based on methylation levels of 353 CpGs, can be used to estimate the age of most human cell types, tissues, and organs (Sebastiani et al., Aging Cell. 2017). The first generation of DNAm based biomarkers of aging were developed using chronological age as a surrogate measure for biological age. While the current epigenetic age estimators exhibit statistically significant associations with many age-related diseases and conditions, the effect sizes are typically small to moderate. While chronological age is arguably the strongest risk factor for aging-related death and disease, it is important to distinguish chronological time from biological aging. Individuals of the same chronological age may exhibit greatly different susceptibilities to age-related diseases and death, which is likely reflective of differences in their underlying biological aging processes (Ferrucci L et al., Public Health Reviews. 2010; 32(2):475-488). Using chronological age as the reference in the developing of epigenetic biomarkers of aging, by definition, may exclude CpGs whose methylation patterns don't display strong time-dependent changes, but instead signal the departure of biological age from chronological age. Thus, we hypothesized that a more powerful epigenetic biomarker of aging could be generated from DNAm by replacing chronological age with a surrogate measure of “phenotypic age” that, in and of itself, differentiates morbidity and mortality risk among same-age individuals.

Using a novel two-step method, we were successful in developing a DNAm based biomarker of aging that is highly predictive of nearly every morbidity and mortality outcome we tested. Our study demonstrates that DNAm PhenoAge greatly outperforms the first generation of DNAm based biomarkers of aging from Hannum (Hannum et al., Mol Cell. 2013; 49) and Horvath (Horvath S., Genome Biol. 2013; 14(R115), in terms of both its predictive accuracy for time to death and its associations with various other aging measures, including disease incidence/prevalence and physical functioning. Most surprisingly, DNAm PhenoAge is associated with age-related conditions in samples other than whole blood, for instance obesity in liver.

Our applications demonstrate that the combination of advanced machine learning methods, relevant functional genomic data (DNA methylation), and large sample sizes resulted in an epigenetic biomarker that outperforms existing molecular biomarkers of aging in terms of its strong relationship with a host of age related conditions. The new DNAm PhenoAge measure performs better than any of molecular biomarker of human aging, when it comes to predicting healthspan and lifespan.

Our results also demonstrate the utility of a novel method for building DNAm based biomarkers of aging. Our development of the new epigenetic biomarker of aging proceeded along two main steps. In step 1, a novel measure of phenotypic age was developed using clinical data. A Cox penalized regression model—where the hazard of aging-related mortality was regressed on clinical markers and chronological age—was used to select variables for inclusion in our phenotypic age score. In step 2, phenotypic age is regressed on DNA methylation data from the same individuals. The regression produced a model in which phenotypic age is predicted by DNAm levels. The linear combination of the weighted CpGs yields a DNAm based estimator of phenotypic age that we refer to as ‘DNAm PhenoAge’ in contrast to the previously published measures of ‘DNAm Age’.

### Practicing the Invention of DNAm PhenoAge

To use the epigenetic biomarker one needs to extract DNA from cells or fluids, e.g. human blood cells, saliva, liver, brain tissue. Next, one needs to measure DNA methylation levels in the underlying signature of 513 CpGs (epigenetic markers) that are being used in the mathematical algorithm. The algorithm leads to a “phenotypic age” (the apparent age of an individual resulting from the interaction of its genotype with the environment) for each sample or human subject. The higher the value, the higher the risk of death and disease.

As noted above, embodiments of the present invention relate to methods for estimating the biological age of an individual human tissue or cell type sample based on measuring DNA Cytosine-phosphate-Guanine (CpG) methylation markers that are attached to DNA. In a general embodiment of the invention, a method is disclosed comprising a first step of choosing a source of DNA such as specific biological cells (e.g. T cells in blood) or tissue sample (e.g. blood) or fluid (e.g. saliva). In a second step, genomic DNA is extracted from the collected source of DNA of the individual for whom a biological age estimate is desired. In a third step, the methylation levels of the methylation markers near the specific clock CpGs are measured. In a fourth step, a statistical prediction algorithm is applied to the methylation levels to predict the age. One basic approach is to form a weighted average of the CpGs, which is then transformed to DNA methylation (DNAm) age using a calibration function. As used herein, “weighted average” is a linear combination calculated by giving values in a data set more influence according to some attribute of the data. It is a number in which each quantity included in the linear combination is assigned a weight (or coefficient), and these weightings determine the relative importance of each quantity in the linear combination.

DNA methylation of the methylation markers (or markers close to them) can be measured using various approaches, which range from commercial array platforms (e.g. from Illumina™) to sequencing approaches of individual genes. This includes standard lab techniques or array platforms. A variety of methods for detecting methylation status or patterns have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application Publication Nos. 20030148327, 20030148326, 20030143606, 20030082609 and 20050009059, each of which are incorporated herein by reference. Other array-based methods of methylation analysis are disclosed in U.S. patent application Ser. No. 11/058,566. For a review of some methylation detection methods, see, Oakeley, E. J., Pharmacology & Therapeutics 84:389-400 (1999). Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, and methylation sensitive single nucleotide primer extension.

The methylation levels of a subset of the DNA methylation markers disclosed herein are assayed (e.g. using an Illumina™ DNA methylation array, or using a PCR protocol involving relevant primers). To quantify the methylation level, one can follow the standard protocol described by Illumina™ to calculate the beta value of methylation, which equals the fraction of methylated cytosines in that location. The invention can also be applied to any other approach for quantifying DNA methylation at locations near the genes as disclosed herein. DNA methylation can be quantified using many currently available assays which include, for example:

a) Molecular break light assay for DNA adenine methyltransferase activity is an assay that is based on the specificity of the restriction enzyme DpnI for fully methylated (adenine methylation) GATC sites in an oligonucleotide labeled with a fluorophore and quencher. The adenine methyltransferase methylates the oligonucleotide making it a substrate for DpnI. Cutting of the oligonucleotide by DpnI gives rise to a fluorescence increase.

b) Methylation-Specific Polymerase Chain Reaction (PCR) is based on a chemical reaction of sodium bisulfite with DNA that converts unmethylated cytosines of CpG dinucleotides to uracil or UpG, followed by traditional PCR. However, methylated cytosines will not be converted in this process, and thus primers are designed to overlap the CpG site of interest, which allows one to determine methylation status as methylated or unmethylated. The beta value can be calculated as the proportion of methylation.

c) Whole genome bisulfite sequencing, also known as BS-Seq, is a genome-wide analysis of DNA methylation. It is based on the sodium bisulfite conversion of genomic DNA, which is then sequencing on a Next-Generation Sequencing (NGS) platform. The sequences obtained are then re-aligned to the reference genome to determine methylation states of CpG dinucleotides based on mismatches resulting from the conversion of unmethylated cytosines into uracil.

d) The Hpall tiny fragment Enrichment by Ligation-mediated PCR (HELP) assay is based on restriction enzymes' differential ability to recognize and cleave methylated and unmethylated CpG DNA sites.

e) Methyl Sensitive Southern Blotting is similar to the HELP assay but uses Southern blotting techniques to probe gene-specific differences in methylation using restriction digests. This technique is used to evaluate local methylation near the binding site for the probe.

f) ChIP-on-chip assay is based on the ability of commercially prepared antibodies to bind to DNA methylation-associated proteins like MeCP2.

g) Restriction landmark genomic scanning is a complicated and now rarely-used assay is based upon restriction enzymes' differential recognition of methylated and unmethylated CpG sites. This assay is similar in concept to the HELP assay.

h) Methylated DNA immunoprecipitation (MeDIP) is analogous to chromatin immunoprecipitation. Immunoprecipitation is used to isolate methylated DNA fragments for input into DNA detection methods such as DNA microarrays (MeDIP-chip) or DNA sequencing (MeDIP-seq).

i) Pyrosequencing of bisulfite treated DNA is a sequencing of an amplicon made by a normal forward primer but a biotinylated reverse primer to PCR the gene of choice. The Pyrosequencer then analyses the sample by denaturing the DNA and adding one nucleotide at a time to the mix according to a sequence given by the user. If there is a mismatch, it is recorded and the percentage of DNA for which the mismatch is present is noted. This gives the user a percentage methylation per CpG island.

In certain embodiments of the invention, the genomic DNA is hybridized to a complimentary sequence (e.g. a synthetic polynucleotide sequence) that is coupled to a matrix (e.g. one disposed within a microarray such as on a DNA chip). Optionally, the genomic DNA is transformed from its natural state via amplification by a polymerase chain reaction process. For example, prior to or concurrent with hybridization to an array, the sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070, which is incorporated herein by reference.

In addition to using art accepted modeling techniques (e.g. regression analyses), embodiments of the invention can include a variety of art accepted technical processes. For example, in certain embodiments of the invention, a bisulfite conversion process is performed so that cytosine residues in the genomic DNA are transformed to uracil, while 5-methylcytosine residues in the genomic DNA are not transformed to uracil. Kits for DNA bisulfite modification are commercially available from, for example, MethylEasy™ (Human Genetic Signatures™) and CpGenome™ Modification Kit (Chemicon™). See also, WO04096825A1, which describes bisulfite modification methods and Olek et al. Nuc. Acids Res. 24:5064-6 (1994), which discloses methods of performing bisulfite treatment and subsequent amplification. Bisulfite treatment allows the methylation status of cytosines to be detected by a variety of methods. For example, any method that may be used to detect a SNP may be used, for examples, see Syvanen, Nature Rev. Gen. 2:930-942 (2001). Methods such as single base extension (SBE) may be used or hybridization of sequence specific probes similar to allele specific hybridization methods. In another aspect the Molecular Inversion Probe (MIP) assay may be used.

The 513 CpG sites discussed herein are found in Table 5 that is included with this application. The Illumina method takes advantage of sequences flanking a CpG locus to generate a unique CpG locus cluster ID with a similar strategy as NCBI's refSNP IDs (rs#) in dbSNP (see, e.g. Technical Note: Epigenetics, CpG Loci Identification ILLUMINA Inc. 2010). Further information on the present invention can be found in Levine et al., Aging, 2018 Apr. 18; 10(4):573-591 which is incorporated herein by reference.

### Examples

Estimating Phenotypic Age from Clinical Biomarkers

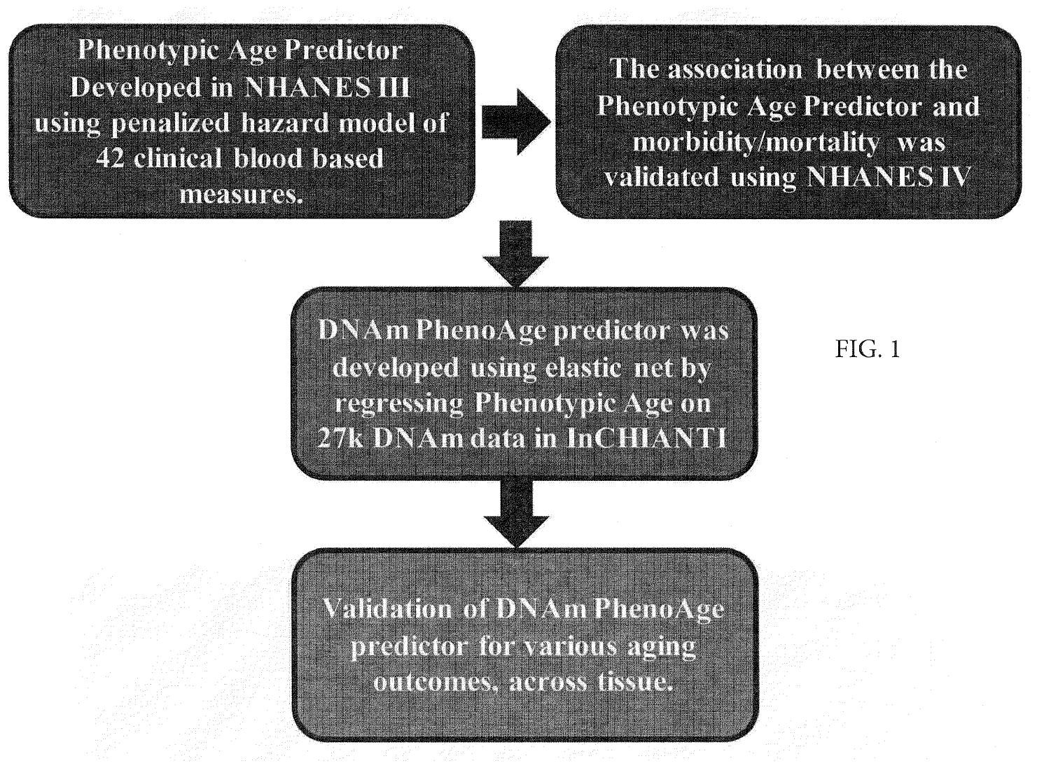

Our development of the new epigenetic biomarker of aging proceeded along three main steps (FIG. 1). In step 1, a novel measure of phenotypic age was developed using clinical data from the third National Health and Nutrition Examination Survey (for step 2 III, n=9,926). NHANES III is a nationally-representative sample, with over twenty-three years of mortality follow-up. A Cox penalized regression model—where the hazard of aging-related mortality was regressed on forty-two clinical markers and chronological age—was used to select variables for inclusion in our phenotypic age score. Of the forty-two biomarkers included in the penalized Cox regression model, ten variables (including chronological age) were selected for the phenotypic age predictor (Table 4). These nine biomarkers and chronological age were then combined in a phenotypic age estimate (in units of years) as detailed in Methods.

Validation data for phenotypic age came from the fourth National Health and Nutrition Examination Survey (NHANES IV), and included up to 17 years of mortality follow-up for n=6,209 national representative US adults. Mortality results show (Table 1) that a one year increase in phenotypic age is associated with a 9% increase in the risk of all-cause mortality (HR=1.09, p=3.8E-49), a 9% increase in the risk of aging-related mortality (HR=1.09, p=4.5E-34), a 10% increase in the risk of CVD mortality (HR=1.10, p=5.1E-17), a 7% increase in the risk of cancer mortality (HR=1.07, p=7.9E-10), a 20% increase in the risk of diabetes mortality (HR=1.20, p=1.9E-11), and a 9% increase in the risk of lung disease mortality (HR=1.09, p=6.3E-4). Finally, in the proportional hazard model, phenotypic age completely accounted for the effect of chronological age, such that chronological age no longer exhibited a significant positive association with mortality.

We further tested whether the phenotypic age associations held-up when examining mortality among three age strata-young and middle-aged adults (20-64 years at baseline), older adults (65-79 years at baseline), and the oldest-old (80+ years at baseline). Results showed consistent findings for all-cause, aging-related, CVD, cancer, diabetes, and lung disease within all age strata (Table 1). Finally, to ensure that phenotypic age didn't simply represent an end-of-life marker, we removed participants who died within five years of baseline, and then re-examined mortality associations. Again, we find significant associations for all mortality outcomes, except Alzheimer's disease (Table 1).

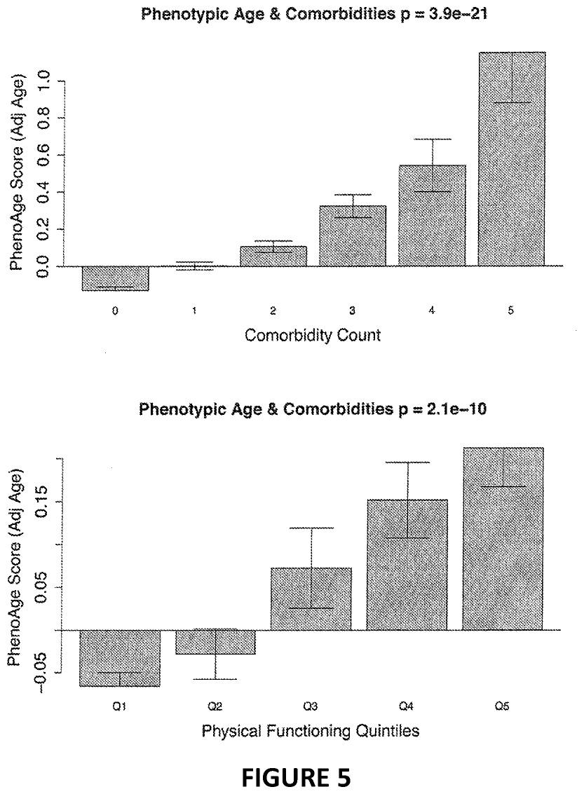

Finally, as shown in FIG. 5, we tested the association between phenotypic age and 1) the number of coexisting morbidities a participant had been diagnosed with, and 2) levels of physical functioning problems. Results showed that after adjusting for chronological age, persons with more coexisting morbidities also displayed higher phenotypic ages on average (p=3.9E-21). Similarly, those with worse physical functioning tended to have higher phenotypic ages (p=2.1E-10).

### An Epigenetic Biomarker of Aging (DNAm PhenoAge)

In step 2 (FIG. 1), data from the Invecchiare in Chianti (InCHIANTI) study was used to relate blood DNAm levels to phenotypic age. Elastic net regression produced a model in which phenotypic age is predicted by DNAm levels at 513 CpGs. The linear combination of the weighted 513 CpGs yields a DNAm based estimator of phenotypic age (mean=58.9, s.d.=18.2, range=9.1-106.1), that we refer to as ‘DNAm PhenoAge’ in contrast to the previously published Hannum and Horvath ‘DNAm Age’ measures.

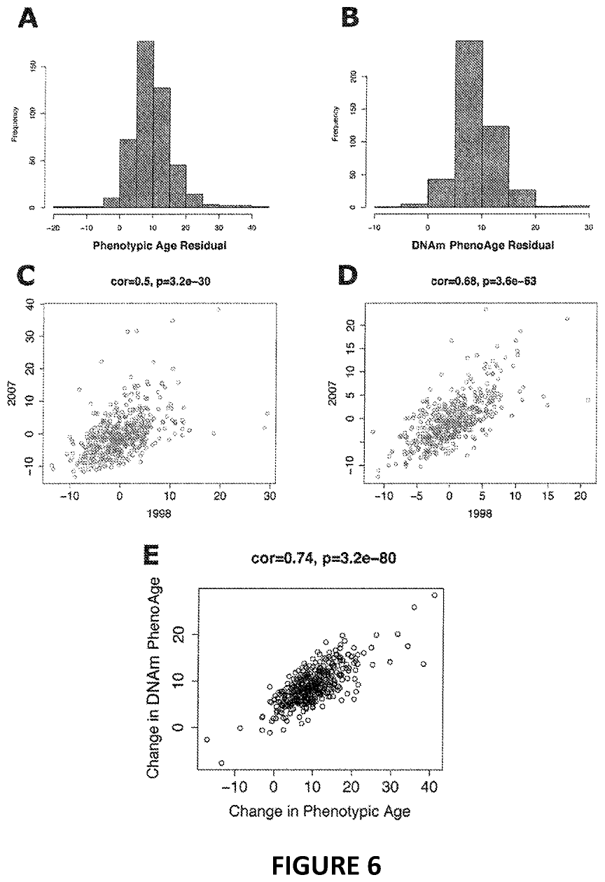

While our new clock was trained on cross-sectional data in InCHIANTI, we capitalized on the repeated time-points to test whether changes in DNAm PhenoAge are related to changes in phenotypic age. As expected, between 1998 and 2007, mean change in DNAm PhenoAge was 8.51 years, whereas mean change in phenotypic age was 8.88 years. Moreover, participants' phenotypic age (adjusting for chronological age) at the two time-points was correlated at r=0.50, whereas participants' DNAm PhenoAge (adjusting for chronological age) at the two time-points was correlated at r=0.68 (FIG. 6). Finally, as shown in FIG. 6, we find that the change in phenotypic age between 1998 and 2007 is highly correlated with the change in DNAm PhenoAge between these two time-points (r=0.74, p=3.2E-80).

### DNAm PhenoAge Strongly Relates to Aging Outcomes

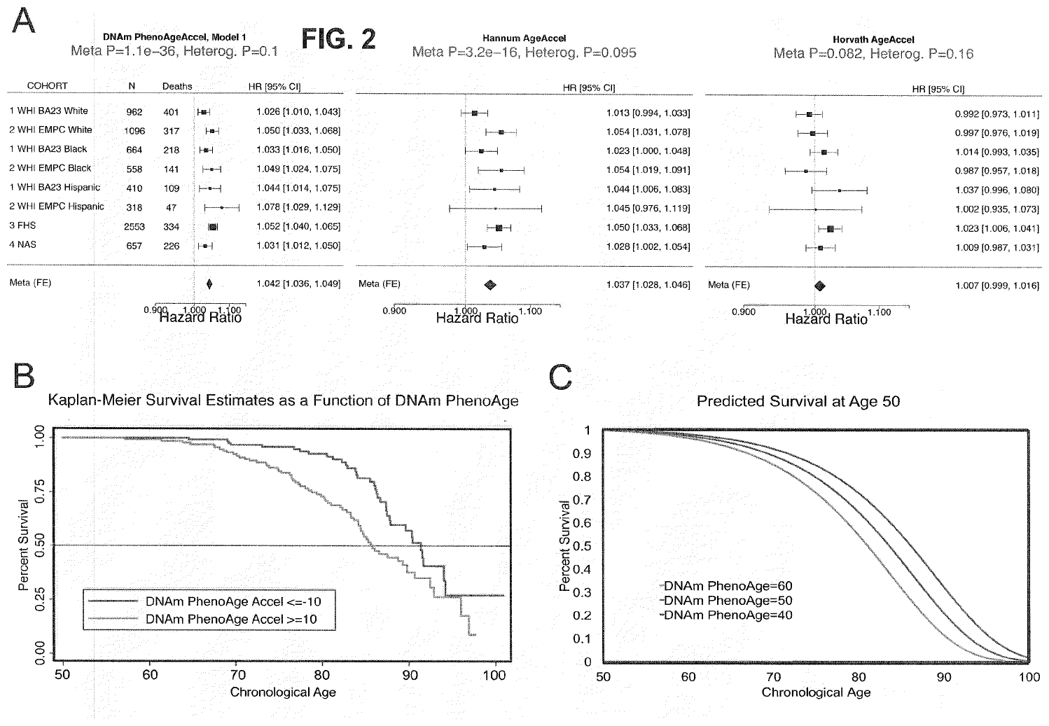

In step 3 (FIG. 1), DNAm PhenoAge was calculated in four independent large-scale samples-two samples from Women's Health Initiative (WHI) (n=2,016; and n=2,191), the Framingham Heart Study (FHS) (n=2,553), and the Normative Aging Study (n=657). In these studies, DNAm PhenoAge correlated with chronological age at r=0.67 in WHI (Sample 1), r=0.69 in WHI (Sample2), r=0.78 in FHS, and r=0.62 in the Normative Aging Study. The four validation samples were then used to assess the effects of DNAm PhenoAge on mortality in comparison to the Horvath and Hannum DNAm Age measures. As shown in FIG. 2, DNAm PhenoAge was significantly associated with subsequent mortality risk in all studies (independent of chronological age), such that, a one year increase in DNAm PhenoAge is associated with a 4% increase in the risk of all-cause mortality (Meta(FE)=1.042, Meta p=1.1E-36). To better conceptualize what this increase represents, we compared the predicted life expectancy and mortality risk for person's representing the top 5% (fastest agers), the average, and the bottom 5% (slowest agers). Results suggest that those in the top 5% of fastest agers have a mortality hazard of death that is about 1.57 times that of the average person, i.e. your hazard of death is 57% higher than that of an average person. Further, contrasting the 5% fastest agers with the 5% slowest agers, we find that the hazard of death of the fastest agers is 2.47 times higher than that of the bottom 5% slowest agers (HR=1.042^{11.0}/1.042^{-10.5}). Finally, both observed and predicted Kaplan-Meier survival estimates showed that faster agers had much lower life expectancy and survival rates compared to average and/or slow agers (FIG. 2).

We also observe strong association between DNAm PhenoAge and a variety of other aging outcomes (Table 2). For instance, independent of chronological age, higher DNAm PhenoAge is associated with an increase in a person's number of coexisting morbidities (Meta P-value=4.56E-15), a decrease in likelihood of being disease-free (Meta P-value=1.06E-7), an increase in physical functioning problems (Meta P-value=2.05E-13), an increase in the risk of CHD risk (Meta P-value=2.43E-10, an earlier age at menopause (Meta P-value=8.22E-4)—suggesting that women were epigenetically older if they had entered menopause earlier.

Additional replication data was used to test for associations with other aging outcomes, which have previously been shown to relate to the first generation of epigenetic biomarkers^{14,15,23-26} For instance, we find that among the 527 women who were cancer free at age 50, accelerated DNAm PhenoAge predicts incident breast cancer (p=0.033, OR: 1.037). We also find a marginally significant reduction of approximately 2.4 years for the DNAm PhenoAge of semi-super centenarian offspring, relative to controls (P=−2.40, p=0.065). Using blood methylation data, we evaluated whether DNAm PhenoAge relates to clinically diagnosed dementia in living individuals. Results suggest that those with presumed Alzheimer's disease (AD, n=154) and/or frontotemporal dementia (FTD, n=116) have significantly higher DNAm PhenoAge compared to non-demented (n=334) individuals (P=2.2E-2), and the strength of the association is further increased (P=9.4E-3) when limiting the sample to those ages 75 and older. We also find that DNAm PhenoAge, relates to Down syndrome in two separate blood methylation datasets (p=0.0046 and p=4.0E-11), and similarly relates to HIV infection in two blood datasets (p=6E-6 and p=8.6E-6). We observe a suggestive relationship between DNAm PhenoAge in blood and Parkinson's disease status (p=0.028) for individuals from European ancestry.

### DNAm PhenoAge Versus Behavioral and Demographic Characteristics

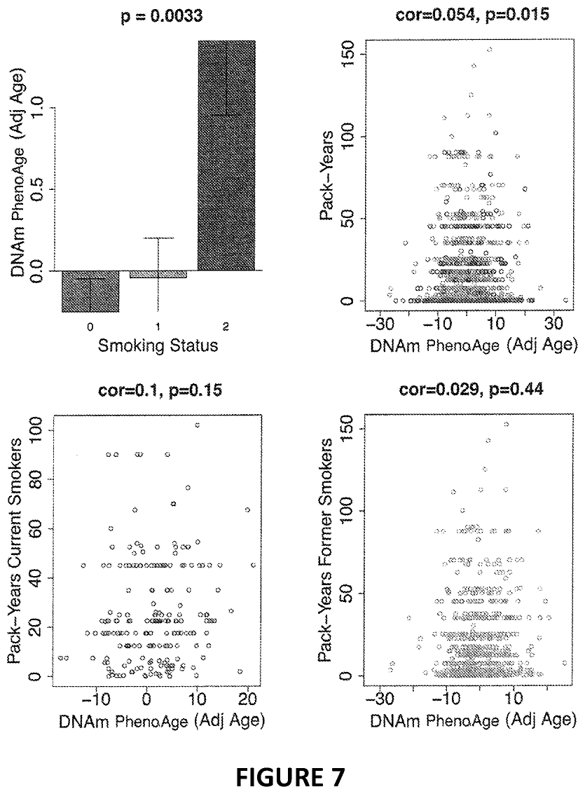

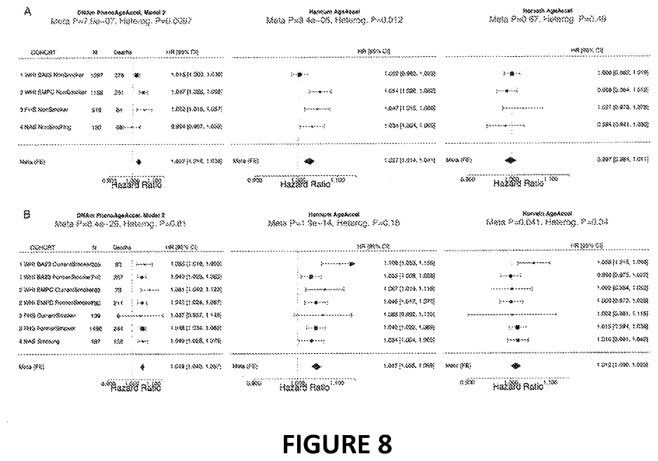

Given the recent study in which Zhang and colleagues²⁷ developed an epigenetic mortality predictor that turned out to be an estimate of smoking habits, we examined the association between DNAm PhenoAge and smoking. As shown in FIG. 7, we find that DNAm PhenoAge also significantly differs by smoking status (p=0.0033). Next, we re-evaluated the morbidity and mortality associations (fully-adjusted) in our four samples, stratifying by smoking status (smokers vs. non-smokers) (FIG. 8 & Table 4). We find that DNAm PhenoAge is associated with mortality both among smokers (adjusted for pack-years) (Meta(FE)=1.041, Meta p=2.6E-14), and among persons who have never smoked (Meta(FE)=1.027, Meta p=7.9E-7). Moreover, as shown in Table 4, among never smokers, DNAm PhenoAge relates to the number of coexisting morbidities (Meta P-value=7.83E-6), physical functioning status (Meta P-value=2.63E-3), disease free status (Meta P-value=4.38E-4), and CHD (Meta P-value=1.80E-4), while among current smokers, it relates to the number of coexisting morbidities (Meta P-value=4.61E-5), physical functioning status (Meta P-value=1.01E-4), and disease free status (Meta P-value=0.0048), but only exhibits a suggestive association with CHD (Meta P-value=0.084). We previously reported that Horvath DNAm age of blood predicts lung cancer risk in the first WHI sample²⁸. Using the same data, we replicate this finding for DNAm PhenoAge. After adjusting for chronological age, race/ethnicity, pack-years, and smoking status, results showed that a one year increase in DNAm PhenoAge is associated with a 5% increase in lung cancer risk (HR=1.05, p=0.031), and when restricting the model to current smokers only, we find that the effect of DNAm PhenoAge on lung cancer mortality is even stronger (HR=1.10, p=0.014).

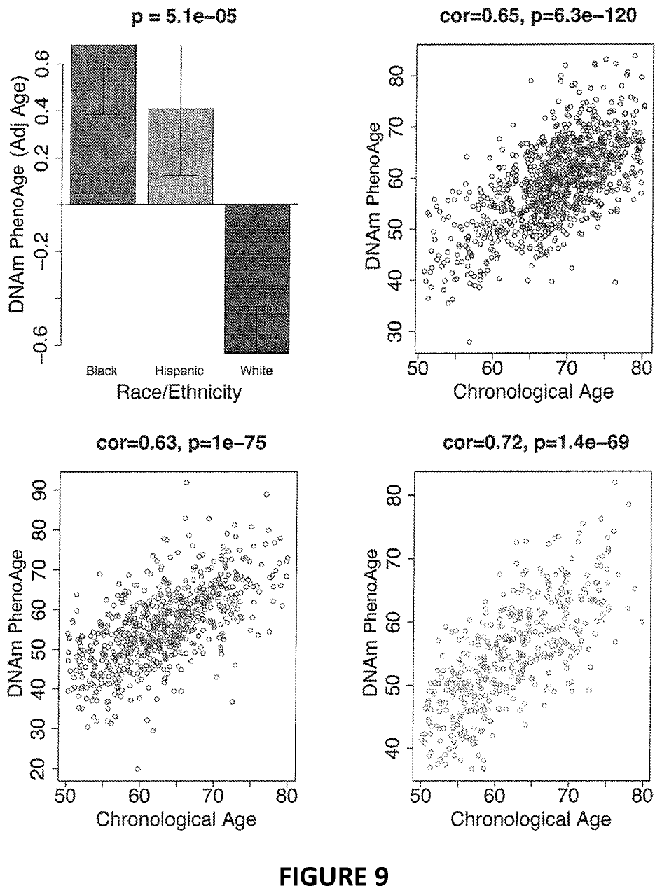

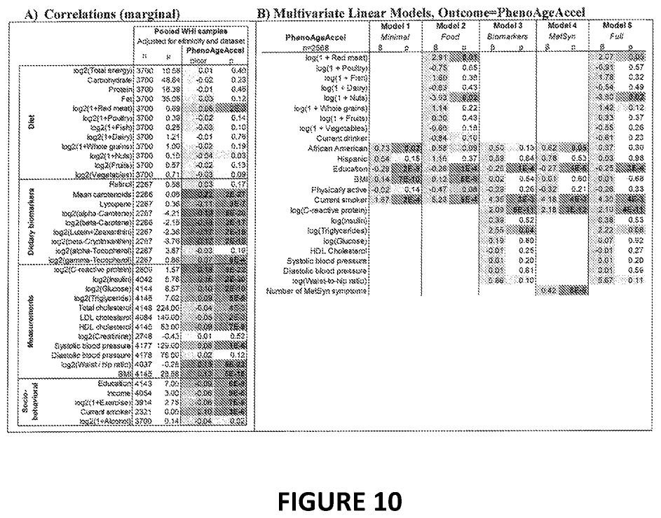

In evaluating the relationship between DNAm PhenoAge and social, behavioral, and demographic characteristics we observe significant differences between racial/ethnic groups (p=5.1E-5), with non-Hispanic blacks having the highest DNAm PhenoAge on average, and non-Hispanic whites having the lowest (FIG. 9). We also find evidence of social gradients in DNAm PhenoAge, such that those with higher education (p=6E-9) and higher income (p=9E-5) appear younger. DNAm PhenoAge relates to exercise and dietary habits, such that increased exercise (p=7E-5) and markers of fruit/vegetable consumption (such as carotenoids, p=5E-22) are associated with lower DNAm PhenoAge, whereas smoking (p=3E-6) was associated with increased DNAm PhenoAge (FIG. 10A). Finally, these associations were re-examined in step-wise multivariate models. Overall, we find that associations for race/ethnicity, education, smoking, CRP, triglycerides, protein consumption, and metabolic syndrome are generally maintained (FIG. 10B).

### DNAm PhenoAge in Other Tissues

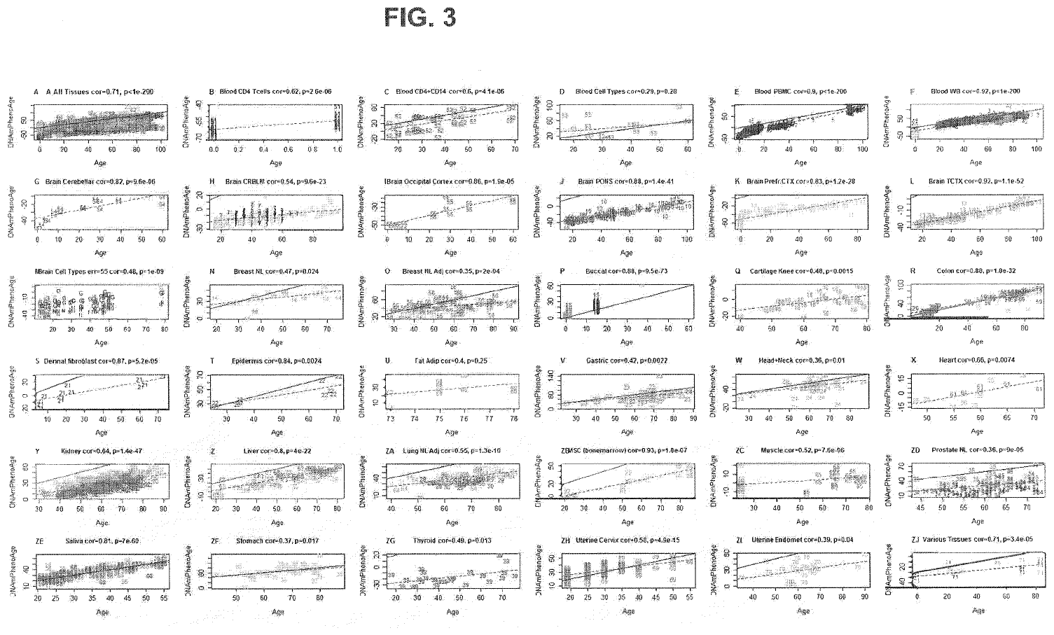

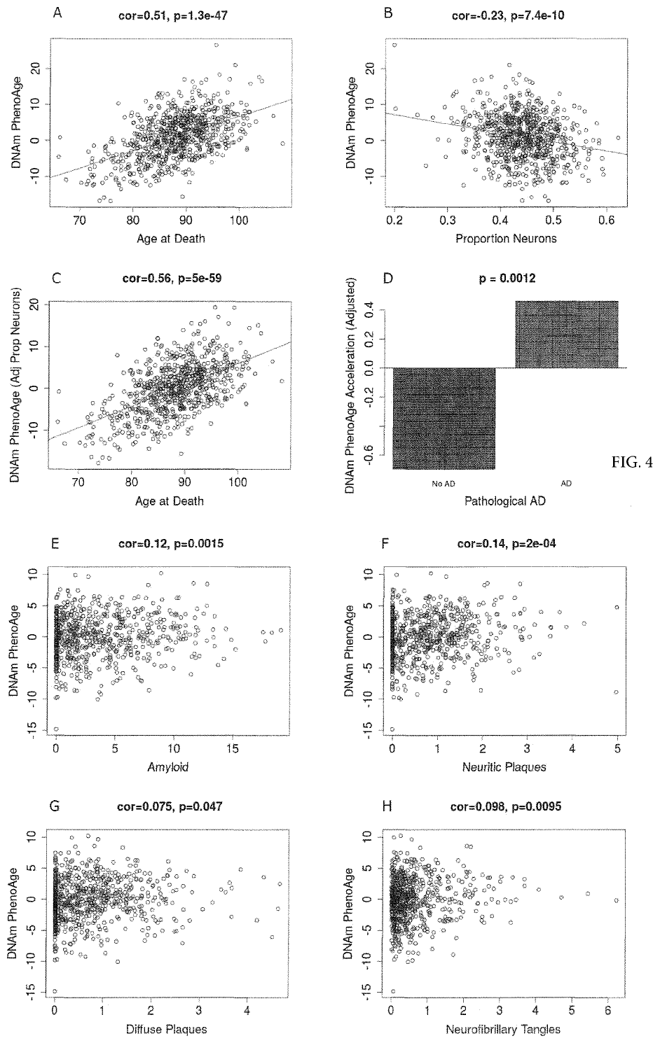

Although DNAm PhenoAge was developed from DNAm levels assessed in whole blood, our empirical results show that it strongly correlates with chronological age in a host of different tissues (FIG. 3). For instance, when examining all tissue concurrently, the correlation between DNAm PhenoAge and chronological age was 0.71. Age correlations in brain tissue ranged from 0.54 to 0.92. Consistent age correlations were also found in breast (r=0.47), buccal cells (r=0.88), dermal fibroblasts (r=0.87), epidermis (r=0.84), colon (r=0.88), heart (r=0.66), kidney (r=0.64), liver (r=0.80), lung (r=055), and saliva (r=0.81).

Using the Horvath DNAm age measure, we previously found that body mass index is correlated with epigenetic age acceleration in two independent human liver samples (r=0.42 and r=0.42 in liver data sets 1 and 2, respectively)²⁹. Using the same data, we replicated this finding using the new measure of PhenoAge acceleration (r=0.32, p=0.011 and r=0.48 p=7.7E-6 in liver data set 1 and 2, respectively. Interestingly we also find a significant correlation between BMI and DNAm PhenoAge acceleration in the first adipose data set (r=0.43, p=1.2E-23 using n=648 adipose samples from the Twins UK study) but not in a second smaller adipose data set (n=32 samples).

### Biological Interpretation of DNAm PhenoAge

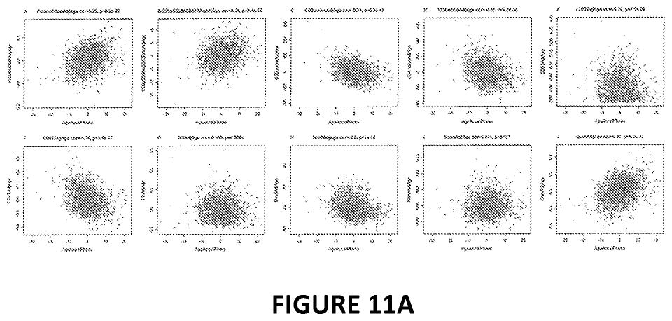

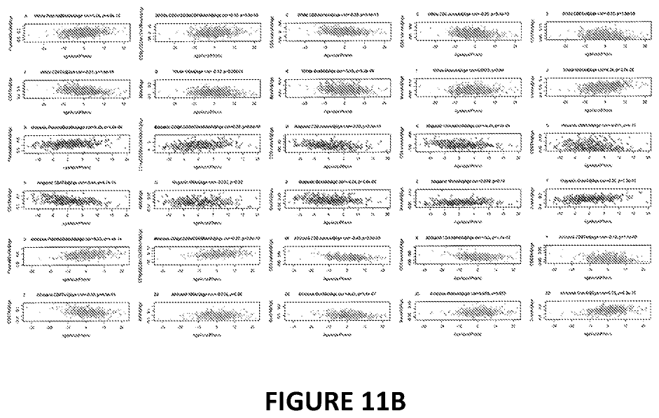

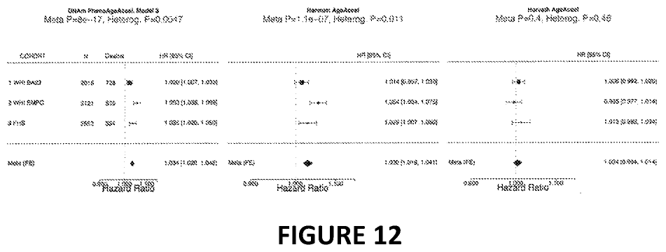

To test the hypothesis that DNAm phenotypic age acceleration captures aspects of the age-related decline of the immune system, we correlated DNAm PhenoAge acceleration with estimated blood cell count (FIG. 11). After adjusting for age, we find that DNAm PhenoAge acceleration is negatively correlated with naïve CD8+ T cells (r=−0.34, p=5.3E-47), naïve CD4+ T cells (r=−0.29, p=4.2E-34), CD4+ helper T cells (r=−0.34, p=5.3E-47), and B cells (r=−0.20, p=1E-16). Further, phenotypic age acceleration is positively correlated with the proportion of granulocytes (r=0.34, p=5.3E-47), exhausted CD8+(defined as CD28-CD45RA−) T cells (r=0.21, p=2.7E-18), and plasma blast cells (r=0.28, p=8.2E-32). These results are consistent with age related changes in blood cells.³⁰ However, the strong association between DNAm PhenoAge and mortality/morbidity outcomes does not simply reflect changes in blood cell composition as can be seen from the fact that the associations between DNAm PhenoAge and morbidity and mortality held-up even after adjusting for estimates of seven blood cell count measures (FIG. 12).

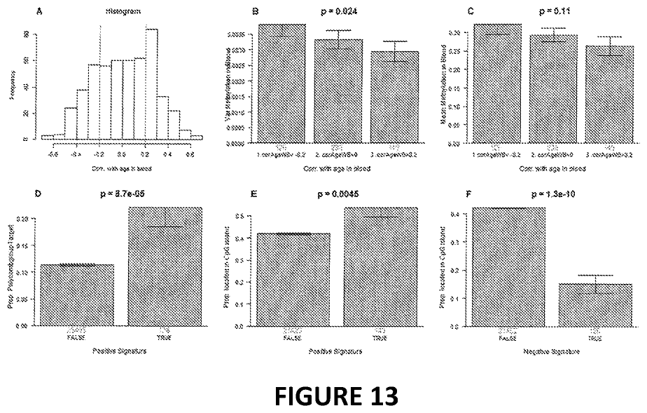

In our functional enrichment analysis of the chromosomal locations of the 513 CpGs, we found that 149 CpGs whose age correlation exceeded 0.2 tended to be located in CpG islands (p=0.0045, FIG. 13) and were significantly enriched with polycomb group protein targets (p=8.7E-5, FIG. 13) which echoes results of epigenome wide studies of aging effects^{4,31,32}.

Our heritability analysis of the DNAm PhenoAge acceleration used the SOLAR polygenic model to estimate the proportion of phenotypic variance explained by family relationship in the Framingham Heart Study pedigrees. The model assumes additive genetic heritability in a polygenic model, adjusting for chronological age and sex. The heritability estimated by the SOLAR polygenic model was (h²=0.33) among persons of European ancestry. Similarly, a heritability estimate from SNP data was calculated from WHI data using GCTA-GREML analysis. In this model, we find that heritability is estimated at h²=0.51 for participants of European ancestry.

### Conclusion

Using a novel two-step method, we were successful in developing a DNAm based biomarker of aging that is highly predictive of nearly every morbidity and mortality outcome we tested. Our study demonstrates that DNAm PhenoAge greatly outperforms the first generation of DNAm based biomarkers of aging from Hannum⁹ and Horvath¹⁰, in terms of both its predictive accuracy for time to death and its associations with various other aging measures, including disease incidence/prevalence and physical functioning. Most surprisingly, DNAm PhenoAge is associated with age-related conditions in samples other than whole blood, for instance obesity in liver.

Our applications demonstrate that the combination of advanced machine learning methods, relevant functional genomic data (DNA methylation), and large sample sizes resulted in an epigenetic biomarker that outperforms existing molecular biomarkers. However, the unbiased, data-driven approach used in its construction entails that it is challenging to understand the molecular causes and consequences of DNAm PhenoAge. To partially address this challenge, we employed three approaches: i) study on the relationship between phenotypic aging and changes in blood cell counts, ii) functional enrichment studies of the underlying CpGs, iii) heritability analysis. Although DNAm PhenoAge captures some aspects of the age-related decline in the immune system, these changes in cell composition do not explain the strong association between DNAm PhenoAge and mortality/morbidity outcomes. Our functional enrichment study demonstrates that age related DNA methylation changes in polycomb group protein targets must play a role, which echoes results from previous epigenome wide studies of aging effects^{4,31,32} Our heritability analysis suggests that there is a genetic basis for differences in DNAm PhenoAge, after adjusting for chronological age. Our results also suggest DNAm PhenoAge may respond to modifiable lifestyle factors. In moving forward, it will be important to establish causative pathways to test whether DNAm PhenoAge mediates the links between these precipitating factors and aging-related outcomes (i.e. social, behavioral, environmental conditions→DNAm PhenoAge→morbidity/mortality).

Overall, we expect that DNAm PhenoAge will become a useful molecular biomarker for human anti-aging studies because it is a highly robust, blood based biomarker that captures organismal age and the functional state of many organ systems and tissues.

### Methods

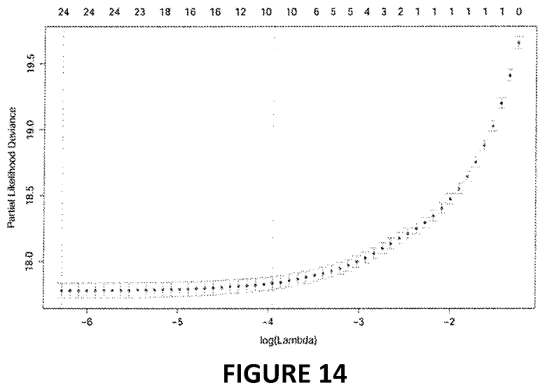

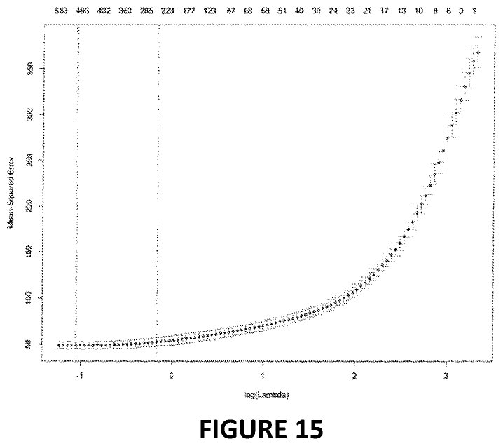

Using the NHANES training data, we applied a Cox penalized regression model—where the hazard of aging-related mortality (mortality from diseases of the heart, malignant neoplasms, chronic lower respiratory disease, cerebrovascular disease, Alzheimer's disease, Diabetes mellitus, nephritis, nephrotic syndrome, and nephrosis) was regressed on forty-two clinical markers and chronological age to select variables for inclusion in our phenotypic age score. Ten-fold cross-validation was employed to select the parameter value, lambda, for the penalized regression. In order to develop a sparse phenotypic age estimator (the fewest biomarker variables needed to produce robust results) we selected a lambda of 0.0192, which represented a one standard deviation increase over the lambda with minimum mean-squared error during cross-validation (FIG. 14). Of the forty-two biomarkers included in the penalized Cox regression model, this resulted in ten variables (including chronological age) that were selected for the phenotypic age predictor.

These nine biomarkers and chronological age were then included in a parametric proportional hazards model based on the Gompertz distribution. Based on this model, we estimated the 10-year (120 months) mortality risk of the j-the individual. Next, the mortality score was converted into units of years The resulting phenotypic age estimate was regressed DNA methylation data using an elastic net regression analysis. The penalization parameter was chosen to minimize the cross validated mean square error rate (FIG. 14), which resulted in 513 CpGs.

As noted above, these nine biomarkers and chronological age were then included in a parametric proportional hazards model based on the Gompertz distribution. Based on this model, we estimated the 10-year (120 months) mortality risk of the j-the individual based on the cumulative distribution function

MortalityScoreⱼ=CDF(120,Xⱼ)=1−e^{-e}^{^{x}}^{ⱼ}^{^{b}}^{(exp(120*y)-1)/y}

where xb=represents the linear combination of biomarkers from the fitted model (Table 4):

WeightedAverage=(−Albumin*0.0336+log(Creatinine)*0.0095+Glucose*0.1953+C−reactiveProtein*0.0954−LymphocytePerc*0.0120−+MeanCellVolume*0.0268+RedBloodCellDistributionWidth*0.3306+AlkalinePhosphatase*0.0019+WhiteBloodCellCount*0.0554+age*0.0804−19.9067).

Next, the mortality score was converted into units of years using the following equation

PhenotypicAgeⱼ=141.50225+ln(−0.00553*ln(1−MortalityScoreⱼ)))/0.090165

Statistical Details on the Gompertz Proportional Hazards Model for Phenotypic Age Estimation

The Gompertz regression is parameterized only as a proportional hazards model. This model has been extensively used extensively for modeling mortality data. The Gompertz distribution implemented is the two-parameter function as described in Lee and Wang (2003)¹, with the following hazard and survivor functions:

h(t)=λexp(γt)

S(t)=exp{−λγ⁻¹(e^{γt}−1)}

The covariates of the j-th individual are including in the model using the following parametrization: λⱼ=exp(xⱼβ) which implies that the baseline hazard is given by h₀(t)=exp(μt) where γ is an ancillary parameter to be estimated from the data.

The cumulative distribution function of the Gompertz model is given by

CDF(t,x)=1−exp(−exp(xb)(exp(γt)−1)/γ)

where t denotes time (here in units of months) and xb=Σᵤ₌₁^{p} x^{u}b^{u}+b⁰.

We used the STATA software (StataCorp. 2001. Statistical Software: Release 7.0) to carry out the Gompertz regression analysis.

In step 1, we fit a parametric proportional hazards model analysis with Gompertz distribution using the STATA commands

stset person_months [pweight=wt], failure(mortstat==1)
streg var1 var2 var3 . . . vark,dist(gomp)

The Gompertz regression analysis resulted in coefficient values and parameter values (Table 1) and γ=0.0076927.

In step 2, we used the cumulative distribution function of the Gompertz model to estimate the 120-month mortality risk of each individual. Thus, CDF(t=120,xⱼ) denotes the probability that the j-th individual will die within the next 120 months. In step 3, carried out another parametric proportional hazards model analysis with Gompertz distribution, but only including chronological age as a IV. We will refer to this analysis as the univariate Gompertz regression model since it only involved one covariate (age). The resulting estimate of the cumulative distribution function CDF·univariate(t,age)

$$CDF . univariate ( t , age ) = 1 - {e}^{\{ - {e}^{( age * {b}^{1} + {b}^{0} )} {γ}^{- 1} ( {e}^{γ t} - 1 ) \}}$$

allowed us to estimate the probability that the j-th individual with die within 120 months as follows CDF·univariate(120,ageⱼ) where ageⱼ is the age of the j-th individual.
In step 4, we solved the equation CDF(120,xⱼ)=CDF·univariate(120,ageⱼ) for the variable ageⱼ. The resulting solution for the j-th individual, referred to as PhenotypicAge, is given by

$${PhenotypicAge}_{j} = 14 \; 1.50225 + \frac{ln ( - 0.00553 * ln ( 1 - CDF ( 120 , {x}_{j} ) ) )}{0.090165}$$

### Data Used to Generate DNAM Phenoage

Participants ages 20 and over in NHANES III (1988-94) were used as the training sample to develop a new and improved measure of phenotypic aging (n=9,926), while participants ages 20 and over in NHANES IV (1999-2014) were used to validate the association between phenotypic aging and age-related morbidity and mortality (n=6,209). Overall, NHANES III had available mortality follow-up for up to 23 (n=deaths) and NHANES IV had available mortality follow-up for up to 17 years (n=deaths). InCHIANTI included longitudinal (two time-points-1998 and 2007) phenotypic and DNAm data on n=456 male and female participants, ages 21-91 in 1998, and 30-100 in 2007. Participants from WHI included 2,107 post-menopausal women, who were ages 50-80 at baseline and were followed-up for just over 20 years.

Steps for Measuring the DNA Methylation PhenoAge of a Tissue Sample and Estimating DNA Methylation-Based Predictors of Mortality

Step 1: Obtain Cells from Blood, Saliva, or Other Sources of DNA from an Individual.
There are several options.
Blood tubes collected by venipunture: Blood tubes collected by venipuncture will result in a large amount of high quality DNA from a relevant tissue. The invention applies to DNA from whole blood, or peripheral blood mononuclear cells or even sorted blood cell types.
Saliva spit kit:
Dried blood spots can be easily collected by a finger prick method. The resulting blood droplet can be put on a blood card, e.g. http://www.lipidx.com/dbs-kits/.

### Step 2: Generate DNA Methylation Data

This step will be carried out by the lab that collects the samples.

Step 2a: Extract the genomic DNA from the cells
Step 2b: Measure cytosine DNA methylation levels.
Several approaches can be used for measuring DNA methylation including sequencing, bisulfite sequencing, arrays, pyrosequencing, liquid chromatography coupled with tandem mass spectrometry.

Our invention applies to any platform used for measuring DNA methylation data. In particular, it can be used in conjunction with the latest Illumina methylation array platform the EPIC array or the older platforms (Infinium 450K array or 27K array). Our coefficient values used pertain to the “beta values” whose values lie between 0 and 1 but it could be easily adapted to other metrics of assessing DNA methylation, e.g. “M values”.

### Step 3: Estimate the DNA Methylation PhenoAge Estimate

The DNAm PhenoAge estimate can be estimated as a weighted linear combination of 513 CpGs in Table 5. This table also includes the probe designation/identifier used in the Illumina Infinium 450K array.

### REFERENCES

1 Kennedy, B. K. et al. Geroscience: Linking Aging to Chronic Disease. Cell 159, 709-713, doi:10.1016/j.cell.2014.10.039 (2014).
2 Burch, J. B. et al. Advances in geroscience: impact on healthspan and chronic disease. J Gerontol A Biol Sci Med Sci 69 Suppl 1, S1-3, doi:10.1093/gerona/glu041(2014).
3 Fraga, M. F. & Esteller, M. Epigenetics and aging: the targets and the marks. Trends in Genetics 23, 413-418, doi:10.1016/j.tig.2007.05.008 (2007).
4 Rakyan, V. K. et al. Human aging-associated DNA hypermethylation occurs preferentially at bivalent chromatin domains. Genome research 20, 434-439, doi:10.1101/gr.103101.109 (2010).
5 Teschendorff, A. E. et al. Age-dependent DNA methylation of genes that are suppressed in stem cells is a hallmark of cancer. Genome research 20, 440-446, doi:10.1101/gr.103606.109 (2010).
6 Jung, M. & Pfeifer, G. P. Aging and DNA methylation. BMC biology 13, 1-8, doi:10.1186/s12915-015-0118-4 (2015).
7 Zheng, S. C., Widschwendter, M. & Teschendorff, A. E. Epigenetic drift, epigenetic clocks and cancer risk. Epigenomics 8, 705-719, doi:10.2217/epi-2015-0017 (2016).
8 Bocklandt, S. et al. Epigenetic predictor of age. PLoS One. 6, doi:10.1371/journal.pone.0014821(2011).
9 Hannum, G. et al. Genome-wide methylation profiles reveal quantitative views of human aging rates. Mol Cell. 49, doi:10.1016/j.molcel.2012.10.016 (2013).
10 Horvath, S. DNA methylation age of human tissues and cell types. Genome Biol 14, doi:DOI: 10.1186/10.1186/gb-2013-14-10-r115 (2013).
11 Levine, M., Lu, A., Bennett, D. & Horvath, S. Epigenetic age of the pre-frontal cortex is associated with neuritic plaques, amyloid load, and Alzheimer's disease related cognitive functioning. Aging (Albany N.Y.) December (2015).
12 Marioni, R. E. et al. The epigenetic clock is correlated with physical and cognitive fitness in the Lothian Birth Cohort 1936. Int J Epidemiol 44, doi:10.1093/ije/dyu277 (2015).
13 Marioni, R. et al. DNA methylation age of blood predicts all-cause mortality in later life. Genome Biol. 16, 25 (2015).
14 Horvath, S. & Levine, A. J. HIV-1 infection accelerates age according to the epigenetic clock. J Infect Dis, doi:10.1093/infdis/jiv277 (2015).
15 Horvath, S. et al. Accelerated Epigenetic Aging in Down Syndrome. Aging Cell 14, doi:DOI: 10.1111/acel.12325 (2015).
16 Levine, M. E. et al. Menopause accelerates biological aging. Proc Natl Acad Sci USA 113, 9327-9332, doi:10.1073/pnas.1604558113 (2016).
17 Chen, B. H. et al. DNA methylation-based measures of biological age: meta-analysis predicting time to death. Aging (Albany N.Y.) 8, 1844-1865, doi:10.18632/aging.101020 (2016).
18 Levine, M. E. Modeling the Rate of Senescence: Can Estimated Biological Age Predict Mortality More Accurately Than Chronological Age? The Journals of Gerontology Series A: Biological Sciences and Medical Sciences 68, 667-674, doi:10.1093/gerona/gls233 (2013).
19 Belsky, D. W. et al. Quantification of biological aging in young adults. Proceedings of the National Academy of Sciences 112, E4104-E4110, doi:10.1073/pnas.1506264112(2015).
Li, S. et al. Genetic and Environmental Causes of Variation in the Difference Between Biological Age Based on DNA Methylation and Chronological Age for Middle-Aged Women. Twin Res Hum Genet 18, 720-726, doi:10.1017/thg.2015.75(2015).
21 Sebastiani, P. et al. Biomarker signatures of aging. Aging Cell, n/a-n/a, doi:10.1111/acel.12557 (2017).
22 Ferrucci, L., Hesdorffer, C., Bandinelli, S. & Simonsick, E. M. Frailty as a Nexus Between the Biology of Aging, Environmental Conditions and Clinical Geriatrics. Public Health Reviews 32, 475-488, doi:10.1007/BF03391612 (2010).
23 Ambatipudi, S. et al. DNA methylome analysis identifies accelerated epigenetic ageing associated with postmenopausal breast cancer susceptibility. Eur J Cancer 75, 299-307, doi:10.1016/j.ejca.2017.01.014 (2017).
24 Horvath, S. et al. Decreased epigenetic age of PBMCs from Italian semi-supercentenarians and their offspring. Aging (Albany N.Y.) 7, doi:10.18632/aging.100861(2015).
25 Li, Y. et al. An epigenetic signature in peripheral blood associated with the haplotype on 17q21.31, a risk factor for neurodegenerative tauopathy. PLoS Genet 10, e1004211, doi:10.1371/journal.pgen.1004211 (2014).
26 Horvath, S. & Ritz, B. R. Increased epigenetic age and granulocyte counts in the blood of Parkinson's disease patients. Aging (Albany N.Y.) 7, 1130-1142, doi:10.18632/aging.100859 (2015).
27 Zhang, Y. et al. DNA methylation signatures in peripheral blood strongly predict all-cause mortality. Vol. 8 (2017).
28 Levine, M. E. et al. DNA methylation age of blood predicts future onset of lung cancer in the women's health initiative. Aging (Albany N.Y.) 7, 690-700 (2015).
29 Horvath, S. et al. Obesity accelerates epigenetic aging of human liver. Proc Natl Acad Sci USA 111, 15538-15543, doi:10.1073/pnas.1412759111 (2014).
30 Franceschi, C. et al. Inflamm-aging. An evolutionary perspective on immunosenescence. Ann N Y Acad Sci 908, 244-254 (2000).
31 Teschendorff, A. E. et al. Age-dependent DNA methylation of genes that are suppressed in stem cells is a hallmark of cancer. Genome research 20, 440-446 (2010).
32 Horvath, S. et al. Aging effects on DNA methylation modules in human brain and blood tissue. Genome Biol. 13, R97 (2012).

### Tables

TABLE 1
Mortality Validations for Phenotypic Age

| Mortality Cause | Cases | HR | P-Value |
|---|---|---|---|
| Full Sample | | | |
| All-Cause | 1052 | 1.09 | 3.8E−49 |
| Aging-Related | 661 | 1.09 | 4.5E−34 |
| CVD | 272 | 1.10 | 5.1E−17 |
| Cancer | 265 | 1.07 | 7.9E−10 |
| Alzheimer's | 30 | 1.04 | 2.6E−1 |
| Diabetes | 41 | 1.20 | 1.9E−11 |
| Lung | 53 | 1.09 | 6.3E−4 |
| 80 Years and Over | | | |
| All-Cause | 398 | 1.07 | 8.8E−15 |
| Aging-Related | 165 | 1.08 | 6.1E−10 |
| CVD | 112 | 1.08 | 9.9E−6 |
| Cancer | 69 | 1.08 | 4.0E−4 |
| Alzheimer's | 11 | 1.00 | 9.6E−1 |
| Diabetes | 9 | 1.14 | 2.5E−2 |
| Lung | 8 | 1.09 | 5.7E−2 |
| 65-79 Years | | | |
| All-Cause | 510 | 1.10 | 6.2E−29 |
| Aging-Related | 343 | 1.10 | 2.4E−19 |
| CVD | 133 | 1.11 | 5.0E−10 |
| Cancer | 99 | 1.07 | 5.0E−5 |
| Alzheimer's | 16 | 1.12 | 1.7E−2 |
| Diabetes | 25 | 1.22 | 5.2E−8 |
| Lung | 28 | 1.07 | 6.4E−2 |
| 20-64 Years | | | |
| All-Cause | 144 | 1.10 | 6.4E−9 |
| Aging-Related | 100 | 1.11 | 7.3E−8 |
| CVD | 27 | 1.14 | 4.4E−4 |
| Cancer | 55 | 1.09 | 2.1E−3 |
| Alzheimer's | 3 | 0.66 | 7.0E−2 |
| Diabetes | 7 | 1.24 | 2.7E−3 |
| Lung | 8 | 1.20 | 6.5E-3 |
| 5 + Years Survival | | | |
| All-Cause | 575 | 1.08 | 9.0E−21 |
| Aging-Related | 350 | 1.09 | 2.0E−16 |
| CVD | 141 | 1.10 | 6.6E−10 |
| Cancer | 128 | 1.05 | 3.7E−3 |
| Alzheimer's | 24 | 1.05 | 2.5E−1 |
| Diabetes | 26 | 1.21 | 4.1E−9 |
| Lung | 31 | 1.08 | 3.3E−2 |

TABLE 2
Morbidity Validation for DNAm PhenoAge

| | | | | | | | Physical | |
|---|---|---|---|---|---|---|---|---|
| | Comorbidity Count | | Disease Free Status | | CHD Risk | | Functioning | |
| Sample | Coefficient | P−value | Coefficient | P−value | Coefficient | P−value | Coefficient | P−value |
| DNAm PhenoAge | | | | | | | | |
| WHI Sample 1 | 0.008 | 2.38E−1 | −0.002 | 3.82E−1 | 0.016 | 5.36E−2 | −0.396 | 1.04E−4 |
| (Non-Hispanic White) | | | | | | | | |
| WHI Sample 2 | 0.031 | 2.95E−7 | −0.026 | 1.63E−2 | 0.023 | 1.89E−1 | −0.361 | 3.81E−5 |
| (Non-Hispanic White) | | | | | | | | |
| WHI Sample 1 | 0.013 | 6.15E−2 | −0.006 | 2.40E−2 | 0.021 | 2.02E−2 | −0.423 | 4.50E−4 |
| (Non-Hispanic Black) | | | | | | | | |
| WHI Sample 2 | 0.014 | 7.67E−2 | −0.023 | 6.98E−2 | 0.048 | 2.27E−2 | −0.473 | 3.75E−4 |
| (Non-Hispanic Black) | | | | | | | | |
| WHI Sample 1 (Hispanic) | 0.024 | 1.64E−2 | −0.004 | 3.67E−1 | 0.033 | 5.07E−2 | −0.329 | 7.37E−2 |
| WHI Sample 2 (Hispanic) | 0.003 | 7.83E−1 | 0.002 | 9.28E−1 | 0.073 | 1.98E−1 | −0.377 | 6.54E−2 |
| FHS | 0.022 | 3.93E−7 | −0.034 | 1.59E−3 | 0.028 | 5.47E−6 | −0.016 | 4.60E−1 |
| NAS | 0.023 | 7.59E−6 | −0.062 | 2.00E−4 | 0.03 | 2.27E−2 | NA | NA |
| Meta P-value (Stouffer) | | 4.56E−15 | | 1.06E−7 | | 2.43E−10 | | 2.05E−13 |
| DNAm Age (Hannum) | | | | | | | | |
| WHI Sample 1 | 0.007 | 3.90E−1 | −0.003 | 3.48E−1 | 0.013 | 2.36E−1 | −0.399 | 2.90E−3 |
| (Non-Hispanic White) | | | | | | | | |
| WHI Sample 2 | 0.025 | 1.53E−3 | −0.02 | 1.55E−1 | 0.022 | 3.30E−1 | −0.284 | 1.43E−2 |
| (Non-Hispanic White) | | | | | | | | |
| WHI Sample 1 | 0.022 | 2.72E−2 | −0.007 | 6.03E−2 | 0.015 | 2.67E−1 | −0.345 | 4.29E−2 |
| (Non-Hispanic Black) | | | | | | | | |
| WHI Sample 2 | 0.022 | 6.34E−2 | −0.008 | 6.62E−1 | 0.055 | 6.12E−2 | −0.323 | 9.56E−2 |
| (Non-Hispanic Black) | | | | | | | | |
| WHI Sample 1 (Hispanic) | 0.01 | 4.33E−1 | −0.01 | 6.24E−2 | 0.011 | 6.10E−1 | −0.599 | 1.16E−2 |
| WHI Sample 2 (Hispanic) | −0.012 | 4.17E−1 | 0.035 | 0.209 | −0.012 | 0.885 | 0.04 | 0.348 |
| FHS | 0.019 | 5.94E−4 | −0.03 | 0.026 | 0.022 | 0.015 | 0.002 | 0.928 |
| NAS | 0.009 | 2.19E−1 | −0.026 | 0.226 | 0.025 | 0.183 | NA | NA |
| Meta P-value (Stouffer) | | 6.76E−6 | | 2.03E−3 | | 1.10E−3 | | 2.03E−5 |
| DNAm Age (Horvath) | | | | | | | | |
| WHI Sample 1 | 0.007 | 3.49E−1 | −0.004 | 0.169 | 0.001 | 0.912 | −0.08 | 0.071 |
| (Non-Hispanic White) | | | | | | | | |
| WHI Sample 2 | 0.006 | 4.54E−1 | −0.006 | 0.676 | −0.02 | 0.382 | −0.078 | 0.001 |
| (Non-Hispanic White) | | | | | | | | |
| WHI Sample 1 | 0.018 | 3.96E−2 | −0.006 | 0.062 | 0.009 | 0.407 | −0.141 | 0.004 |
| (Non-Hispanic Black) | | | | | | | | |
| WHI Sample 2 | −0.008 | 4.20E−1 | 0.002 | 0.905 | 0.004 | 0.875 | 0 | 0.998 |
| (Non-Hispanic Black) | | | | | | | | |
| WHI Sample 1 (Hispanic) | 0.012 | 3.65E−1 | −0.007 | 0.186 | −0.001 | 0.978 | −0.014 | 0.841 |
| WHI Sample 2 (Hispanic) | −0.025 | 6.69E−2 | −0.013 | 0.619 | −0.024 | 0.757 | 0.045 | 0.332 |
| FHS | 0.011 | 5.82E−2 | −0.021 | 0.083 | 0.007 | 0.519 | 0.01 | 0.673 |
| NAS | 0.011 | 7.90E−2 | −0.039 | 0.045 | 0.006 | 0.714 | NA | NA |
| Meta P-value (Stouffer) | | 4.54E−2 | | 1.31E−3 | | 7.51E−1 | | 4.66E−4 |

TABLE 4
Phenotypic Aging Measures and Gompertz Coefficients

| Variable | | Units | Weight |
|---|---|---|---|
| Albumin | Liver | g/L | −0.0336 |
| Creatinine (log) | Kidney | umol/L | 0.0095 |
| Glucose, serum | Metabolic | mmol/L | 0.1953 |
| C-reactive protein | Inflammation | mg/dL | 0.0954 |
| Lymphocyte percent | Immune | % | −0.0120 |
| Mean cell volume | Immune | fL | 0.0268 |
| Red cell distribution width | Immune | % | 0.3306 |
| Alkaline phosphatase | Liver | U/L | 0.0019 |
| White blood cell count | Immune | 1000 cells/uL | 0.0554 |
| Age | | Years | 0.0804 |

| | |
|---|---|
| Constant | −19.9067 |
| Gamma | 0.0077 |

TABLE 5
513 Polynucleotides having CpGM ethylation Sites Useful in
Embodiments of the Invention.

| | | SEQ ID |
|---|---|---|
| Probe | Sequence With [CpG] Marked | NO |
| cg000 | TAAAAATGATCATTTCTGCTTACGTTTACAGCTCATTTCATATTCTGCAAAATGT | 1 |
| 79056 | TTTCC[CG]TCTGCTATCACCGCCGCCATCCTCACAGCAGCCTGGGAGAAAGGCA | |
| | GAGCCAAAAGTCTC | |
| cg000 | GCCCTGCGGGAAGGGACTGGGGTTGGGAGGACGCTGGGCCTCTGGGTTTAGG | 2 |
| 83937 | CCTCACTC[CG]CCGGAGAGGGGGAGACAAACAGGCCAGACTCTCTTCCCAGA | |
| | GCAGGAGCGACCCCTCCCC | |
| cg001 | GCGTTTGTAGGCAGTGATGTCACAGAGTGCCTTCATGCTCCTCGGGTCTCCGGT | 3 |
| 13951 | TCTCCC[CG]GACCTCTGTAGTCCTCATTGCCAAAGTTGTACCCCCTGGGGAGTG | |
| | CACCCTGCCTGCATT | |
| cg001 | CTTTGCTTTCTTATCTCCAGCTCACACCTTTAAGTCTTATGTAGTTAAAGGACATT | 4 |
| 68942 | TATC[CG]CCTCCTTGGAGAACACAGCCCTCCAGTGTCTCCTGCAGCCTGGAGCC | |
| | TGGGACATTCTGG | |
| cg001 | TTATTGTAAACCCATTTTACCAGTGATGTGAATGAGCCGCAATGAAGGCTAAG | 5 |
| 94146 | GGACTTG[CG]CAAGGTGACATATATAAGCAACAGGCCTGCGATTGGAATCCAG | |
| | GCCCCAGAGTCTGGGCA | |
| cg002 | AGGGGGATGGAGCTTCTACACAGGGCCCCAGCGCTGTCGCTGTGGCTGCTGCT | 6 |
| 30271 | GCCGCTA[CG]GCTTAGTGCACCAGACGCTGCATTTCAGGTGCTCCTACAAAAG | |
| | AGGCCACTCCTGGAACG | |
| cg002 | AAGGTCCCGCGGCCTCGGGCCCCGCCCCGCCCCGGGGCCTCGAGCGCCAGGC | 7 |
| 61781 | CGGCCCGG[CG]AACCCCGCCCAAGGCCAACAAGGAGCCTTGTCCGCGCATTCC | |
| | AGCGGCAGAAACGGAATG | |
| cg002 | CCTCAGATGCACAGTGACACCCACCTTGGAGAGTTTCTGTGTCTCTTAAATGAC | 8 |
| 97600 | CGAATC[CG]TGTAGAAGGCTTATTACCACAATCTGTAGCTACTTGGTAAACGGC | |
| | AGCTCTTATTTTGAC | |
| cg003 | GCCAAACCAGTGGCTGTTTCTGAAATGTGAGCTTCCGCCCCAAGCTAAAAAGT | 9 |
| 35286 | GTTCACA[CG]TGGGTGGTCTGGAAAAGACCAAAGAGAGAGACCTGAGTTGAA | |
| | TTTGCCAGGCGGGTAAAC | |
| cg003 | CAGAACACCGAATAAATACCAGTTCTTACATGACATTTCACTCCACGGAAAAAT | 10 |
| 38702 | CTGGAG[CG]CACACTGCACCGCCGCCCGTGTGGCCTGCCCGCAACCCGGTGGC | |
| | TCTGCCCGGCCCCGGC | |
| cg003 | GAAGCAGTTCGATGCCTACCCCAAGACTTTGGAGGACTTCCGGGTCAAGACCT | 11 |
| 50702 | GCGGGGG[CG]CCACCGGTAGGCCGCAGCGGGGCCGGGGTCGCGTGGAGGG | |
| | GGGCGTCCTAGAGCTTAGCC | |
| cg004 | CTTGCACCTCTGGCTTTTGCAAACTGGGGGCCCAAGAGCTGCACCCAGGGATT | 12 |
| 10898 | TTATAGC[CG]TTCTTATCGGTCCTCAGGATCAAGGACCAATCAGGTCCCTCAAC | |
| | TGGTCTGGTGAGCCAA | |
| cg004 | TTGGGTGGGGCGTCTCAGCATTCCTCCAACGGGCAGGTCTCAGCGCTCCTCCCC | 13 |
| 12772 | CTGCTC[CG]CTCCTCTGCAGGGCCCAGGCGCCCTTGGCCTTAGGACCCAACTTC | |
| | TCTTACCGCCATGGA | |
| cg004 | TTTCTCTGGGAGGGGGCCTCTGCCCAGCTGTCCCCTGTGCGTCATGTGCAGGA | 14 |
| 12805 | GGCCAGG[CG]GCTCGCCTTACAGGGACCCGGCCACCTCTATATATAGCCCCTC | |
| | GAAGACAGCTGCTCAGT | |
| cg004 | CAGAGGAGACTCCTGGTCCCCTGTCCGGACCCCGCCCCGACCAGGTCCAGCCC | 15 |
| 62994 | CGCCCAA[CG]GCAAGTTAAGAGCCCCCCAGTGCCAGACGCTCCAGACAGACTG | |
| | CCACTCTTGGGGGGCAA | |
| cg005 | CTGGAGGCATCTTCGGACCTCTGGGCGGCCCAGCCCTGCCTGGCGTCTCCCCG | 16 |
| 03840 | CCGCTTG[CG]GCCTACCGCCAAGAAGCTATGCCTTAGGCAAACCATGGAGCTC | |
| | TGGCCCCAGAGGGCGCC | |
| cg005 | GGTGCCAGTGAAGGCCGGGTGCCTGGTCCCCCCAGGAGGCTGGTCTTGGAGC | 17 |
| 15905 | AGGTGGTC[CG]GTGCTGGTGGTGGAAGGACAGCAGCTTCTCTGCTAGTGGCC | |
| | ACAGGCAGAGCCTGCCTTT | |
| cg005 | AAAACATGCCCCAGCTTTCCCAAGATAACCAAGAGTGCCTCCAGAAACATTTCT | 18 |
| 82628 | CCAGGC[CG]TCTATATGGACACAGTTTCTGCCCCTGTTCAGGGCTCAGAGATAT | |
| | AATACAGACATTCAC | |
| cg006 | CTACAACTATGGCTTGTCTGAGTCCTGAGCCAGCAGAGCTCAGGCCACAGCAC | 19 |
| 87674 | CTGCACC[CG]TTTTCTGCTGCTGCACACAAGGGCTCTGTGCATTCCGCATCCAG | |
| | GTGTGCCCCTCCTCTT | |
| cg007 | TTCTCCAAGTAATTTTCATGTGCAGCCAGAATTGCAACTCACCAGGCTAAACTG | 20 |
| 44433 | CAGTTG[CG]CAATTCTGGTCTTCTTGATACCTGATTTCTTTGCCCCTTCTCTTTTC | |
| | TGGTTCAATGCAT | |
| cg008 | AATCCCCCTACCTTGATGTCTTCTCTTAGTAATCCCACTGATCCTCTCTGTTTTCT | 21 |
| 45900 | TTGC[CG]TATTCAGTGTTAAGCACAGTAAGTCTTTCCTACTGAAATAGCCATGG | |
| | TCCTAATCATAAT | |
| cg008 | ACTCAGTTCAAGGTTTATAAGAAGAGGAAATGTTTTGCCCTGGCCGCGTTTCCT | 22 |
| 62290 | TTTCCA[CG]TATTGTCTGTTAGAGTGCAAGCTGAAATAATGGGTTTTCTAGTTA | |
| | ATGGCATGTTCCAAT | |
| cg009 | GCCCGGATGCGTCCCTCTTTCTCCACCCCGCCGAGCCTAAACTAGTGACGGGG | 23 |
| 43950 | AGGGAGA[CG]GGATAGTGTTTCTGTTTCGTGGTCTTTGAATCCACAACCTCTAG | |
| | TCTGAACACAGAGAAC | |
| cg009 | CCCACTTTTCCAGATTGCTCTGAATGTCCTAGTGAGCTGCTCCCGTTGGGTAGG | 24 |
| 55230 | CTCCTG[CG]CCTCAACCGCGCTCGGTACTCGACGTTTATTATCAGGGAATTCTC | |
| | GGCTGCAAGATGGGA | |
| cg010 | AGAAGGAACTCTGAAGACTCCGTAGATTGCTCTAGACCGCCTCAGACACTCTC | 25 |
| 56568 | GGCGCAG[CG]TGGAGAGGATTTGTGCAAACATTTCCTCTGTGGACCAAGAGG | |
| | AATGCAAGAGGAGGCTGC | |
| cg011 | TCGCGGGTGATCTCCTGGCTCAGGGCCCGCATGCGGGAGTAGCAGGTCGGGG | 26 |
| 14088 | GAGTGGGC[CG]CGCGGCGGGGGCTCCCGCCAGGAGCAGCAGCAGCACGGGC | |
| | AGAGGCCCAGGCGTCCTCAT | |
| cg011 | TTGCCAGCTTAGTTGTAATTTCTTGTATCCATCTTGGTCCTCTTCAGTGCCCAGC | 27 |
| 28603 | CAGAG[CG]CTGGCAGACAGGCACTGGGTACGTTTTGTTGAATGAATTGGGAG | |
| | CGAACGTCGTTTAGTG | |
| cg011 | CTCCGTCGGCCCGGGCTCCTGCCTTGGGGGTGTCCCCTAGGTAGAGAATGCGT | 28 |
| 31735 | CGGGGAG[CG]CTTCCCGCCAGAGATGGGAAGCCCAGGAAGCCCCTCCCCATG | |
| | CAAACAGTGCCCCCGCCT | |
| cg011 | CGTGTATATTTTTAAACTGTGTGCTGACGACAGTTAAGTAAATGTGATTCAGAA | 29 |
| 37065 | CTTCTG[CG]TATTTTGCAGGACAGTTTTGACACAATGACATGACTCGCTAGCCA | |
| | GGAAAGATAACGACA | |
| cg012 | TTCTTAATAATGAATGAACCAACGACCCCCAAGGCTGGTTTGCCCGTGCACACG | 30 |
| 11097 | CACGCA[CG]TGTGCAACACGTAGCACTTGCTGAGTGTTTGCTACTTGCCAGGCC | |
| | TCATGTCAAGCACTT | |
| cg012 | GGCCAGGAGAGGGAGACTTGGCCCAAATAAAGTGACTCAGGCACCCTCAGGA | 31 |
| 21637 | ACTCTCGG[CG]CCCGGGGCCCCTTCGGGCAGCCTTCGACCCCCATGCGTCTTTC | |
| | GGGTCCCCAGGGACGCG | |
| cg012 | CACTTTTCCTCCCCAGTACGTGGGAGCCCTAGAGGACATGTTGCAGGCCCTGAA | 32 |
| 52496 | GGTCCA[CG]CGAGGTGAGTGCAGGCAGCCTCAGGGCTTTCACATCAGCACGT | |
| | GGCTGTGCTACTGGACA | |
| cg012 | GGCGAACCCACCCCTCCAGGCAGGGTTTCGCCCCTCGCCCCGCCCCTTCCCCCG | 33 |
| 54459 | CCCGGA[CG]GCCATGGCCATTCCCGGCATCCCCTATGAGAGACGGCTTCTCATC | |
| | ATGGCGGACCCTAGA | |
| cg012 | CAGTTTTAGTCCTTTACGGTGATTTGTAAGCCCAGGCCTTCTTAACTAGGCAAA | 34 |
| 61503 | TGCTGC[CG]CCAGGTGGCCTAGGCCTAACCCCAGAGCCGTTGTCTTGACGCTT | |
| | AAGCTTCCGGGGAGGG | |
| cg013 | ACGGGCAGGAATCTGTTGTAGAAGAGTTGCTGCCGGGACCTGCTGGTGAATT | 35 |
| 35367 | GGCTCCAC[CG]GATCCGGCTCCGCAGAAAGCTCACTGCTTCCTGTGGCTCCTG | |
| | GATTTCCAAGCCTCTGGG | |
| cg014 | AAGAAGCTAGGAGGGGAAATAAATTGAGTGGGGGTGGGGTTTCCCAAGAATC | 36 |
| 00401 | GGAGGAAC[CG]AGAACGAAGAGGGGTGGGGGAACGGGGAAAGAGAGAGGA | |
| | AAATCAAGTTTTCTTCAGCAC | |
| cg014 | TATGACACACCTATATTCACACAGTTGTGACTGTGGACACGCAAAATGCCTGAG | 37 |
| 41777 | GCCCTG[CG]TCCAATCCCGGAAGCACAGTTCCTGGGAGGAGTCACTTCTATAA | |
| | TAGCCGTATCTTCCCT | |
| cg014 | GGTGGTGGACTTTGGGACTGGACAGACCTGGTCACAGTCTAGGTTCTACATCT | 38 |
| 50842 | TACTGGT[CG]AGCAACTTTAGGCAAGTAGCTTAACTCCTCCGAACTTATTTTCCT | |
| | TTTCTACCAAATAAT | |
| cg014 | GCAAGTTTAAAAGTACTCACAAAATCTAATAGGCAATTCAACATAAAACTCCAT | 39 |
| 59453 | GGCTAT[CG]CTGTTCCTCACTTTCTGAACCTTTACCTGCCTGACTTTACTCCATA | |
| | CCACTCCAACTCAC | |
| cg015 | GTAGTTTTATTGTATCAGACTTAGTACAGGGGTGGGGTGGGGGTGTGTATTGG | 40 |
| 11567 | AATGATG[CG]TGCCCGTTTCTCTGCAAAATAGTTTCTATGTCATGGAAAGGAGT | |
| | CGATGGGACAAGAAGA | |
| cg015 | CAGCCCCGCGCCGCATCCTCCGGCCGCCCCCTCCCCGCTGCGAGCTTACGCCGC | 41 |
| 19742 | TGTCGC[CG]CCGCCACCGCCTTAAAAAGGACAAAACGGAACAGAAAATGAAT | |
| | GCATGCACAAAAAAAAT | |
| cg016 | ATGATAGGTGTGAGCCCCTGCGCTTTGCCAGGGCTGGTTTTTGGATGTGATTCT | 42 |
| 23187 | CAGGGC[CG]TCTTTCTTTACCCTTCTGCTCTGCTGAGGCCCACAGCAGCCTAGT | |
| | CTCCTTGGGTGTGGG | |
| cg016 | TGCTGGGTATCCGCGCCGGAACCGCGAGGGGGTTGGTTCAGGCCTAGGCGCG | 43 |
| 26227 | GGGCAGGA[CG]GGACCGGTGAGTGGCTCCTCCAAACAGCTATAGAGACCCAG | |
| | AAATGCCTGTGGAAAGCTA | |
| cg016 | CAGAACCTGCAGGAGCAGATCAATCCCCTCTTGGTAACACACCAGAGCCTGCG | 44 |
| 51821 | GATACCG[CG]ACTCCGAATCTAGTTCTACTGCCCGCTTTAGCACAGTGGCTGCA | |
| | GCTGTGCTCTGCGGGT | |
| cg019 | CTTCTAGTGGCAAATTTCTCCCTGCTGTGGCAGGAGGACGGCTCGGGGGAGCT | 45 |
| 18706 | CTGACCA[CG]ATTTCATGCAAAGATACGGTGAGACCCTCCGCTCAACAGTGGC | |
| | TTTTCTAAGGCTCTCCT | |
| cg019 | ACATGGGCTTCTCTTCGAGGAGGTAACATGTCCGCGCCCTGAGCCACGGCTCT | 46 |
| 30621 | CTGGGCG[CG]GCCATCTTGGTAGATCTGCCGTACAGAAGGGAAACAGTTGTTC | |
| | TTGTGTCATTAAACCGG | |
| cg019 | GCTTTTTTGGATTGTGTGAATGCTTCATTCGCCTCACAAACAACCACAGAACCA | 47 |
| 46401 | CAAGTG[CG]GTGCAAACTTTCTCCAGGAGGACAGCAAGAAGTCTCTGGTTTTT | |
| | AAATGGTTAATCTCCG | |
| cg020 | AGAGGCCTCGGTGATTTCCCGACCTCTCCTGTGAAGCCTGATTCGGAACTCTTC | 48 |
| 16419 | CAGCTG[CG]AAGAACTTGGCCGATTCTAAGGCACATCAGGGCTGCCTGGAACC | |
| | CTAACACCTGCCTAGG | |
| cg020 | TGCCTGATGGATAATCCATCACTTGCTTTTCTAGTATGAATGGTCTATTTACGGG | 49 |
| 71305 | TCCAG[CG]CCCCTGCTGGCTTACGACCTTTTCCAGGGCGGGGAGGGGCTGTCC | |
| | TCATCTCTGTGACCC | |
| cg021 | ACTGCGCCCAGCCCATTTTACAGACTTTTATTTTGTTCAGTTTCTTTATTGTCTTC | 50 |
| 51301 | CCAA[CG]TCCCCCCACACACACTGCACTAAAATGCAAACTTCACGAAGGCAAG | |
| | GAGGAACTTTTGCC | |
| cg021 | TGGGGAACGCGAGTGGGGACAGGGGGGCCTTCAGCTGGGCCCCAGGGAACC | 51 |
| 54074 | GCCCCGTGG[CG]CTCTCGGCCTCGCTCTCACTCACGGTGCTACAGGTGGTAAG | |
| | CAAATTGACTATGTTGTGG | |
| cg021 | CAGCCCCCCTCGGCGGCCGCACCGACACCGCACCCCAAGTCCTACCCCGGGGC | 52 |
| 97293 | CTGGCGG[CG]CTCCTCGCCGGGATGCCCTAGCTGTGCCGCAAGCTCCCCACGC | |
| | CCCTCTGCGTCCTTTTT | |
| cg022 | AGGAACCCATGGGAATGAGCTAACCGGAGTATTTCTGGTTAAGCATTGGCTAG | 53 |
| 28185 | AGAATGG[CG]CTGAGATTCAGAGAACAGGGCTGGAGGTAAAACCATTTATTA | |
| | CTAACCCCAGAGCAGTGA | |
| cg022 | GTGTGCAGAATTTATATATATAAATATATCTCCTCCAACCCCTCCCAATGAAGCA | 54 |
| 29946 | AGTCA[CG]TGAGTCAATCCTACCCTAAGATATTAGGGATTGAGCCTCCTGGGA | |
| | CATTTGGTGGCTTAG | |
| cg023 | GTGGGAGGTCCTTATGCTAGGAGACCTAATGTCTGTGCCTCAGTTTCTCTATCG | 55 |
| 09431 | GAGAGG[CG]ATGTCTCAAGAGGCCTTTCAGGGCTCAGAGTTGAGCTTTCTGAG | |
| | TTCCACATGGAAGTGA | |
| cg024 | GAACGACTCAGTCTCTCAAATCATAGCTAATTCTCCTCTGAGGGCCTTGCTGAA | 56 |
| 80835 | GTTCTG[CG]TTTGCTTGCTCCGCTTTCCTCTCATTTTGGACCTCCAGCCTTCCTGT | |
| | AGTCCGAGGCCCT | |
| cg025 | CCTCCGGTCTAGGGCTCTTTGTCTTTGCAAAGTGTCGAAACTGTCTGGCATAGT | 57 |
| 03970 | GGGCTC[CG]CCGGCGGAGGCTGGAGCCGAGGAAGCGAGGAGGCGGGATGA | |
| | GGGTGGGAGAGGGCTCGGG | |
| cg026 | TGCCCGAGGCAGAAGGATGTTTGACCTCCGGATAAGCGAGGCGCTGCTGTGC | 58 |
| 31957 | ATTCATTC[CG]GGCTGCATCGGTGGCGACAGCAGAGGCTCGGGCGGCGACTCT | |
| | CCGGCCAGCGGCGGCGGT | |
| cg027 | ACTGTTCAAAATGATGAACGAAGATGCAGCTCAGAAAAGCGACAGTGGAGAG | 59 |
| 35486 | AAGTTCAA[CG]GCAGTAGTCAGAGGAGAAAAAGACCCAAGAAGGTAAATCGC | |
| | CGGAATTAGGAATGTCTGT | |
| cg028 | TCCAGTTTTAATCTTTAAAAAGAAGAAGAAGCAGCAATGCATAAGCTGAGTGA | 60 |
| 02055 | TTCCCCG[CG]GAATCCAAAGCTAACAGAGCCAATAAGGCACCTTCGAGGGCAT | |
| | CCCAGCCCAGCTACTGA | |
| cg029 | CTCTTGCAGGAAGCCAGTTGAGGGAAGTTCTCCATGAATGTACGTCACAATGA | 61 |
| 76574 | TGATGAC[CG]ACCAAATCCCTCTGGAACTGCCACCATTGCTGAACGGAGAGGT | |
| | AGCCATGATGCCCCACT | |
| cg030 | GGTGACAGGGACCTAGGGCCTGGGCTGGGAGGAGGCGGGGCTAGTCCAGGA | 62 |
| 07010 | AGGGACCCG[CG]CCACCCAAGTGGCCCCTGCAGGGGCCTCCTGAGGCTCCTG | |
| | GGTCCTTCCCCAGCTCCCAT | |
| cg031 | TTCTTTCTCCTCCACTGCAAAGTTAAATGCGAGAAGGTAGAAACCCAGAGGCCA | 63 |
| 12869 | TGCTGG[CG]CTGAGAGATGAGCCCCACTCACCAGATTCAAGATCCCAAGGTAG | |
| | GCACAGACACAGGGCA | |
| cg031 | TGGAAGGTGTCAGCGTGTGGCTGTGTGATCTTGCATGTGTCTGTGTTCTGCAG | 64 |
| 72991 | GAACATG[CG]TCAGTGTGTGTGCATCAGTGTGCATCTCTATGTGTCATGCACTG | |
| | GTGTGTCTTCGTGTAT | |
| cg032 | AAAGTGTTGGGATTACAGGCGTGAGCCATTGCGCCTGGGCAGGTATTTTTTCT | 65 |
| 58472 | CATTAAG[CG]CTCCCCATCCAAGTCTGCCCTAGGCAGGAGTGCCTAGTGCACG | |
| | GGTACATACATACCCCG | |
| cg033 | AGGGCGTTTGCCACAGCCCCTTAACTCCTTCCAAAACACTCCGCTTAGATACTG | 66 |
| 40261 | ATAAGG[CG]CCAACTGCAGCCTGGAGAACCCCTATGCGCCATCTTGGCTTCCC | |
| | GCAGGCCTCTGCGCCG | |
| cg033 | CAGCCTCTTCCTGCTGTGTCACCATCTGCGGGAGGTGGTACTCTAGTCTCCCCT | 67 |
| 87497 | AAGACT[CG]GCTTGCCACCTGCACCAGCTCCCTGGGCAAAGGTCACCTGTGTTC | |
| | TTAATAGAGCAGAGA | |
| cg035 | ATGGAGTATGTATATGTTCAGCTTTACTAATGCCAAAATGTTTTCCAACATAGTT | 68 |
| 35648 | GTAAG[CG]ATTTGTGCCCTCGATAAGCAGGGTATGAGAATTTCCATTGTTCCAT | |
| | GTCCTAGCTGACTC | |
| cg035 | CAAATCTCTCTGCTTCCTTCGATGTTGCCTGTGGCAGAAATTTACATTATCCCTT | 69 |
| 65081 | CAGCC[CG]CTTAAAAAATTCTGTACTTCCCAAGCGGCTAAATTTTTAAAGTCCCT | |
| | CAACCACAAAAAT | |
| cg036 | AAGTAGAGAGGCAGCCGGGAGCCTGCCTTCTGTGTTCTCGGTGCAGGGGTATT | 70 |
| 23878 | CTGAGAA[CG]GCCCCTGCTCACACGGGTTTAAAAGGAACTCAGTGACCACAGA | |
| | CGGATGAGAACAGCGGA | |
| cg037 | TCCGCAGGGGTCTCCTGGGAGGAACCCACCAGCGATAGGAACACTGAAGCTG | 71 |
| 03325 | GGCTACGG[CG]TCCGCCCGAGCCTTTTCTTAAAGGCGCCGACCCCGGAAGCGG | |
| | GGCGTCCGAGGGAGCGCG | |
| cg037 | ACCTGCGCCCACAGGGCCTGGGGAAACCTTGAGTACGAATGCCACGCCGCGG | 72 |
| 24882 | CTTGTGGG[CG]ACACCACCGCTGTCACCATGCCCCAGGGCCACCTGGCAATGC | |
| | TGCTCTTTTCCCGTGACG | |
| cg038 | AAATTGATCAATACGAATGATCACGCCCCATGTGCATCTCCTCAGCAGCCACAA | 73 |
| 19692 | GAGAGA[CG]AGGTCACTGGAGGATAAACATCCGTGACTGCACCTCATGATCCA | |
| | TCACGCACGACGGCCG | |
| cg039 | TCTTCCTCCATGTCCAAGACACCCAGCTTAACAACCCTGTAGCCCCCAACTTGGC | 74 |
| 29796 | CCTAG[CG]GCACCTCGCCTCGACCTTGCCATTTTATACTCAATTGGGGCGTAGG | |
| | GTTCTGAAGCCCAG | |
| cg039 | CATCTCCACTTCTCCAGTCCGCCCTACTCTCCACCCGTGACCTCCAGTGGAGACC | 75 |
| 77782 | CCAGG[CG]GCAGCATCAGTATTTGATCGGCCCTTCGTCAGCACGCTGCCAGCC | |
| | CTGGCCGGCTGGGTT | |
| cg039 | AGTTGCCACAGGGTAAGCCCAGTGCCCTTTTGCCCAAGGTCAGGTCACTTGGT | 76 |
| 91512 | GCTGGGG[CG]TCACAGAGCCCAGGAAACTTGGGATCAGAACCCCCTGCTCCCC | |
| | GCTCCCCACCTCATCCC | |
| cg040 | CTGACCCTCACGCAGTGTCCGCCTCCAGGGAACTGTGGAACACGTCGCAGAGA | 77 |
| 07936 | GCTCAAG[CG]CCACGTTTGGATCCCTGAGCAGCTGTCACAAGCCTGCACCCAG | |
| | GACTGGGGGGCCTGCTG | |
| cg040 | GAGGGAGCAAAGGTCTCCGGTGTGGCAGGCAGGTTTTTCCAGGCAGCTGGCA | 78 |
| 14889 | GGTGTGCT[CG]CGCAGCTGACACTGCCTTGGGAGCACAGAAGGTGGCAGCAA | |
| | AGATCATGCGGTCTTTTGA | |
| cg040 | AGGGTGCCTGCCTCTCCCGGCCTGCGCCTGCGCGCTGGGGCCTTCGGCTGAAG | 79 |
| 84157 | GGGTGTG[CG]CTAGCGGAGCTCCGGGAAATGAATGAATGAATGAATGAATGA | |
| | AATGCTGAAGCGGGCAGG | |
| cg040 | TAAGGCATCTGCTGAGTGTATAACCATTTTACCTCTTGTTTTTAGCCCTCTTCTG | 80 |
| 87608 | GGTCA[CG]CTAGAATCAGATCTGCTCTCCAGCATCTTCTGTTTCCTGGCAAGTG | |
| | TTTCCTGCTACTTT | |
| cg041 | GACGCCGGCCCGAGGTGGCGCCGGAGCTGCTGGCAGAGGGGCGGCGGGCGG | 81 |
| 69469 | CGGCGGCGG[CG]GCTACAGGAGGGACTGACAAAGCCCCACGGCACGCCGCTC | |
| | CCTACTTATAGCACCGGCGG | |
| cg043 | CCCCGGTCCGCCTGGCCCCTCGCCCGCCCGCCAGGCCCGCCGAATGCGGCCTC | 82 |
| 33463 | CGCCCCG[CG]CGCCTAAAGGAGGAGCGTCGCGGGGGATGGAGGCGGCGCGC | |
| | GGTGGGACCTGGGGAGATG | |
| cg043 | CGAACTCCTCACCTCAAATGATCCGCCCACCTCAGCCTCCCAAAGTGCTGGGAT | 83 |
| 59302 | TATAGG[CG]TAAGCCACTGCACCTGACCAATACAGTCTTAATAGGGCTATTTGG | |
| | ACCTCCTTGGAGACA | |
| cg044 | GAGACCTCCTGCCAACCCAATTCCCAGTGCGCAGATGGGGAGGAAGAGGCAG | 84 |
| 16752 | CGAGGAGG[CG]CCCCCAGCTCAAGGTCACCCATCAGGTCTGGGGCAGAGAGA | |
| | GCCAGAAGCCCGGAATTCC | |
| cg044 | CACCCTACTGCATGTTGCAAAGTATTCCTTTAAAATGAAGTGAGTAAAATACTG | 85 |
| 24621 | GGATGA[CG]TTATCTGGAGCCCAAGAAAGATGGCTCATTTGGAAAGGCCTAAT | |
| | ATCCCAAGTTGCTTAC | |
| cg044 | GGCCGGGTGGGGGGAGGTGACTTGATGTCATCCTGAGCAGCTGGGCGGCGG | 86 |
| 80914 | GTGCCGGTG[CG]CACGGAGCCGAGCCGGGGCTCCCGTTGCGCTGCACCGCGT | |
| | TGGGTCGGAGTCCCAGGACT | |
| cg045 | GCAGCCCGGGAAGGGGCATTGGTGGCGCTTGGCAGCAGGTGTGACAGACCTC | 87 |
| 28819 | CTCCGGGG[CG]CCTGATCCGCGGCGGGGGCGGGGCCTGCCCCTAGGGCCCCT | |
| | CCAGAGAACCCACCAGAGG | |
| cg046 | CAAGGGTCTAGGGTCCTGGGTATCTCTAGGTACTGAGACAGCTGTGTGGTCTG | 88 |
| 01137 | CTGCATC[CG]TGCCCCTCTCTGAGCCTAGAGCCTGGGCTGGCCCAGGAAGCAG | |
| | GAAGAAGTCTGCACCAG | |
| cg046 | TCAGCTCGTGGGCTGCCAGCGTGCAACCTCTCACCTAGATAATGGTATATAATA | 89 |
| 16566 | TAAATA[CG]TTTCCCTTCCCCCCTTTTTTCTCTTCCTCCTCTTTCTCCTTTCCCTCCC | |
| | ATTTTCCACAT | |
| cg047 | GGTGGGTGGTCCGGCCCCGAGCCCTCCTGACTCTCTCGCCAATGCCCAGAGGC | 90 |
| 18414 | GCCGCAG[CG]ATTCCAGGGAGGCCGCGCTCTCGCCCCAAGGCAACCAGAAGC | |
| | CCACGTGCCAGGAGAGGC | |
| cg047 | GTTCTCATCCCATATGCCTTTGTCCAAAGGTTGCACGGGGGTTAAGCTTGGCCC | 91 |
| 36140 | AGAAGG[CG]CCGAGGGCTGGTCGAGTTCTCCCCTTTCCAAGAACCAGCCGAAT | |
| | CTCCCTCCCGCAGATC | |
| cg047 | TGATCGGGACGAGGAAGGGTCACTCCGTGACCCGGGATAGGGCCGGGATGCA | 92 |
| 55031 | GCCTTGGA[CG]GGGCTGGGCCCAAATGTGGGCTCTGGAGGGAGCCGGGCTG | |
| | GGGCTGGTGCTGGTGCTCCC | |
| cg048 | CAGCGGGAGATCAAGTGAGCCTCAAAACATTAGAAAAACCCAAGCCAGTCTGC | 93 |
| 18845 | AGAGCAC[CG]CAGCCGCCTCAGGGCCGGTTACCATAGCTACCCTTGGCTTCCC | |
| | AGCCCAGCACATGTCTG | |
| cg048 | CTCTGCGGGGACAGAGGTCTCAGGAAAGTAGCCTTTATTTATGTGGCACCGAT | 94 |
| 36038 | CGGAACC[CG]CGGCCGGCCAGGCGGACCTGGACGGAGCGTCCCTGCTCGGAA | |
| | CCTGGCGCGGGGCGCCGC | |
| cg050 | AGGGTAGGGAGAGCGGGAGGCTGCTGGCCTGAGGCTAAAGCTAGTCACTGAC | 95 |
| 87948 | CTCTATCA[CG]TGCTTGTTATATGTTAGGCATGATATGCCAGCTCCTTTTATTCG | |
| | GCGTAGCAATCTCTGA | |
| cg050 | TCCACAAAGTACTTTCCATCAGATACACTTTTCTGATGGAAACCAGGTGTGTGA | 96 |
| 89968 | TGGTTA[CG]GCCCCAGGTTAGCTCCAGAGCACATTCAACTGTGGGTAAACACA | |
| | AATGTGCCCTGTGCCA | |
| cg051 | TTAATGCCCCCCAGAATCAGCACCATGTCATCACAGGCTTGGGTCAAGGGGCG | 97 |
| 25838 | GGTCAGA[CG]CCAGTCACATCCGCTCACTGCCCACAGCCACCCCCCCACAGTG | |
| | AGTCATCTGCCAGGGTG | |
| cg052 | GGTGTCCTGCCTGGGGTATCCCCAGAGTTTGGCACACGGTGATAGCCAACATT | 98 |
| 28408 | CACTGAG[CG]CCAAAGGGCCAGGTGCTGCCACTCTCTCAAAATAAGCCTCTGC | |
| | CACTTACTGAACAACTA | |
| cg052 | CTGCAAGATCGTGGTGGTGGGAGACGCAGAGTGCGGCAAGACGGCGCTGCTG | 99 |
| 70634 | CAGGTGTT[CG]CCAAGGACGCCTATCCCGGGGTGAGGGACCTGCGTCTTGGG | |
| | AGGGGGACGCTAAGGCTGC | |
| cg052 | GATGTCTCCAGGCACCCCCGACCTGGGCTTGGCCCTCTGCTTGGGGCGGAGCT | 100 |
| 94243 | TCCAGGA[CG]TGCTGGGACCTAGGTCTGACCCCGCCCAAGGCAGAGTTGAACC | |
| | CACTGTGAACTTTCAGG | |
| cg053 | CTGAGCAAGTCTTTGATTCATGGATTCCCAGCAACTCTAGCTGGAACAACTTCT | 101 |
| 16065 | TTGGCT[CG]TATTCCTCTGGTATATGTGCTGAATTTAGAATTCAATCACTGGAC | |
| | ACCAGGAAAGGCAAC | |
| cg054 | CTTAGTTAACTCACCTGAAAAATGGGAACAATAATACAAGCCACAGTTATGAG | 102 |
| 22352 | AATTCAA[CG]AGATAATGCATGTACAGCACCTGGCACATGGTAAAACGCTCAA | |
| | TAAGTGGTAGTTAGTAG | |
| cg054 | GAGTCTGGTAAGTGTCGGATGGTAGAACCAGGGTTGGGACTCGGGACCTCCA | 103 |
| 40289 | ACAGCATA[CG]ATGTGGTGGGGGTGGGCAGCCTGGGTGGGGGTGGGCATTA | |
| | CTCTGGGGCTGGATTCAGCT | |
| cg054 | CTCTCACCCGCTGCCGGGCTGGATTGTCCTCCACTTGTGCTTATCTGGTCCTCGA | 104 |
| 41133 | TGCCG[CG]CTCCGACGTCTTATCTGAGGGAGCCTTCCGTTAATGAAGGCTCTAT | |
| | AAACATCTGACAAA | |
| cg054 | GCCAGGTCACCCTCTCACTCTGTGCCTCTTAGTTATCTTGCATGCTCTGGTCTTT | 105 |
| 42902 | GCATA[CG]CTGCTCCCTGCACCAGGAACCTCCATCCCCATCTTTGTCTGCTTGTC | |
| | GAACTTCAGAAAT | |
| cg054 | GGTCCTGCCCCTCACCCCTCTCGCGGGGGGTCGACCTGCTCGTGGATGGGGAC | 106 |
| 73871 | CCTGGCG[CG]CCTGGGCTCCCATCCGGGGGTTCCCCGACCCAGGTCCCGGTCA | |
| | CCCCCAGCGCAGGGCCC | |
| cg054 | GAGCCGCCGATTGGCTAGGAGCACTTGAGCAGCGGAAGCAGCTGGCTCGCGC | 107 |
| 92270 | GGGGACTG[CG]GTGAGGGGGCGAGCCGTGAAGATGGCGGCAGTGGTGGAG | |
| | GTGGAGGTTGGAGGTGGTGCT | |
| cg055 | GCATTACCCCTTGTGGGAGCCATATTTTTCTAGAAGGCATTTTGATCAAGACAG | 108 |
| 01584 | GCCTCC[CG]CGGTTATTGATCTTAGGGTCATTGAGAGTCCAAGAACTGGGGAG | |
| | ATGAAGGCCACCCGGC | |
| cg055 | TGGGGGGCGCTGGCTGCCTGCTGGCCCTGGGGTTGGATCACTTCTTTCAAATC | 109 |
| 32892 | AGGGAAG[CG]CCTCTTCATCCTCGACTGTCCAGTGCCGCCGAAGAAAAAGTGC | |
| | CTGTGATCCGACCCCGG | |
| cg056 | ACCCGGAAATGCACAAGCCTCTTGATGCATAAAAACAGCTGGGCTCCCTTGGA | 110 |
| 97249 | GACAGAG[CG]CCATGGGAAACCGGGTCTGCTGCGGAGGAAGCTGGTGAGTA | |
| | GGCTGGAAGGGCAAAGGGG | |
| cg057 | CCAAGTAAAAAAAGCCAGATTTGTGGCTCACTTCGTGGGGAAATGTGTCCAGC | 111 |
| 59269 | GCACCAA[CG]CAGGCGAGGGACTGGGGGAGGAGGGAAGTGCCCTCCTGCAG | |
| | CACGCGAGGTTCCGGGACC | |
| cg058 | CTCCGACCCTGCCGCCCCCATTCTCCGCTCCCCGCTCTGGGGCTGAGTGAGGCA | 112 |
| 51163 | GGATGG[CG]AGAGACCCCTGAGCCACCAAGTCCGCTTACCTCAGGCAGATCCC | |
| | GACGGGGGCTCGGCGC | |
| cg058 | AAAGAGACGGTTTGGGAATTGCTCTGAGGATGCTATGCAAGTCACTAATAAAG | 113 |
| 98102 | GAAGACA[CG]GACAGATGAACTTAAAAGAGAAGCTTTAGCTGCCAAAGATTG | |
| | GGAAAGGGAAAGGACAAA | |
| cg061 | GAGTTTTCCTCTCACACTTGACCCTGATTTTGTTTTGCAGAAGCGACAGGCTGT | 114 |
| 34964 | GGACAC[CG]TGAGTAAGAGTCCTGGCAAAGGGGTCTGTGACAGAGCCCTTTTT | |
| | ACAGGCTTGCTTTCCC | |
| cg061 | CTGACCTCACCACCCACCAGGGAGGTGGGTCTTATTCTGGGCATCGTGCCAAG | 115 |
| 44905 | TTCTTAG[CG]GGGCCCTCTAGAATCTCTAAAGCAAATCAGGCTGAAGAGGGGA | |
| | AAACCAGCAGGGGGAGG | |
| cg061 | ACTTTCCAGCTCTTCCGAAGTTCGTTCTTGCGCAAAGCCCAAAGGCTGGAAAAC | 116 |
| 71242 | CGTCCA[CG]ATGACCAGCATGACTCAGTCTCTGCGGGAGGTGATAAAGGCCAT | |
| | GACCAAGGCTCGCAAT | |
| cg061 | CTGTGGGTTCGGCACTAGGTCCTCCTCCCCGTGGCTTCCTAGTAGGCATGTGGT | 117 |
| 89653 | GGTGTA[CG]CCTGCTGGGCACCTAGCGAGAGGGGTCGTGAGTTGGGAGGGA | |
| | GCCACGTTGGGGTGCCTG | |
| cg062 | CCGCAGCCGAGCAGGAAGAGCGAGCCGGGGGATTGAGACTGTCCGATCCAAC | 118 |
| 95856 | CTAGGGCA[CG]AGCCTGGTATAAATCGCGGACTAACAGAGACTATCTGATGAA | |
| | GAGACTAACGGAGAGAGA | |
| cg063 | GGCTCTTTTGTGGCTAATCTAGCGAGGGACCTAGGGCTGGGGGTGGAGGAGC | 119 |
| 27515 | TGTCTTCA[CG]TGAAGCCCGGGTAGTGTCTGATGATAATAAAAAGTATTTGCAC | |
| | CTTGATTTGCTGACTGG | |
| cg063 | AAATTGCCTGAAATTTCAGAGTTGGACTTCATCACTTGTCTGTGAGCCGACGCA | 120 |
| 63129 | GGCAGG[CG]TATTCTATATCAACGACAGACTCTCCTCTGCCATTTCCTTTCCTGA | |
| | ATCTAGTTAACATT | |
| cg064 | GGAGAGCAAGTCAAGAAATACGGTGAAGGAGTCCTTCCCAAAGTTGTCTAGGT | 121 |
| 93994 | CCTTCCG[CG]CCGGTGCCTGGTCTTCGTCGTCAACACCATGGACAGCTCCCGGG | |
| | AACCGACTCTGGGGCG | |
| cg065 | TGTCTTTCGGTTCATAATTGCGATTGTTAGCGAAGTGGTCTCGAATTCCATTTCA | 122 |
| 33629 | CTCCC[CG]TTCGCCGCTCTCAGACTAAATTGCAAATATCCCCAAGTCTGTAGCA | |
| | AAAAAAGTTTTCTC | |
| cg066 | AGCGCCTGGGCGAGTGACATCTGGGCCGGACCAGCTGGTGCTGCGCGGCGCA | 123 |
| 37774 | GGTAAGGG[CG]TGCGCGGGCAGGGACAGGGGTAAGGGGTGCCGGGGCGCG | |
| | GGGATACAGGGAGGCCTGCCC | |
| cg066 | TTATTCTGGTATCAATAAAAAGGAACTGTTACTATAGTAACAGATATTCCACTT | 124 |
| 38451 | GGTGCA[CG]GCCACTTCCACGATGCGGAACATCATGTCCAAGCCACACGCTTG | |
| | AGAGGCACAAATAAAT | |
| cg066 | GAAGCAATTTGAGGGTGTTCCAGATCACACCAACAGCGGATGCTGCATCTGGG | 125 |
| 90548 | TAGTTCA[CG]TACCCGAACAAAAATTTTAAAAATTTGGTGTGGCCTTTGCCATC | |
| | CATTCACTCCTCAAAA | |
| cg069 | TGAGTCAGAGGCAGGTGCTGCAAGGTAGGGCCGAGGCGGGCAGGTGCCCTA | 126 |
| 08778 | ACTAGCTGG[CG]CCGAGGAGACCCGGGTGCGGTGGGCTCCACCGACTCTCTCT | |
| | CCCGCAGTGTTCGAGCAAT | |
| cg069 | AACCGGGACGGAGGCTGGCCCTGGGACAGCAGGCGGCTCCGAGAACGGGTCT | 127 |
| 58034 | GAGGTGGC[CG]CGCAGCCCGCGGGCCTGTCGGGCCCAGCCGAGGTCGGGCC | |
| | GGGGGCGGTGGGGGAGCGCA | |
| cg069 | CTCGCCTGGGTCTCTCTCGCCCCGTCGCCCCCATTCCCCCACCCTCGGAATGAG | 128 |
| 75499 | GAGGGG[CG]CCTGCTACCCCCGGCCAGGCAGGCAGTGTGTCCCTCGGATTCCT | |
| | TCCAATTTCCTGATCC | |
| cg069 | GTAAGACAGGAAATCAATCAGAGGCAGAGCGACGCCTCTGGCTCTGGTCTAGT | 129 |
| 94793 | GGTGCAG[CG]TCTCTAGCCCTCGCCCCGCCCACCGTCCCCGCGAGGCGTCCACT | |
| | CGCCGAGCCCCGCCCT | |
| cg070 | GTGGCGCAGGTGCAGGACTGTGGGAAGACAGGAGCGCCAGGGAATGTCTGG | 130 |
| 38400 | CCAGCAGCG[CG]CTGCCCTCAAGGGGCCTCCTTGAAGGCCCCTTGAAGAGGGC | |
| | AACACAACTAATGACGATA | |
| cg070 | CCCCAGCTCAGGGTCCGTGTACTTGGGGACCATTTCCTGCTCTGCTGTGGTCTA | 131 |
| 73964 | CTGGAC[CG]TCTGGCATCGCTGTGACCGCATGGGCCGTGCTCCATCAATATTGT | |
| | TTTTTTGTGTGTGGG | |
| cg071 | TACAGCCTTCCGGGAGCTGGACGGGGCCTCCCCAGCTTTGGGCAGCTTGGGAC | 132 |
| 80649 | AGTGGCC[CG]AGACTGTGGGAATCCGAAACCTCGCTTCTGGCTAGCCACAAGG | |
| | TCTGGGCGCGCCCCAGG | |
| cg072 | TCCCATTCACAGACAAACTGCTAAAAGCAAAACCAAAACTTTCCAAATAAGCCA | 133 |
| 11259 | GGCTTT[CG]TCAGTTCCTCAGAACTAGTTCTGGTTTGACTCACTCTCATGTTACG | |
| | GCAAACCTTAAGCT | |
| cg072 | AACGTGCGGTTGCCGTGACTAAACGCATTCATTCACCCTACAAGATTTAGGAAA | 134 |
| 36943 | ATGTAA[CG]TTGCAAGGGAAGCAAGGTCTCTGTGTAAACCTCGTAATCGCCAC | |
| | CAAAAGTCGGTAGCTG | |
| cg072 | CACATTTCCCGCACAAGTCCCCAAGCCTTGGACCCCCCTCATCAGGACCTCCGG | 135 |
| 65300 | CACAGG[CG]CCCGTTTCCCGCCACTGCCTTCCAGTGGTTTGGTCCCCGAGCAGG | |
| | ACCCAAGGCGGGGCA | |
| cg074 | GCCCCTCCCTCTCTGCCCTTTCATTAGCTTAATTACACCGTGCCTATGACAACAG | 136 |
| 84827 | AGCAA[CG]GAAACTGATACCTCGGGCCTCTGGGGCTTGAATTATTCAAACTCT | |
| | GTAAAGCAGCACACA | |
| cg074 | CGTGGGGCTGGCGGCGCGGATGCCCTGGGGCGCTGCAGACCCCGAGAGGCC | 137 |
| 94518 | GCTTGCCCG[CG]GGGACGTCAGCCGCTTTTGCTGTTAAAATCTGAAATGTTCAG | |
| | CAAGTTAGAAACTTGAAA | |
| cg076 | CCGGATGAGCAGTGACTTCAGGGCTTGGGCTACTCTGGCTTAACGGGACCAGT | 138 |
| 54934 | AGCAGAG[CG]CCGCCCGTCCTGCTTGCTGCTGGGTCCGGTTGCCGAGGCGGA | |
| | AAAGTCGCAAGCTCCTTC | |
| cg078 | TCGGTCAGGCGTGTGCAGACAGCGCCTGCAGGTCTGGGTGGGTGCTGATCTG | 139 |
| 17698 | AGTGTCTG[CG]CCTGGGCCATGTTTTTGAGCCTGGCACAGGGGTGCTTAGTGA | |
| | ACACATGACCGCCTAGCG | |
| cg078 | TAGTTCATCTGCTGGCCGGCTCTCAGTCCCCGTGGCGCCCCCTTTCCTCTTGTCC | 140 |
| 50604 | CAGAG[CG]CTCTCGACTCCACCATGCCAAGGGGATTCCTGGTGAAGCGAACTA | |
| | AACGGACAGGCGGCT | |
| cg079 | AGGGAAACCGAGGGCCAGGAAACAACTAGAATCCGACGGTATTTCCTAGCTCC | 141 |
| 29310 | CTGATGG[CG]CTTCCCATGCCCCCAACTAAATCATGAAATAACCCACTCACCTG | |
| | TTTGCACCGGGCCTGC | |
| cg080 | CCTTTAATCTTTTTGTTTTGGTTTGAATCTGCTCGGCGCAGACTGGCCAAGGATC | 142 |
| 35942 | CTCTC[CG]CCCTCCCCCTTCCTCCTGGCGCGGGAGAGGCACCGGATATCCCCAC | |
| | CCTCCCCGAGCTCT | |
| cg080 | TGTGCCTCAGGCTTATAATAGGGCCGGTGCTGCCTGCCGAAGCCGGCGGCTGA | 143 |
| 67365 | GAGGCAG[CG]AACTCATCTTTGCCAGTACAGGAGCTTGTGCCGTGGCCCACAG | |
| | CCCACAGCCCACAGCCA | |
| cg080 | CAGACCTGCCTTAAAAGCAGCTTGCCCGCCTTCTCTCCTCCCCTCCGGGCGGGC | 144 |
| 74477 | CCTGCA[CG]TGGCCCTGACAGCAGTAGGCCCCACCCCTGCTGGATCCAGTGAG | |
| | CTCAGGTGGGGCTGGC | |
| cg081 | AATGTGCTGGGTGCAGCTTTGGGTAATACATATGCCGAACCTTCTCTTTAAGGG | 145 |
| 69325 | TCCACG[CG]CAGCCTCGGGTGTGAATGAAGGAGAAGAGATCGTGTACCACAC | |
| | ATGATGCTTACGGAGCA | |
| cg082 | GCGCGGCAGTGGCCTCGCAGGGCGCTGGGTCCCTCTCCCCAGCTCTCCTCCCCC | 146 |
| 12685 | TGGCCC[CG]TCGCCCCGCCCTCGCCGGGCTGGGCTGCGGGGTCAGGGGCCGA | |
| | GCGGAGAGGGGTGAGTA | |
| cg082 | GTGAGTGAGGGGCTCAAGAAACTCTACAAGAGCAAGCTGCTGCCCTTGGAAG | 147 |
| 51399 | AGCATTAC[CG]CTTCCACGAGTTCCACTCGCCCGCCCTGGAGGATGCCGACTTC | |
| | GACAACAAGCCCATGGT | |
| cg083 | TCGGGGTCCCTTGGCCTGGAGACCCTTTGTCCAACCCGTCGCCCACCTCAAGAC | 148 |
| 31960 | CTGCCT[CG]ATGCTGCGCATACAGTAGGTATCCAATAAATGTTCCTGGGATAG | |
| | AAGGCAAAGGCGCTGG | |
| cg084 | AGCCAGCAGCAGTGCCATCATCCCGTGCCCACCCACACGCCCCATCCAGGGTG | 149 |
| 24423 | CCCGAGA[CG]AGCCCATCTCGGACTGCACGGCCTCCTGACTGATGGCAGCTCA | |
| | AGGACACCCGGGTCCTT | |
| cg084 | CCACTATGTTCAGTCTAGTGAGTCTGAGCAATTAACTCACATTTTGAATTTCAAG | 150 |
| 75827 | TCTCT[CG]CCTTAGGCAAAACACCACCACCTGATGCTCACCAGAGGGGCGTGA | |
| | CGCGGCAGCTGGGCA | |
| cg084 | TGGGCAGTGGCGGGGCACGCAGGCGGCGATCAGAGGCTGTCCCGTCCTCTCC | 151 |
| 87374 | GGGGGCCG[CG]GCTCATCCTGCCAGGCATCTCCGAGGAAAGTTTGCTCTCCGG | |
| | AAAAGAAGAAACCCGCGC | |
| cg085 | AAACATGGATCAAGAAACTGTAGGCAATGTTGTCCTGTTGGCCATCGTCACCCT | 152 |
| 29529 | CATCAG[CG]TGGTCCAGAATGGTAAGGAAAGCCCTTCACTCAGGGAAGAACA | |
| | GAAGGGGAGATTTTCTT | |
| cg085 | AGCGACAGAGCAACGTCGCACTGCATTCTTACCAAACACCCAGGTGAACGACG | 153 |
| 86737 | CATCCAA[CG]ATTTGGGAGCTCAGGACCCATGGTCCCTAAAAGGCAACAATTA | |
| | AGACTCCCATTTAGACC | |
| cg085 | GAAGAGAGGAGAGGTTTAGAGTCAAAGAGCCCCAAACATTAGTGAGAGTATA | 154 |
| 87542 | TGTATGAA[CG]TTTGGTCATCTTAGAACAGTGGTTGGCATCCACAGGAGACCA | |
| | GCAGAATCACATGGGCGC | |
| cg086 | GGGGATCCCCAGTTGCCAAAGGATGGAGGGCGGAGCTGGAGGACCTCAGGCT | 155 |
| 54655 | AGTGAGCA[CG]CCCTTGCCCAGGCCTGCAGTGGCTGCACTCGCCAGCTGGCCC | |
| | ATGGCCCTGTCCGACTCC | |
| cg086 | TCTTTTTGTGACTCTCAAGGAAAGTCGGTTTTCTGAGCTCTTACTGGCTTAGTAG | 156 |
| 68790 | CGTGG[CG]TTCAACGCAGAGCATTCTAGGTAATGTAGTTTTCATAGATCCCGA | |
| | GGTGGGTGCCGGGGA | |
| cg086 | GAGAAGGGAGGCAGCTGCGGCAAAGTTAGAGCAAGTACTGCAGCAGCCAGG | 157 |
| 94544 | TTGGGTCCG[CG]CCGTCGGGTTTCTGAGAAAAGGGAGGAAAGAGGCGGGGC | |
| | CTGCACGGTGTGTCCCCGCCC | |
| cg088 | TTCCTATCCCACTGATCGTTTTAGAGCCTGAACAGACAAAACATCCTGGTTACC | 158 |
| 72493 | AAGACT[CG]AAGAATGCATAAGCTGGGACCAGGCAAAACAAACAGATCACTG | |
| | TGGGCTCACAGAGCAGG | |
| cg088 | GGGCAACGCGGCCGGATCCTGGAGTTCCCCTCCGTGCTGTGGAATTGGGTCAG | 159 |
| 96629 | GCGTGTA[CG]GTCCTGACCCTAGGACACAGCTGCATGTCCTCACCTCGGTGTTC | |
| | AAAGCTGCACCGGCCA | |
| cg088 | GGGCGAGGCGTGAGAACGAGCATTTCTAAGTTCCCAGGTGATGCCCCTAGTGT | 160 |
| 99632 | TGGTCGG[CG]TCCACACTCTGAGGACAGTGACCTCTCTGCTCTGTCCCTCATGT | |
| | CTTACTACTACTGTCT | |
| cg089 | AATCATCAAGGCCATTTTCAAATCCCATTGGTCTAGCCGTCACATGGTGAGAAC | 161 |
| 00043 | CGAATG[CG]CGGATAATTACGGAGCTGATATTTCCCCCCCTCCCCTTCTTTTT CC | |
| | TCCCTCCCCTCCAA | |
| cg090 | ACCGCATACAGCACAACTCAAGTTTGCATCAGACTGGGAAGCGAACTTAAGCC | 162 |
| 45681 | AGCGGTG[CG]TGGCCCAGGAGTGGGAAAGGAAATGGATGCCTGAAGTGGAA | |
| | GAGGTGGTGCAGAGGGGGC | |
| cg090 | TTTTATCTGCCCTCGGTACGCTGATTTCCAAAACCCAGCCTCATATTCTATACTC | 163 |
| 96950 | CAAAG[CG]CACTGCCAGGTGGGCCAACTCCAGCCCCCACAATCCGATGCCAAG | |
| | GCCACTTCTTGCCAC | |
| cg091 | GTTTATAGTGGTCTGGCTTTTGGCCATGACAATGACACCTTGCCCTTTTAATTTG | 164 |
| 96959 | GGGCC[CG]TGCAAATATTCACTGAAAGCTGTCAAGAGGAAAACAGAATTGGTT | |
| | ATTGAATCACTTGCT | |
| cg092 | GCCCCCTGGCGGCCACAGCGCAAGCCCGGTCTCTCCTCCTGCTGGAAGGACAC | 165 |
| 54939 | CGGGGAC[CG]CACCTCCAGCTGTGGGAGTTCCGAGAGACCCCGCCCTGCCCGC | |
| | TCCTCCCTGGAGGCCGC | |
| cg092 | AAGTGCGGCCCTTGGGCCCGCAGCATTAGCCTCATCAGGGTGCTGGTTAAACA | 166 |
| 94589 | CACAAAT[CG]TCAGACCTCCACCCCAGACTTTCTGAATCAGAAACTCTGGGGGC | |
| | ACAGCCCAGGAATCTT | |
| cg093 | CAGGGAAACGCGGGAAGCAGGGGCGGGGCCTCTGGTGGCGGTCGGGAACTC | 167 |
| 04040 | GGTGGGAGG[CG]GCAACATTGTTTCAAGTTGGCCAAATTGACAAGAGCGAGA | |
| | GGTATACTGCGTTCCATCCC | |
| cg093 | ATTTCCATGATAAAGTATCGTTTCCCTGGTAACAATAGCATTGGTCTTGAGAAG | 168 |
| 22949 | CTTCTC[CG]ATTGCAGCAGGACCTTTAAGCTGAGAACTGAAAAACGAATGGGA | |
| | AGTGTTATGAGCAGAA | |
| cg094 | TCCTCGGGAGACAGGGTCTCCAGCAGGCTGTCGATGTCGGGGTCTTCACTCAC | 169 |
| 04633 | CTGCCGG[CG]ATATTTGGCTACTCTAGACATCTTGGCAAAATGGGCTGTGGCT | |
| | GCCAGGGGCTATCAGAG | |
| cg094 | CGGGATGGGGGAGCCCAGCAGTGCCCACTGCACGCCTGGTGACGAGTCTCCC | 170 |
| 13557 | CTCATCTG[CG]CAGCTCAGTTTGCTCAGTTTGCTCTTCGTGACACGTGACTCGG | |
| | CAAGGGGAGCAGGAGGA | |
| cg094 | AAAAAAAAGAAAAGAAAATACTTGATGGAAGGCTGCCATCACCATGCTGCAAA | 171 |
| 34995 | ATCTCCA[CG]CCCCTGCTGCCCGCACCTGTCCTTCCTCCCTCCCTCCTCCCCTGG | |
| | CCTGGGGAAGCCCCT | |
| cg094 | GAGGGAACACATATAGAAGGGATTAAGGGGTAGTTGATGACTCTTTGGGAAA | 172 |
| 80837 | AGAGGGTA[CG]GGAGAAGCAAGGGGAAGAAAGACATCTATTTGTCAAAGAG | |
| | CAAAGGCAAGGCAAAGCTGG | |
| cg095 | GAGCCGCTCGCTCCCGACACGGCTCACGATGCGCGGCGAGCAGGGCGCGGCG | 173 |
| 48179 | GGGGCCCG[CG]TGCTCCAGTTCACTAACTGCCGGATCCTGCGCGGAGGGAAA | |
| | CTGCTCAGGTGGGCGCGGG | |
| cg095 | GGAAGCCCGGAGCCGCCCCTCCCCGCTCCCCCGCCCCGCCGCCCCGGACGGAC | 174 |
| 56292 | GGGCGCG[CG]GAGCCAACCCCGCTGCCGCTGGCTGTCCAAATCCCACCAGAGC | |
| | CAATGGGAGCGCGAGGG | |
| cg096 | GGAACGGTTCCGGCAGGGTTGGGTTTCCAGAGCTGTCCAGGGGCGCCTGGTG | 175 |
| 30437 | CTGAATCC[CG]CTTGGAAAGAGGCTTGGAGGTGGATGGGAAGGGATTTCCAA | |
| | CGGAGGCGGCTCCTCTCTC | |
| cg097 | CGCCTCTCTGGACCTCTTTTT1CATCTGTAGCTTGGGGATAACACTGACTAACAT | 176 |
| 99873 | GGCCA[CG]CTGAGCACTGCAAATCTAGCCTGATTGCCAGTCAGAATGCACGCC | |
| | CGGCCTCGCTGTTTC | |
| cg098 | CCCCAGAGAGCTTTCATCTAGAAGGTTTGACTCTGGCCAGACAACCAGCGAGC | 177 |
| 09672 | ATCTTCT[CG]CAATCTGTTGCTTCTTCCATGGCAAACTCCAGAGAATTAAGAAG | |
| | CCAAACTCAACATCGC | |
| cg098 | CCAACGGGTGAAGAGCCTAGGTGIIII1GATCTGTGCCTTCTCTGTTCCTCAGA | 178 |
| 51465 | GATATG[CG]GGCGTCCTTCTAGAAGCCCATCTCGCTCACCTGTGTGGTCACCCT | |
| | TGTCCCGCCCTTCCT | |
| cg098 | TCGCCGCCTTCTCGCTCATGGCCATCGCCATCGGCACCGACTACTGGCTGTACT | 179 |
| 92203 | CCAGCG[CG]CACATCTGCAACGGCACCAACCTGACCATGGACGACGGGCCCCC | |
| | GCCCCGCCGCGCCCGC | |
| cg100 | CTGTTGACCCGCAGGACTCGCTGGATGTTGAGGTCGTCAGCACCTTCTGCGGG | 180 |
| 52840 | GGTCAGG[CG]TCCGGGCCCGCTGCCCACAAACACGGGATAGTGGTTCAGGTCT | |
| | GAGTGAGGGGGTGGAGA | |
| cg101 | GGCGGCGGCGCCAGGACATGGAGCTCGAGAACATCGTGGCCAACTCGCTGCT | 181 |
| 58181 | GCTGAAAG[CG]CGTCAAGGTGGGTGCGCGGCAGGCGCCCCCGACCCCCCCCC | |
| | CAGAGAACCCCGAATCCCG | |
| cg102 | TCGCCCCCAGCCCACTTCACTCCATCACTGTCTTCCTTAGAGTTTATCCAGAAGG | 182 |
| 02457 | CAAGA[CG]TGGTATCCAAGCTCAGAACCAAGAGCCCACAGCATGGTGTGAGCT | |
| | CTTTCTGCCTCTTGC | |
| cg102 | GTGATGTTTAGAACCTTTTGGGGGATTCCTTCTCTCTCAGAATTTAACCTGGCA | 183 |
| 25525 | AGAGAA[CG]ACTGAGTTCTAGGAATTTTCTTGTCTGGAGAGAGTAAAATAAAT | |
| | GTATTTTTTAAAAGCT | |
| cg105 | CTCGCTGCTTCTCCCCTAGTCTTCGGGTCCCTTGAACGCAGGTCGCTTGTTTGCC | 184 |
| 23019 | TTACG[CG]TAGTCAGCGGCCAGTGGCTATTTATGGCAGTAAGGAATATTATCC | |
| | ACATTTCACATGGAG | |
| cg105 | TAAGCTGTCCAGACCTGGCTTGAAAACCCATCCCATGGCAAGGCAGGGATTCG | 185 |
| 70177 | CTGGCCG[CG]GTTGGCTCTATCTTGATCTGAGCAAGCCGCTGGACGTCCCTAG | |
| | TTATCTTCTTCCTATCC | |
| cg105 | CTCTCTGCAGCCCAGGAACAATAAATACTTCCTCCCCATGTTTAAAAATAACCCC | 186 |
| 91174 | ATGAC[CG]CTTTTGGCAGTCATAGGTGAGGCGGGCACCACCTAAGGCCCCCCC | |
| | ACCCCATGCCGTTCT | |
| cg106 | GAGAATCTGAAAATGAGACCCAAGCGAAAGTATAGACATTTTATTGTGGAGCA | 187 |
| 36246 | AAACCAA[CG]ACACCCTCAAGGGAGGAGTGCAGGCACTCAAAGATTTGAGTC | |
| | ACAGGCAATGTGGTTCAC | |
| cg106 | CTCAACAAGGCCTGCATCTCCGGACTGGAGCTCAAGTATAGCCCAGCGAGTGT | 188 |
| 54016 | CAAGAAA[CG]AAATTCTCCAAGGGTGGCGGAATCAAGCCCCAAGTCCCATGTG | |
| | TCACTGGACCGGTGAGG | |
| cg106 | TAGCCACCTCCTAGCACCTCAGGTTTTTTACCTTTGAGTCTATGAAGCCTGCGG | 189 |
| 67970 | AGGTCA[CG]CCCTAGGGAAAGAAGGAGCCCACTGGGTGTCAGGTCCTGCCTCT | |
| | AGGGAGGGGACCGCGG | |
| cg106 | GAGAAGGGCGGTGGAGTGGGACTTCCCGCTGGCCTAGAAAACTTCAGCTAGG | 190 |
| 69058 | GCTGGGGG[CG]GTGGCTCCTGCCTGTAATCTCAGCACTTTGGGAGGCTGAGG | |
| | CTGGAGGATCGCTTAAGGC | |
| cg107 | ATATAGTCCTATTGGAACCCAGATAAGCTTAGTCTCAAAGCCTCCCCTCTTGTCA | 191 |
| 95646 | CCACC[CG]ACTCTGCCTTACTCTTGGTAGAACCACAGCGATGACAGCTGCTTGG | |
| | GAACATAACCACAA | |
| cg108 | GGCTTTTCCCTTTGACCTTAACACTTTTGGGGTTATCTCTGAGGCGAATGCTAAA | 192 |
| 78896 | GGAGA[CG]CTCCAGGACTCGACCTCTGAAGGTCCTTGGAGCCAATTCCGTAAT | |
| | ATGATCATGGAAACT | |
| cg109 | AGAGACTGTGTCCACCGTCATTGAAAGGGTAATGCTTGAGAAAGGCCTGAAG | 193 |
| 00550 | GATATGGG[CG]GACAGAGTGTGTGTCTAGGGCAATAAAAAGTAACTGCTCCA | |
| | GATGTTGAAGAAAATAATG | |
| cg109 | AAGAGGGCCCCTCCAGGCCAGTCTGGGCACCCTGGGATAGCGGCTGCAGGTA | 194 |
| 17602 | GGCAGAGG[CG]CTGCCAGTGCCCAGGTGGCCTTTCCCTCCATCCGGCCCTTCCC | |
| | ACCTTCCTATAACCTTC | |
| cg109 | GAGGCAGCAGTAGAAACAGTTTGCTCCAAGGACCAAACTTATTCTGGTGTGCA | 195 |
| 22280 | GCTCACT[CG]CCCCTACTCATCTCCAGTGTATTTCAAGAGTATGCAGGGAAGGA | |
| | AAAAGTCAGGCTGAGT | |
| cg111 | AGCGGACGCCTGGGCCAGGCCTCACAACGTCCAGAGCTGGAATGGGTCTTTTG | 196 |
| 77450 | CTTTCGG[CG]CAGGGGTGACGGGATCAGCGGAGGGTAGGGGTGTACACTAGC | |
| | TGCGGTCTGATTTAGCCC | |
| cg112 | AGGGCTGTCGCGCCTGCCGTGTGGTCCTGGAGAATGAGGCTTACCAAAGGCTC | 197 |
| 33384 | AAGACAG[CG]TCCCCATGGAGTGACATGGTTAAAGTGTTGAAAGAAAAGAAC | |
| | TGTTGGCATTGAATTCTG | |
| cg112 | GGTTCGCTGACGCTCAGTGTTTTGGCCCGGACGGTCACATGTTTCCTTTGTTGT | 198 |
| 37115 | GAGCTG[CG]GCAGAGACTGGTGGCTGGAGGAGACGCCGGCGCTGGAGAGTG | |
| | CGCTGCGCCGCCCGCCGC | |
| cg114 | CCAGGGAAGCGAAGCCCAGCTGTTCCTTCGGGGTGTGTACTTGGAACTGCATC | 199 |
| 26590 | CAGGTCT[CG]CTTAGGGTCCCCGCGGCGAGGCGGAGCAGCTAATTTGAGAGC | |
| | ACAACAAATAAACAAGGA | |
| cg114 | TGGGGTTGGAGCTGGGCTGTGGCACTGGACTGCGTTCGGGGACGGGGGACG | 200 |
| 59714 | CAGCCAGAA[CG]CGAGGGTGGTAGGGAAATATTGGGGGTTTCGCGTGCACCG | |
| | AAGGGAATGGGAGGAGAAGA | |
| cg114 | CGGATCGCGGGGAAGTTCCTCTCAGCGCCTCAGGTGTCTGGGCGTGTGCAGCT | 201 |
| 87705 | GTGTTGG[CG]CACACTTGCCGCTACAGCCCTTCTGTCAGCCCTTTAGCTTCGAT | |
| | GGGGCGCTGGTGGCCG | |
| cg114 | TAAAGAAATGACAGGTGTTAAATTTAGGATGGCCATCGCTTGTATGCCGGGAG | 202 |
| 90446 | AAGCACA[CG]CTGGGCCCAATTTATATAGGGGCTTTCGTCCTCAGCTCGAGCA | |
| | GCCTCAGAACCCCGACA | |
| cg116 | CATAAAAGAGGAGACATAGGGGGCTTGGTGAGATACCCTGAAACCTCCCCCCT | 203 |
| 00161 | CTGACCC[CG]CAGCCAGGCCCCAGGCTGGCCGGGAGTGGCCCCTCACACTGGT | |
| | TCTCCCCACTTTCTCTG | |
| cg116 | CCTCGCGCTGATCTTGGTGGGCCACGTGAACCTGCTGCTGGGGGCCGTGCTGC | 204 |
| 18577 | ATGGCAC[CG]TCCTGCGGCACGTGGCCAATCCCCGCGGCGCTGTCACGCCGGA | |
| | GTACACCGTAGCCAATG | |
| cg116 | GAGTGGGTGGGTGGGTCTGGAGAAGCTATGTGTACCAACCAGGTTCACATATT | 205 |
| 31518 | TTTCTTC[CG]TGAAGCTCTGTCTCCACCCTCTCTGGAGCTTCTGCCTGCCTTATTT | |
| | ACACCCCACTCTCC | |
| cg118 | GGGGGAAAGTAAGGGAGAGAGAAAGAGACGGAGAAAAACAGGAAAACTTAC | 206 |
| 33861 | TCTTCAGTA[CG]CAGGGAAGAATAGAGAAAGAAAAACACAAAGAAACGCCAC | |
| | GCAGACTGCAGAGAAGGACC | |
| cg118 | TCGTCGGGGAGTGAAAGCAGGCGCAGGGAAATAAAAAGAAGGAAAGGGAGA | 207 |
| 96923 | CAGACCAGG[CG]CCTAACAGATGGGGACCAAGAAACAAGAGATAGCTGAGAG | |
| | GTGCAAACAGAAGAGAAAAA | |
| cg119 | GCCAGCCCAACTGTTGTATTTTCAGTTCTTCCAGTGTGAATCAGTTAATATTCTC | 208 |
| 03057 | GGGAA[CG]AGGGAGAGGTTGATCCTATGAGGAAATCAACCACAGTGAAAAGG | |
| | CTTGGGCCGCTTTTGT | |
| cg121 | TGGCGGTGGGCTACCCTTTTGTTCCTCTTTTACCACCTGGGTTACGTTTGTGGGC | 209 |
| 45907 | AGATC[CG]CTACCCGGTCCCAGAGGAGTCACAGGAAGGGACTTTTGTAGGGA | |
| | ATGTCGCTCAAGATTT | |
| cg121 | CCTTGCTGGCTCTGTCTGCTGAGGTTTTACCCAAGTGACTCCATTTTGAATCTTA | 210 |
| 77001 | CAACT[CG]CACACTACTCATGTGGAAGATTTAAATGTACATTCCAGGACCTGGT | |
| | GCTTTCTCTTCCGC | |
| cg121 | GTGGCCACAGAATCCCCTTCCTACAACTGGCAGGGGTCGGCATGGGCTGGAGC | 211 |
| 88560 | TCAGAGA[CG]GCCAGCTAGGACTTCAGGACACACAGCAAACTAGCTGCGCCCC | |
| | GCTGAGGGTCAGCGCAC | |
| cg122 | CTGACCTCCAGGAAGCTGAGCGTGGTGGATGGAACTCTACGATCTCTTTCTCTC | 212 |
| 38343 | CAAGGA[CG]GAAACCTCATCCAAGCAGTCCCAGAGGAAACGGATAAAGGTAT | |
| | TTGAAAGGGAGCGAGCG | |
| cg122 | GCCCTGGCAGTGCTCTCGCGGTGGCCTGGCTCTCTCTCTCCGGCCTGAAGGAG | 213 |
| 47247 | AGCAAAG[CG]CCCCAGCTGCCTAGGGCCACCGCTCCTGACGAATCCGCCAGCC | |
| | ACTGCACGACAGATGGT | |
| cg122 | TATCAACAAAAATACCCACTTCAGGAGGTGGTTGTAAAGATTATACAAGAGAC | 214 |
| 61786 | TGCAGAG[CG]TTAGGCAGCACCTGGCACAAGACAAATGCTCAGTAAAAGACC | |
| | ACTGCTGTCATTAAGGTC | |
| cg122 | CTGTCACAATTGTTAACACCTTCTTTGACCAGCCTTTTTACATTTGACAATTCTCT | 215 |
| 65604 | TCAG[CG]CCTCTTTCCTGCCAGCAGGAAGGTTTTGCTGCCTTGGCTTTCGGGAG | |
| | CCCCCTAGACAGC | |
| cg122 | CACGACTCACGGACATGGCCCCAGCTAATTGGTAGCCCCTGGGTTCAACCGGA | 216 |
| 69343 | ATCAGCG[CG]TGAGTCCAAGACTGGGAGAAAGAGGCTCATCCGAGACTACAA | |
| | TTCCCAGAATGCGCTTCA | |
| cg122 | GTAAACAAGCAAACAAAAACACATACACAAACCGGTCACTGTCAGACTGTCTG | 217 |
| 89045 | TGAGAAG[CG]CTCCACAGGACACAGCTGGAGAATGTGTCACAAAGGAACTCA | |
| | GAGGGGGGCGGTCAGGGA | |
| cg123 | CAAGAACCTGGACACCTTCTACCGGTAACAATGGGGGTGTGGCTTGCTTCTTTG | 218 |
| 24144 | GTGCTC[CG]CTGCTCAAACCTCTAGGGGGAGCATGCAGACGGGCAGGTTGTG | |
| | GGGCACGTGGGCTCCGA | |
| cg123 | TGGCGATCCAGGAGCACCAGTACAGGTCGGTGACGGCGATGAGGTACAGGTC | 219 |
| 73771 | CAGCAGGC[CG]CCCTGCGCCAGCAGCAGCACCACGGACAGCGCCTGGTAGCC | |
| | CCAGCGGCACCTGGGACTG | |
| cg124 | GGAGGGATAATGGGATCAGGAGGCTCAGAAAAGGGCAAAGAATGGGAAGGG | 220 |
| 02251 | GCATGGAAA[CG]GGTCTTGAAACAGTTAAAAAGAGAAGATAATCACCGTCAG | |
| | CGTCGAAATGGAGCCAGATC | |
| cg124 | GCCTAACCCGGCCTCCGAGGGGTGTCCCAGCGGGGCCTGGGGTCCAGGGCAG | 221 |
| 73775 | AGTTCTTC[CG]CCCCAGCCATTGGGAATGAAGGCCTCAGTGATGTTATCTGTAA | |
| | AGCCGGAGGAATGGCAT | |
| cg127 | GAGGGGGATTTCCAGCTGCTGGCCGGGGCCTCTCACCCCTACCCCCGCGTAGT | 222 |
| 43894 | TCATCTG[CG]ACGCAACGCCTTGTGTCAAAGCCCAGCACAGGTTCTGCCGCCTC | |
| | TGACCTCTCTGAGGGT | |
| cg128 | GGTGACGGTTACAGGCGAGTCCTCTCTTTGACATACTCAATTAAGCTCTGTACA | 223 |
| 13792 | CTTGAG[CG]TCTGTCCACTCGTAGGTGTGCATACTTCCACTGCGGATTTAAACT | |
| | TTCAAAGAAGTCTAG | |
| cg128 | ACTCAGACAGGCAGGAAGCTGAAGGCAAAGGAACTCTCTATCTGATTGGTTTC | 224 |
| 64235 | CATTCAG[CG]TTTCTGATTAATAAGAGACGTCCCTCAAATAGGAAGATATTGCC | |
| | GCTGATGGCGCTGCAG | |
| cg129 | ATTCACATTTAGTTCGCCTAGGAAAACTAGCAGTTAGTGAAAAACTGGCCACAT | 225 |
| 85418 | CACAGC[CG]CACAGCTCCAGCAGCCCGGGTAGCTTCCCCACCCTCACTTTCTCC | |
| | AGCCCCGCCTCCAGG | |
| cg129 | GGCATGCCTGCACCCTCAGGGCAGCCCCGGACATCGGCGTCAGGTTGCTTGAG | 226 |
| 91365 | TCAGGGG[CG]TGGGAATCAGACAGACCCGGTCTCAGATGCCACCCTGTACTGT | |
| | TGGTTCTGTCATTTATG | |
| cg130 | GACTGGGCAAAAATTAACCAGGGCTCCAACAGGCGAAGGTCACTGGACTGGG | 227 |
| 42288 | CAGGGGCA[CG]CTCCGCCTGGGGAGAGGAGATCCAGGAACGGTGTGTGGAG | |
| | CTGGGCTCGGGGGGTGCCTG | |
| cg131 | ATGGGGGTTGTGGCTGTGGAGCGGAAGTGGGTCTCAACCACTATAAATCCTCT | 228 |
| 19609 | CTGTGCC[CG]TCCGGAGCTGGTGAGGACAGCCTGCCAGAGTCTGGTAAGAAA | |
| | GGGACTCAGGGTGCGGGG | |
| cg131 | GACTCAGCCACTGGTGTAAGTCAGGCGGGAGGTGGCGCCCAATAAGCTCAAG | 229 |
| 20519 | AGAGGAGG[CG]GGTTCTGGAAAAAGGCCAATAGCCTGTGAAGGCGAGTCTAG | |
| | CAGCAACCAATAGCTATGA | |
| cg132 | GGTGGCCGTCCGCGTGGCAGGGCGGGGGTCCCAGGGCGGCTGTGCTTGGTGC | 230 |
| 18906 | TGGGAGGC[CG]CCCGAGGGCAGCGCCGGCCCCGAGTCAGCAGCCGCAGGGC | |
| | ACCCTGGAGATGCGGAACGC | |
| cg132 | AGACCCTACGTAGGATTGCATCTTTACGTCGTAGGCTTGGTCTCGTGTATTTTTA | 231 |
| 58700 | TTGAG[CG]TGTTTAATTAGCTGAGGTTACTCGCTTTGGCACCCCAGTGATCGTT | |
| | TTTGCCACCAAGCT | |
| cg132 | CTCTCCCAGCCGTTTCCCAGCGTGTCATGTGCTGAGAAATGGTGGGCTTAGCCA | 232 |
| 96371 | CGCAAA[CG]TTTACTGAGCATCTACTATTTGGTAGGAGCTGTTAGGCACCATGC | |
| | TAGCAGTGGAGATAA | |
| cg133 | GGGGACCAGTTTCCCCTCCTGGGATATTTGGTGTGCGACATGCCCTTCCCCCAG | 233 |
| 07384 | CCCCAG[CG]CCCGTTCCCTTTGGAAGCCTGGGTGCTCCTCAGACCACTTGGGG | |
| | ACTCCCTGCTTCACCT | |
| cg133 | CTGTCTTACCTGCAGCAGCCTCAGTTATGTTTTTGACAACTATAGCAACCAACTA | 234 |
| 23474 | CCTCT[CG]CGAGAACTTACTTTTGGCCATCGCACGAGCAAGTTTATTCCAAGAC | |
| | TCGCGATAACCCTC | |
| cg133 | CAAAGTCACTCAGCTCGCCAGGGGCAGAGCCAGGGTGCTCACAGGGTGGCCA | 235 |
| 51161 | ACCTTCCA[CG]TCTGCCCTGGACACGGGACTTTCAGTACTAAAATGTGCGGAC | |
| | GTCCTTCTCCCGGACGTC | |
| cg134 | TTTTCCCGGGCAGCTTACCTGCTCGGCCTGGGTCTTTCTGGACAGCAGGCGCTG | 236 |
| 09216 | GAGGTG[CG]CGTCACTGTCCGCCGCCGTGTCCGCGGCTGCGCCAGACAGTGTA | |
| | GAACCTGCGGCCTCGA | |
| cg134 | AGAGGCTAAATGCCAGGGGGATGGAGTGAGCTACGAGGAAACCACTATTCCC | 237 |
| 49372 | CGACCCAG[CG]CCTACCACAATCTGTTTGGATTACCACTGATTAGTCGTCGAGA | |
| | TGCTGAGGTGGTACTGA | |
| cg134 | ATCTCTCACCTTGCTACTTTCTCGGTAGCCGTTTCTGTTGTCCCTGGATTGGGGG | 238 |
| 60409 | CTCGG[CG]TTCGCTGTCCCTGGGCACCAACCCTTTTAAAGACAGTAACGTTGTA | |
| | GGAAATCAAATTAG | |
| cg135 | CTGTCTCTCCACCCTGTCCACCAATCAGCACCTGGAGGTGGGCTGGGAGCTGC | 239 |
| 09147 | CTGTGAC[CG]CTTCAGCATCTTTTGGGAGTGGTGACAGAGCCACAGAGGGCTG | |
| | TGAGCTTGCCCGGCCCC | |
| cg135 | CGGTCCGGGTTCGCTTGCCTCGTCAGCGTCCGCGTTTTTCCCGGCCCCCCCCAA | 240 |
| 10262 | CCCCCC[CG]GACAGGACCCCCTTGAGCTTGTCCCTCAGCTGCCACCATGAGCG | |
| | GTAAGGATGAGTCCAC | |
| cg135 | CGGGCAGCCGCGGGAAGCTGGTGATGCTCATGTAGTCCACTGGCGAGTAGGC | 241 |
| 14050 | GCCCAGGG[CG]CTCTCCTGGCTGGCCTCGTTCTCCGCCGCCATCCTCGCCCGCG | |
| | CCCCCCAGCAGCGCAGC | |
| cg135 | CCAGATTACAGTCTCTAAGTCTTAAAGAGGCCAGCCCCACTTAGAGGTTTTCCT | 242 |
| 50877 | GAGCTG[CG]TATCAGGACATGAGTTCCTTCCACTATTTCTAGGAGTACTACACT | |
| | AGAGCAGTATGAGAC | |
| cg135 | TAGGGACTGTTCATCCATTGGTGTTTGTGTGCAAACTAAGACGACTCTGTTCTG | 243 |
| 64075 | CGCAGG[CG]TGTTGGGGGTGCTCCCCCTTCCTCTCCATAACACAGACGCCTCCC | |
| | GCGCAGGCGTATTGC | |
| cg135 | ATTTCCATATATCCTTTATCCAGATTCCCTAAATTATTACTGCATTTGCTTTATCC | 244 |
| 71802 | TTCT[CG]ATCAGTGGACAGATAGATGATATAGACAGATAGATAGACAAATATA | |
| | TATTTTTTGAACTC | |
| cg135 | ACCTGCCTGGGTGCAGGACCCCAGAGGGACCCCAGGCCACCCCTGGCCTGCCC | 245 |
| 87552 | ATGCCCA[CG]GGAATCCCGACCTTGGGCTGCCTGTCTATTGCACCAGAACCGTC | |
| | CCAGGGCTGACTCAGA | |
| cg136 | GACAAAGTGCAGGGGATATAGACCAACCGCTTGTGAAGGCTGCTGGTTCTGTT | 246 |
| 13532 | AGAAGCC[CG]CTTTCGATTGTCAGTGGCTTTGAGGCAAAGGATTTTGGAAGGG | |
| | AAAGCAAAGTGATTGTC | |
| cg136 | GGACGTTGGACGTCAGCAAGGCCTGGGTGGTGCTGGTGAACGGTGATGCTTG | 247 |
| 31913 | CGGCCACA[CG]CGGGGTGGCTAAGCCAGGGACCCGAACTCATATGAGGTCTG | |
| | GAAGGTGTGTTGAGACACT | |
| cg136 | AGGCGGTGAGGGGCTTCCGGTTGGGGTGGCAGGGTGGTGGATCTGTCGGTCC | 248 |
| 54195 | CGTTTTCC[CG]TCGCACGTGGTGGCCACTGTTGGCTTCTGAATGGTTTGCAAGG | |
| | CGGATATCCACGCCAAG | |
| cg136 | AGTCCAGAAAGGCCCAGCCTGAATCACTGTGAGGTTGCCAGGGGCTTGGTTTC | 249 |
| 56062 | AGTTACT[CG]GAAACCACCCATCCTCCAGGCCAGCACCCAGGAGCTGCGTGGG | |
| | CCTTGGGCAGTGCCCCT | |
| cg136 | CCCATCCGGGATTGAGGAGCATCCCAATTCTGGGACCATCTCGGGGTCCCTGA | 250 |
| 56360 | CCCGGGG[CG]AATGGCTCTCCCATCTTGGGACCCCCATGCAGGGCTGCAGACC | |
| | CCCAGGCGCCCCCACCC | |
| cg137 | GAGCGAATCCTCTTCGGGCTTTCCAGAGTGCGGGGGATAGATAAAGAGTAGCT | 251 |
| 00897 | GGGGAGA[CG]CCCCCTGACCTTGCTGGGTCCCAGAACCCGGCTGCTCACCCCC | |
| | AAGGGGTCCTCTCCAGC | |
| cg137 | GCCACTACAGCACTGGTGCCCAAACCTGGCACACTAGGAACAAAGTCCTTGTT | 252 |
| 18960 | ACTTCTT[CG]TGGGCGTTTTCACCAAGTAGACTTGGAGGTTAACAAAGGACGC | |
| | AAAGGAGAGGTTCTAAT | |
| cg138 | TTTTCACAGGAGTGAGGCAGAAGACAGAAACTGCAACAAACCGCCGGGGGGT | 253 |
| 43773 | GGGATTAA[CG]TCCAAAGCTCACACCGGCTTCAACTGATTGGCAGGAACGAAG | |
| | TGGGTGGAGCCTCCTGAC | |
| cg138 | AATAATAAATAATAATGAATCCATTCTTCCTTCGGTCGTGGGTCTGGCAGGCAT | 254 |
| 54874 | AAATTC[CG]GCCGGGATTCCGACCCCAGGGCCAGAGCAGGACTCGCCTTGGC | |
| | GTCTATGAGTGGGCGGG | |
| cg138 | CTTGGGGGGCCAGGGGCAGGGCCTGTGGGGCGGGGCGGGCCTGGCTTGTTG | 255 |
| 61644 | GGCCTGTGC[CG]GGTGTCCGGGAGGGGCCAGACGGGGTCTTGGAGGGGCGG | |
| | GGCCGGGGCCTGTGGGGCGGG | |
| cg138 | GGGCTGAAGAGACCCCCCCCCAACACACCAGCCCCGAAAACCGTCTGCCGTCC | 256 |
| 99108 | CCTATAG[CG]CTGCATGGAAAAGAACCAAGACAAGGACTTGGAGTGGAGAAG | |
| | ACAGAAATTGTCCACTGA | |
| cg139 | CCATTTGAGGGCAAGGGCTGTGTCTTTGGGTACTTCGCTCCTCGCAGTCACAAG | 257 |
| 75369 | TACTGG[CG]TGCGTACGCGGGGAGAGATCGCTCCTCAAAACGGGGTCCTGAA | |
| | CGCTGCCCCGCGGCCCC | |
| cg139 | GGACGGCGCGGAGGAGCTGGAGGATCTGGTGCACTTCTCCGTGTCTGAGTTG | 258 |
| 94175 | CCTAGTCG[CG]GCTACGGCGTCATGGAGGAGATCCGGCGGCAGGGCAAGCTG | |
| | TGCGACGTGACCCTCAAGG | |
| cg140 | CATGCCTAGGGAATGACAGGCATCTCCACAGGCAGGCTGCATCCACCTTGGCT | 259 |
| 09688 | GGGGTGT[CG]TCATTGGCTGCCTATTAGAAAAACGACAGGACAATGCATACCA | |
| | CCGCCTCCCGACTGTAA | |
| cg141 | CAACTGCTTGCCAATTTAAATTTCTGGAGAGAAAAATGCACCCACTACAAAACG | 260 |
| 05047 | GACGAG[CG]GAGGGTTAGACCTTTGCCAGGTAGCGCTCAAAATCCGCTAAGA | |
| | CTACTCCCACCGAAACT | |
| cg141 | GCTGATCTCCAGTCTGCACACTGTTGGCAAATTAATCTTTCTGAGCTCTTGTTTT | 261 |
| 59818 | CATCG[CG]TCCCTCTCCTGCTCCAAAGCCCTCTGGGACTGCCTCCAGTAGCGCT | |
| | TCACAAACTTCAGC | |
| cg141 | CGCACAAAATCCCAGCCTCAAGGGCAGAACATTTTAAATGACCCACCCATCCTA | 262 |
| 75438 | GAGATG[CG]CCAGTTAGGTCATCTTATATATCTTGAGATAGCTGAGATGGTCA | |
| | GATCAACCAAGGACCT | |
| cg142 | TACCCCTCTGCCTAGCTACCTGGAGCCGGACTTTGGCCGTGTCCAGCGGGAAG | 263 |
| 23995 | GTGATCA[CG]TCCGCCAAGCACGCCGCTATTCCAGCTGAGAAGAGCTGGACCC | |
| | CCAGGGTCGGGTGTACG | |
| cg142 | CATGATACTCATGTATTTCCTTAGATCAAGTCAGCCTAGATCAGGTGTTCTTCCA | 264 |
| 81160 | GAGGC[CG]TATTGAGATCATTTTTATTTGCAGCCTGTGCATTTTCTCATCTCGG | |
| | GTGATGGCCTCACA | |
| cg143 | AGCCCGGCTGCAGACTCGTTAGCAGCGAGGCTTTAAATACAAAAGTGGGCCG | 265 |
| 50002 | GGAGCCCC[CG]CGTGGTGCCGCGGTGCCCCCTCATTATGCATGCATGGAAAAG | |
| | CAAACAAACAAAAACATT | |
| cg144 | GTCAGTGTTCTTTTAGTTTGCTTAAACTGTGTGGGTACTTGAGTCCTTTTAAACG | 266 |
| 23778 | ATTAA[CG]CTGGGAAGAGGCACCATTTAATTAATTAATTTGTTCTGGAAGGGA | |
| | TCAGTGTACAATTTT | |
| cg144 | CCACATTTGCAACCTTGGCCATCTGTCCAGAACCTGCTCCCACCTCAGGCCCAG | 267 |
| 67840 | GCCAAC[CG]TGAGTACCCTGCCCCACTGGGCTAGTCCCTGGCCTGCCAGCTTCA | |
| | GGGAGAGGGGTCTTC | |
| cg144 | CACCACAGACTCTGGGAGGCTCGGCGGCTGGAGCAGCAGGCAGCTCCCCGCA | 268 |
| 73016 | GCTCCCGG[CG]CTTCCAGGCAGCTCTCTGAGCCGTGCCAGAGGCCCGGCCCGC | |
| | CATTCCCAGGTAGGAGAC | |
| cg145 | GAAGAGTGTCGGGATCCACACAGGAACACACAGGAGAAATTCACCACTGTGC | 269 |
| 50518 | AGGAGGGA[CG]TGGTTAAGGCGAGTTCTGCCATTAACGTGTAATTAGACAACA | |
| | CTTTTACCCCGCCCCTCC | |
| cg146 | CGCCGCCGCCTCTTCGTCGCCTCAGCCTGGCGTTTTGTTCCGAGAGACGGGAG | 270 |
| 89355 | AGGCGAG[CG]GAGCTGACAGTGATTTTGACAGTGATTTAAACCCGCTTTTGTT | |
| | GTTGTTGGCTTTTCGTT | |
| cg147 | AGTGAACTGAGCAACAGCAAGTGCAAAGGCCCTGCTAGCACCATGAGCACGA | 271 |
| 47225 | TGAGAGAT[CG]TCCAGGAGGCGGTGTTGATGCGGCAAAGGGCAACAGGAAG | |
| | GGCATTAGGACTTGAAATCG | |
| cg147 | AATCAAAGGCGGGGTACAGGGCCAGAGGGAGGAGGAAACAACTTCCCGGTT | 272 |
| 54581 | GCTTTCAGA[CG]CTTCAGAGATCCTCTGGAGGCCTGGGGGAGCTTTTGAGTAC | |
| | TTTATTTCAGTTGGTCCCT | |
| cg149 | CGGAATACTGTCTGGCTGTGCACGTGGAGGTGGCGAAATGTGGAAGCTTAAC | 273 |
| 16213 | GAAGTTGG[CG]CCATGAAGCTAAAGACTGCTACCCCGGGGCTCTAGCTCGCTC | |
| | CGCCTAATGGCGGGCCGC | |
| cg149 | CAGCATGCAGGCACCGCCTCCTCATCTGCATAGAGCCGCCCTCCTCTCAGCCAA | 274 |
| 18082 | ACCTGC[CG]CTCATCTGCATAAGCCCCGCCTGCTGAAGGCTCTGCAGCTTGAAT | |
| | ATTTTCTGAGCGGAA | |
| cg149 | GAGGGACGACAGCTTTTACTGTCCCAAATCCTGAGATTAAGACCTCAGGGCTA | 275 |
| 72143 | AATCTTG[CG]TGGCGGTAAAAATTATTTGGAAGTTCTGTGCAACCGTTCCAATA | |
| | TTCCGCTTTTACGTGC | |
| cg150 | TCAGGGCTGGCAGTCTGGGCCAGGGTGTGGTTTCCAGCGTCAGGCCAGGCTCT | 276 |
| 13019 | GCTGTCT[CG]CGAGGGTCCGGCCTCAATGCTCCAGGCCCCGGCGTTGGGCCGC | |
| | GCCTCCTCGTGGGCCTC | |
| cg151 | TTCTTGACATTCAGAAGTCAGATTCAGGGACCCCATGGCAGAGCCTGTTTCTAA | 277 |
| 71237 | CACTTG[CG]CCTATTCGACTATAGGGACTATATTCTGCACAGAAATATACTTAG | |
| | TTTTATATATGGTTA | |
| cg152 | GGACAGGTACACGACGATGACGACCGGGGTGGTGAGAAGCTGCCCGACCAG | 278 |
| 01877 | GTCGGTGAG[CG]CCAGCCAGCCGATGCACAGCAGGAAGGACTTCTTGCGCTT | |
| | GCTCTCCCGGCGCCGGTAGC | |
| cg153 | TTCCAGCAAATAGAAAACAACCGAGAGCCTGAATTCACTGTCAGCTTTGAACAC | 279 |
| 44028 | TGAACG[CG]AGGACTGTTAACTGTTTCTGGCAAACATGAAGTCAGGCCTCTGG | |
| | TATTTCTTTCTCTTCT | |
| cg153 | CTTTCAAAGGCAAGCTGCAGGGCTCCTTGGTTTTGTCACATTCCTCATTCTGGG | 280 |
| 81313 | GCTTTG[CG]GTTTTGTCTTGGGAATCTCGAGGCTCTCCCAAGGTTCCTTTCTATG | |
| | TTTATATCATTTAG | |
| cg154 | CTGGTCCCCCCGGCGGGCGGCGGCGCGGGCAGGGGCAAGGGCTCCGGGCTC | 281 |
| 27448 | CTGCGGCTG[CG]TTGGCTGCTCAGGCCACCATAATCCAGCTCGCGGCTCGCAG | |
| | CTCCCGGGCGGGCTGGGGA | |
| cg154 | GTCTCCTAGGGCTGAAGACAACTTGGATTGCGAGGCTAGGGCTTGGGGAGTC | 282 |
| 47479 | GTGCATCC[CG]TTCCGGGCCTCCGCAGCCCAACATGGGCCCCGGGTTCCAAAG | |
| | TTTGCGAAGTTGGGCGCC | |
| cg154 | TTAAAACTGTTTTCCAGGGCAGTTTCTCTCTCTGGTTCTAGGACACTTAATTGGG | 283 |
| 89301 | CTCAA[CG]TTTCCCCCAAGTCTTGGCTGTGGGTTTGTTGTGGGCTGGGTGGTTG | |
| | AGGAGAGGATGCAG | |
| cg154 | CCGCAGACCCCTCGGTCTTGCTATGTCGAGCTCACCCGTGAAGCGTCAGAGGA | 284 |
| 98283 | TGGAGTC[CG]CGCTGGACCAGCTCAAGCAGTTCACCACCGTGGTGGCCGACAC | |
| | GGGCGACTTCCACGGTG | |
| cg155 | CGTGTCACAGACTCTCAGAAAGCACAGTGAGAGTTCCCCTGGTTGAGAATCGC | 285 |
| 51881 | AGGCTGC[CG]CTGGCTTCCCCCACTTCCCTGGGCACATCGGAGGAGGGGGCCA | |
| | CAGCTGGTCCCTGGTCT | |
| cg155 | CCCCGAGGCTCCCGCATCAACGCCCTCAGTCGGATGGGACTGAGGGTGCCGCC | 286 |
| 69512 | GCCACCA[CG]CCGGGGACTGTTGACAGCCAGAACCTTTAAGCGTAACAGAGTC | |
| | ACCTGGCAAGTTTGTAC | |
| cg156 | ATTGGCAGGTCCTCGATTATCGGCGAGTCACTGAGGTTCCGAGAGGGGCGTCT | 287 |
| 11364 | CTGCTCA[CG]CAAACAGCTACCCAGCCGCCTCCCACGGTCTGACCTCAGCCAAG | |
| | GTGACGCGGCTTAAAG | |
| cg156 | CTGTATCTCTTTGTCTCTCCCTGTGTGTGTGTGGGGTCTCTCTCAGTCTTTGTCCC | 288 |
| 42326 | TCTG[CG]TCTCTGGGTCTCTCTGTCTCTGTCTCCCTGCGTCTCTCTCTCTCTGTCA | |
| | TCTGGCTCTTC | |
| cg158 | AGAGGGCAGGGCTGTATTCCGCTACTGGGTCCTATGCACCATGCAGAACCAGT | 289 |
| 11427 | GTCTTCA[CG]TGGAGACTCATCACTGATCCGAAAGGTGACTGCTTCTGTATTAC | |
| | ACTCATTTCCCCATGA | |
| cg158 | AGAGACACTATCTCCAAAAGAAAAAAAGAGAAACGTATGGTTACATGATTTCC | 290 |
| 56055 | TTCTGTG[CG]ATCATCAGAGTCACTCGCAGACCTGGATGGTGTACGGCCTACA | |
| | ATGCACCTAAACCACCT | |
| cg158 | TCCGGGCGAGGAGATCAGCAGGGGTTTTCGAGGGAGCCTGGGGCCCAGGGC | 291 |
| 81088 | AGGGGTACG[CG]GGTCAACTCAACAGATGTAAGGCGTGGCCGAACCCCATTC | |
| | AGCTAGCAGTACCCAGCCTC | |
| cg158 | ACTTCTCCGAGGTTACACAGCTAGGAAATGGTGGCAACAGTAAGAGCCCACGA | 292 |
| 87846 | AGAGCTG[CG]GTTGGTAGTTCATTCTGGACAGCCCTCCCGTGAACCGTCCCTGT | |
| | ACTGGCACTTGTTGCT | |
| cg159 | CGATTAGTAAATACCAACCCATGCTAGAGAGTGAAGAGCTCTGGAGGAGAGG | 293 |
| 03282 | CACGGGTG[CG]CCCCTGGAGTTGCTCTAAACAGGGTAGGCAGGGTGCTCTTGT | |
| | CACAGAGAAGATGAACGA | |
| cg159 | CCCAAGCCCCGTCGATTAGACAGGTTTAGGCACTTCCGGGACTCTCAGAAGCCT | 294 |
| 63417 | GGGAAG[CG]AGTTCTCTGCAATTGGACTAAGCCTGCGACCGTCTGGTATAACA | |
| | ATTATATGAATAATCC | |
| cg159 | CAGGTCGGGCCAGTTGCTGGTGAGCTTATGAAGTGTGGTCTCCTCCCCGGAGC | 295 |
| 66757 | TCATGTG[CG]CTTCCCACCTGGTGAGCTCAGGGTCTCTCTGGAGGGATCCTGCC | |
| | TCCCACCCCTGTCTCC | |
| cg160 | TTTTCCCTTAGAGGCCAAGGCCGCCCAGGCAAAGGGGCGGTCCCACGTGTGAG | 296 |
| 85042 | GGGCCCG[CG]GAGCCATTTGATTGGAGAAAAGCTGCAAACCCTGACCAATCG | |
| | GAAGGAGCCACGCTTCGG | |
| cg161 | CAGGGTGAGAGCAGGTCTCACTCATCCCAATCCCAGCCAGGATTGGGTCAGGG | 297 |
| 73067 | CCCCCAG[CG]CTTACCTGCAGGCAAGGTGCTGCTCCACGACCTTCTCCAGCTGC | |
| | TGCCGCTGCTGAATCT | |
| cg162 | GGCTGGGCCAGGGTGGGGCGTGGCCCGGGGCGGGGGAGGGGCGGGGCTGC | 298 |
| 95988 | CAGGCAGGGG[CG]GGACGGAGAACACCTGGGTCCCTAGCACCAAGACTGGCT | |
| | TTTTATTCATTGCCACCGCCT | |
| cg163 | TGGTTACCCGTGAGTCACCTCGCTGTGCCCCCTGCCCAGAGCGGGAACCCTGG | 299 |
| 13343 | CTGCGCA[CG]CCCTCAAATATCTGCAGGTGCTGTTCACAATCGCCATAGGGCC | |
| | GGTGACATACCCAGGAA | |
| cg163 | GGGAGCTGAGTTGCTGGTAGTGCCCGTGGTGCTTGGTTCGAGGTGGCCGTTA | 300 |
| 19578 | GTTGACTC[CG]CGGAGTTCATCTCCCTGGTTTTCCCGTCCTAACGTCGCTCGCCT | |
| | TTCAGTCAGGATGTCT | |
| cg163 | GGCTAGGGACGTTATGTAAGTTGAGCCACGCTACGCTAAAAGTTCCACACTCA | 301 |
| 40918 | ATTCTAG[CG]TCTCGGCTCTGGACTACCAAGTTCCGGAGCAAGCAGACAGACC | |
| | ACCTCTTTACGTTCCCG | |
| cg163 | TTAGAAACCTCTCAGTGGGGTTTTTCGAAATGAAAGTCTAACTCCTTGTCTCTTT | 302 |
| 54207 | CTGAC[CG]TTTCCATGCTGAACCTCATCTTTCTAATGGCCCACTCCTCCAGGGGC | |
| | CTGCCTGACGCCC | |
| cg163 | CGCCGCCCCCCCCACCCCTCCCCCAGACAAACGATATGACGCACTTAACTATAA | 303 |
| 57381 | ACCCCC[CG]ACCCCCCGTGGCTTCTGGGAATTTCCCAGAAGGTTCTGCATGGGC | |
| | AGATGCTGAGGGAGT | |
| cg163 | TATTTAAAGATTGTGGCTAATAGTAGAGTAGATACCCCTGGTATTTCCCAGAGC | 304 |
| 72520 | AGAGCT[CG]CATTCTGGGAATCTTAGGTCCATGTGACTTCCTGAGTCAGTGATT | |
| | CACACTGAGAAAAGA | |
| cg164 | CGGAGGGGGAAACAAAACTACAGCAAGACCACCTTGAGTACCTTGGGAAGGG | 305 |
| 08970 | CAGCCCCG[CG]ATCCCTAATAAATGAATTAGCATCTCAAGGAGGAGATCACTG | |
| | CGGGGCTGATATTGATCA | |
| cg164 | TCTATGTTGTCTCTATGCCTTGCTGTCTTGCCTGCCTCCTTGTAGGTCCAACCTC | 306 |
| 66334 | GGGAG[CG]CAGCTTTTAAAGAGTGACAGTGTTTGTTTGGATCACCCGCAGCTT | |
| | GACTCATCCTTGCTT | |
| cg165 | CTTAATCCCAGGTTTGTTTATCCAAGCAGTGGTGTCAGCTGCCTGGCCAAACCA | 307 |
| 43027 | CACAGG[CG]CCTGGATCCTAGGAGACATAAACCAATCCTCCCACCCAAGCAAA | |
| | GCCCCGTAGCAGCCCG | |
| cg166 | GCCGCCCGGGGTCCGAATTGGGGGGGGCGGCTGTGTGACCTTGGGCGAATCG | 308 |
| 12562 | CCGCACTG[CG]CTGGGTCTGCGCTCCGCATCCATCACAGGCAGACTCCTCAAG | |
| | AGGCTCCAACCTTTTCTT | |
| cg166 | GGTAACTGCACAGGAGAAGGTGAACCAGTAAGTGGGCCATATGTCTCTGCAA | 309 |
| 48841 | AACTTGCA[CG]TAGGAATCACCTGCTGGGGAACTAAGACACTTTTATGTTTGCA | |
| | GCAGAGGCTGTGTTAGA | |
| cg167 | CCCAGGGTCCAGGCCCGCCCTCGGCTGGCAGGTGTGGGCACAGAGGCAGCTG | 310 |
| 13727 | GGATTGGT[CG]CAGCTGGCGGAGGCGCGTCCCAGGCTCCGGCAGACCGCTGG | |
| | AACAGCTGAGCAGAGCAGG | |
| cg167 | CCCCCCGCCTGCCGAGGGGGCTGGCGGGGGGGCATTCCTGGGTCCCTGGAAC | 311 |
| 18891 | TCTGAGCC[CG]CGTCCCCCACCCCTAAGGGGCGTGGGGGGGGGGCGCACCCC | |
| | TCCAACCCCCTTTCCCCAG | |
| cg167 | AGCCTGGATTCTAGTGAAGCCCAATTCACCAGCCATTTGGTCTTAGTAAGGTCA | 312 |
| 28114 | TTACCG[CG]CTCTAGGTTTGAGTCTCATTTGTAAAATGAAGGGAGTGGAGGGG | |
| | CTTATAGAGCTCGAAC | |
| cg167 | GTGCGCGCTATGTGACCTCTCAGGGGTCGCTGCCTTGGACGATCTGTAAAGCT | 313 |
| 43289 | GAGTGCG[CG]CTATGTGACCTCTCAGGGGTTGTTTCCAACCGTGTTGTTGACAT | |
| | CTTGAGCCTGCCAAGG | |
| cg168 | GAGCTAAAAGGTAGTATCCCACCCTCTCCATAAACAGACACCTAAGTTATAAAA | 314 |
| 16226 | CTTATG[CG]CTCGATATGCAAAAATAGCTCGTTTTATACAGAAACGATCCTTTC | |
| | CTTCTTTTCCTTATA | |
| cg168 | GGCAGCTGGGGATGGGCAGGCTGCAGCGTGGGCAAGACGAGGTGGCTGCTG | 315 |
| 54606 | TGACTCTGC[CG]CTGAACCCTCAGGAAGTGATCCAGGGGATGTGTAAGGCTGT | |
| | GCCCTTCGTTCAGGTGAGT | |
| cg169 | GGTCAGTCGGGGCCTGCAGACCGTGACTCCGTCACGAACCCCAAATTCGCTTC | 316 |
| 33388 | TCCCCAA[CG]CTCGGGCCTGACTGCTCAGGAGGGGCTTATGTAACCTTAACCT | |
| | GGTCCCTCCGCACAGGA | |
| cg169 | TTTCTTCAAATTAAATTGCTACAGCAGGAAATTACTGAACTGTGGCTCTTCTCCT | 317 |
| 84944 | ACGTC[CG]CCTTCCCTATGTCAATTCCCATTTCCCTTGCTTTCTCCAATAGTTAG | |
| | GACTGTAAATTCT | |
| cg170 | GGACAGATGGATGGACGCTCGCGGGCAATGAATGGGCGCTGCGCTCAACCAA | 318 |
| 09433 | GACACTCG[CG]CAAAGTTGTGGCTCCACCCAAGGCACCTGCTCCGCACACTTTA | |
| | AGCGGCGCCCTGGAGGC | |
| cg170 | TATGCGATGATGTTTGTTTGCCCTTGACGCACTTACTCATGGATGGTACTTCTTC | 319 |
| 38116 | AGCCT[CG]TTAGACAGCCTGGTGATGGAGGATGAAGAAACCATGTGCIIII CA | |
| | TTCAGTTCTGGACTT | |
| cg171 | TGTGTGGGACAGTCAGGTCGGCAGGAGTGCATGAGAACGGTGTGGGCACACG | 320 |
| 29388 | TAAGTGCA[CG]ATCACACATACAAGTGAGCTTGAGAGTGTGTATTCCTGTGCA | |
| | CTGTGTGCACACCTGTGA | |
| cg171 | GCAGAGTCCAATTATGTGTTTTCTGATAAAAGCATATGTTCATTGAAAACACTG | 321 |
| 33388 | GAAGAG[CG]GCATACTGGAATACTGGTTTATCTGGTGTATTTCGGGAGTTTAC | |
| | AGATCACGAAAGTTGC | |
| cg173 | CCCTCCCCCGCCAGCCTGGCGCATTGCGGGCCTCGGGCTCATTGCTGAGAGGG | 322 |
| 24128 | GGCACTG[CG]CCTGGCACCTCTGTTAAGCAATTTAGGGGCTACAACCTGAGCA | |
| | AGACAGATGAGCCCGGC | |
| cg174 | TATGTTGAGTGAAAGAAGCCAGACAAAATCAAGTACATATGGGATGATTCCAT | 323 |
| 31739 | TTATGGA[CG]ACTCTAGAAAATGCAAACTAAAACAGATCAGTGTTTGGGCTGC | |
| | GGATGAGTGGAGTTGGG | |
| cg175 | CCTACGAAGAGGTAGGGCTTGGCAAGGACCCACGGGGCGTGTCCTAGGACTC | 324 |
| 26300 | GGTGAGGG[CG]TGACCTCGGGCCAGGGGCGGGGAGAGAACCAGAGGGCGA | |
| | AGTGGGAGGGCACAGGGGAGA | |
| cg175 | CCTTCCGGTAGCTCGGTCACTAGGGTCAGTTTTATGACTCTCAGTGGACCCTAA | 325 |
| 36848 | ACAGCA[CG]TAATATATGTATTTTTCACCGCCAAATATATCAAACACAATAATTC | |
| | ACCCTCCGTTCCCT | |
| cg176 | ACCCATGAGCCAATTGCAGAGGCAACAGAAGACCAGTGCACCAACCAGGCTG | 326 |
| 05084 | GGTCCCTC[CG]CCAGAGGGTGTCACCATCTAAGCTGAAAGTGTTTGGGGAGAT | |
| | CAGACATTGCTGTCTGGT | |
| cg176 | GGAAGCTGGGCTGTGCGTGTATGCGTCTACCATGTGGGGGTGCCTGTGAGTGT | 327 |
| 27559 | GCTGGGG[CG]TCTGCAGTGAAGGCCTCCTGAGACCACTCCACGGAAACACCG | |
| | GGAATCCCTGCAGCTGAG | |
| cg176 | GCGTCGCTTTCACACTCGGCGGCTGCGGATTGACGCCTCCGCCTGTTCCCCGGA | 328 |
| 41104 | GGAGAG[CG]AGTGCAAGAGAAAAAACACTTTTATTGAAACGATCCAACCAGC | |
| | GGCGGCGGAGAAAAGCG | |
| cg177 | TCCGGGGTTTTTACCCTCGGCAGTTTGATGTCCTTTGTGTCAAGGTCTGGCTGC | 329 |
| 26022 | GGAGGC[CG]GGAAAATGTGGCCCCCGTCAGTAAGGGTTGGGCAGGGAGCTT | |
| | GGCGTGGCCTGGCGGATT | |
| cg177 | GCGTTACTTGCAGGATGCAGGAGTGATGCGATCAGAGCCAGCCGGAACCGAG | 330 |
| 49443 | TTCCGTTA[CG]CACTACAGGACTGACCTGGGCCTGACAACCCACTGCCGGAGT | |
| | TCGGATCGCATCACTGCC | |
| cg177 | ATGGTTACATGATTTCCTTCTGTGCGATCATCAGAGTCACTCGCAGACCTGGAT | 331 |
| 70886 | GGTGTA[CG]GCCTACAATGCACCTAAACCACCTAGAGGAGCCTCTTGCTCGTG | |
| | GGCTACAAACCTGCCC | |
| cg178 | GCGGACTTGTCCGGATCCGAATAGAAGCGCTGTTGGATGCGGATGGGGCGCC | 332 |
| 61230 | GGGGTTGC[CG]CCACAGGTGCTTCGGGGCTCTGGTCATGCTGTGGCGGCCGC | |
| | GAGAGCGACTCAACCTGCT | |
| cg178 | AGGCTGGACATTTGCTACTGGTCCCTGAAGTTTTGCGGCTGCACCCACAGACA | 333 |
| 96249 | GCAATAG[CG]CCACGTTCCCTGGAAGGCGCACGGGACGGAAGCGGAAGCAGT | |
| | AACGCTGGCTCCGGCTGC | |
| cg179 | GGAGATGGCAACAGGGCAAGCGTCCAGCAATGGGTAAGCGGTGGGGTCGGT | 334 |
| 03544 | GCACGCAGG[CG]TCCAGCAATGGGTAAGCGGTGGGGTCGGTCCACCCAGGGA | |
| | GCGCTGGTCCCCCTGGAAGG | |
| cg179 | GGAGGTGCTGCGGTACCTACCATGGTATTCTTGTCCCGGAACGTAGTAGGTGG | 335 |
| 23358 | GGTTGCC[CG]CAATATGCAGGGAAATGAGCACCTCGCCCTGCTCCCCATCCCCT | |
| | TCCAGCTCCCCGTGGT | |
| cg179 | GCCTCTGGGAGGGCAAGACCGGGAGGGGTCGGCCTGTGTCGGGGGCTCCTG | 336 |
| 40013 | GAAAAGCAG[CG]CCACCGCCACCCACCTGACGACATGGAAGGCCCAAAGCAG | |
| | GCGATCTGTGCGAGGCCCGC | |
| cg179 | CTGGCAGATGTTTGTACTGGGAGATTCAGATCCATCCAGGCCCCCACTGTTAAT | 337 |
| 66192 | AGCCCA[CG]GGAAAGTCCCTGCAGTCTCTCAGGGAAGTCATTCTGTGTAGAAT | |
| | CTGTAATTTCACAGGC | |
| cg180 | AGCTGGGGCTCGCCTGTTGGGAGCCGCGTCCGCCGGTGTTGGTGTCTGCACTT | 338 |
| 01427 | GGAAGGA[CG]TAGGGAATGCGTTGTCCCTGCTAGGTACTTTTCAGTCGCAGAG | |
| | TTCTCTTCTTCTTCTTT | |
| cg180 | CTTGGTGTTCAGCACCAGCCGCCCCCCCAGCCGCATCATCTTTTCTTTCAACAAC | 339 |
| 03795 | AGATG[CG]CCCGTGTTTCATCTATGGATAGAGCTGAGCCGAAGAAAGACATTG | |
| | CCACAGCCAACAGCA | |
| cg181 | TGATAGTATTTTCTACTGTCCTATACACATCAGGCAAGACTTCATGGAGAGCAC | 340 |
| 17393 | TGAACA[CG]TACTCACTATGTGCCTAGCATTGTTGTTAATCACTTTACATGAATT | |
| | AGTTCATTTAATTC | |
| cg182 | CAAGAGCGACCCTCGTTCTTCACACGAGGAGAAGAAATGGACACGTGATTGAC | 341 |
| 41647 | CATTAGG[CG]CCACCAGGGCCAAACTATCTTATGGAAGGAGGAAAAGAAGCA | |
| | CAGAAAGGGCATGAAATT | |
| cg182 | GGGGCCCTGGCCCGGGACCAGCGCCGCGGCTATAAATGGGCTGCGGCGAGGC | 342 |
| 67374 | CGGCAGAA[CG]CTGTGACAGCCACACGCCCCAAGGCCTCCAAGATGAGCTACA | |
| | CGTTGGACTCGCTGGGCA | |
| cg183 | TCACCCCCGTCTTGGGGACATCAGGTCTGTGAGCACCCATACCCCAGCCAGGC | 343 |
| 84097 | ACTGTGG[CG]CCCCACTCGCCCTCCCGCACTCCCTCCTAGAGATGCCCTCTTATA | |
| | TCCCCGGAGTTCGCA | |
| cg183 | AGCAAGGGAAGTTGGATGAGAATTTGAATCCAAAGCGTGCCATGGGACCACA | 344 |
| 92482 | ATTGCACA[CG]ATCAATGAGTCTCACAAACTGACCACGGCTTATCTGAGGCAG | |
| | TTTAGGGTTGTGCAAGAG | |
| cg184 | AAAAGACGAGATGACAAGACACAGACAGCGAGCATGTGCCTGTGCACATTTG | 345 |
| 68844 | GGTCTGTG[CG]TCTCTGGATGGGGGTGAGAGAGAAAATAAAAGAAGGGGAG | |
| | TGGAGGAAAGAGAATGCCCG | |
| cg185 | AGGTTAAACGGCACTGACCATGCTGAGCCACAGCCGGTAAAGATGGCGGTGG | 346 |
| 87364 | CACACTGA[CG]TCACTTCCGCTCCGAGCCTCCGGCCGGGTGGGGCTCCAGGGC | |
| | TTGAGTTTCAGGCACGTA | |
| cg186 | AAGCCCACGTGAGAGGGCAGGACGCCTGAGAGCTTGAGGCCACACGAGGCTG | 347 |
| 91434 | TGGAGCGG[CG]TGACTCAAACGTGGCGCGCATCAGCTCGCACACTTCCAAACC | |
| | TCGCGATAGCTACTGGCC | |
| cg186 | GACCCAGGCGACTGACATGTTCCTCTCCTCTCAGCTGAAAAGCTTTGCTAGCTC | 348 |
| 93704 | TGTCTA[CG]CATAAAGTAAGGTTAAACACAGATTTTGCCCCGAAGGGCATTAA | |
| | TTAGGGACCAATTTAC | |
| cg187 | CTGGAGGGAGGAAGGTGTGGGGGGACCCAGGGGTCCTGTCTCCAAGCCTGGT | 349 |
| 32541 | TGCTCTTA[CG]CGAAAAGTTGGGACACTGAGGTGTCACAGCTTCTCTTTTGAAA | |
| | TGGAGAGGAGGTAGGAG | |
| cg187 | TGCCGTGGGGAAAACCTGCCTGCTGATGAGCTACGCCAACGACGCCTTCCCAG | 350 |
| 71300 | AGGAATA[CG]TGCCCACTGTGTTTGACCACTATGCAGGTAAGAAAAAGTGGGA | |
| | AACTCTCTGCATCCAGA | |
| cg188 | CTGGCAGCCAGTGGTTCGCCGGCACTGACGACTACATCTACCTCAGCCTCGTG | 351 |
| 09289 | GGCTCGG[CG]GGCTGCAGCGAGAAGCACCTGCTGGACAAGCCCTTCTACAAC | |
| | GACTTCGAGCGTGGCGCG | |
| cg188 | TCAGTGCGTGTTAGCGAGCAGCGCCGGGAGATAGCTGTCACCGCCGCCCGCTC | 352 |
| 81501 | ACAGATG[CG]TAGACTGAGGCTCAGGTGTCACCACCTGACCAAGGCTAGTTCC | |
| | GCTACAAAGCTGCCGAC | |
| cg189 | TGGGTGGAAAAGGAAAGGGCCCATTAGACGAATCTGATTCATCTTCTGTGACT | 353 |
| 96776 | AAGCACC[CG]CAACAGTTAGGAATTTAGGCAGAGCTGGTGATCCTGGGACAAT | |
| | AGCACTTCCTAGGTAAT | |
| cg190 | GCGCGCGTGCCGCCGCCGCGGGCACTGCGCCCGTTTGCCTGCCCCTCGTCGGG | 354 |
| 08809 | GATCGGG[CG]CTCCCTCTGAGACCTGAAAGGGCACCCAAGTGCCCCCTGTCTG | |
| | CGAAGTCCGGCGCGGGC | |
| cg190 | GACCCCCGGCAGGGACGTTTTTCTGCAAACTCACAGCATTTGACAAAGTTACAT | 355 |
| 28160 | AAACGG[CG]CCCGGCCGGCCCCGGCGCCCGCCCGCCCCCGCCCTCACTCCCGG | |
| | CGGCCCGGAGCCCACC | |
| cg191 | CGTGCGTGGCCAGGATCACATCGTTGGGGTCCATGGTGGTCTTCAGCAGGCCC | 356 |
| 04072 | CTGTAGA[CG]CGGTAATCGCCGCTAGCGTCCAGGACGCCTCCAGAGGCCAGC | |
| | GCGGTGCGGAGCTGCGCC | |
| cg191 | GAGCCTCAGGGGCGGAGTCTTAGTGTCCAGAGGGGAGTCAGGGCAGCTGGA | 357 |
| 49785 | GGTCCAGGG[CG]GGAACCATTGAGGCTGGGACCCTACGAGAACCCCCTACCC | |
| | CGTGCCCTTCGGCCTCTCTT | |
| cg192 | GGAAGCAATCCGGCCCCTTTTTGGCAGCGAGTTGGCCCGGTCTTTGGCTGCCTC358 | |
| 87114 | AGACCG[CG]TTGCCCTCCAGCCTCGAGGCAGAGAGCTGCCTCGGTGCCACAGC | |
| | TAAATAAGCCCGGCGC | |
| cg192 | GGCAAGCAGGTTTGGTTCCTGCCCAGCAAAGGTGAGGGAGGACGGAGGAGA | 359 |
| 97232 | CTCTCCCAC[CG]CATTCAGAACTTTATTCCTTTATTTTTGTCTCAATCTTGTCATA | |
| | GAGGAGCGCTTCACTT | |
| cg193 | CCGCTGCCTAGTCTGCATCTGAGTAACATGGCGGCGGCGGCGGTAGCCAGGCT | 360 |
| 45165 | GTGGTGG[CG]CGGGATCTTGGGGGCCTCGGCGCTGACCAGGGGTGAGCACG | |
| | GGCAGCCAGCTGAGACCGG | |
| cg193 | CAGGAACATCACTTGGTAATTAAGAGATCGCCTTGCTTCAGATCCTTGCTCTCC | 361 |
| 56189 | TAGCCA[CG]TGACTGTGAGCAAGTGACTTTGCTTCTCTGTGTCTGTTTCTTCAAC | |
| | TATAAAATAGGTAT | |
| cg193 | CAGACGCTTCTGAAAGGGCAAAGACGACGCCAAAGAAGACGCCGGAGACCTC | 362 |
| 71795 | GAATAGGG[CG]CAGGTGGACATCTCTGATTTTCAGCAGACCAGCCTGTATGTG | |
| | TCTGAAGTCTAGCAACGA | |
| cg193 | GAGGAGGGCGCTGGTGCTCAATGAGTGAGCCCACCTGGGGACTACCAGGACG | 363 |
| 78133 | AGGACGGG[CG]CAGGTGAAAGTCCTGGGCTCATTGCCCCAGCATCCAACTTTC | |
| | ACCCTCTGTCCCCTTTAG | |
| cg193 | AACAAACAAAGCTAAGGTTCTTACCCCACGGCTTGCACTCTCTCAGCAGAGCTG | 364 |
| 98783 | CAGGTG[CG]TGGATGATTCGTTGACACGGTCAGAATTGGCTGCAGGAGGGAA | |
| | TTGAATCGAGGTTTTCT | |
| cg194 | ACCGGCGCGAGTTGGAAAGTTTGCCCGAGGGCTGGTGCAGGCTTGGAGCTGG | 365 |
| 39331 | GGGCCGTG[CG]CTGCCCTGGGAATGTGACCCGGCCAGCGGTGAGTTGGGGCC | |
| | GGGGCAGAGGGCAGGGGTG | |
| cg195 | CGGGGCAGCCCGCCCCACCCCTCCCCCCAGGCTCCTCCCCATCCCTCCCTGCCC | 366 |
| 14469 | AGGCCG[CG]AGAATGACCACTCCACTTGCAGGCGAAGCCCCTGGCCGCTGTGC | |
| | TGAAGGAGGTGTGCGA | |
| cg195 | GTGTGGTGCTTCCTTCTGACCTTGGGCACCTCCGTCTTCAGTTGCCCCTCCTGTG | 367 |
| 56572 | AAAGG[CG]AAATGTATCGTTGGGTTCTTTGAGGCCCTTTACAGCTCTGACATCC | |
| | TATAACATTCTGTA | |
| cg195 | CGGGCACTATGCTGAGCAACTGCAGCTCAGGTCCTGCAGAGTCCCCGAGAGTA | 368 |
| 60210 | CTTTGCA[CG]AAGAGAGCTCGAGTTCTGTAGTCAGGCATATCTGACCTACCGA | |
| | ACAGGTGCCCTGGTCAA | |
| cg195 | TGGAGCAGGACCAACTTACCAGCTCGCGGTGCTCCCTAGAAGCTGGATTCTTC | 369 |
| 66405 | GCAGGTG[CG]AGCACACCCCAGATGCCAGCGTGGACCCTTGAGCAACTGGAA | |
| | GTTAAAAACCCACGAAAA | |
| cg195 | ATCATTCATTCATTCATTCATTCACCCATTCACTAATCAGTAAAATTTAAGTGTCC | 370 |
| 73166 | ACTA[CG]TTCCAGGACTTGCACTAGACTTTAAGGATAACAGGGTGGACAAGTT | |
| | CCACTTTGGAGACC | |
| cg195 | GGAAAAGTCATTTTAAGTAAAGACAACGAGTTAATCAGGAGGCGATGAGCCC | 371 |
| 86576 | AGTCCTTC[CG]CCCCGCTTTCCCGCTTCCAGCCCTCGAACGAACCCTCCTCTAAC | |
| | CCCCGGGAGGCAGGAG | |
| cg196 | TGGCTTGGGGTCTCAGGGAACCGAACCGCCCTCCCCCCAGACCTGCTACCCCA | 372 |
| 15059 | GGCCCCA[CG]TTGGTGCCCATTTCACAGGTGATAAAACCGAGACCCAAAGAGC | |
| | CGGTGTCCGGCCCAAGG | |
| cg196 | AAATAATCAGCAGTTCCTGGTGGCATGTAACCAAGTAAAAACCAGTTACACAG | 373 |
| 32206 | AGAGCCA[CG]AACCCCCAAGGCAAGAAAGCAGAATGTGAAAATGCTTTATATG | |
| | GGGGGGTGGGGAATGGT | |
| cg196 | GGCATGGGGGCTGGGGGCCGAGATGCCCAGGTTTCTGGGTGTAAGGACTCAC | 374 |
| 63795 | CATGACTC[CG]CCAGCCATCACTGCACCTGCCGTCTCTCCCCACTTCCTCTGGTG | |
| | GGGCAGGAAGCTGAGT | |
| cg196 | TTGTGAGACACTGTTTCTGAGAGCAGCTTTTGTGGCATCTTACAGGGCAGATTT | 375 |
| 85066 | CTGGTA[CG]TTCTAAAAGTTGAATTTCTAACTTTGGCTGGTTGTGGCCCCTGAC | |
| | TGTTTTTTTTTTTTT | |
| cg196 | ATTCCTTTACTTTTCTATAACTCTGTCATGACCAGTTTAAAGGCCCCAATGTCAT | 376 |
| 86152 | GTCCT[CG]CATTAACAACCAAGGCTACAATGCAAGCCCTGCCATGTGCGCTTCT | |
| | TTACAAAAGGTCAA | |
| cg197 | TCTGCTTACAGCTGCTTCCAAATTAAGCATATCTGGATGGTGTGACACTTTTTGT | 377 |
| 22847 | TAGTC[CG]AGAACTGTATGGGCATCGCAACTGGGCCTGTTCCAAGATAGACTT | |
| | GTTGGGACCTTCAAA | |
| cg197 | CATTCTTATGCGACTGTGTGTTCAGAATATAGCTCTGATGCTAGGCTGGAGGTC | 378 |
| 24470 | TGGACA[CG]GGTCCAAGTCCACCGCCAGCTGCTTGCTAGTAACATGACTTGTG | |
| | TAAGTTATCCCAGCTG | |
| cg197 | AGTTTGAGAGACCAGGGCTGCTGGGGCCTGGTCATGCAGGGCCCGGACAGGG | 379 |
| 31122 | GGCTGTCC[CG]CTGTGAGGAAGCTCTTGGCTTACCCTCCTCTGAGCCTCAGAGC | |
| | TTGTGAGGTTAGTTCCT | |
| cg198 | CCGTCTCCTCACCTGCCCCACCCGTGGCCTGGGTTTAAAATCCACATACCCGTCT | 380 |
| 83905 | TTCCG[CG]GCCAAAGTGATGCTGCCAGGATTGGTTATGACCCCAACTGCCCCG | |
| | ACCCCCAGAAGTGCA | |
| cg200 | AAGAAGCCCTCACCGAGAGCTGTGGGAACAAGAGCTGCCGGGAACAAGAGCT | 381 |
| 66677 | GCGGGAAG[CG]GCTCCTACGAATTGGTGGCAGGAGGCACAAAAACGAAATAC | |
| | CTATTTTTGGAATACGGAA | |
| cg200 | TAAACCAGAGACTTGAATTATTGGCAAATGTCCAGACAACATTCACAATGCTTA | 382 |
| 90497 | CTAGCA[CG]CTATTGCCATATGTACCTGGAAAGAGCAGCATAAAGAAGCCATC | |
| | TAATGATATTACACAC | |
| cg201 | GGTGCCTCCAGGCCACGTGGGCTGGCAGTCAACTCACCTGTTTCTCAGAGGAG | 383 |
| 62159 | TCCAGGA[CG]CACAGAAGGTGCCGGTCACTGCCCTCTGCCGGACCCATGGAG | |
| | GGGTAAGGGTGTCCGGCC | |
| cg201 | AGCCCCACCTCTCCCTTAGGGACCTCCGCCCACCCTACCCTCAAGCCAGGATGC | 384 |
| 73259 | CCGGAG[CG]TCCCCGGAAGTGGGTGTGGTTCAGGTGATTTAACTCATTATTTA | |
| | ATACGCCCGCAGGGTG | |
| cg202 | CAGAGTAATTTAACCCAGGATTGCTGACTTTTTAAGAGCTGAGAAAGCATAGCT | 385 |
| 34170 | ATGGAG[CG]CAAGGCCCCACCCAGCAGGGTCTAAGTATTCCGTCTGCAAAACT | |
| | GGCAGGCCACCAACGG | |
| cg204 | CCTGGGGTGTAAGTACTGCTTGTGGGAGAGCCCCACAGGAAATCCAGAGTATT | 386 |
| 92933 | GCGCATG[CG]TGCTGTCCAGAAGGCGCTTGAACTCGGCGGCTTCCGTAGCGG | |
| | GAGGGCGAAAGATGGCGG | |
| cg205 | GCTCGGTGCCCATGGCCCACTGCTGCTGGAGGAACCTGTGTCTCCCTTTGCAGC | 387 |
| 50118 | CTGTGG[CG]CGCCTTCCTTGCAGGGTGTGTACACTGGCTGTTTGCAGAGGGGG | |
| | TTTGTGCATCCTAGTT | |
| cg205 | GCGCCCGGAGCCGGGCTGCTTGGTTCCAGTGTTGGGCCACATACTGCTTGCGT | 388 |
| 70279 | GCTAGGT[CG]CCCCTCCGGGTGGCTCAGCCTCTTCCCCTCTCTCACAATCCCTG | |
| | AATCCCTCTGTCCCTT | |
| cg205 | CCTGAACACCGCTCTGCAGAATCTTGGTGGCTAAGGTGTCCAGGAGCCTCTGC | 389 |
| 72838 | AGCGGAC[CG]CCAGCCTGAGAGGCGCAGAGCTTGTCGGGCAGGGGCCCGCTT | |
| | GTCCCACTCCCCTGATTT | |
| cg206 | GCCCGCCCGGGGCTAGAGGCGGCCGCCGGGAGGGCGCGCGGCGCCGGAGAC | 390 |
| 52640 | ATGTCCAGG[CG]GAAACAGAGCAACCCCCGGCAGATCAAGCGTGAGTCAAAC | |
| | TTTGCCCGCGGTCCCCTCCG | |
| cg206 | CCTCCCAGTGGCCACGCGCCTTCTCACGCCCCTCTCCCGTGACGTCATGCTCCTC | 391 |
| 74577 | TCGCG[CG]GCATGATGGGAGAATCCTAATGTTTTCCAACAGATGCTCCAAGAA | |
| | CAGCTTTCAGATTAA | |
| cg207 | CACCTGGTAGTTGTCTAGCTGCTCTTCGGTGAAGATGGTCTGCTTGTTCCCCAT | 392 |
| 61322 | GGTGGC[CG]CCGCGCCGCCGCTCGCCCGCCCGGGCTCCGACTCCCATCAGCGG | |
| | CCGCCAGACCCGGAGC | |
| cg208 | GACTCCATATGCCCTAGGGATGTGTTGTGATGAACTTTTCCTACTGGTACTGTTT | 393 |
| 28084 | CCTCC[CG]CGAGGGAATGTCTAGACCAGCCGCACCTTCTTGCTTTGACCCTTCA | |
| | GAACTTTGGCCTGT | |
| cg208 | TCTGCCGTACTGTAACTGAAACACAGGTTCAGTTGCTCACTGCTTGCAGAGTCC | 394 |
| 91917 | AGTTAA[CG]AGAGCGGGATCTGTTATAAAGAAAGTGATTTATTCCAAAGCTTA | |
| | GCTTATGAGAAGAAAT | |
| cg209 | AGGGAAGAAATCAACTCCGACTTCTTTGCAAAACTGAAATCTCTGTGAAATAGC | 395 |
| 67028 | CAGATG[CG]CACACCAAATAAGGGTTTCTAAAGAGAACCCAAGTTACTTTTCA | |
| | ATTAAAAAAATAAAAT | |
| cg210 | ATAAACCCGACTCAAAATCTGTCTTTTCCTGGGCAGATTGCAAAGGATTTTGCA | 396 |
| 06686 | TCTCCC[CG]TTGCTGTTGCTGCTGCTCACACAGTCTTGGGAAAACGGGGGAAA | |
| | ATCAAGGAAAGAGAGG | |
| cg210 | CCGGAGGCAGCAGACAAAGACTGGGCAGCACCGGGCACGTTCCCGCTCCTGG | 397 |
| 53529 | CCCCTCCC[CG]GGCCGCACTTCCAGAATGGGAGTGAATTGCCTCCCAATTAAA | |
| | GAAGCAATTTTTTAAAAA | |
| cg210 | GTCTTTCCAAAAGGCATAGGAAATCAGCAAGTTTCCACCAAATATACCAAAACC | 398 |
| 81971 | CTAAGA[CG]CGAGCCAGCCCAAGGGTGCAAGGTTCTGCGGCTGCAGGTGATG | |
| | TGCGTGTGTGCGAGTGT | |
| cg210 | GGTGTGGTTGGTGCGCAGGTCGGCGGGTGACGCGCGGTCTTTGCACACTGGG | 399 |
| 99326 | CAGGTGGG[CG]ACACCTGCACCTCCCAGCAGCGGCTCACGCACCCGCGGCAG | |
| | AAGTTGTGGCCGCAGCGCA | |
| cg211 | AGGACAAATGGGTGCAGAGATTCAGGCTGGCCAAGGCTGGCACAAGGACATT | 400 |
| 20249 | CCCAGTGG[CG]AGAGCATGAGCAAGGGTCACGGATGTGCCAGGAGGGGAGG | |
| | CGGAGAGATGCCTGGGACCA | |
| cg211 | TCATCTATCAACGTAGTAGGCACTGTCCTAGGCGCTAGGGATTCCATGCAGAG | 401 |
| 37706 | CAAAAAA[CG]TCACAGTCCATGCCTTCACATGGCCTTCATGGACCACCGCGGG | |
| | TGTTCTTTTTCCCCCGA | |
| cg211 | CTCTGAAACGGACAAGATGGCTGCCACCTCTTCGCGCCTCTTAGTCCCACCCAC | 402 |
| 84495 | TCAGGG[CG]GAGGTCTGCGTCATGTGACCCTCCCCTTCTTGGCTCCGCCTCCTA | |
| | CCGCAGTGCTTGACG | |
| cg212 | CACTTAATTCTTGCAAATACCTCTCGGTGCTGACTTCAAGGAACTTGGCTGGCT | 403 |
| 00703 | TTGGGC[CG]CAGAAGTGAAAAACACAAAGCTCTCCACAATGTTCAAGTTGTTTT | |
| | CTTCTTAATGTTACG | |
| cg212 | AGCCTAACATCAACTCTTTTAATTGTCATGACAATTCTATGAGATGGGCACTTAT | 404 |
| 01109 | CGCCC[CG]TTTCACAGACAGGGGATGCAGAGGGTACAGAAAGGTACAGTGGC | |
| | TTCCTCGGGGTCACTG | |
| cg212 | CTCGGCCCACACAGCCTCCGGGTGGACCTGCAGGGGCCTGTTTGTGCTGTAGG | 405 |
| 07418 | CTTGACA[CG]TCCAGGTATCTCTGTGTGTCTGTGTATCTCAGTGTGAGTGTGTG | |
| | TGTGTGTGCACACTTG | |
| cg212 | GGTGCGTTGTTCGCGGGGGTGAATTGTGAAGAACCATCGCGGGGTCCTTCCTG | 406 |
| 96230 | CTGAGGC[CG]CGGACACCGTGACCTCGCTGCTCTGGGTCTGCAGGGAAACGTA | |
| | GGAAAAAAAGTTGTCAG | |
| cg213 | TTGCATTCAGGTAGATTATTTGGAAGATGATTTAAGGACGTACCAGTGCAGGA | 407 |
| 63706 | GTTGTCG[CG]GGACAGTGAGACCAGGGCAGTTTGACAATCAATAAAGGGTGC | |
| | ATCATTGGCAAGCTACCT | |
| cg216 | CCAATGGGGAAAGGCAGTGTCGGGACTAAGCAATGAATGGCTCTTCAATGGC | 408 |
| 49520 | CAGCTGCC[CG]CCCAATAGGATAAAAGAAAACCCCACATAATACTTCCCTTTGT | |
| | CTCCAAAAAAATTTATA | |
| cg217 | TGGAGCCCGAAGGCGCCGGGCAGCCTGAAAGGGAGAGGTGGGTCCGGAACC | 409 |
| 12685 | ACACCCAGG[CG]GGTAGCCTGGGGCATCCTCAGACGGACTTCAAAAGCCGCTT | |
| | CACTTTCCCCTGGTGGCCT | |
| cg217 | AGTCTTTCTTCTTGAAAGCATTGTTGATCCAAATCCAAGTGTCAAGGTGCGCCC | 410 |
| 62589 | CAGAAA[CG]CTGCTTCCCAGACAGTCGTGTCTGGTCTTGCGGGAAAGGAGGA | |
| | GGCGTCCCGCCAAGGAA | |
| cg218 | CCACGAAGAGCTTGATGGCGTCGTGGTCCTTCATGGGTACGGCGGGACCGGG | 411 |
| 01378 | GTTTAGCC[CG]CTCATGCCGACGCCGCTGTCCGCGGTGCTGAAACCCAGGCGC | |
| | GGGCCGGGGCCAGCGGGC | |
| cg218 | CGCCTGTTTCCCGCCTGCTCTCAGGAGCGACCGCCAGGGGGCGCCCGAGATGG | 412 |
| 35643 | CAGGGGG[CG]TGGGAAGCCCACATCTGCCCAGCAGGTGCGCCCACCCCGAGC | |
| | AAACAGGGGGCCGGGGCC | |
| cg219 | AAATATTACTGTTTATTACCAGGCATACCCCAGTAAAATAAAGAGGCAACCAG | 413 |
| 07579 | GCGATAG[CG]ACTATCTCACCAGCCGCTGCACCTATAGGACTTGGAGACGTCA | |
| | CGAGTCACGCAACCGGC | |
| cg219 | AGCTGCCAAACATCTGGATCAACCTGGGCACTACGAGGGGTTGAATTTCTACC | 414 |
| 26612 | ATTATCG[CG]CCTTTTGATATTTTTTTCCAGACCTCCTGCTCACATCCGTAAAGC | |
| | CCACTGATTCTTTTA | |
| cg219 | AAGAAAGCTCAAAGGTACCCTGCAGACACTCAAAACTTGAGGGCACGCAACTC | 415 |
| 93406 | TCAGTTA[CG]AGTGGTGGCAATCATAATGACAGAATGAAGTACCAGTGCAAGA | |
| | AACTGGAAGCGTGTGGA | |
| cg220 | CCATGGTGCCCTGGGGCCCTGCTACAGGTGCTCAGGTAGGGAGGTAGGGTGC | 416 |
| 90592 | CTGCTGTA[CG]CTGGACCTGGACCTACTGGGCCCCAGGCAGGACATCCTTTAG | |
| | ACCCTCTGGGAGGCTCCA | |
| cg221 | AACACAGGGTAGGACTTCAAAACACCAGCGTGAGCGAGGCAGGCACACACGG | 417 |
| 79082 | ACTCGCGG[CG]GTCTGTTTGCAACAGCGCTGGGAATGCACATTGGAAAATCAC | |
| | ATCTTGCATGCTGAAAAC | |
| cg221 | CCCGGTTGGTGAGGGAGGGAGTCCCAACCCAGGGTTATGGGTGGCTGGACAC | 418 |
| 94129 | ACAACACC[CG]ACACTGGACAGATAAGACTGACAGCAGTTCAGCTGCATGTAC | |
| | TCACGGCCTGAGGCAGGA | |
| cg221 | GAAGGCTCCTGGGCCTTTCTGGCTCTGGGAATGAAGCGTGGAAAACCCTCCTT | 419 |
| 97830 | AGGCGGG[CG]CAGTGCTTCAAGTAGCCAAGCTCTGACTTCCGAGGGAAGAAA | |
| | GGAGGCCATGGGCCTCTG | |
| cg222 | AACTCAGTCCCGTCCCTTTTGTTGACAGGTTGCCAGGATACATCCAGGCAACAA | 420 |
| 82672 | AGACTG[CG]GTTCCTGTTACTCAGCAGCCTCAAAAACTCACACCAGCTCCTGCA | |
| | AGGAATGTGAATCTT | |
| cg223 | CTCGCCAGGCGGCGCTGTGCCTGGGAGGACTTTCCCGCTCATCGCGGGGGCTG | 421 |
| 95019 | CACGTGG[CG]CTGAAGCCGGGGTCCCACCCCCAATGTGCTCGTCCTACCACAG | |
| | CCAAGGCTGGGATTCCA | |
| cg223 | TGTGCGGAGCCATTCGCTGCGCTGAAGCAGTGCGCATGCGCACTGGACGCTTC | 422 |
| 96353 | TTACCAG[CG]TCCTGACTACAATACCCAGGACGCACCCAGCCCGCCGCCTCTCG | |
| | GAGCCCTTTTCAAACC | |
| cg224 | GCGGCGGAGCGGCGGGTTGGGGCGTCGCACGGTGAGAAAGGCCGGGGCCTG | 423 |
| 07458 | AGAACAAAC[CG]CCGCGGTCGCCGGGGCAACGGGACGGGGCACGTGCCCCCC | |
| | CCGCCAGAGCCGGAAGCGGC | |
| cg224 | GTAGTTGCGGGGACCTGGGAGGCCGGGCTCTTTCCTCCTTGGCCTGCCTTCCG | 424 |
| 73095 | CTGGCTG[CG]TGGGGCAGCCAAGAACAAAGCCTGCGAGCTTCCATCAATTGTA | |
| | AAGCAAAGCACCCTTTA | |
| cg224 | GGCAGGCAGGCTCCATAGTGCCAGGCATCTGGCTGGCTCAGCAGCAGGGGGC | 425 |
| 84793 | GATGGCAT[CG]TCTTCCTGCCCACCTGGGAGCCAATGTTTCGGCTGGGCAAGG | |
| | ACAAGCCTCCTCTGGGTC | |
| cg224 | GAAGGCCCTGACCCTGCTGAGCAGTGTCTTTGCTGTCTGTGGCTTGGGCCTCCT | 426 |
| 95124 | GGGTAT[CG]CGGTCAGCACCGACTACTGGCTGTACCTGGAGGAGGGTGTGAT | |
| | TGTGCCCCAGAACCAGA | |
| cg225 | TATTAGTAAAGCGTTTACTAAATTACCGAATCAAACCGAACTGGCTTAGGTTCT | 427 |
| 11262 | CAATAG[CG]TGGAAATCCACTGAAAATAAATGAAGAGGGCAAACTACAGGGG | |
| | CTCCGCAGGTTCGGGTC | |
| cg225 | CAATGGCTAAGGAGTATAGAAAGGATCATTATAGTGTGTGTCTCTGTGGGTCC | 428 |
| 12531 | TATGTTA[CG]GCAAGATGAAACAAGCTTATTAGGCTCTGTCTTTTAAGGGCATA | |
| | CCAGTTGAAAGAGCAT | |
| cg225 | ACTTGCCCAACATGAGCCCTGGTCTTGTCTGACCCCAAAGCCCATGGGAAGTTT | 429 |
| 80353 | AGGCTG[CG]TGGAAGGACAGCCTGGTGGGCTCAGGATCTGTCCCATCACGAG | |
| | TTGGAACCTCAGCTCTG | |
| cg225 | ACCTAGGAAGTAAGATAATTTTAAAAAGAGAGCACTTTGGCAGTGGTGAAGCA | 430 |
| 82569 | GGTGAAA[CG]GTTGAATACAACACCTGTGGTTTCAAAGAAAAGTTCCCACAGA | |
| | GCGGATACACTACTCGT | |
| cg225 | GGACGGCAAGGACGCGTGGCTGGCGACGGTTTCGCAGGGGCGCCCGTTCCCC | 431 |
| 94309 | TGGGGGCG[CG]AAGTCCCCGCTCCACCGCTGCCCCAACTCGGCTCCGAAGTGC | |
| | CTTTGCCGCAAGACTTGC | |
| cg227 | TGCGCCAGGGCGGCCACGCAGGCCAGGCAGACCACGTGGCCGCAGGACAGGT | 432 |
| 36354 | TGCGCGGG[CG]CCGCTGCTGCCGGTGGCCAAACTTCTCAAAGCACACCTTGCA | |
| | CTCGAGCAGGCTGATCTC | |
| cg228 | TCACATCTGTCATCTCTCAGGTCATATCCAACACACTGGGCCACCCACGCACAG | 433 |
| 09047 | GGACGA[CG]CGACAGCCCTGTGGCTCCACCGCACAGGACAGCCACGACTGGC | |
| | AATCCTGTGCCGGCCCT | |
| cg229 | TAGCTATGACACATGGCTTGGAAATTAACCTTTAACCAAACATCTTATAAGTAA | 434 |
| 47000 | CGCCAG[CG]CAGCTTCCCTTGTGAATGTAAAGAGATCCAGGGCTCTTGGAGAG | |
| | GGACAAGTGAGAGCCA | |
| cg229 | AGGGGGATTCCAAGAGAGATTTTTGTAAATGTCAAATAGTCGACCTCATGCTG | 435 |
| 71191 | GGCAGAA[CG]CTGTATTTCAGTATACAGGGAAGATAAAGAAAGAGGTAGAGA | |
| | AGAGATTGTCCTGTTTTC | |
| cg229 | GACGAGGACAGGACCTCCTGGATGCACTGGAAAGTCGAAGAGACATGGTATC | 436 |
| 83092 | AGGGCAAA[CG]CGTTGCAGAGCTGTATTTGTGAAAGCCAGAATGGAGTGCCTT | |
| | CTTGTCTAAAAGGTTTGG | |
| cg229 | GAGGCCCAGCAGGTAAGCACTTGTGGAGGCCCCGGTGGCTGCTGGTTAGCTCT | 437 |
| 91148 | TGAAGCT[CG]TCCCCACCCTGCGTGCGTTCTAAAGAGCCGCGTTTCTATTGCAA | |
| | CTGCCTGCCCTGCGCT | |
| cg231 | TCAGTCTCCCCATATTTACAATAAAAGGGGAGCGAGGTGGGATGGCGCTGAG | 438 |
| 24451 | GATCCCTA[CG]TCCGATCCTAATCTCCAGCTCAGGCAGGCTCGGCCGCCACTAG | |
| | CATCCTGGAGCGACAAC | |
| cg231 | AGCTGTAATTCCATTGACAGTGAATTGGAGTAATAGCCCTCCCCCGTCTCCCAA | 439 |
| 27998 | GCTCTG[CG]TCCAGTCCACACAAAGCCCACGGCAGCTGCAGGCTGAGCTTGTC | |
| | CTGCTTCAGATCACTC | |
| cg231 | AAACGGAGACTCAGCAACGGGGCTGATTTGTCTGTGGACACACAGCGAACTGT | 440 |
| 52772 | AAGTCCC[CG]CCTCCCTCTGCACCCGCGTGCACCAGGGGGCTGCTGGGGGTGC | |
| | GGGGACGCGGGAGACCT | |
| cg231 | TCCTTGAGCACACACCTTCTCTCAACAAATGACAATACTTGGCAAACTGAACTC | 441 |
| 59337 | CTCCCA[CG]AGTCGCCCTCTGCTAGGAGGAATTGCTGGCTGCTCCCTGCTTATT | |
| | GCATTCTCTCAGAGC | |
| cg231 | CCGGGGCTGCCTGGCCTCCTGGGTGCGGGAGGTGCCTCCAGATTGGCCTGGCT | 442 |
| 73910 | TCTGTGA[CG]CTGGCCCAGATCACACACCAGAGCCCTTGGTGGGCAGCGGCAC | |
| | CTGCAAGCATACTGCAG | |
| cg231 | TTCCGTGTCTCAGATGGGGCCTGGGTCAAGTCCTGGGAGTTGATGGAGCGTTT | 443 |
| 91950 | CCCAAAT[CG]CAAAAGGAGAGGAGCTAGACTTACCTCCCCCTCCTGGGAAGTA | |
| | ATGCGCGACAAGAATTT | |
| cg232 | ATAAATTAACAGTCAGATCTAGGGGCTCGATCAGATTTGTGTGTGTGTGTGTG | 444 |
| 13217 | CCGTGTG[CG]CGTGCACAGCATGTTCTTTGACTAGGAGGCACACCTGCTTTGG | |
| | TTATCTTCTTTTTGTAA | |
| cg232 | GAGGCCTGCCCCAGCCTCAGGAGGAGGAGCCTGGCCCAGTCCGTTGCCAAGC | 445 |
| 34999 | CGAAGCAG[CG]GCATTTGGACAAAGCAGATCATCTGCAGGTATTATATACATG | |
| | GGCAGTGCAAGGAGGGGG | |
| cg232 | TGGAGGTGCTGGGCAGGGGCGGCGCCCCCTTCCCTGGCCGCGGTGCGCCCTT | 446 |
| 39039 | GCGCCCGG[CG]CTTGGGTCCTGCGAGATGAGGGTCTAGAAATACACAGCACC | |
| | ACCCGACCCCCGCATCGGG | |
| cg233 | CAATATTCATTTTATTAGGCCATTGTGAGAGATCTCAGCTCAGCATAATGGGCA | 447 |
| 38195 | ACTTCC[CG]TGACTCTGGGCCACTGGGTTATTCTGGGACTTAACTACTCTGAGT | |
| | TTTCTCACTAGAAAG | |
| cg233 | CTTCCGGCGGACTTGGCCTTTGCGGTGCGAGCTCTGTGCTGCAAAAGGGCTCT | 448 |
| 76526 | TCGAGCT[CG]CGCCCTGGCCGCGGCTGCCGCCGACCCGGAAGGTCCCGAGGG | |
| | GGGCTGCAGCCTGGCCTG | |
| cg235 | TTTATCACCCTTTCGGTAAATAGTGGTCCCACGGCTCGGCCTGCTTTTGGAATG | 449 |
| 68913 | AAGCTA[CG]CTTGGTAAGTTCAACTCTCTTTCACAGCCCTCTCCACAGAAAGAA | |
| | CTCTGGAGTTCGTTC | |
| cg236 | AGAGGGAACTCAGCAGGACAGTGAGGTGACCTTCGCTGTGGCTGTTCCTGGG | 450 |
| 68631 | GACTCTGC[CG]CCACCTCTTCCCCTAACGCCTCCGCGTGTGAATCCTCTGGCAC | |
| | CACCACTTGCCCCATAT | |
| cg237 | GCTGACCCCGGGGAGCGTGGACTACGAGTTGGCGCCCAAGTCCAGAATCCGC | 451 |
| 10218 | GCGCACCG[CG]GTAAGCTGCGCCTTTTGAAAAGGCTATCTGTACTCCTTGGAA | |
| | CAAACCACCCCGGGCAAA | |
| cg238 | TGTGCTCTGGAAAACACATCCCATCAGAGCTGAATCACCCACATGGACTGTTAG | 452 |
| 18978 | CTCAGG[CG]GGGAAACATTCAAGTCATTCAGGCCCAAGGAATAATCTATAGAA | |
| | GTCAAAGGCAAGAGGA | |
| cg238 | GAGAGCGGGTAGCGGGGAGGGCCGCCCACGACGGAGGTTTCTCTGTGGTTAC | 453 |
| 32061 | CTCAGCGG[CG]CTCTTCGCAATCTGAAAGTTGGGGCAGCTGAAGAGCCCCACC | |
| | ACCTTCACCTGCAGCGGC | |
| cg241 | CTGGGGCCTGGGGTCACCTCCCCTCTCTGGGCCAATCACCTGTTGAGTCTGGA | 454 |
| 10063 | GCACTGG[CG]GCTATTCTTAGGGGTTTCTATATTTAAAATGGGGCCTGACTGG | |
| | CTTGAGGTCATCTCCAG | |
| cg241 | GGGACTATTCCTAGTTTATGAGGTGGTTAAGGATATCGGTGGGGTGGGCTGG | 455 |
| 25648 | AGCGGTGT[CG]GGTTAGGTCTGAGAGAAGGCCTCGCACAAAACACTGTACAA | |
| | ACCCGAAAGGAAGTCTGAG | |
| cg242 | AAAATAAAATCCCGCCATCCTCCCCCCTCCCCGCCCCACCCCCGCCAGGTTTCAA | 456 |
| 08206 | CAGCA[CG]GACTCCAGTCCAGTGCAGTGCCGCCACACCAGAGACAACAGGTGT | |
| | TTCGGGAAAAGACCC | |
| cg243 | AGGCGCCATGTCAGCCCGGGAAGTGGCCGTGCTGCTGCTGTGGCTGAGCTGCT | 457 |
| 04712 | ATGGCTC[CG]CCCTTTGGAGGTAGAGAGACGCCAGTCGCAGGCGAGCGACTA | |
| | GGCGGGGATTACCCCCGG | |
| cg243 | CCCGCACACGTGGCCCTCCCGCCTCCGGGCCCCGCCCCCTTGGCCGCAACTGGC | 458 |
| 32433 | AACTCC[CG]CCTGAAGAATAGATTCTCTGGTTCACAGCCGTCTGCAGGCTCAG | |
| | GAACAGATCTGGGCGG | |
| cg244 | GTCGCGCAGCCCTGGCCCGAGGGTTCCCGGGGCACGGCCGCTGGGCCCCCGG | 459 |
| 07308 | TGGAGGAG[CG]TTTCCGCCAGCTGCACCTACGAAAGCAGGTGTCTTACAGGTA | |
| | AGGAGGACGTGGGCAGAG | |
| cg244 | CCACAAAGCGAGGAAGGGCAGGGGCTACGGAGTGGGGGCACCCCGAAAGCC | 460 |
| 93940 | TTGAGCCCC[CG]AGTTTGCTCGGTTGAGGGTGTTGGGGGCACAGGGATGCTG | |
| | GCCCCCAGCTCCCCACTGGA | |
| cg245 | AATGGAAACTGCTAATTTTTGAAGCAGAAGGTTGACAGCTTCAGTAAGATCTC | 461 |
| 05122 | AAGAGAG[CG]AGAAGACTGGAATCAGGTGAGGCCATAACTTCTTATCTAAACT | |
| | TAGTTTCTGGGGTGGAA | |
| cg245 | GTGGGGGCTGGGCAGCGTGTTTGTCCCACCTGTGTAAACTCTGATTCCAGCAA | 462 |
| 05341 | CTTATTC[CG]CATGCGCCCAGTCTAATTAAAATAAAAGTGAATCAAATTTTGAA | |
| | TGGATTGGTGTTTCGA | |
| cg245 | GTGTGAATTGATGACCAAGGCATGGCAGAGCCTCTCTCATCTTTATAATCAGTT | 463 |
| 56026 | CAGCGG[CG]GCCTCCACTACAGGGAACTCCCAGCCAGTCCCGAGGCCTAGGG | |
| | ACATCCAGGGAGAAACG | |
| cg246 | CAGGCGCTTCCCACCAGCTACAGTCGGAGATTTGGAGCGCTTGTGTCTGAGGC | 464 |
| 51706 | TCAATCC[CG]TCAGGTGCCGCGCAACTCAGCGGCGCATTCTCTTTGGACCCGA | |
| | GGCACCACCATACTTTC | |
| cg246 | GCCTGCTCCCCGTCCCACCCCTCCCTGAGCACGCCACCCCGCCCTCTCCCTCTCT | 465 |
| 74703 | GAGAG[CG]AGATACCCGGCCAGACACCCTCACCTGCGGTGCCCAGCTGCCCAG | |
| | GCTGAGGCAAGAGAA | |
| cg249 | CTCTGCGGTGGCCCGAGCCCCAGCGGCCTCAGGTGAGCGGGCAGCATCCCGA | 466 |
| 21089 | TTCCCTGG[CG]GCCTAGAATGGAATCGCAAGGTTTAGAGAAATTAAGGGACCT | |
| | GGGACTTGCCACCCTGGG | |
| cg250 | ATACACATTTTTGGCCCCAACCTGCATCGACCAAGTCAGAAATTCTGCAGTGTG | 467 |
| 22327 | TGTTTT[CG]TAAGTCCTCCAGGTGACTCTGATGTACTCTCAGGGTTCAGAACCA | |
| | TTGAGAGAGAGCAGT | |
| cg250 | TGACAGCCGGAGGTTCCAGCTGCGCGCCCACAGCCCCTCGGTAGCGCCGCCGA | 468 |
| 92328 | CTCGTGG[CG]TCTATAGGCTGTTTCTGCGTCACTCATGCATGGAAGACCAATCA | |
| | GAGAGCGTACTTGTCT | |
| cg251 | GTGCCCCCTCCTCTTTGCTGCTGCAGTGTCTGCGCCGGGCCATTTAATGAGATT | 469 |
| 36687 | TATTCA[CG]CACGGCTCTTCTCAGCTTTGCGAGGGGTTGGCAGATCCAGTGCAC | |
| | AGGGATTTCCCACTA | |
| cg252 | GCACAGCTGCCCTTTGAAGTACGGTCTATTATATCTCTTTTACAGACCCAGAAA | 470 |
| 29964 | CTGAGG[CG]CAGAAGTTAGGGTCAGCCCCAGGTCACACAGCTAACAAGAGCT | |
| | GGCCTAGGCACCCAGGG | |
| cg252 | GGGGCGTGGGTGGGTCAGCGTTCCTTGGGGACCCGTGAAGCCTGGGCTTAGG | 471 |
| 51635 | GCTCACAG[CG]TGGGTCCCCAGCACAGACAGGAGGCGGACAGCTTCCCGTGA | |
| | ACTGCAGGGGAGTCCCGGG | |
| cg252 | CAAACTAGTGACTGTTTTACTGCAGGTGAAGAAGGGGCAGAGATCAGAGGCT | 472 |
| 56723 | CTAGCAGG[CG]GGACAATGCCCAGGGATTCATGAGCCGGACAAAGCTGTATC | |
| | CCTCCATTTCCACCTGCCA | |
| cg254 | GGAGCCCCTGGGATGACCCATCCCAAGGTCCCAGCCTAAGTCTGAGGTTCCAG | 473 |
| 28451 | GGCTGGT[CG]CAGGCCGTCCTTGCAGCCCTCGCCAGAGCGTTGTCTGCACCTC | |
| | CGACACTAGGTGGCGCC | |
| cg254 | GAGGGATGGTTGTCCTCACCCCTGTGAGGCAATATGCTGTCCATTAGTATCCAC | 474 |
| 59323 | TGAATG[CG]TGAAATTTTTTTCTAATGGGCAAACTGAGGCTCAGAGAAGTTCCT | |
| | GTCTGGCTCAAGGTT | |
| cg255 | TCCCGGGCGCGGAGGATGGAAACCTGGCGGTAACCTCTGCAGGTCGTGCCAC | 475 |
| 36676 | TCGGTGTG[CG]CAAGGTCTCCAGAGGCATCTTTTCATTTTTAGGGGGCACTTTC | |
| | CACGAATTCATTTGAGC | |
| cg257 | GCGCTTCCAGAAGGCTGCAAATGGGAATTCCAGACAAACCCACTTGGGTGAAT | 476 |
| 13185 | CCCAGCA[CG]CGGGCTGCGGCGTAGGGGGAGAGCTCCTCACGCGGCTCAGAG | |
| | TGTAGCCCAGGCCCGCAG | |
| cg257 | GGGAAAGTCTCAAAACTGTCAACTCTGATAGAAAGCTCATGTCAGAGACCTGA | 477 |
| 69980 | AGCTCAG[CG]ATGTAGTTCTGAGACATATCTAAGACTTTGGTTTTCAGCGGTAG | |
| | GTCTTTTGGAACATGA | |
| cg258 | TCTACCTAGTAACAGCTGAGAAATAAGGCTCGAGACACCATTGGTTGGTTCAG | 478 |
| 81193 | CCTCACT[CG]GCCAATCCTGGGCTCTAAACTGCTCAGTGGAAATCTTGGGACTT | |
| | TTTGGACACCCAGAGA | |
| cg258 | CAGCCCACGTGACTACAGGGGCACTTGATGGGAATCATGGCAGCATCCAGGCC | 479 |
| 98500 | ATTGTCC[CG]CTTCTGGGAGTGGGGAAAGAACATCGTCTGCGTGGGGAGGAA | |
| | CTACGCGGACCACGTCAG | |
| cg260 | AGGAGGATCTCTGTAAATTGTTTTCTTAGGGAGAAGGATAGGGTGAAGGAGT | 480 |
| 22315 | AGAATCGA[CG]ACTGTAGATTTGTGAGTAGAATCCCATTTGTAGTTAAACTTGG | |
| | GTAAATGGGAGAAAGGG | |
| cg260 | GCCTCTCTGTGGTTCTGCCTGGAAGACGGAAGGCAGGTGGTTGGCTCTAGTCA | 481 |
| 91688 | TCCACGA[CG]GGCTGGCACCTCTCCAGCTGCGGCCAGTCTAACCCCAGGGCCT | |
| | GCTGGGAAATGTAGTTC | |
| cg260 | GCGCCCCTGGCGTCCGGGCAGGTGCCAGGTGAGGAAAGAAATGGGGGCCGCT | 482 |
| 96837 | CCATGAAG[CG]GTTCCTGCCAATAAAGAAAACGACATCCAGAGAATACCCAGG | |
| | CGGGGAATAAAGGGGTCC | |
| cg261 | CAGCACGGGCGGGGGGCAGGGGCTGGGGCCGACCGGGAGGCCGGTGCCAA | 483 |
| 04204 | GGATGGGGGC[CG]CCCGGCTGCCCCGCGCGTGAGGAGGCCGAGGGGCGCGC | |
| | CACCCCGGCCCGGGGCGGCCGC | |
| cg261 | TAATCTCTTCTTTGGACGTTTGGCAGCTCCATTTCACCTCCCCTTAACTCTGTTTG | 484 |
| 09803 | GGAT[CG]CTTACACACCAAGGAAGTTGGGCTTTGAGAATTCCATCCCACTGGC | |
| | ACTGAGGAGAATAT | |
| cg262 | CAGCCTTTCCCCGGGCCTGGGGTTCCTGGACTAGGCTGCGCTGCAGTGACTGT | 485 |
| 01213 | GGACTGG[CG]TGTGGCGGGGGTCGTGGCAGCCCCTGCCTTACCTCTAGGTGCC | |
| | AGCCCCAGGCCCGGGCC | |
| cg262 | GGCTTTCCCGAATGGCGCGCCCAGGACGGCTCTTGCGGCTGGCTGTCCAAACT | 486 |
| 12924 | GGGCCCG[CG]TCCTGAAGTGACCCCAGCCTGATCTCGGCCAGCTGCTTGTGAC | |
| | CTTGGCCTGTCCCAGCA | |
| cg262 | CCTGGCCGGCCGGCTCGCTAGGCGCGGGGTCTAGGCCAGGCTGGGGCTGCTT | 487 |
| 19051 | GGAGGCTG[CG]CCCTCCCCTGCCCGCGGCGCCCCGGCCCCCGCCGTCGAGAGT | |
| | GGACGCCCCTCTGGGGTA | |
| cg263 | CTCTAAAAAGTGACATTGATGCCAACTGCCAGAGCTGGTACCCATGCCATCTGC | 488 |
| 12920 | TAGTGA[CG]TCACAGGGCAGAGAGAGCCATGTGATCCTCTCTCTTGGGACCTT | |
| | CATTCTGCACTGATCA | |
| cg263 | GTTTGCACTGAAAGTTGTGTTGGCTCAGGAGCTGCTTTTCCGGGGATCTGCAGT | 489 |
| 50286 | TGCCCC[CG]CCACCTCCTGGCTGCGGTTGGCAGGTCCCTCCCTCAGCAGTTCGT | |
| | CCTCCGCCTGCGCCG | |
| cg263 | CAAGGAGGGAGCAGGAGCATTCGAACGCGGAAATCGAGGTGCTAGTCCAAAC | 490 |
| 57744 | TGCTCGGT[CG]GCTTTAGTCATAGCTGGATAATGCCCGGCTCAGGTCTACCACA | |
| | AGCCATACAGCTGCTTT | |
| cg263 | TTGTTACGGGCGCGGTGGTGCAGGGGCAAATCGGGACTGGGATTTGGTCCTT | 491 |
| 82071 | ACCCTTAA[CG]TGGCTCTAAGACCAGAAGGGAACACCTGACTTGTGTTGACCT | |
| | CTTCAGTTAGCTGCAGGT | |
| cg263 | TGAAAACACAGCAAGGGCCCCACTAGCTGAAACCAAGTTGCAGAGTTTTGAGG | 492 |
| 94737 | GTCCCAC[CG]CCGACCGCCGGCCCGCCGCGAGCCCTGCCCCCTGCGCGGCCAC | |
| | GCCCCCTTGCTCCCCGC | |
| cg263 | TAAATAAATAAGGGCTTTTGTTTGTTTGCCGGCTCCTGCACATGGCTGCTGGGA | 493 |
| 94940 | CTCAAG[CG]CTCGTGTTGTCTGCGCCTCTGTGGGACTCTGGGGACGGGAGGCA | |
| | GGGGAGGCCCCCGCAG | |
| cg265 | GAGGCTCTGAGGCTGCAACAGTCTCCCTCCTATTGAAGCTAGAACAGCACCCC | 494 |
| 81729 | GAGCCTG[CG]CCATAAGTGCCCCCAGAACTTCAGCGCCCACCATGGCGCACAA | |
| | GGCCGGTGCCCAGCGCC | |
| cg266 | CTTGGGCAACGTAGGAGACCTCCGTCTCCACAAGTAAAATTAATTAGCCGGCT | 495 |
| 14073 | GTGGTGG[CG]CGCACCTGTGGTCCCAGCTACTCAGGAGGCTGAGGTAGGAGG | |
| | ATCACCTGAGCCCGGGAG | |
| cg266 | GAGGCTGAGGCAGGAGAATGGCGTGAACCCGGGAGAATGGTGTGCCCCGAG | 496 |
| 65419 | CCTACTTCC[CG]CGCAGTCCTCCAAGACGCGGCCTCCAGCAGGGGTCGCTGCTT | |
| | CGCTGCCGCCCTGGCCTC | |
| cg267 | GCCAGGACCAAATGCCCCCGGAAGCGGGGAGCGACAGCAGCGGAGAGGAAC | 497 |
| 11820 | ATGTCCTGG[CG]CCCCCGGGCCTGCAGCCTCCACACTGCCCCGGCCAGTGTCT | |
| | GATCTGGGCTTGCAAGACC | |
| cg267 | GCTTGGACACTTGCAGCATGTTGCCTCTGCCAACTGCCTAGAATTTAAGCCTGA | 498 |
| 46469 | CTTTGC[CG]CCTTACTGACCCCAGCAACTTAACCTGTCTGTGCCTTAATTTTCTT | |
| | ATCTACAGAATGGA | |
| cg268 | AAAGTTAAGTACTAAGTATGTGGTGAACAAATAAATCCACCCTTCTGAAACACA | 499 |
| 15229 | TCTAAA[CG]AGGTCCTGTATTTGAAAGTGTCTGGAAGATTAAAAGGCACTACA | |
| | CCAAAGCTGCTAGCAC | |
| cg268 | GGACTGGTACAGGACAGGCATCTTTGAACCTATTTCTGGGAGTTCTGAAACTAC | 500 |
| 24091 | TGTTCT[CG]TGGGCCTTGGCGACTGATTTGGGAAAGCTGACCCTGGGTTGGCC | |
| | TGGCTTCCAGCCACCG | |
| cg268 | CGACGACGACCTCAACAGCGTGCTGGACTTCATCCTGTCCATGGGGCTGGATG | 501 |
| 42024 | GCCTGGG[CG]CCGAGGCCGCCCCGGAGCCGCCGCCGCCGCCCCCGCCGCCTG | |
| | CGTTCTATTACCCCGAAC | |
| cg268 | ACAAAGTGATCTGGCACAGCTGCAGGGTGGCATTGAGTCTGAGGCTTATGGTG | 502 |
| 66325 | CAGAAGC[CG]AAGTTAAAGATGTTTCTAGAGCCTGAAGACTTCCTCTTGAGGG | |
| | TGAGTTGCTGCCTACAA | |
| cg268 | CGCGGGTGGAAGGTGAAGGTCGAGGGAGGTCAGGCTGCTTCTGCGTGTCCTG | 503 |
| 98166 | ACGGCTGG[CG]TGTTCTCTTGAGATGGGCTCGGGCTACTTGGCCAGCTTCAAT | |
| | TTAAGCCACAGTGTCTCC | |
| cg269 | CCCAGCCCACGGCGGCCCGCGAGGGACAAAACGCGCCGCGCCTGGTTCCCCG | 504 |
| 32976 | CCCACGGA[CG]CGGTGACTTTCCAGAACGTCTTAAAGGCAACGCACTCTGACT | |
| | CAAGGCCCAGGGAGGCTG | |
| cg270 | TGTTTTTGTGGGAGGCCTTCTGCATGGTCCCGGGAGGTCAGGCAGCCCGGGAG | 505 |
| 15931 | GGCCTCC[CG]GAGCAGAGGCTGGAGTCAGTCCCAATGCCAACAGTTTCGAACC | |
| | TTGCCCGCGGGCACTGC | |
| cg271 | CCTGTCTTCAGCAGCATCGCTCTGGACTCAGCTTCCGAGGACCTGACCAGATCT | 506 |
| 87881 | GGTCTG[CG]TGTATCAGCTGTATGTGTTGGGCTCTGGAAGCTAAGAAACGTCT | |
| | GAAAAGCACTGGGGTC | |
| cg272 | TGCCCCGGTAACTGCCTCCCCAACACCTGCCTGCCTTCCACTGCGAAACCTGCTC | 507 |
| 44482 | TCGGA[CG]CCCTGACCATACCGCACACAATACTGCAAGCCTGTGTGGGCCTGG | |
| | GGGTGGGATGGACCC | |
| cg273 | GATGGCCCTTTAAGAGGCACTGTCCAGCTCTGGTTGCCATGGAGACAGCTGGA | 508 |
| 67952 | CACAGAC[CG]GGTAGAGGCAGGCCCACAGCATGTCCTCCAAGGTTTACTCCAC | |
| | AGGTGGGAAGAGGACTG | |
| cg274 | CTGGGATTACAGGCGTGAGCCACTGCGCCTGGCCTTTGCAAGGTTTTGAGGAA | 509 |
| 40834 | AGTGAAG[CG]TTCTGTTGAAGCAGGGCTTGAGTTCTGTTGTAAGTGTTTCATG | |
| | AAGCCCTGGAGACCTCT | |
| cg274 | TCAGGTTCTGGAACCAAGACAAGTCCAGGGACAACCCCAAAGCTGGCCTGGGC | 510 |
| 93997 | TCCCGCG[CG]GACAGCTTTTATACCCTGTACGGAACCGCCCCTGCCCAGGATTG | |
| | AAGTGGCCCCGCCTCC | |
| cg275 | AGGTGGAAATACTTTCGGGCGATGGTGGGGGCCTGGTGCTTCTTGGACTCGG | 511 |
| 14224 | AAGATGAC[CG]CTTGGCATTCTGGTACAGCACCACCAGGCAGGCCAAGGTGG | |
| | CCAGCAGAGACCAATAGGC | |
| cg276 | GAACCAGGGCCCTTGGCGAGAGTTGGGGTGGGAATCGCGTAAGAAAAGCAAT | 512 |
| 26102 | TTCTAGAG[CG]GAAAGGTGACCCCACATTACAAAAAGAAATGGAGTAGAAAA | |
| | ATAGGCTTGACTATTCTAA | |
| cg276 | CTATCAGCCTAACGATTAAGTCAACATGCTAAGCAGCCACACGGGGGCTACTA | 513 |
| 55905 | AGTGACT[CG]CACGGGGGAAGCAGGCAGGGAGACAGATGGGCAGGGGAGG | |
| | GAATCTGGGGCAATGCACAA | |

Note:
This application references a number of different publications as indicated throughout the specification by reference numbers. A list of these different publications ordered according to these reference numbers can be found above.

All publications mentioned herein are incorporated herein by reference to disclose and describe the methods and/or materials in connection with which the publications are cited (e.g. U.S. Patent Publication 20150259742). Publications cited herein are cited for their disclosure prior to the filing date of the present application. Nothing here is to be construed as an admission that the inventors are not entitled to antedate the publications by virtue of an earlier priority date or prior date of invention. Further, the actual publication dates may be different from those shown and require independent verification.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching.

1. A method of obtaining information on a phenotypic age of an individual, the method comprising observing methylation of genomic DNA obtained from the individual, wherein:
methylation is observed in at least 10 CpG methylation markers in polynucleotides having SEQ ID NO: 1-SEQ ID NO: 513;
said observing comprises performing a bisulfite conversion process on the genomic DNA so that cytosine residues in the genomic DNA are transformed to uracil, while 5-methylcytosine residues in the genomic DNA are not transformed to uracil; and/or
said observing comprises hybridizing genomic DNA from the individual to a methylation array comprising the polynucleotides having sequences of SEQ ID NO: 1-SEQ ID NO: 513 coupled to a matrix;
such that information on the phenotypic age of the individual is obtained.

2. The method of claim 1, wherein the method comprises observing a clinical variable in the individual comprising at least one of: concentrations of albumin in the individual, concentrations of creatine in the individual, concentrations of glucose in the individual, concentrations of c-reactive protein in the individual, concentrations of alkaline phosphatase in the individual lymphocyte percentage in the individual, mean cell volume in the individual, red blood cell distribution width in the individual, white blood cell count in the individual, and age of the individual at the time of assessment.

3. The method of claim 1, wherein methylation is observed in genomic DNA obtained from leukocytes or epithelial cells obtained from the individual.

4. The method of claim 1, further comprising comparing the chronological age of the individual at the time of assessment and the phenotypic age so as to obtain information on life expectancy of the individual.

5. The method of claim 4, further comprising using information on the phenotypic age obtained by the method to predict an age at which the individual may suffer from one or more age related diseases or conditions.

6. The method of claim 1, wherein the phenotypic age of the individual is estimated using a weighted average of methylation markers within the set of 513 methylation markers.

7. The method of claim 6, further comprising assessing a plurality of methylation markers in a regression analysis.

8. The method of claim 1, wherein methylation is observed by a process comprising hybridizing genomic DNA obtained from the individual with at least 100, 200, 300, 400 or 500 polynucleotides comprising SEQ ID NO: 1-SEQ ID NO: 513 disposed in an array.

9. The method of claim 1, further comprising:
comparing the CG locus methylation observed in the individual to the CG locus methylation of genomic DNA having SEQ ID NO: 1-SEQ ID NO: 513 present in white blood cells or epithelial cells derived from a group of individuals of known ages; and
correlating the CG locus methylation observed in the individual with the CG locus methylation and known ages in the group of individuals.

10. A method of observing a phenotypic age of an individual, the method comprising observing methylation of genomic DNA obtained from the individual, wherein:
methylation is observed in 513 CpG methylation markers in polynucleotides having SEQ ID NO: 1-SEQ ID NO: 513; and
said observing comprises hybridizing genomic DNA from the individual to a methylation array comprising the polynucleotides of SEQ ID NO: 1-SEQ ID NO: 513 coupled to a matrix;
such that the phenotypic age of the individual is observed.

11. The method of claim 10, wherein the method comprises observing a clinical variable in the individual comprising at least one of: concentrations of albumin in the individual, concentrations of creatine in the individual, concentrations of glucose in the individual, concentrations of c-reactive protein in the individual, concentrations of alkaline phosphatase in the individual lymphocyte percentage in the individual, mean cell volume in the individual, red blood cell distribution width in the individual, white blood cell count in the individual, and age of the individual at the time of assessment.

12. The method of claim 11, wherein at least 3, 4, 5, 6, 7 or 8 clinical variables are observed.

13. The method of claim 11, further comprising observing at least one factor selected from individual diet history, individual smoking history and individual exercise history.

14. The method of claim 10, further comprising using the observed phenotypic age to assess a risk of a cancer mortality in the individual.

15. The method of claim 14, wherein the cancer is a breast cancer.

16. The method of claim 14, wherein the cancer is a lung cancer.

17. The method of claim 10, further comprising using the observed phenotypic age to assess a risk of diabetes mortality in the individual.

18. The method of claim 10, further comprising using the observed phenotypic age to assess a risk of dementia in the individual.

19. The method of claim 11, wherein methylation is observed by a process comprising treatment of genomic DNA from the population of cells from the individual with bisulfite to transform unmethylated cytosines of CpG dinucleotides in the genomic DNA to uracil.

20. A tangible computer-readable medium comprising computer-readable code that, when executed by a computer, causes the computer to perform operations comprising:
a) receiving information corresponding to methylation levels of a set of methylation markers in a biological sample, wherein the set of methylation markers comprises 513 methylation markers having SEQ ID NO: 1-SEQ ID NO: 513;
b) applying a statistical prediction algorithm to methylation data obtained from the set of methylation markers; and
c) determining a phenotypic age using a weighted average of the methylation levels of the 513 methylation markers.


SEQUENCE LISTING (513 sequences)

SEQ ID NO: 1 - DNA, 122 bp, Artificial Sequence
  Description of Artificial Sequence Synthetic polynucleotide
taaaaatgatcatttctgcttacgtttacagctcatttcatattctgcaaaatgttttcccgtctgctatcaccgccgccatcctcacagcagcctgggagaaaggcagagccaaaagtctc

SEQ ID NO: 2 - DNA, 122 bp, Artificial Sequence
  Description of Artificial Sequence Synthetic polynucleotide
gccctgcgggaagggactggggttgggaggacgctgggcctctgggtttaggcctcactccgccggagagggggagacaaacaggccagactctcttcccagagcaggagcgacccctcccc

SEQ ID NO: 3 - DNA, 122 bp, Artificial Sequence
  Description of Artificial Sequence Synthetic polynucleotide
gcgtttgtaggcagtgatgtcacagagtgccttcatgctcctcgggtctccggttctccccggacctctgtagtcctcattgccaaagttgtaccccctggggagtgcaccctgcctgcatt

SEQ ID NO: 4 - DNA, 122 bp, Artificial Sequence
  Description of Artificial Sequence Synthetic polynucleotide
ctttgctttcttatctccagctcacacctttaagtcttatgtagttaaaggacatttatccgcctccttggagaacacagccctccagtgtctcctgcagcctggagcctgggacattctgg

SEQ ID NO: 5 - DNA, 122 bp, Artificial Sequence
  Description of Artificial Sequence Synthetic polynucleotide
ttattgtaaacccattttaccagtgatgtgaatgagccgcaatgaaggctaagggacttgcgcaaggtgacatatataagcaacaggcctgcgattggaatccaggccccagagtctgggca

SEQ ID NO: 6 - DNA, 122 bp, Artificial Sequence
  Description of Artificial Sequence Synthetic polynucleotide
agggggatggagcttctacacagggccccagcgctgtcgctgtggctgctgctgccgctacggcttagtgcaccagacgctgcatttcaggtgctcctacaaaagaggccactcctggaacg

SEQ ID NO: 7 - DNA, 122 bp, Artificial Sequence
  Description of Artificial Sequence Synthetic polynucleotide
aaggtcccgcggcctcgggccccgccccgccccggggcctcgagcgccaggccggcccggcgaaccccgcccaaggccaacaaggagccttgtccgcgcattccagcggcagaaacggaatg

SEQ ID NO: 8 - DNA, 122 bp, Artificial Sequence
  Description of Artificial Sequence Synthetic polynucleotide
cctcagatgcacagtgacacccaccttggagagtttctgtgtctcttaaatgaccgaatccgtgtagaaggcttattaccacaatctgtagctacttggtaaacggcagctcttattttgac

SEQ ID NO: 9 - DNA, 122 bp, Artificial Sequence
  Description of Artificial Sequence Synthetic polynucleotide
gccaaaccagtggctgtttctgaaatgtgagcttccgccccaagctaaaaagtgttcacacgtgggtggtctggaaaagaccaaagagagagacctgagttgaatttgccaggcgggtaaac

SEQ ID NO: 10 - DNA, 122 bp, Artificial Sequence
  Description of Artificial Sequence Synthetic polynucleotide
cagaacaccgaataaataccagttcttacatgacatttcactccacggaaaaatctggagcgcacactgcaccgccgcccgtgtggcctgcccgcaacccggtggctctgcccggccccggc

SEQ ID NO: 11 - DNA, 122 bp, Artificial Sequence
  Description of Artificial Sequence Synthetic polynucleotide
gaagcagttcgatgcctaccccaagactttggaggacttccgggtcaagacctgcgggggcgccaccggtaggccgcagcggggccggggtcgcgtggaggggggcgtcctagagcttagcc

SEQ ID NO: 12 - DNA, 122 bp, Artificial Sequence
  Description of Artificial Sequence Synthetic polynucleotide
cttgcacctctggcttttgcaaactgggggcccaagagctgcacccagggattttatagccgttcttatcggtcctcaggatcaaggaccaatcaggtccctcaactggtctggtgagccaa

SEQ ID NO: 13 - DNA, 122 bp, Artificial Sequence
  Description of Artificial Sequence Synthetic polynucleotide
ttgggtggggcgtctcagcattcctccaacgggcaggtctcagcgctcctccccctgctccgctcctctgcagggcccaggcgcccttggccttaggacccaacttctcttaccgccatgga

SEQ ID NO: 14 - DNA, 122 bp, Artificial Sequence
  Description of Artificial Sequence Synthetic polynucleotide
tttctctgggagggggcctctgcccagctgtcccctgtgcgtcatgtgcaggaggccaggcggctcgccttacagggacccggccacctctatatatagcccctcgaagacagctgctcagt

SEQ ID NO: 15 - DNA, 122 bp, Artificial Sequence
  Description of Artificial Sequence Synthetic polynucleotide
cagaggagactcctggtcccctgtccggaccccgccccgaccaggtccagccccgcccaacggcaagttaagagccccccagtgccagacgctccagacagactgccactcttggggggcaa

SEQ ID NO: 16 - DNA, 122 bp, Artificial Sequence
  Description of Artificial Sequence Synthetic polynucleotide
ctggaggcatcttcggacctctgggcggcccagccctgcctggcgtctccccgccgcttgcggcctaccgccaagaagctatgccttaggcaaaccatggagctctggccccagagggcgcc

SEQ ID NO: 17 - DNA, 122 bp, Artificial Sequence
  Description of Artificial Sequence Synthetic polynucleotide
ggtgccagtgaaggccgggtgcctggtccccccaggaggctggtcttggagcaggtggtccggtgctggtggtggaaggacagcagcttctctgctagtggccacaggcagagcctgccttt

SEQ ID NO: 18 - DNA, 122 bp, Artificial Sequence
  Description of Artificial Sequence Synthetic polynucleotide
aaaacatgccccagctttcccaagataaccaagagtgcctccagaaacatttctccaggccgtctatatggacacagtttctgcccctgttcagggctcagagatataatacagacattcac

SEQ ID NO: 19 - DNA, 122 bp, Artificial Sequence
  Description of Artificial Sequence Synthetic polynucleotide
ctacaactatggcttgtctgagtcctgagccagcagagctcaggccacagcacctgcacccgttttctgctgctgcacacaagggctctgtgcattccgcatccaggtgtgcccctcctctt

SEQ ID NO: 20 - DNA, 122 bp, Artificial Sequence
  Description of Artificial Sequence Synthetic polynucleotide
ttctccaagtaattttcatgtgcagccagaattgcaactcaccaggctaaactgcagttgcgcaattctggtcttcttgatacctgatttctttgccccttctcttttctggttcaatgcat

SEQ ID NO: 21 - DNA, 122 bp, Artificial Sequence
  Description of Artificial Sequence Synthetic polynucleotide
aatccccctaccttgatgtcttctcttagtaatcccactgatcctctctgttttctttgccgtattcagtgttaagcacagtaagtctttcctactgaaatagccatggtcctaatcataat

SEQ ID NO: 22 - DNA, 122 bp, Artificial Sequence
  Description of Artificial Sequence Synthetic polynucleotide
actcagttcaaggtttataagaagaggaaatgttttgccctggccgcgtttccttttccacgtattgtctgttagagtgcaagctgaaataatgggttttctagttaatggcatgttccaat

SEQ ID NO: 23 - DNA, 122 bp, Artificial Sequence
  Description of Artificial Sequence Synthetic polynucleotide
gcccggatgcgtccctctttctccaccccgccgagcctaaactagtgacggggagggagacgggatagtgtttctgtttcgtggtctttgaatccacaacctctagtctgaacacagagaac

SEQ ID NO: 24 - DNA, 122 bp, Artificial Sequence
  Description of Artificial Sequence Synthetic polynucleotide
cccacttttccagattgctctgaatgtcctagtgagctgctcccgttgggtaggctcctgcgcctcaaccgcgctcggtactcgacgtttattatcagggaattctcggctgcaagatggga

SEQ ID NO: 25 - DNA, 122 bp, Artificial Sequence
  Description of Artificial Sequence Synthetic polynucleotide
agaaggaactctgaagactccgtagattgctctagaccgcctcagacactctcggcgcagcgtggagaggatttgtgcaaacatttcctctgtggaccaagaggaatgcaagaggaggctgc

SEQ ID NO: 26 - DNA, 122 bp, Artificial Sequence
  Description of Artificial Sequence Synthetic polynucleotide
tcgcgggtgatctcctggctcagggcccgcatgcgggagtagcaggtcgggggagtgggccgcgcggcgggggctcccgccaggagcagcagcagcacgggcagaggcccaggcgtcctcat

SEQ ID NO: 27 - DNA, 122 bp, Artificial Sequence
  Description of Artificial Sequence Synthetic polynucleotide
ttgccagcttagttgtaatttcttgtatccatcttggtcctcttcagtgcccagccagagcgctggcagacaggcactgggtacgttttgttgaatgaattgggagcgaacgtcgtttagtg

SEQ ID NO: 28 - DNA, 122 bp, Artificial Sequence
  Description of Artificial Sequence Synthetic polynucleotide
ctccgtcggcccgggctcctgccttgggggtgtcccctaggtagagaatgcgtcggggagcgcttcccgccagagatgggaagcccaggaagcccctccccatgcaaacagtgcccccgcct

SEQ ID NO: 29 - DNA, 122 bp, Artificial Sequence
  Description of Artificial Sequence Synthetic polynucleotide
cgtgtatatttttaaactgtgtgctgacgacagttaagtaaatgtgattcagaacttctgcgtattttgcaggacagttttgacacaatgacatgactcgctagccaggaaagataacgaca

SEQ ID NO: 30 - DNA, 122 bp, Artificial Sequence
  Description of Artificial Sequence Synthetic polynucleotide
ttcttaataatgaatgaaccaacgacccccaaggctggtttgcccgtgcacacgcacgcacgtgtgcaacacgtagcacttgctgagtgtttgctacttgccaggcctcatgtcaagcactt

SEQ ID NO: 31 - DNA, 122 bp, Artificial Sequence
  Description of Artificial Sequence Synthetic polynucleotide
ggccaggagagggagacttggcccaaataaagtgactcaggcaccctcaggaactctcggcgcccggggccccttcgggcagccttcgacccccatgcgtctttcgggtccccagggacgcg

SEQ ID NO: 32 - DNA, 122 bp, Artificial Sequence
  Description of Artificial Sequence Synthetic polynucleotide
cacttttcctccccagtacgtgggagccctagaggacatgttgcaggccctgaaggtccacgcgaggtgagtgcaggcagcctcagggctttcacatcagcacgtggctgtgctactggaca

SEQ ID NO: 33 - DNA, 122 bp, Artificial Sequence
  Description of Artificial Sequence Synthetic polynucleotide
ggcgaacccacccctccaggcagggtttcgcccctcgccccgccccttcccccgcccggacggccatggccattcccggcatcccctatgagagacggcttctcatcatggcggaccctaga

SEQ ID NO: 34 - DNA, 122 bp, Artificial Sequence
  Description of Artificial Sequence Synthetic polynucleotide
cagttttagtcctttacggtgatttgtaagcccaggccttcttaactaggcaaatgctgccgccaggtggcctaggcctaaccccagagccgttgtcttgacgcttaagcttccggggaggg

SEQ ID NO: 35 - DNA, 122 bp, Artificial Sequence
  Description of Artificial Sequence Synthetic polynucleotide
acgggcaggaatctgttgtagaagagttgctgccgggacctgctggtgaattggctccaccggatccggctccgcagaaagctcactgcttcctgtggctcctggatttccaagcctctggg

SEQ ID NO: 36 - DNA, 122 bp, Artificial Sequence
  Description of Artificial Sequence Synthetic polynucleotide
aagaagctaggaggggaaataaattgagtgggggtggggtttcccaagaatcggaggaaccgagaacgaagaggggtgggggaacggggaaagagagaggaaaatcaagttttcttcagcac

SEQ ID NO: 37 - DNA, 122 bp, Artificial Sequence
  Description of Artificial Sequence Synthetic polynucleotide
tatgacacacctatattcacacagttgtgactgtggacacgcaaaatgcctgaggccctgcgtccaatcccggaagcacagttcctgggaggagtcacttctataatagccgtatcttccct

SEQ ID NO: 38 - DNA, 122 bp, Artificial Sequence
  Description of Artificial Sequence Synthetic polynucleotide
ggtggtggactttgggactggacagacctggtcacagtctaggttctacatcttactggtcgagcaactttaggcaagtagcttaactcctccgaacttattttccttttctaccaaataat

SEQ ID NO: 39 - DNA, 122 bp, Artificial Sequence
  Description of Artificial Sequence Synthetic polynucleotide
gcaagtttaaaagtactcacaaaatctaataggcaattcaacataaaactccatggctatcgctgttcctcactttctgaacctttacctgcctgactttactccataccactccaactcac

SEQ ID NO: 40 - DNA, 122 bp, Artificial Sequence
  Description of Artificial Sequence Synthetic polynucleotide
gtagttttattgtatcagacttagtacaggggtggggtgggggtgtgtattggaatgatgcgtgcccgtttctctgcaaaatagtttctatgtcatggaaaggagtcgatgggacaagaaga

SEQ ID NO: 41 - DNA, 122 bp, Artificial Sequence
  Description of Artificial Sequence Synthetic polynucleotide
cagccccgcgccgcatcctccggccgccccctccccgctgcgagcttacgccgctgtcgccgccgccaccgccttaaaaaggacaaaacggaacagaaaatgaatgcatgcacaaaaaaaat

SEQ ID NO: 42 - DNA, 122 bp, Artificial Sequence
  Description of Artificial Sequence Synthetic polynucleotide
atgataggtgtgagcccctgcgctttgccagggctggtttttggatgtgattctcagggccgtctttctttacccttctgctctgctgaggcccacagcagcctagtctccttgggtgtggg

SEQ ID NO: 43 - DNA, 122 bp, Artificial Sequence
  Description of Artificial Sequence Synthetic polynucleotide
tgctgggtatccgcgccggaaccgcgagggggttggttcaggcctaggcgcggggcaggacgggaccggtgagtggctcctccaaacagctatagagacccagaaatgcctgtggaaagcta

SEQ ID NO: 44 - DNA, 122 bp, Artificial Sequence
  Description of Artificial Sequence Synthetic polynucleotide
cagaacctgcaggagcagatcaatcccctcttggtaacacaccagagcctgcggataccgcgactccgaatctagttctactgcccgctttagcacagtggctgcagctgtgctctgcgggt

SEQ ID NO: 45 - DNA, 122 bp, Artificial Sequence
  Description of Artificial Sequence Synthetic polynucleotide
cttctagtggcaaatttctccctgctgtggcaggaggacggctcgggggagctctgaccacgatttcatgcaaagatacggtgagaccctccgctcaacagtggcttttctaaggctctcct

SEQ ID NO: 46 - DNA, 122 bp, Artificial Sequence
  Description of Artificial Sequence Synthetic polynucleotide
acatgggcttctcttcgaggaggtaacatgtccgcgccctgagccacggctctctgggcgcggccatcttggtagatctgccgtacagaagggaaacagttgttcttgtgtcattaaaccgg

SEQ ID NO: 47 - DNA, 122 bp, Artificial Sequence
  Description of Artificial Sequence Synthetic polynucleotide
gcttttttggattgtgtgaatgcttcattcgcctcacaaacaaccacagaaccacaagtgcggtgcaaactttctccaggaggacagcaagaagtctctggtttttaaatggttaatctccg

SEQ ID NO: 48 - DNA, 122 bp, Artificial Sequence
  Description of Artificial Sequence Synthetic polynucleotide
agaggcctcggtgatttcccgacctctcctgtgaagcctgattcggaactcttccagctgcgaagaacttggccgattctaaggcacatcagggctgcctggaaccctaacacctgcctagg

SEQ ID NO: 49 - DNA, 122 bp, Artificial Sequence
  Description of Artificial Sequence Synthetic polynucleotide
tgcctgatggataatccatcacttgcttttctagtatgaatggtctatttacgggtccagcgcccctgctggcttacgaccttttccagggcggggaggggctgtcctcatctctgtgaccc

SEQ ID NO: 50 - DNA, 122 bp, Artificial Sequence
  Description of Artificial Sequence Synthetic polynucleotide
actgcgcccagcccattttacagacttttattttgttcagtttctttattgtcttcccaacgtccccccacacacactgcactaaaatgcaaacttcacgaaggcaaggaggaacttttgcc

SEQ ID NO: 51 - DNA, 122 bp, Artificial Sequence
  Description of Artificial Sequence Synthetic polynucleotide
tggggaacgcgagtggggacaggggggccttcagctgggccccagggaaccgccccgtggcgctctcggcctcgctctcactcacggtgctacaggtggtaagcaaattgactatgttgtgg

SEQ ID NO: 52 - DNA, 122 bp, Artificial Sequence
  Description of Artificial Sequence Synthetic polynucleotide
cagcccccctcggcggccgcaccgacaccgcaccccaagtcctaccccggggcctggcggcgctcctcgccgggatgccctagctgtgccgcaagctccccacgcccctctgcgtccttttt

SEQ ID NO: 53 - DNA, 122 bp, Artificial Sequence
  Description of Artificial Sequence Synthetic polynucleotide
aggaacccatgggaatgagctaaccggagtatttctggttaagcattggctagagaatggcgctgagattcagagaacagggctggaggtaaaaccatttattactaaccccagagcagtga

SEQ ID NO: 54 - DNA, 122 bp, Artificial Sequence
  Description of Artificial Sequence Synthetic polynucleotide
gtgtgcagaatttatatatataaatatatctcctccaacccctcccaatgaagcaagtcacgtgagtcaatcctaccctaagatattagggattgagcctcctgggacatttggtggcttag

SEQ ID NO: 55 - DNA, 122 bp, Artificial Sequence
  Description of Artificial Sequence Synthetic polynucleotide
gtgggaggtccttatgctaggagacctaatgtctgtgcctcagtttctctatcggagaggcgatgtctcaagaggcctttcagggctcagagttgagctttctgagttccacatggaagtga

SEQ ID NO: 56 - DNA, 122 bp, Artificial Sequence
  Description of Artificial Sequence Synthetic polynucleotide
gaacgactcagtctctcaaatcatagctaattctcctctgagggccttgctgaagttctgcgtttgcttgctccgctttcctctcattttggacctccagccttcctgtagtccgaggccct

SEQ ID NO: 57 - DNA, 122 bp, Artificial Sequence
  Description of Artificial Sequence Synthetic polynucleotide
cctccggtctagggctctttgtctttgcaaagtgtcgaaactgtctggcatagtgggctccgccggcggaggctggagccgaggaagcgaggaggcgggatgagggtgggagagggctcggg

SEQ ID NO: 58 - DNA, 122 bp, Artificial Sequence
  Description of Artificial Sequence Synthetic polynucleotide
tgcccgaggcagaaggatgtttgacctccggataagcgaggcgctgctgtgcattcattccgggctgcatcggtggcgacagcagaggctcgggcggcgactctccggccagcggcggcggt

SEQ ID NO: 59 - DNA, 122 bp, Artificial Sequence
  Description of Artificial Sequence Synthetic polynucleotide
actgttcaaaatgatgaacgaagatgcagctcagaaaagcgacagtggagagaagttcaacggcagtagtcagaggagaaaaagacccaagaaggtaaatcgccggaattaggaatgtctgt

SEQ ID NO: 60 - DNA, 122 bp, Artificial Sequence
  Description of Artificial Sequence Synthetic polynucleotide
tccagttttaatctttaaaaagaagaagaagcagcaatgcataagctgagtgattccccgcggaatccaaagctaacagagccaataaggcaccttcgagggcatcccagcccagctactga

SEQ ID NO: 61 - DNA, 122 bp, Artificial Sequence
  Description of Artificial Sequence Synthetic polynucleotide
ctcttgcaggaagccagttgagggaagttctccatgaatgtacgtcacaatgatgatgaccgaccaaatccctctggaactgccaccattgctgaacggagaggtagccatgatgccccact

SEQ ID NO: 62 - DNA, 122 bp, Artificial Sequence
  Description of Artificial Sequence Synthetic polynucleotide
ggtgacagggacctagggcctgggctgggaggaggcggggctagtccaggaagggacccgcgccacccaagtggcccctgcaggggcctcctgaggctcctgggtccttccccagctcccat

SEQ ID NO: 63 - DNA, 122 bp, Artificial Sequence
  Description of Artificial Sequence Synthetic polynucleotide
ttctttctcctccactgcaaagttaaatgcgagaaggtagaaacccagaggccatgctggcgctgagagatgagccccactcaccagattcaagatcccaaggtaggcacagacacagggca

SEQ ID NO: 64 - DNA, 122 bp, Artificial Sequence
  Description of Artificial Sequence Synthetic polynucleotide
tggaaggtgtcagcgtgtggctgtgtgatcttgcatgtgtctgtgttctgcaggaacatgcgtcagtgtgtgtgcatcagtgtgcatctctatgtgtcatgcactggtgtgtcttcgtgtat

SEQ ID NO: 65 - DNA, 122 bp, Artificial Sequence
  Description of Artificial Sequence Synthetic polynucleotide
aaagtgttgggattacaggcgtgagccattgcgcctgggcaggtattttttctcattaagcgctccccatccaagtctgccctaggcaggagtgcctagtgcacgggtacatacataccccg

SEQ ID NO: 66 - DNA, 122 bp, Artificial Sequence
  Description of Artificial Sequence Synthetic polynucleotide
agggcgtttgccacagccccttaactccttccaaaacactccgcttagatactgataaggcgccaactgcagcctggagaacccctatgcgccatcttggcttcccgcaggcctctgcgccg

SEQ ID NO: 67 - DNA, 122 bp, Artificial Sequence
  Description of Artificial Sequence Synthetic polynucleotide
cagcctcttcctgctgtgtcaccatctgcgggaggtggtactctagtctcccctaagactcggcttgccacctgcaccagctccctgggcaaaggtcacctgtgttcttaatagagcagaga

SEQ ID NO: 68 - DNA, 122 bp, Artificial Sequence
  Description of Artificial Sequence Synthetic polynucleotide
atggagtatgtatatgttcagctttactaatgccaaaatgttttccaacatagttgtaagcgatttgtgccctcgataagcagggtatgagaatttccattgttccatgtcctagctgactc

SEQ ID NO: 69 - DNA, 122 bp, Artificial Sequence
  Description of Artificial Sequence Synthetic polynucleotide
caaatctctctgcttccttcgatgttgcctgtggcagaaatttacattatcccttcagcccgcttaaaaaattctgtacttcccaagcggctaaatttttaaagtccctcaaccacaaaaat

SEQ ID NO: 70 - DNA, 122 bp, Artificial Sequence
  Description of Artificial Sequence Synthetic polynucleotide
aagtagagaggcagccgggagcctgccttctgtgttctcggtgcaggggtattctgagaacggcccctgctcacacgggtttaaaaggaactcagtgaccacagacggatgagaacagcgga

SEQ ID NO: 71 - DNA, 122 bp, Artificial Sequence
  Description of Artificial Sequence Synthetic polynucleotide
tccgcaggggtctcctgggaggaacccaccagcgataggaacactgaagctgggctacggcgtccgcccgagccttttcttaaaggcgccgaccccggaagcggggcgtccgagggagcgcg

SEQ ID NO: 72 - DNA, 122 bp, Artificial Sequence
  Description of Artificial Sequence Synthetic polynucleotide
acctgcgcccacagggcctggggaaaccttgagtacgaatgccacgccgcggcttgtgggcgacaccaccgctgtcaccatgccccagggccacctggcaatgctgctcttttcccgtgacg

SEQ ID NO: 73 - DNA, 122 bp, Artificial Sequence
  Description of Artificial Sequence Synthetic polynucleotide
aaattgatcaatacgaatgatcacgccccatgtgcatctcctcagcagccacaagagagacgaggtcactggaggataaacatccgtgactgcacctcatgatccatcacgcacgacggccg

SEQ ID NO: 74 - DNA, 122 bp, Artificial Sequence
  Description of Artificial Sequence Synthetic polynucleotide
tcttcctccatgtccaagacacccagcttaacaaccctgtagcccccaacttggccctagcggcacctcgcctcgaccttgccattttatactcaattggggcgtagggttctgaagcccag

SEQ ID NO: 75 - DNA, 122 bp, Artificial Sequence
  Description of Artificial Sequence Synthetic polynucleotide
catctccacttctccagtccgccctactctccacccgtgacctccagtggagaccccaggcggcagcatcagtatttgatcggcccttcgtcagcacgctgccagccctggccggctgggtt

SEQ ID NO: 76 - DNA, 122 bp, Artificial Sequence
  Description of Artificial Sequence Synthetic polynucleotide
agttgccacagggtaagcccagtgcccttttgcccaaggtcaggtcacttggtgctggggcgtcacagagcccaggaaacttgggatcagaaccccctgctccccgctccccacctcatccc

SEQ ID NO: 77 - DNA, 122 bp, Artificial Sequence
  Description of Artificial Sequence Synthetic polynucleotide
ctgaccctcacgcagtgtccgcctccagggaactgtggaacacgtcgcagagagctcaagcgccacgtttggatccctgagcagctgtcacaagcctgcacccaggactggggggcctgctg

SEQ ID NO: 78 - DNA, 122 bp, Artificial Sequence
  Description of Artificial Sequence Synthetic polynucleotide
gagggagcaaaggtctccggtgtggcaggcaggtttttccaggcagctggcaggtgtgctcgcgcagctgacactgccttgggagcacagaaggtggcagcaaagatcatgcggtcttttga

SEQ ID NO: 79 - DNA, 122 bp, Artificial Sequence
  Description of Artificial Sequence Synthetic polynucleotide
agggtgcctgcctctcccggcctgcgcctgcgcgctggggccttcggctgaaggggtgtgcgctagcggagctccgggaaatgaatgaatgaatgaatgaatgaaatgctgaagcgggcagg

SEQ ID NO: 80 - DNA, 122 bp, Artificial Sequence
  Description of Artificial Sequence Synthetic polynucleotide
taaggcatctgctgagtgtataaccattttacctcttgtttttagccctcttctgggtcacgctagaatcagatctgctctccagcatcttctgtttcctggcaagtgtttcctgctacttt

SEQ ID NO: 81 - DNA, 122 bp, Artificial Sequence
  Description of Artificial Sequence Synthetic polynucleotide
gacgccggcccgaggtggcgccggagctgctggcagaggggcggcgggcggcggcggcggcggctacaggagggactgacaaagccccacggcacgccgctccctacttatagcaccggcgg

SEQ ID NO: 82 - DNA, 122 bp, Artificial Sequence
  Description of Artificial Sequence Synthetic polynucleotide
ccccggtccgcctggcccctcgcccgcccgccaggcccgccgaatgcggcctccgccccgcgcgcctaaaggaggagcgtcgcgggggatggaggcggcgcgcggtgggacctggggagatg

SEQ ID NO: 83 - DNA, 122 bp, Artificial Sequence
  Description of Artificial Sequence Synthetic polynucleotide
cgaactcctcacctcaaatgatccgcccacctcagcctcccaaagtgctgggattataggcgtaagccactgcacctgaccaatacagtcttaatagggctatttggacctccttggagaca

SEQ ID NO: 84 - DNA, 122 bp, Artificial Sequence
  Description of Artificial Sequence Synthetic polynucleotide
gagacctcctgccaacccaattcccagtgcgcagatggggaggaagaggcagcgaggaggcgcccccagctcaaggtcacccatcaggtctggggcagagagagccagaagcccggaattcc

SEQ ID NO: 85 - DNA, 122 bp, Artificial Sequence
  Description of Artificial Sequence Synthetic polynucleotide
caccctactgcatgttgcaaagtattcctttaaaatgaagtgagtaaaatactgggatgacgttatctggagcccaagaaagatggctcatttggaaaggcctaatatcccaagttgcttac

SEQ ID NO: 86 - DNA, 122 bp, Artificial Sequence
  Description of Artificial Sequence Synthetic polynucleotide
ggccgggtggggggaggtgacttgatgtcatcctgagcagctgggcggcgggtgccggtgcgcacggagccgagccggggctcccgttgcgctgcaccgcgttgggtcggagtcccaggact

SEQ ID NO: 87 - DNA, 122 bp, Artificial Sequence
  Description of Artificial Sequence Synthetic polynucleotide
gcagcccgggaaggggcattggtggcgcttggcagcaggtgtgacagacctcctccggggcgcctgatccgcggcgggggcggggcctgcccctagggcccctccagagaacccaccagagg

SEQ ID NO: 88 - DNA, 122 bp, Artificial Sequence
  Description of Artificial Sequence Synthetic polynucleotide
caagggtctagggtcctgggtatctctaggtactgagacagctgtgtggtctgctgcatccgtgcccctctctgagcctagagcctgggctggcccaggaagcaggaagaagtctgcaccag

SEQ ID NO: 89 - DNA, 122 bp, Artificial Sequence
  Description of Artificial Sequence Synthetic polynucleotide
tcagctcgtgggctgccagcgtgcaacctctcacctagataatggtatataatataaatacgtttcccttccccccttttttctcttcctcctctttctcctttccctcccattttccacat

SEQ ID NO: 90 - DNA, 122 bp, Artificial Sequence
  Description of Artificial Sequence Synthetic polynucleotide
ggtgggtggtccggccccgagccctcctgactctctcgccaatgcccagaggcgccgcagcgattccagggaggccgcgctctcgccccaaggcaaccagaagcccacgtgccaggagaggc

SEQ ID NO: 91 - DNA, 122 bp, Artificial Sequence
  Description of Artificial Sequence Synthetic polynucleotide
gttctcatcccatatgcctttgtccaaaggttgcacgggggttaagcttggcccagaaggcgccgagggctggtcgagttctcccctttccaagaaccagccgaatctccctcccgcagatc

SEQ ID NO: 92 - DNA, 122 bp, Artificial Sequence
  Description of Artificial Sequence Synthetic polynucleotide
tgatcgggacgaggaagggtcactccgtgacccgggatagggccgggatgcagccttggacggggctgggcccaaatgtgggctctggagggagccgggctggggctggtgctggtgctccc

SEQ ID NO: 93 - DNA, 122 bp, Artificial Sequence
  Description of Artificial Sequence Synthetic polynucleotide
cagcgggagatcaagtgagcctcaaaacattagaaaaacccaagccagtctgcagagcaccgcagccgcctcagggccggttaccatagctacccttggcttcccagcccagcacatgtctg

SEQ ID NO: 94 - DNA, 122 bp, Artificial Sequence
  Description of Artificial Sequence Synthetic polynucleotide
ctctgcggggacagaggtctcaggaaagtagcctttatttatgtggcaccgatcggaacccgcggccggccaggcggacctggacggagcgtccctgctcggaacctggcgcggggcgccgc

SEQ ID NO: 95 - DNA, 122 bp, Artificial Sequence
  Description of Artificial Sequence Synthetic polynucleotide
agggtagggagagcgggaggctgctggcctgaggctaaagctagtcactgacctctatcacgtgcttgttatatgttaggcatgatatgccagctccttttattcggcgtagcaatctctga

SEQ ID NO: 96 - DNA, 122 bp, Artificial Sequence
  Description of Artificial Sequence Synthetic polynucleotide
tccacaaagtactttccatcagatacacttttctgatggaaaccaggtgtgtgatggttacggccccaggttagctccagagcacattcaactgtgggtaaacacaaatgtgccctgtgcca

SEQ ID NO: 97 - DNA, 122 bp, Artificial Sequence
  Description of Artificial Sequence Synthetic polynucleotide
ttaatgccccccagaatcagcaccatgtcatcacaggcttgggtcaaggggcgggtcagacgccagtcacatccgctcactgcccacagccacccccccacagtgagtcatctgccagggtg

SEQ ID NO: 98 - DNA, 122 bp, Artificial Sequence
  Description of Artificial Sequence Synthetic polynucleotide
ggtgtcctgcctggggtatccccagagtttggcacacggtgatagccaacattcactgagcgccaaagggccaggtgctgccactctctcaaaataagcctctgccacttactgaacaacta

SEQ ID NO: 99 - DNA, 122 bp, Artificial Sequence
  Description of Artificial Sequence Synthetic polynucleotide
ctgcaagatcgtggtggtgggagacgcagagtgcggcaagacggcgctgctgcaggtgttcgccaaggacgcctatcccggggtgagggacctgcgtcttgggagggggacgctaaggctgc

SEQ ID NO: 100 - DNA, 122 bp, Artificial Sequence
  Description of Artificial Sequence Synthetic polynucleotide
gatgtctccaggcacccccgacctgggcttggccctctgcttggggcggagcttccaggacgtgctgggacctaggtctgaccccgcccaaggcagagttgaacccactgtgaactttcagg

SEQ ID NO: 101 - DNA, 122 bp, Artificial Sequence
  Description of Artificial Sequence Synthetic polynucleotide
ctgagcaagtctttgattcatggattcccagcaactctagctggaacaacttctttggctcgtattcctctggtatatgtgctgaatttagaattcaatcactggacaccaggaaaggcaac

SEQ ID NO: 102 - DNA, 122 bp, Artificial Sequence
  Description of Artificial Sequence Synthetic polynucleotide
cttagttaactcacctgaaaaatgggaacaataatacaagccacagttatgagaattcaacgagataatgcatgtacagcacctggcacatggtaaaacgctcaataagtggtagttagtag

SEQ ID NO: 103 - DNA, 122 bp, Artificial Sequence
  Description of Artificial Sequence Synthetic polynucleotide
gagtctggtaagtgtcggatggtagaaccagggttgggactcgggacctccaacagcatacgatgtggtgggggtgggcagcctgggtgggggtgggcattactctggggctggattcagct

SEQ ID NO: 104 - DNA, 122 bp, Artificial Sequence
  Description of Artificial Sequence Synthetic polynucleotide
ctctcacccgctgccgggctggattgtcctccacttgtgcttatctggtcctcgatgccgcgctccgacgtcttatctgagggagccttccgttaatgaaggctctataaacatctgacaaa

SEQ ID NO: 105 - DNA, 122 bp, Artificial Sequence
  Description of Artificial Sequence Synthetic polynucleotide
gccaggtcaccctctcactctgtgcctcttagttatcttgcatgctctggtctttgcatacgctgctccctgcaccaggaacctccatccccatctttgtctgcttgtcgaacttcagaaat

SEQ ID NO: 106 - DNA, 122 bp, Artificial Sequence
  Description of Artificial Sequence Synthetic polynucleotide
ggtcctgcccctcacccctctcgcggggggtcgacctgctcgtggatggggaccctggcgcgcctgggctcccatccgggggttccccgacccaggtcccggtcacccccagcgcagggccc

SEQ ID NO: 107 - DNA, 122 bp, Artificial Sequence
  Description of Artificial Sequence Synthetic polynucleotide
gagccgccgattggctaggagcacttgagcagcggaagcagctggctcgcgcggggactgcggtgagggggcgagccgtgaagatggcggcagtggtggaggtggaggttggaggtggtgct

SEQ ID NO: 108 - DNA, 122 bp, Artificial Sequence
  Description of Artificial Sequence Synthetic polynucleotide
gcattaccccttgtgggagccatatttttctagaaggcattttgatcaagacaggcctcccgcggttattgatcttagggtcattgagagtccaagaactggggagatgaaggccacccggc

SEQ ID NO: 109 - DNA, 122 bp, Artificial Sequence
  Description of Artificial Sequence Synthetic polynucleotide
tggggggcgctggctgcctgctggccctggggttggatcacttctttcaaatcagggaagcgcctcttcatcctcgactgtccagtgccgccgaagaaaaagtgcctgtgatccgaccccgg

SEQ ID NO: 110 - DNA, 122 bp, Artificial Sequence
  Description of Artificial Sequence Synthetic polynucleotide
acccggaaatgcacaagcctcttgatgcataaaaacagctgggctcccttggagacagagcgccatgggaaaccgggtctgctgcggaggaagctggtgagtaggctggaagggcaaagggg

SEQ ID NO: 111 - DNA, 122 bp, Artificial Sequence
  Description of Artificial Sequence Synthetic polynucleotide
ccaagtaaaaaaagccagatttgtggctcacttcgtggggaaatgtgtccagcgcaccaacgcaggcgagggactgggggaggagggaagtgccctcctgcagcacgcgaggttccgggacc

SEQ ID NO: 112 - DNA, 122 bp, Artificial Sequence
  Description of Artificial Sequence Synthetic polynucleotide
ctccgaccctgccgcccccattctccgctccccgctctggggctgagtgaggcaggatggcgagagacccctgagccaccaagtccgcttacctcaggcagatcccgacgggggctcggcgc

SEQ ID NO: 113 - DNA, 122 bp, Artificial Sequence
  Description of Artificial Sequence Synthetic polynucleotide
aaagagacggtttgggaattgctctgaggatgctatgcaagtcactaataaaggaagacacggacagatgaacttaaaagagaagctttagctgccaaagattgggaaagggaaaggacaaa

SEQ ID NO: 114 - DNA, 122 bp, Artificial Sequence
  Description of Artificial Sequence Synthetic polynucleotide
gagttttcctctcacacttgaccctgattttgttttgcagaagcgacaggctgtggacaccgtgagtaagagtcctggcaaaggggtctgtgacagagccctttttacaggcttgctttccc

SEQ ID NO: 115 - DNA, 122 bp, Artificial Sequence
  Description of Artificial Sequence Synthetic polynucleotide
ctgacctcaccacccaccagggaggtgggtcttattctgggcatcgtgccaagttcttagcggggccctctagaatctctaaagcaaatcaggctgaagaggggaaaaccagcagggggagg

SEQ ID NO: 116 - DNA, 122 bp, Artificial Sequence
  Description of Artificial Sequence Synthetic polynucleotide
actttccagctcttccgaagttcgttcttgcgcaaagcccaaaggctggaaaaccgtccacgatgaccagcatgactcagtctctgcgggaggtgataaaggccatgaccaaggctcgcaat

SEQ ID NO: 117 - DNA, 122 bp, Artificial Sequence
  Description of Artificial Sequence Synthetic polynucleotide
ctgtgggttcggcactaggtcctcctccccgtggcttcctagtaggcatgtggtggtgtacgcctgctgggcacctagcgagaggggtcgtgagttgggagggagccacgttggggtgcctg

SEQ ID NO: 118 - DNA, 122 bp, Artificial Sequence
  Description of Artificial Sequence Synthetic polynucleotide
ccgcagccgagcaggaagagcgagccgggggattgagactgtccgatccaacctagggcacgagcctggtataaatcgcggactaacagagactatctgatgaagagactaacggagagaga

SEQ ID NO: 119 - DNA, 122 bp, Artificial Sequence
  Description of Artificial Sequence Synthetic polynucleotide
ggctcttttgtggctaatctagcgagggacctagggctgggggtggaggagctgtcttcacgtgaagcccgggtagtgtctgatgataataaaaagtatttgcaccttgatttgctgactgg

SEQ ID NO: 120 - DNA, 122 bp, Artificial Sequence
  Description of Artificial Sequence Synthetic polynucleotide
aaattgcctgaaatttcagagttggacttcatcacttgtctgtgagccgacgcaggcaggcgtattctatatcaacgacagactctcctctgccatttcctttcctgaatctagttaacatt

SEQ ID NO: 121 - DNA, 122 bp, Artificial Sequence
  Description of Artificial Sequence Synthetic polynucleotide
ggagagcaagtcaagaaatacggtgaaggagtccttcccaaagttgtctaggtccttccgcgccggtgcctggtcttcgtcgtcaacaccatggacagctcccgggaaccgactctggggcg

SEQ ID NO: 122 - DNA, 122 bp, Artificial Sequence
  Description of Artificial Sequence Synthetic polynucleotide
tgtctttcggttcataattgcgattgttagcgaagtggtctcgaattccatttcactccccgttcgccgctctcagactaaattgcaaatatccccaagtctgtagcaaaaaaagttttctc

SEQ ID NO: 123 - DNA, 122 bp, Artificial Sequence
  Description of Artificial Sequence Synthetic polynucleotide
agcgcctgggcgagtgacatctgggccggaccagctggtgctgcgcggcgcaggtaagggcgtgcgcgggcagggacaggggtaaggggtgccggggcgcggggatacagggaggcctgccc

SEQ ID NO: 124 - DNA, 122 bp, Artificial Sequence
  Description of Artificial Sequence Synthetic polynucleotide
ttattctggtatcaataaaaaggaactgttactatagtaacagatattccacttggtgcacggccacttccacgatgcggaacatcatgtccaagccacacgcttgagaggcacaaataaat

SEQ ID NO: 125 - DNA, 122 bp, Artificial Sequence
  Description of Artificial Sequence Synthetic polynucleotide
gaagcaatttgagggtgttccagatcacaccaacagcggatgctgcatctgggtagttcacgtacccgaacaaaaattttaaaaatttggtgtggcctttgccatccattcactcctcaaaa

SEQ ID NO: 126 - DNA, 122 bp, Artificial Sequence
  Description of Artificial Sequence Synthetic polynucleotide
tgagtcagaggcaggtgctgcaaggtagggccgaggcgggcaggtgccctaactagctggcgccgaggagacccgggtgcggtgggctccaccgactctctctcccgcagtgttcgagcaat

SEQ ID NO: 127 - DNA, 122 bp, Artificial Sequence
  Description of Artificial Sequence Synthetic polynucleotide
aaccgggacggaggctggccctgggacagcaggcggctccgagaacgggtctgaggtggccgcgcagcccgcgggcctgtcgggcccagccgaggtcgggccgggggcggtgggggagcgca

SEQ ID NO: 128 - DNA, 122 bp, Artificial Sequence
  Description of Artificial Sequence Synthetic polynucleotide
ctcgcctgggtctctctcgccccgtcgcccccattcccccaccctcggaatgaggaggggcgcctgctacccccggccaggcaggcagtgtgtccctcggattccttccaatttcctgatcc

SEQ ID NO: 129 - DNA, 122 bp, Artificial Sequence
  Description of Artificial Sequence Synthetic polynucleotide
gtaagacaggaaatcaatcagaggcagagcgacgcctctggctctggtctagtggtgcagcgtctctagccctcgccccgcccaccgtccccgcgaggcgtccactcgccgagccccgccct

SEQ ID NO: 130 - DNA, 122 bp, Artificial Sequence
  Description of Artificial Sequence Synthetic polynucleotide
gtggcgcaggtgcaggactgtgggaagacaggagcgccagggaatgtctggccagcagcgcgctgccctcaaggggcctccttgaaggccccttgaagagggcaacacaactaatgacgata

SEQ ID NO: 131 - DNA, 122 bp, Artificial Sequence
  Description of Artificial Sequence Synthetic polynucleotide
ccccagctcagggtccgtgtacttggggaccatttcctgctctgctgtggtctactggaccgtctggcatcgctgtgaccgcatgggccgtgctccatcaatattgtttttttgtgtgtggg

SEQ ID NO: 132 - DNA, 122 bp, Artificial Sequence
  Description of Artificial Sequence Synthetic polynucleotide
tacagccttccgggagctggacggggcctccccagctttgggcagcttgggacagtggcccgagactgtgggaatccgaaacctcgcttctggctagccacaaggtctgggcgcgccccagg

SEQ ID NO: 133 - DNA, 122 bp, Artificial Sequence
  Description of Artificial Sequence Synthetic polynucleotide
tcccattcacagacaaactgctaaaagcaaaaccaaaactttccaaataagccaggctttcgtcagttcctcagaactagttctggtttgactcactctcatgttacggcaaaccttaagct

SEQ ID NO: 134 - DNA, 122 bp, Artificial Sequence
  Description of Artificial Sequence Synthetic polynucleotide
aacgtgcggttgccgtgactaaacgcattcattcaccctacaagatttaggaaaatgtaacgttgcaagggaagcaaggtctctgtgtaaacctcgtaatcgccaccaaaagtcggtagctg

SEQ ID NO: 135 - DNA, 122 bp, Artificial Sequence
  Description of Artificial Sequence Synthetic polynucleotide
cacatttcccgcacaagtccccaagccttggacccccctcatcaggacctccggcacaggcgcccgtttcccgccactgccttccagtggtttggtccccgagcaggacccaaggcggggca

SEQ ID NO: 136 - DNA, 122 bp, Artificial Sequence
  Description of Artificial Sequence Synthetic polynucleotide
gcccctccctctctgccctttcattagcttaattacaccgtgcctatgacaacagagcaacggaaactgatacctcgggcctctggggcttgaattattcaaactctgtaaagcagcacaca

SEQ ID NO: 137 - DNA, 122 bp, Artificial Sequence
  Description of Artificial Sequence Synthetic polynucleotide
cgtggggctggcggcgcggatgccctggggcgctgcagaccccgagaggccgcttgcccgcggggacgtcagccgcttttgctgttaaaatctgaaatgttcagcaagttagaaacttgaaa

SEQ ID NO: 138 - DNA, 122 bp, Artificial Sequence
  Description of Artificial Sequence Synthetic polynucleotide
ccggatgagcagtgacttcagggcttgggctactctggcttaacgggaccagtagcagagcgccgcccgtcctgcttgctgctgggtccggttgccgaggcggaaaagtcgcaagctccttc

SEQ ID NO: 139 - DNA, 122 bp, Artificial Sequence
  Description of Artificial Sequence Synthetic polynucleotide
tcggtcaggcgtgtgcagacagcgcctgcaggtctgggtgggtgctgatctgagtgtctgcgcctgggccatgtttttgagcctggcacaggggtgcttagtgaacacatgaccgcctagcg

SEQ ID NO: 140 - DNA, 122 bp, Artificial Sequence
  Description of Artificial Sequence Synthetic polynucleotide
tagttcatctgctggccggctctcagtccccgtggcgccccctttcctcttgtcccagagcgctctcgactccaccatgccaaggggattcctggtgaagcgaactaaacggacaggcggct

SEQ ID NO: 141 - DNA, 122 bp, Artificial Sequence
  Description of Artificial Sequence Synthetic polynucleotide
agggaaaccgagggccaggaaacaactagaatccgacggtatttcctagctccctgatggcgcttcccatgcccccaactaaatcatgaaataacccactcacctgtttgcaccgggcctgc

SEQ ID NO: 142 - DNA, 122 bp, Artificial Sequence
  Description of Artificial Sequence Synthetic polynucleotide
cctttaatctttttgttttggtttgaatctgctcggcgcagactggccaaggatcctctccgccctcccccttcctcctggcgcgggagaggcaccggatatccccaccctccccgagctct

SEQ ID NO: 143 - DNA, 122 bp, Artificial Sequence
  Description of Artificial Sequence Synthetic polynucleotide
tgtgcctcaggcttataatagggccggtgctgcctgccgaagccggcggctgagaggcagcgaactcatctttgccagtacaggagcttgtgccgtggcccacagcccacagcccacagcca

SEQ ID NO: 144 - DNA, 122 bp, Artificial Sequence
  Description of Artificial Sequence Synthetic polynucleotide
cagacctgccttaaaagcagcttgcccgccttctctcctcccctccgggcgggccctgcacgtggccctgacagcagtaggccccacccctgctggatccagtgagctcaggtggggctggc

SEQ ID NO: 145 - DNA, 122 bp, Artificial Sequence
  Description of Artificial Sequence Synthetic polynucleotide
aatgtgctgggtgcagctttgggtaatacatatgccgaaccttctctttaagggtccacgcgcagcctcgggtgtgaatgaaggagaagagatcgtgtaccacacatgatgcttacggagca

SEQ ID NO: 146 - DNA, 122 bp, Artificial Sequence
  Description of Artificial Sequence Synthetic polynucleotide
gcgcggcagtggcctcgcagggcgctgggtccctctccccagctctcctccccctggccccgtcgccccgccctcgccgggctgggctgcggggtcaggggccgagcggagaggggtgagta

SEQ ID NO: 147 - DNA, 122 bp, Artificial Sequence
  Description of Artificial Sequence Synthetic polynucleotide
gtgagtgaggggctcaagaaactctacaagagcaagctgctgcccttggaagagcattaccgcttccacgagttccactcgcccgccctggaggatgccgacttcgacaacaagcccatggt

SEQ ID NO: 148 - DNA, 122 bp, Artificial Sequence
  Description of Artificial Sequence Synthetic polynucleotide
tcggggtcccttggcctggagaccctttgtccaacccgtcgcccacctcaagacctgcctcgatgctgcgcatacagtaggtatccaataaatgttcctgggatagaaggcaaaggcgctgg

SEQ ID NO: 149 - DNA, 122 bp, Artificial Sequence
  Description of Artificial Sequence Synthetic polynucleotide
agccagcagcagtgccatcatcccgtgcccacccacacgccccatccagggtgcccgagacgagcccatctcggactgcacggcctcctgactgatggcagctcaaggacacccgggtcctt

SEQ ID NO: 150 - DNA, 122 bp, Artificial Sequence
  Description of Artificial Sequence Synthetic polynucleotide
ccactatgttcagtctagtgagtctgagcaattaactcacattttgaatttcaagtctctcgccttaggcaaaacaccaccacctgatgctcaccagaggggcgtgacgcggcagctgggca

SEQ ID NO: 151 - DNA, 122 bp, Artificial Sequence
  Description of Artificial Sequence Synthetic polynucleotide
tgggcagtggcggggcacgcaggcggcgatcagaggctgtcccgtcctctccgggggccgcggctcatcctgccaggcatctccgaggaaagtttgctctccggaaaagaagaaacccgcgc

SEQ ID NO: 152 - DNA, 122 bp, Artificial Sequence
  Description of Artificial Sequence Synthetic polynucleotide
aaacatggatcaagaaactgtaggcaatgttgtcctgttggccatcgtcaccctcatcagcgtggtccagaatggtaaggaaagcccttcactcagggaagaacagaaggggagattttctt

SEQ ID NO: 153 - DNA, 122 bp, Artificial Sequence
  Description of Artificial Sequence Synthetic polynucleotide
agcgacagagcaacgtcgcactgcattcttaccaaacacccaggtgaacgacgcatccaacgatttgggagctcaggacccatggtccctaaaaggcaacaattaagactcccatttagacc

SEQ ID NO: 154 - DNA, 122 bp, Artificial Sequence
  Description of Artificial Sequence Synthetic polynucleotide
gaagagaggagaggtttagagtcaaagagccccaaacattagtgagagtatatgtatgaacgtttggtcatcttagaacagtggttggcatccacaggagaccagcagaatcacatgggcgc

SEQ ID NO: 155 - DNA, 122 bp, Artificial Sequence
  Description of Artificial Sequence Synthetic polynucleotide
ggggatccccagttgccaaaggatggagggcggagctggaggacctcaggctagtgagcacgcccttgcccaggcctgcagtggctgcactcgccagctggcccatggccctgtccgactcc

SEQ ID NO: 156 - DNA, 122 bp, Artificial Sequence
  Description of Artificial Sequence Synthetic polynucleotide
tctttttgtgactctcaaggaaagtcggttttctgagctcttactggcttagtagcgtggcgttcaacgcagagcattctaggtaatgtagttttcatagatcccgaggtgggtgccgggga

SEQ ID NO: 157 - DNA, 122 bp, Artificial Sequence
  Description of Artificial Sequence Synthetic polynucleotide
gagaagggaggcagctgcggcaaagttagagcaagtactgcagcagccaggttgggtccgcgccgtcgggtttctgagaaaagggaggaaagaggcggggcctgcacggtgtgtccccgccc

SEQ ID NO: 158 - DNA, 122 bp, Artificial Sequence
  Description of Artificial Sequence Synthetic polynucleotide
ttcctatcccactgatcgttttagagcctgaacagacaaaacatcctggttaccaagactcgaagaatgcataagctgggaccaggcaaaacaaacagatcactgtgggctcacagagcagg

SEQ ID NO: 159 - DNA, 122 bp, Artificial Sequence
  Description of Artificial Sequence Synthetic polynucleotide
gggcaacgcggccggatcctggagttcccctccgtgctgtggaattgggtcaggcgtgtacggtcctgaccctaggacacagctgcatgtcctcacctcggtgttcaaagctgcaccggcca

SEQ ID NO: 160 - DNA, 122 bp, Artificial Sequence
  Description of Artificial Sequence Synthetic polynucleotide
gggcgaggcgtgagaacgagcatttctaagttcccaggtgatgcccctagtgttggtcggcgtccacactctgaggacagtgacctctctgctctgtccctcatgtcttactactactgtct

SEQ ID NO: 161 - DNA, 122 bp, Artificial Sequence
  Description of Artificial Sequence Synthetic polynucleotide
aatcatcaaggccattttcaaatcccattggtctagccgtcacatggtgagaaccgaatgcgcggataattacggagctgatatttccccccctccccttctttttcctccctcccctccaa

SEQ ID NO: 162 - DNA, 122 bp, Artificial Sequence
  Description of Artificial Sequence Synthetic polynucleotide
accgcatacagcacaactcaagtttgcatcagactgggaagcgaacttaagccagcggtgcgtggcccaggagtgggaaaggaaatggatgcctgaagtggaagaggtggtgcagagggggc

SEQ ID NO: 163 - DNA, 122 bp, Artificial Sequence
  Description of Artificial Sequence Synthetic polynucleotide
ttttatctgccctcggtacgctgatttccaaaacccagcctcatattctatactccaaagcgcactgccaggtgggccaactccagcccccacaatccgatgccaaggccacttcttgccac

SEQ ID NO: 164 - DNA, 122 bp, Artificial Sequence
  Description of Artificial Sequence Synthetic polynucleotide
gtttatagtggtctggcttttggccatgacaatgacaccttgcccttttaatttggggcccgtgcaaatattcactgaaagctgtcaagaggaaaacagaattggttattgaatcacttgct

SEQ ID NO: 165 - DNA, 122 bp, Artificial Sequence
  Description of Artificial Sequence Synthetic polynucleotide
gccccctggcggccacagcgcaagcccggtctctcctcctgctggaaggacaccggggaccgcacctccagctgtgggagttccgagagaccccgccctgcccgctcctccctggaggccgc

SEQ ID NO: 166 - DNA, 122 bp, Artificial Sequence
  Description of Artificial Sequence Synthetic polynucleotide
aagtgcggcccttgggcccgcagcattagcctcatcagggtgctggttaaacacacaaatcgtcagacctccaccccagactttctgaatcagaaactctgggggcacagcccaggaatctt

SEQ ID NO: 167 - DNA, 122 bp, Artificial Sequence
  Description of Artificial Sequence Synthetic polynucleotide
cagggaaacgcgggaagcaggggcggggcctctggtggcggtcgggaactcggtgggaggcggcaacattgtttcaagttggccaaattgacaagagcgagaggtatactgcgttccatccc

SEQ ID NO: 168 - DNA, 122 bp, Artificial Sequence
  Description of Artificial Sequence Synthetic polynucleotide
atttccatgataaagtatcgtttccctggtaacaatagcattggtcttgagaagcttctccgattgcagcaggacctttaagctgagaactgaaaaacgaatgggaagtgttatgagcagaa

SEQ ID NO: 169 - DNA, 122 bp, Artificial Sequence
  Description of Artificial Sequence Synthetic polynucleotide
tcctcgggagacagggtctccagcaggctgtcgatgtcggggtcttcactcacctgccggcgatatttggctactctagacatcttggcaaaatgggctgtggctgccaggggctatcagag

SEQ ID NO: 170 - DNA, 122 bp, Artificial Sequence
  Description of Artificial Sequence Synthetic polynucleotide
cgggatgggggagcccagcagtgcccactgcacgcctggtgacgagtctcccctcatctgcgcagctcagtttgctcagtttgctcttcgtgacacgtgactcggcaaggggagcaggagga

SEQ ID NO: 171 - DNA, 122 bp, Artificial Sequence
  Description of Artificial Sequence Synthetic polynucleotide
aaaaaaaagaaaagaaaatacttgatggaaggctgccatcaccatgctgcaaaatctccacgcccctgctgcccgcacctgtccttcctccctccctcctcccctggcctggggaagcccct

SEQ ID NO: 172 - DNA, 122 bp, Artificial Sequence
  Description of Artificial Sequence Synthetic polynucleotide
gagggaacacatatagaagggattaaggggtagttgatgactctttgggaaaagagggtacgggagaagcaaggggaagaaagacatctatttgtcaaagagcaaaggcaaggcaaagctgg

SEQ ID NO: 173 - DNA, 122 bp, Artificial Sequence
  Description of Artificial Sequence Synthetic polynucleotide
gagccgctcgctcccgacacggctcacgatgcgcggcgagcagggcgcggcgggggcccgcgtgctccagttcactaactgccggatcctgcgcggagggaaactgctcaggtgggcgcggg

SEQ ID NO: 174 - DNA, 122 bp, Artificial Sequence
  Description of Artificial Sequence Synthetic polynucleotide
ggaagcccggagccgcccctccccgctcccccgccccgccgccccggacggacgggcgcgcggagccaaccccgctgccgctggctgtccaaatcccaccagagccaatgggagcgcgaggg

SEQ ID NO: 175 - DNA, 122 bp, Artificial Sequence
  Description of Artificial Sequence Synthetic polynucleotide
ggaacggttccggcagggttgggtttccagagctgtccaggggcgcctggtgctgaatcccgcttggaaagaggcttggaggtggatgggaagggatttccaacggaggcggctcctctctc

SEQ ID NO: 176 - DNA, 122 bp, Artificial Sequence
  Description of Artificial Sequence Synthetic polynucleotide
cgcctctctggacctcttttttcatctgtagcttggggataacactgactaacatggccacgctgagcactgcaaatctagcctgattgccagtcagaatgcacgcccggcctcgctgtttc

SEQ ID NO: 177 - DNA, 122 bp, Artificial Sequence
  Description of Artificial Sequence Synthetic polynucleotide
ccccagagagctttcatctagaaggtttgactctggccagacaaccagcgagcatcttctcgcaatctgttgcttcttccatggcaaactccagagaattaagaagccaaactcaacatcgc

SEQ ID NO: 178 - DNA, 122 bp, Artificial Sequence
  Description of Artificial Sequence Synthetic polynucleotide
ccaacgggtgaagagcctaggtgtttttgatctgtgccttctctgttcctcagagatatgcgggcgtccttctagaagcccatctcgctcacctgtgtggtcacccttgtcccgcccttcct

SEQ ID NO: 179 - DNA, 122 bp, Artificial Sequence
  Description of Artificial Sequence Synthetic polynucleotide
tcgccgccttctcgctcatggccatcgccatcggcaccgactactggctgtactccagcgcgcacatctgcaacggcaccaacctgaccatggacgacgggcccccgccccgccgcgcccgc

SEQ ID NO: 180 - DNA, 122 bp, Artificial Sequence
  Description of Artificial Sequence Synthetic polynucleotide
ctgttgacccgcaggactcgctggatgttgaggtcgtcagcaccttctgcgggggtcaggcgtccgggcccgctgcccacaaacacgggatagtggttcaggtctgagtgagggggtggaga

SEQ ID NO: 181 - DNA, 122 bp, Artificial Sequence
  Description of Artificial Sequence Synthetic polynucleotide
ggcggcggcgccaggacatggagctcgagaacatcgtggccaactcgctgctgctgaaagcgcgtcaaggtgggtgcgcggcaggcgcccccgaccccccccccagagaaccccgaatcccg

SEQ ID NO: 182 - DNA, 122 bp, Artificial Sequence
  Description of Artificial Sequence Synthetic polynucleotide
tcgcccccagcccacttcactccatcactgtcttccttagagtttatccagaaggcaagacgtggtatccaagctcagaaccaagagcccacagcatggtgtgagctctttctgcctcttgc

SEQ ID NO: 183 - DNA, 122 bp, Artificial Sequence
  Description of Artificial Sequence Synthetic polynucleotide
gtgatgtttagaaccttttgggggattccttctctctcagaatttaacctggcaagagaacgactgagttctaggaattttcttgtctggagagagtaaaataaatgtattttttaaaagct

SEQ ID NO: 184 - DNA, 122 bp, Artificial Sequence
  Description of Artificial Sequence Synthetic polynucleotide
ctcgctgcttctcccctagtcttcgggtcccttgaacgcaggtcgcttgtttgccttacgcgtagtcagcggccagtggctatttatggcagtaaggaatattatccacatttcacatggag

SEQ ID NO: 185 - DNA, 122 bp, Artificial Sequence
  Description of Artificial Sequence Synthetic polynucleotide
taagctgtccagacctggcttgaaaacccatcccatggcaaggcagggattcgctggccgcggttggctctatcttgatctgagcaagccgctggacgtccctagttatcttcttcctatcc

SEQ ID NO: 186 - DNA, 122 bp, Artificial Sequence
  Description of Artificial Sequence Synthetic polynucleotide
ctctctgcagcccaggaacaataaatacttcctccccatgtttaaaaataaccccatgaccgcttttggcagtcataggtgaggcgggcaccacctaaggcccccccaccccatgccgttct

SEQ ID NO: 187 - DNA, 122 bp, Artificial Sequence
  Description of Artificial Sequence Synthetic polynucleotide
gagaatctgaaaatgagacccaagcgaaagtatagacattttattgtggagcaaaaccaacgacaccctcaagggaggagtgcaggcactcaaagatttgagtcacaggcaatgtggttcac

SEQ ID NO: 188 - DNA, 122 bp, Artificial Sequence
  Description of Artificial Sequence Synthetic polynucleotide
ctcaacaaggcctgcatctccggactggagctcaagtatagcccagcgagtgtcaagaaacgaaattctccaagggtggcggaatcaagccccaagtcccatgtgtcactggaccggtgagg

SEQ ID NO: 189 - DNA, 122 bp, Artificial Sequence
  Description of Artificial Sequence Synthetic polynucleotide
tagccacctcctagcacctcaggttttttacctttgagtctatgaagcctgcggaggtcacgccctagggaaagaaggagcccactgggtgtcaggtcctgcctctagggaggggaccgcgg

SEQ ID NO: 190 - DNA, 122 bp, Artificial Sequence
  Description of Artificial Sequence Synthetic polynucleotide
gagaagggcggtggagtgggacttcccgctggcctagaaaacttcagctagggctgggggcggtggctcctgcctgtaatctcagcactttgggaggctgaggctggaggatcgcttaaggc

SEQ ID NO: 191 - DNA, 122 bp, Artificial Sequence
  Description of Artificial Sequence Synthetic polynucleotide
atatagtcctattggaacccagataagcttagtctcaaagcctcccctcttgtcaccacccgactctgccttactcttggtagaaccacagcgatgacagctgcttgggaacataaccacaa

SEQ ID NO: 192 - DNA, 122 bp, Artificial Sequence
  Description of Artificial Sequence Synthetic polynucleotide
ggcttttccctttgaccttaacacttttggggttatctctgaggcgaatgctaaaggagacgctccaggactcgacctctgaaggtccttggagccaattccgtaatatgatcatggaaact

SEQ ID NO: 193 - DNA, 122 bp, Artificial Sequence
  Description of Artificial Sequence Synthetic polynucleotide
agagactgtgtccaccgtcattgaaagggtaatgcttgagaaaggcctgaaggatatgggcggacagagtgtgtgtctagggcaataaaaagtaactgctccagatgttgaagaaaataatg

SEQ ID NO: 194 - DNA, 122 bp, Artificial Sequence
  Description of Artificial Sequence Synthetic polynucleotide
aagagggcccctccaggccagtctgggcaccctgggatagcggctgcaggtaggcagaggcgctgccagtgcccaggtggcctttccctccatccggcccttcccaccttcctataaccttc

SEQ ID NO: 195 - DNA, 122 bp, Artificial Sequence
  Description of Artificial Sequence Synthetic polynucleotide
gaggcagcagtagaaacagtttgctccaaggaccaaacttattctggtgtgcagctcactcgcccctactcatctccagtgtatttcaagagtatgcagggaaggaaaaagtcaggctgagt

SEQ ID NO: 196 - DNA, 122 bp, Artificial Sequence
  Description of Artificial Sequence Synthetic polynucleotide
agcggacgcctgggccaggcctcacaacgtccagagctggaatgggtcttttgctttcggcgcaggggtgacgggatcagcggagggtaggggtgtacactagctgcggtctgatttagccc

SEQ ID NO: 197 - DNA, 122 bp, Artificial Sequence
  Description of Artificial Sequence Synthetic polynucleotide
agggctgtcgcgcctgccgtgtggtcctggagaatgaggcttaccaaaggctcaagacagcgtccccatggagtgacatggttaaagtgttgaaagaaaagaactgttggcattgaattctg

SEQ ID NO: 198 - DNA, 122 bp, Artificial Sequence
  Description of Artificial Sequence Synthetic polynucleotide
ggttcgctgacgctcagtgttttggcccggacggtcacatgtttcctttgttgtgagctgcggcagagactggtggctggaggagacgccggcgctggagagtgcgctgcgccgcccgccgc

SEQ ID NO: 199 - DNA, 122 bp, Artificial Sequence
  Description of Artificial Sequence Synthetic polynucleotide
ccagggaagcgaagcccagctgttccttcggggtgtgtacttggaactgcatccaggtctcgcttagggtccccgcggcgaggcggagcagctaatttgagagcacaacaaataaacaagga

SEQ ID NO: 200 - DNA, 122 bp, Artificial Sequence
  Description of Artificial Sequence Synthetic polynucleotide
tggggttggagctgggctgtggcactggactgcgttcggggacgggggacgcagccagaacgcgagggtggtagggaaatattgggggtttcgcgtgcaccgaagggaatgggaggagaaga

SEQ ID NO: 201 - DNA, 122 bp, Artificial Sequence
  Description of Artificial Sequence Synthetic polynucleotide
cggatcgcggggaagttcctctcagcgcctcaggtgtctgggcgtgtgcagctgtgttggcgcacacttgccgctacagcccttctgtcagccctttagcttcgatggggcgctggtggccg

SEQ ID NO: 202 - DNA, 122 bp, Artificial Sequence
  Description of Artificial Sequence Synthetic polynucleotide
taaagaaatgacaggtgttaaatttaggatggccatcgcttgtatgccgggagaagcacacgctgggcccaatttatataggggctttcgtcctcagctcgagcagcctcagaaccccgaca

SEQ ID NO: 203 - DNA, 122 bp, Artificial Sequence
  Description of Artificial Sequence Synthetic polynucleotide
cataaaagaggagacatagggggcttggtgagataccctgaaacctcccccctctgaccccgcagccaggccccaggctggccgggagtggcccctcacactggttctccccactttctctg

SEQ ID NO: 204 - DNA, 122 bp, Artificial Sequence
  Description of Artificial Sequence Synthetic polynucleotide
cctcgcgctgatcttggtgggccacgtgaacctgctgctgggggccgtgctgcatggcaccgtcctgcggcacgtggccaatccccgcggcgctgtcacgccggagtacaccgtagccaatg

SEQ ID NO: 205 - DNA, 122 bp, Artificial Sequence
  Description of Artificial Sequence Synthetic polynucleotide
gagtgggtgggtgggtctggagaagctatgtgtaccaaccaggttcacatatttttcttccgtgaagctctgtctccaccctctctggagcttctgcctgccttatttacaccccactctcc

SEQ ID NO: 206 - DNA, 122 bp, Artificial Sequence
  Description of Artificial Sequence Synthetic polynucleotide
gggggaaagtaagggagagagaaagagacggagaaaaacaggaaaacttactcttcagtacgcagggaagaatagagaaagaaaaacacaaagaaacgccacgcagactgcagagaaggacc

SEQ ID NO: 207 - DNA, 122 bp, Artificial Sequence
  Description of Artificial Sequence Synthetic polynucleotide
tcgtcggggagtgaaagcaggcgcagggaaataaaaagaaggaaagggagacagaccaggcgcctaacagatggggaccaagaaacaagagatagctgagaggtgcaaacagaagagaaaaa

SEQ ID NO: 208 - DNA, 122 bp, Artificial Sequence
  Description of Artificial Sequence Synthetic polynucleotide
gccagcccaactgttgtattttcagttcttccagtgtgaatcagttaatattctcgggaacgagggagaggttgatcctatgaggaaatcaaccacagtgaaaaggcttgggccgcttttgt

SEQ ID NO: 209 - DNA, 122 bp, Artificial Sequence
  Description of Artificial Sequence Synthetic polynucleotide
tggcggtgggctacccttttgttcctcttttaccacctgggttacgtttgtgggcagatccgctacccggtcccagaggagtcacaggaagggacttttgtagggaatgtcgctcaagattt

SEQ ID NO: 210 - DNA, 122 bp, Artificial Sequence
  Description of Artificial Sequence Synthetic polynucleotide
ccttgctggctctgtctgctgaggttttacccaagtgactccattttgaatcttacaactcgcacactactcatgtggaagatttaaatgtacattccaggacctggtgctttctcttccgc

SEQ ID NO: 211 - DNA, 122 bp, Artificial Sequence
  Description of Artificial Sequence Synthetic polynucleotide
gtggccacagaatccccttcctacaactggcaggggtcggcatgggctggagctcagagacggccagctaggacttcaggacacacagcaaactagctgcgccccgctgagggtcagcgcac

SEQ ID NO: 212 - DNA, 122 bp, Artificial Sequence
  Description of Artificial Sequence Synthetic polynucleotide
ctgacctccaggaagctgagcgtggtggatggaactctacgatctctttctctccaaggacggaaacctcatccaagcagtcccagaggaaacggataaaggtatttgaaagggagcgagcg

SEQ ID NO: 213 - DNA, 122 bp, Artificial Sequence
  Description of Artificial Sequence Synthetic polynucleotide
gccctggcagtgctctcgcggtggcctggctctctctctccggcctgaaggagagcaaagcgccccagctgcctagggccaccgctcctgacgaatccgccagccactgcacgacagatggt

SEQ ID NO: 214 - DNA, 122 bp, Artificial Sequence
  Description of Artificial Sequence Synthetic polynucleotide
tatcaacaaaaatacccacttcaggaggtggttgtaaagattatacaagagactgcagagcgttaggcagcacctggcacaagacaaatgctcagtaaaagaccactgctgtcattaaggtc

SEQ ID NO: 215 - DNA, 122 bp, Artificial Sequence
  Description of Artificial Sequence Synthetic polynucleotide
ctgtcacaattgttaacaccttctttgaccagcctttttacatttgacaattctcttcagcgcctctttcctgccagcaggaaggttttgctgccttggctttcgggagccccctagacagc

SEQ ID NO: 216 - DNA, 122 bp, Artificial Sequence
  Description of Artificial Sequence Synthetic polynucleotide
cacgactcacggacatggccccagctaattggtagcccctgggttcaaccggaatcagcgcgtgagtccaagactgggagaaagaggctcatccgagactacaattcccagaatgcgcttca

SEQ ID NO: 217 - DNA, 122 bp, Artificial Sequence
  Description of Artificial Sequence Synthetic polynucleotide
gtaaacaagcaaacaaaaacacatacacaaaccggtcactgtcagactgtctgtgagaagcgctccacaggacacagctggagaatgtgtcacaaaggaactcagaggggggcggtcaggga

SEQ ID NO: 218 - DNA, 122 bp, Artificial Sequence
  Description of Artificial Sequence Synthetic polynucleotide
caagaacctggacaccttctaccggtaacaatgggggtgtggcttgcttctttggtgctccgctgctcaaacctctagggggagcatgcagacgggcaggttgtggggcacgtgggctccga

SEQ ID NO: 219 - DNA, 122 bp, Artificial Sequence
  Description of Artificial Sequence Synthetic polynucleotide
tggcgatccaggagcaccagtacaggtcggtgacggcgatgaggtacaggtccagcaggccgccctgcgccagcagcagcaccacggacagcgcctggtagccccagcggcacctgggactg

SEQ ID NO: 220 - DNA, 122 bp, Artificial Sequence
  Description of Artificial Sequence Synthetic polynucleotide
ggagggataatgggatcaggaggctcagaaaagggcaaagaatgggaaggggcatggaaacgggtcttgaaacagttaaaaagagaagataatcaccgtcagcgtcgaaatggagccagatc

SEQ ID NO: 221 - DNA, 122 bp, Artificial Sequence
  Description of Artificial Sequence Synthetic polynucleotide
gcctaacccggcctccgaggggtgtcccagcggggcctggggtccagggcagagttcttccgccccagccattgggaatgaaggcctcagtgatgttatctgtaaagccggaggaatggcat

SEQ ID NO: 222 - DNA, 122 bp, Artificial Sequence
  Description of Artificial Sequence Synthetic polynucleotide
gagggggatttccagctgctggccggggcctctcacccctacccccgcgtagttcatctgcgacgcaacgccttgtgtcaaagcccagcacaggttctgccgcctctgacctctctgagggt

SEQ ID NO: 223 - DNA, 122 bp, Artificial Sequence
  Description of Artificial Sequence Synthetic polynucleotide
ggtgacggttacaggcgagtcctctctttgacatactcaattaagctctgtacacttgagcgtctgtccactcgtaggtgtgcatacttccactgcggatttaaactttcaaagaagtctag

SEQ ID NO: 224 - DNA, 122 bp, Artificial Sequence
  Description of Artificial Sequence Synthetic polynucleotide
actcagacaggcaggaagctgaaggcaaaggaactctctatctgattggtttccattcagcgtttctgattaataagagacgtccctcaaataggaagatattgccgctgatggcgctgcag

SEQ ID NO: 225 - DNA, 122 bp, Artificial Sequence
  Description of Artificial Sequence Synthetic polynucleotide
attcacatttagttcgcctaggaaaactagcagttagtgaaaaactggccacatcacagccgcacagctccagcagcccgggtagcttccccaccctcactttctccagccccgcctccagg

SEQ ID NO: 226 - DNA, 122 bp, Artificial Sequence
  Description of Artificial Sequence Synthetic polynucleotide
ggcatgcctgcaccctcagggcagccccggacatcggcgtcaggttgcttgagtcaggggcgtgggaatcagacagacccggtctcagatgccaccctgtactgttggttctgtcatttatg

SEQ ID NO: 227 - DNA, 122 bp, Artificial Sequence
  Description of Artificial Sequence Synthetic polynucleotide
gactgggcaaaaattaaccagggctccaacaggcgaaggtcactggactgggcaggggcacgctccgcctggggagaggagatccaggaacggtgtgtggagctgggctcggggggtgcctg

SEQ ID NO: 228 - DNA, 122 bp, Artificial Sequence
  Description of Artificial Sequence Synthetic polynucleotide
atgggggttgtggctgtggagcggaagtgggtctcaaccactataaatcctctctgtgcccgtccggagctggtgaggacagcctgccagagtctggtaagaaagggactcagggtgcgggg

SEQ ID NO: 229 - DNA, 122 bp, Artificial Sequence
  Description of Artificial Sequence Synthetic polynucleotide
gactcagccactggtgtaagtcaggcgggaggtggcgcccaataagctcaagagaggaggcgggttctggaaaaaggccaatagcctgtgaaggcgagtctagcagcaaccaatagctatga

SEQ ID NO: 230 - DNA, 122 bp, Artificial Sequence
  Description of Artificial Sequence Synthetic polynucleotide
ggtggccgtccgcgtggcagggcgggggtcccagggcggctgtgcttggtgctgggaggccgcccgagggcagcgccggccccgagtcagcagccgcagggcaccctggagatgcggaacgc

SEQ ID NO: 231 - DNA, 122 bp, Artificial Sequence
  Description of Artificial Sequence Synthetic polynucleotide
agaccctacgtaggattgcatctttacgtcgtaggcttggtctcgtgtatttttattgagcgtgtttaattagctgaggttactcgctttggcaccccagtgatcgtttttgccaccaagct

SEQ ID NO: 232 - DNA, 122 bp, Artificial Sequence
  Description of Artificial Sequence Synthetic polynucleotide
ctctcccagccgtttcccagcgtgtcatgtgctgagaaatggtgggcttagccacgcaaacgtttactgagcatctactatttggtaggagctgttaggcaccatgctagcagtggagataa

SEQ ID NO: 233 - DNA, 122 bp, Artificial Sequence
  Description of Artificial Sequence Synthetic polynucleotide
ggggaccagtttcccctcctgggatatttggtgtgcgacatgcccttcccccagccccagcgcccgttccctttggaagcctgggtgctcctcagaccacttggggactccctgcttcacct

SEQ ID NO: 234 - DNA, 122 bp, Artificial Sequence
  Description of Artificial Sequence Synthetic polynucleotide
ctgtcttacctgcagcagcctcagttatgtttttgacaactatagcaaccaactacctctcgcgagaacttacttttggccatcgcacgagcaagtttattccaagactcgcgataaccctc

SEQ ID NO: 235 - DNA, 122 bp, Artificial Sequence
  Description of Artificial Sequence Synthetic polynucleotide
caaagtcactcagctcgccaggggcagagccagggtgctcacagggtggccaaccttccacgtctgccctggacacgggactttcagtactaaaatgtgcggacgtccttctcccggacgtc

SEQ ID NO: 236 - DNA, 122 bp, Artificial Sequence
  Description of Artificial Sequence Synthetic polynucleotide
ttttcccgggcagcttacctgctcggcctgggtctttctggacagcaggcgctggaggtgcgcgtcactgtccgccgccgtgtccgcggctgcgccagacagtgtagaacctgcggcctcga

SEQ ID NO: 237 - DNA, 122 bp, Artificial Sequence
  Description of Artificial Sequence Synthetic polynucleotide
agaggctaaatgccagggggatggagtgagctacgaggaaaccactattccccgacccagcgcctaccacaatctgtttggattaccactgattagtcgtcgagatgctgaggtggtactga

SEQ ID NO: 238 - DNA, 122 bp, Artificial Sequence
  Description of Artificial Sequence Synthetic polynucleotide
atctctcaccttgctactttctcggtagccgtttctgttgtccctggattgggggctcggcgttcgctgtccctgggcaccaacccttttaaagacagtaacgttgtaggaaatcaaattag

SEQ ID NO: 239 - DNA, 122 bp, Artificial Sequence
  Description of Artificial Sequence Synthetic polynucleotide
ctgtctctccaccctgtccaccaatcagcacctggaggtgggctgggagctgcctgtgaccgcttcagcatcttttgggagtggtgacagagccacagagggctgtgagcttgcccggcccc

SEQ ID NO: 240 - DNA, 122 bp, Artificial Sequence
  Description of Artificial Sequence Synthetic polynucleotide
cggtccgggttcgcttgcctcgtcagcgtccgcgtttttcccggccccccccaacccccccggacaggacccccttgagcttgtccctcagctgccaccatgagcggtaaggatgagtccac

SEQ ID NO: 241 - DNA, 122 bp, Artificial Sequence
  Description of Artificial Sequence Synthetic polynucleotide
cgggcagccgcgggaagctggtgatgctcatgtagtccactggcgagtaggcgcccagggcgctctcctggctggcctcgttctccgccgccatcctcgcccgcgccccccagcagcgcagc

SEQ ID NO: 242 - DNA, 122 bp, Artificial Sequence
  Description of Artificial Sequence Synthetic polynucleotide
ccagattacagtctctaagtcttaaagaggccagccccacttagaggttttcctgagctgcgtatcaggacatgagttccttccactatttctaggagtactacactagagcagtatgagac

SEQ ID NO: 243 - DNA, 122 bp, Artificial Sequence
  Description of Artificial Sequence Synthetic polynucleotide
tagggactgttcatccattggtgtttgtgtgcaaactaagacgactctgttctgcgcaggcgtgttgggggtgctcccccttcctctccataacacagacgcctcccgcgcaggcgtattgc

SEQ ID NO: 244 - DNA, 122 bp, Artificial Sequence
  Description of Artificial Sequence Synthetic polynucleotide
atttccatatatcctttatccagattccctaaattattactgcatttgctttatccttctcgatcagtggacagatagatgatatagacagatagatagacaaatatatattttttgaactc

SEQ ID NO: 245 - DNA, 122 bp, Artificial Sequence
  Description of Artificial Sequence Synthetic polynucleotide
acctgcctgggtgcaggaccccagagggaccccaggccacccctggcctgcccatgcccacgggaatcccgaccttgggctgcctgtctattgcaccagaaccgtcccagggctgactcaga

SEQ ID NO: 246 - DNA, 122 bp, Artificial Sequence
  Description of Artificial Sequence Synthetic polynucleotide
gacaaagtgcaggggatatagaccaaccgcttgtgaaggctgctggttctgttagaagcccgctttcgattgtcagtggctttgaggcaaaggattttggaagggaaagcaaagtgattgtc

SEQ ID NO: 247 - DNA, 122 bp, Artificial Sequence
  Description of Artificial Sequence Synthetic polynucleotide
ggacgttggacgtcagcaaggcctgggtggtgctggtgaacggtgatgcttgcggccacacgcggggtggctaagccagggacccgaactcatatgaggtctggaaggtgtgttgagacact

SEQ ID NO: 248 - DNA, 122 bp, Artificial Sequence
  Description of Artificial Sequence Synthetic polynucleotide
aggcggtgaggggcttccggttggggtggcagggtggtggatctgtcggtcccgttttcccgtcgcacgtggtggccactgttggcttctgaatggtttgcaaggcggatatccacgccaag

SEQ ID NO: 249 - DNA, 122 bp, Artificial Sequence
  Description of Artificial Sequence Synthetic polynucleotide
agtccagaaaggcccagcctgaatcactgtgaggttgccaggggcttggtttcagttactcggaaaccacccatcctccaggccagcacccaggagctgcgtgggccttgggcagtgcccct

SEQ ID NO: 250 - DNA, 122 bp, Artificial Sequence
  Description of Artificial Sequence Synthetic polynucleotide
cccatccgggattgaggagcatcccaattctgggaccatctcggggtccctgacccggggcgaatggctctcccatcttgggacccccatgcagggctgcagacccccaggcgcccccaccc

SEQ ID NO: 251 - DNA, 122 bp, Artificial Sequence
  Description of Artificial Sequence Synthetic polynucleotide
gagcgaatcctcttcgggctttccagagtgcgggggatagataaagagtagctggggagacgccccctgaccttgctgggtcccagaacccggctgctcacccccaaggggtcctctccagc

SEQ ID NO: 252 - DNA, 122 bp, Artificial Sequence
  Description of Artificial Sequence Synthetic polynucleotide
gccactacagcactggtgcccaaacctggcacactaggaacaaagtccttgttacttcttcgtgggcgttttcaccaagtagacttggaggttaacaaaggacgcaaaggagaggttctaat

SEQ ID NO: 253 - DNA, 122 bp, Artificial Sequence
  Description of Artificial Sequence Synthetic polynucleotide
ttttcacaggagtgaggcagaagacagaaactgcaacaaaccgccggggggtgggattaacgtccaaagctcacaccggcttcaactgattggcaggaacgaagtgggtggagcctcctgac

SEQ ID NO: 254 - DNA, 122 bp, Artificial Sequence
  Description of Artificial Sequence Synthetic polynucleotide
aataataaataataatgaatccattcttccttcggtcgtgggtctggcaggcataaattccggccgggattccgaccccagggccagagcaggactcgccttggcgtctatgagtgggcggg

SEQ ID NO: 255 - DNA, 122 bp, Artificial Sequence
  Description of Artificial Sequence Synthetic polynucleotide
cttggggggccaggggcagggcctgtggggcggggcgggcctggcttgttgggcctgtgccgggtgtccgggaggggccagacggggtcttggaggggcggggccggggcctgtggggcggg

SEQ ID NO: 256 - DNA, 122 bp, Artificial Sequence
  Description of Artificial Sequence Synthetic polynucleotide
gggctgaagagacccccccccaacacaccagccccgaaaaccgtctgccgtcccctatagcgctgcatggaaaagaaccaagacaaggacttggagtggagaagacagaaattgtccactga

SEQ ID NO: 257 - DNA, 122 bp, Artificial Sequence
  Description of Artificial Sequence Synthetic polynucleotide
ccatttgagggcaagggctgtgtctttgggtacttcgctcctcgcagtcacaagtactggcgtgcgtacgcggggagagatcgctcctcaaaacggggtcctgaacgctgccccgcggcccc

SEQ ID NO: 258 - DNA, 122 bp, Artificial Sequence
  Description of Artificial Sequence Synthetic polynucleotide
ggacggcgcggaggagctggaggatctggtgcacttctccgtgtctgagttgcctagtcgcggctacggcgtcatggaggagatccggcggcagggcaagctgtgcgacgtgaccctcaagg

SEQ ID NO: 259 - DNA, 122 bp, Artificial Sequence
  Description of Artificial Sequence Synthetic polynucleotide
catgcctagggaatgacaggcatctccacaggcaggctgcatccaccttggctggggtgtcgtcattggctgcctattagaaaaacgacaggacaatgcataccaccgcctcccgactgtaa

SEQ ID NO: 260 - DNA, 122 bp, Artificial Sequence
  Description of Artificial Sequence Synthetic polynucleotide
caactgcttgccaatttaaatttctggagagaaaaatgcacccactacaaaacggacgagcggagggttagacctttgccaggtagcgctcaaaatccgctaagactactcccaccgaaact

SEQ ID NO: 261 - DNA, 122 bp, Artificial Sequence
  Description of Artificial Sequence Synthetic polynucleotide
gctgatctccagtctgcacactgttggcaaattaatctttctgagctcttgttttcatcgcgtccctctcctgctccaaagccctctgggactgcctccagtagcgcttcacaaacttcagc

SEQ ID NO: 262 - DNA, 122 bp, Artificial Sequence
  Description of Artificial Sequence Synthetic polynucleotide
cgcacaaaatcccagcctcaagggcagaacattttaaatgacccacccatcctagagatgcgccagttaggtcatcttatatatcttgagatagctgagatggtcagatcaaccaaggacct

SEQ ID NO: 263 - DNA, 122 bp, Artificial Sequence
  Description of Artificial Sequence Synthetic polynucleotide
tacccctctgcctagctacctggagccggactttggccgtgtccagcgggaaggtgatcacgtccgccaagcacgccgctattccagctgagaagagctggacccccagggtcgggtgtacg

SEQ ID NO: 264 - DNA, 122 bp, Artificial Sequence
  Description of Artificial Sequence Synthetic polynucleotide
catgatactcatgtatttccttagatcaagtcagcctagatcaggtgttcttccagaggccgtattgagatcatttttatttgcagcctgtgcattttctcatctcgggtgatggcctcaca

SEQ ID NO: 265 - DNA, 122 bp, Artificial Sequence
  Description of Artificial Sequence Synthetic polynucleotide
agcccggctgcagactcgttagcagcgaggctttaaatacaaaagtgggccgggagcccccgcgtggtgccgcggtgccccctcattatgcatgcatggaaaagcaaacaaacaaaaacatt

SEQ ID NO: 266 - DNA, 122 bp, Artificial Sequence
  Description of Artificial Sequence Synthetic polynucleotide
gtcagtgttcttttagtttgcttaaactgtgtgggtacttgagtccttttaaacgattaacgctgggaagaggcaccatttaattaattaatttgttctggaagggatcagtgtacaatttt

SEQ ID NO: 267 - DNA, 122 bp, Artificial Sequence
  Description of Artificial Sequence Synthetic polynucleotide
ccacatttgcaaccttggccatctgtccagaacctgctcccacctcaggcccaggccaaccgtgagtaccctgccccactgggctagtccctggcctgccagcttcagggagaggggtcttc

SEQ ID NO: 268 - DNA, 122 bp, Artificial Sequence
  Description of Artificial Sequence Synthetic polynucleotide
caccacagactctgggaggctcggcggctggagcagcaggcagctccccgcagctcccggcgcttccaggcagctctctgagccgtgccagaggcccggcccgccattcccaggtaggagac

SEQ ID NO: 269 - DNA, 122 bp, Artificial Sequence
  Description of Artificial Sequence Synthetic polynucleotide
gaagagtgtcgggatccacacaggaacacacaggagaaattcaccactgtgcaggagggacgtggttaaggcgagttctgccattaacgtgtaattagacaacacttttaccccgcccctcc

SEQ ID NO: 270 - DNA, 122 bp, Artificial Sequence
  Description of Artificial Sequence Synthetic polynucleotide
cgccgccgcctcttcgtcgcctcagcctggcgttttgttccgagagacgggagaggcgagcggagctgacagtgattttgacagtgatttaaacccgcttttgttgttgttggcttttcgtt

SEQ ID NO: 271 - DNA, 122 bp, Artificial Sequence
  Description of Artificial Sequence Synthetic polynucleotide
agtgaactgagcaacagcaagtgcaaaggccctgctagcaccatgagcacgatgagagatcgtccaggaggcggtgttgatgcggcaaagggcaacaggaagggcattaggacttgaaatcg

SEQ ID NO: 272 - DNA, 122 bp, Artificial Sequence
  Description of Artificial Sequence Synthetic polynucleotide
aatcaaaggcggggtacagggccagagggaggaggaaacaacttcccggttgctttcagacgcttcagagatcctctggaggcctgggggagcttttgagtactttatttcagttggtccct

SEQ ID NO: 273 - DNA, 122 bp, Artificial Sequence
  Description of Artificial Sequence Synthetic polynucleotide
cggaatactgtctggctgtgcacgtggaggtggcgaaatgtggaagcttaacgaagttggcgccatgaagctaaagactgctaccccggggctctagctcgctccgcctaatggcgggccgc

SEQ ID NO: 274 - DNA, 122 bp, Artificial Sequence
  Description of Artificial Sequence Synthetic polynucleotide
cagcatgcaggcaccgcctcctcatctgcatagagccgccctcctctcagccaaacctgccgctcatctgcataagccccgcctgctgaaggctctgcagcttgaatattttctgagcggaa

SEQ ID NO: 275 - DNA, 122 bp, Artificial Sequence
  Description of Artificial Sequence Synthetic polynucleotide
gagggacgacagcttttactgtcccaaatcctgagattaagacctcagggctaaatcttgcgtggcggtaaaaattatttggaagttctgtgcaaccgttccaatattccgcttttacgtgc

SEQ ID NO: 276 - DNA, 122 bp, Artificial Sequence
  Description of Artificial Sequence Synthetic polynucleotide
tcagggctggcagtctgggccagggtgtggtttccagcgtcaggccaggctctgctgtctcgcgagggtccggcctcaatgctccaggccccggcgttgggccgcgcctcctcgtgggcctc

SEQ ID NO: 277 - DNA, 122 bp, Artificial Sequence
  Description of Artificial Sequence Synthetic polynucleotide
ttcttgacattcagaagtcagattcagggaccccatggcagagcctgtttctaacacttgcgcctattcgactatagggactatattctgcacagaaatatacttagttttatatatggtta

SEQ ID NO: 278 - DNA, 122 bp, Artificial Sequence
  Description of Artificial Sequence Synthetic polynucleotide
ggacaggtacacgacgatgacgaccggggtggtgagaagctgcccgaccaggtcggtgagcgccagccagccgatgcacagcaggaaggacttcttgcgcttgctctcccggcgccggtagc

SEQ ID NO: 279 - DNA, 122 bp, Artificial Sequence
  Description of Artificial Sequence Synthetic polynucleotide
ttccagcaaatagaaaacaaccgagagcctgaattcactgtcagctttgaacactgaacgcgaggactgttaactgtttctggcaaacatgaagtcaggcctctggtatttctttctcttct

SEQ ID NO: 280 - DNA, 122 bp, Artificial Sequence
  Description of Artificial Sequence Synthetic polynucleotide
ctttcaaaggcaagctgcagggctccttggttttgtcacattcctcattctggggctttgcggttttgtcttgggaatctcgaggctctcccaaggttcctttctatgtttatatcatttag

SEQ ID NO: 281 - DNA, 122 bp, Artificial Sequence
  Description of Artificial Sequence Synthetic polynucleotide
ctggtccccccggcgggcggcggcgcgggcaggggcaagggctccgggctcctgcggctgcgttggctgctcaggccaccataatccagctcgcggctcgcagctcccgggcgggctgggga

SEQ ID NO: 282 - DNA, 122 bp, Artificial Sequence
  Description of Artificial Sequence Synthetic polynucleotide
gtctcctagggctgaagacaacttggattgcgaggctagggcttggggagtcgtgcatcccgttccgggcctccgcagcccaacatgggccccgggttccaaagtttgcgaagttgggcgcc

SEQ ID NO: 283 - DNA, 122 bp, Artificial Sequence
  Description of Artificial Sequence Synthetic polynucleotide
ttaaaactgttttccagggcagtttctctctctggttctaggacacttaattgggctcaacgtttcccccaagtcttggctgtgggtttgttgtgggctgggtggttgaggagaggatgcag

SEQ ID NO: 284 - DNA, 122 bp, Artificial Sequence
  Description of Artificial Sequence Synthetic polynucleotide
ccgcagacccctcggtcttgctatgtcgagctcacccgtgaagcgtcagaggatggagtccgcgctggaccagctcaagcagttcaccaccgtggtggccgacacgggcgacttccacggtg

SEQ ID NO: 285 - DNA, 122 bp, Artificial Sequence
  Description of Artificial Sequence Synthetic polynucleotide
cgtgtcacagactctcagaaagcacagtgagagttcccctggttgagaatcgcaggctgccgctggcttcccccacttccctgggcacatcggaggagggggccacagctggtccctggtct

SEQ ID NO: 286 - DNA, 122 bp, Artificial Sequence
  Description of Artificial Sequence Synthetic polynucleotide
ccccgaggctcccgcatcaacgccctcagtcggatgggactgagggtgccgccgccaccacgccggggactgttgacagccagaacctttaagcgtaacagagtcacctggcaagtttgtac

SEQ ID NO: 287 - DNA, 122 bp, Artificial Sequence
  Description of Artificial Sequence Synthetic polynucleotide
attggcaggtcctcgattatcggcgagtcactgaggttccgagaggggcgtctctgctcacgcaaacagctacccagccgcctcccacggtctgacctcagccaaggtgacgcggcttaaag

SEQ ID NO: 288 - DNA, 122 bp, Artificial Sequence
  Description of Artificial Sequence Synthetic polynucleotide
ctgtatctctttgtctctccctgtgtgtgtgtggggtctctctcagtctttgtccctctgcgtctctgggtctctctgtctctgtctccctgcgtctctctctctctgtcatctggctcttc

SEQ ID NO: 289 - DNA, 122 bp, Artificial Sequence
  Description of Artificial Sequence Synthetic polynucleotide
agagggcagggctgtattccgctactgggtcctatgcaccatgcagaaccagtgtcttcacgtggagactcatcactgatccgaaaggtgactgcttctgtattacactcatttccccatga

SEQ ID NO: 290 - DNA, 122 bp, Artificial Sequence
  Description of Artificial Sequence Synthetic polynucleotide
agagacactatctccaaaagaaaaaaagagaaacgtatggttacatgatttccttctgtgcgatcatcagagtcactcgcagacctggatggtgtacggcctacaatgcacctaaaccacct

SEQ ID NO: 291 - DNA, 122 bp, Artificial Sequence
  Description of Artificial Sequence Synthetic polynucleotide
tccgggcgaggagatcagcaggggttttcgagggagcctggggcccagggcaggggtacgcgggtcaactcaacagatgtaaggcgtggccgaaccccattcagctagcagtacccagcctc

SEQ ID NO: 292 - DNA, 122 bp, Artificial Sequence
  Description of Artificial Sequence Synthetic polynucleotide
acttctccgaggttacacagctaggaaatggtggcaacagtaagagcccacgaagagctgcggttggtagttcattctggacagccctcccgtgaaccgtccctgtactggcacttgttgct

SEQ ID NO: 293 - DNA, 122 bp, Artificial Sequence
  Description of Artificial Sequence Synthetic polynucleotide
cgattagtaaataccaacccatgctagagagtgaagagctctggaggagaggcacgggtgcgcccctggagttgctctaaacagggtaggcagggtgctcttgtcacagagaagatgaacga

SEQ ID NO: 294 - DNA, 122 bp, Artificial Sequence
  Description of Artificial Sequence Synthetic polynucleotide
cccaagccccgtcgattagacaggtttaggcacttccgggactctcagaagcctgggaagcgagttctctgcaattggactaagcctgcgaccgtctggtataacaattatatgaataatcc

SEQ ID NO: 295 - DNA, 122 bp, Artificial Sequence
  Description of Artificial Sequence Synthetic polynucleotide
caggtcgggccagttgctggtgagcttatgaagtgtggtctcctccccggagctcatgtgcgcttcccacctggtgagctcagggtctctctggagggatcctgcctcccacccctgtctcc

SEQ ID NO: 296 - DNA, 122 bp, Artificial Sequence
  Description of Artificial Sequence Synthetic polynucleotide
ttttcccttagaggccaaggccgcccaggcaaaggggcggtcccacgtgtgaggggcccgcggagccatttgattggagaaaagctgcaaaccctgaccaatcggaaggagccacgcttcgg

SEQ ID NO: 297 - DNA, 122 bp, Artificial Sequence
  Description of Artificial Sequence Synthetic polynucleotide
cagggtgagagcaggtctcactcatcccaatcccagccaggattgggtcagggcccccagcgcttacctgcaggcaaggtgctgctccacgaccttctccagctgctgccgctgctgaatct

SEQ ID NO: 298 - DNA, 122 bp, Artificial Sequence
  Description of Artificial Sequence Synthetic polynucleotide
ggctgggccagggtggggcgtggcccggggcgggggaggggcggggctgccaggcaggggcgggacggagaacacctgggtccctagcaccaagactggctttttattcattgccaccgcct

SEQ ID NO: 299 - DNA, 122 bp, Artificial Sequence
  Description of Artificial Sequence Synthetic polynucleotide
tggttacccgtgagtcacctcgctgtgccccctgcccagagcgggaaccctggctgcgcacgccctcaaatatctgcaggtgctgttcacaatcgccatagggccggtgacatacccaggaa

SEQ ID NO: 300 - DNA, 122 bp, Artificial Sequence
  Description of Artificial Sequence Synthetic polynucleotide
gggagctgagttgctggtagtgcccgtggtgcttggttcgaggtggccgttagttgactccgcggagttcatctccctggttttcccgtcctaacgtcgctcgcctttcagtcaggatgtct

SEQ ID NO: 301 - DNA, 122 bp, Artificial Sequence
  Description of Artificial Sequence Synthetic polynucleotide
ggctagggacgttatgtaagttgagccacgctacgctaaaagttccacactcaattctagcgtctcggctctggactaccaagttccggagcaagcagacagaccacctctttacgttcccg

SEQ ID NO: 302 - DNA, 122 bp, Artificial Sequence
  Description of Artificial Sequence Synthetic polynucleotide
ttagaaacctctcagtggggtttttcgaaatgaaagtctaactccttgtctctttctgaccgtttccatgctgaacctcatctttctaatggcccactcctccaggggcctgcctgacgccc

SEQ ID NO: 303 - DNA, 122 bp, Artificial Sequence
  Description of Artificial Sequence Synthetic polynucleotide
cgccgccccccccacccctcccccagacaaacgatatgacgcacttaactataaaccccccgaccccccgtggcttctgggaatttcccagaaggttctgcatgggcagatgctgagggagt

SEQ ID NO: 304 - DNA, 122 bp, Artificial Sequence
  Description of Artificial Sequence Synthetic polynucleotide
tatttaaagattgtggctaatagtagagtagatacccctggtatttcccagagcagagctcgcattctgggaatcttaggtccatgtgacttcctgagtcagtgattcacactgagaaaaga

SEQ ID NO: 305 - DNA, 122 bp, Artificial Sequence
  Description of Artificial Sequence Synthetic polynucleotide
cggagggggaaacaaaactacagcaagaccaccttgagtaccttgggaagggcagccccgcgatccctaataaatgaattagcatctcaaggaggagatcactgcggggctgatattgatca

SEQ ID NO: 306 - DNA, 122 bp, Artificial Sequence
  Description of Artificial Sequence Synthetic polynucleotide
tctatgttgtctctatgccttgctgtcttgcctgcctccttgtaggtccaacctcgggagcgcagcttttaaagagtgacagtgtttgtttggatcacccgcagcttgactcatccttgctt

SEQ ID NO: 307 - DNA, 122 bp, Artificial Sequence
  Description of Artificial Sequence Synthetic polynucleotide
cttaatcccaggtttgtttatccaagcagtggtgtcagctgcctggccaaaccacacaggcgcctggatcctaggagacataaaccaatcctcccacccaagcaaagccccgtagcagcccg

SEQ ID NO: 308 - DNA, 122 bp, Artificial Sequence
  Description of Artificial Sequence Synthetic polynucleotide
gccgcccggggtccgaattggggggggcggctgtgtgaccttgggcgaatcgccgcactgcgctgggtctgcgctccgcatccatcacaggcagactcctcaagaggctccaaccttttctt

SEQ ID NO: 309 - DNA, 122 bp, Artificial Sequence
  Description of Artificial Sequence Synthetic polynucleotide
ggtaactgcacaggagaaggtgaaccagtaagtgggccatatgtctctgcaaaacttgcacgtaggaatcacctgctggggaactaagacacttttatgtttgcagcagaggctgtgttaga

SEQ ID NO: 310 - DNA, 122 bp, Artificial Sequence
  Description of Artificial Sequence Synthetic polynucleotide
cccagggtccaggcccgccctcggctggcaggtgtgggcacagaggcagctgggattggtcgcagctggcggaggcgcgtcccaggctccggcagaccgctggaacagctgagcagagcagg

SEQ ID NO: 311 - DNA, 122 bp, Artificial Sequence
  Description of Artificial Sequence Synthetic polynucleotide
ccccccgcctgccgagggggctggcgggggggcattcctgggtccctggaactctgagcccgcgtcccccacccctaaggggcgtggggggggggcgcacccctccaaccccctttccccag

SEQ ID NO: 312 - DNA, 122 bp, Artificial Sequence
  Description of Artificial Sequence Synthetic polynucleotide
agcctggattctagtgaagcccaattcaccagccatttggtcttagtaaggtcattaccgcgctctaggtttgagtctcatttgtaaaatgaagggagtggaggggcttatagagctcgaac

SEQ ID NO: 313 - DNA, 122 bp, Artificial Sequence
  Description of Artificial Sequence Synthetic polynucleotide
gtgcgcgctatgtgacctctcaggggtcgctgccttggacgatctgtaaagctgagtgcgcgctatgtgacctctcaggggttgtttccaaccgtgttgttgacatcttgagcctgccaagg

SEQ ID NO: 314 - DNA, 122 bp, Artificial Sequence
  Description of Artificial Sequence Synthetic polynucleotide
gagctaaaaggtagtatcccaccctctccataaacagacacctaagttataaaacttatgcgctcgatatgcaaaaatagctcgttttatacagaaacgatcctttccttcttttccttata

SEQ ID NO: 315 - DNA, 122 bp, Artificial Sequence
  Description of Artificial Sequence Synthetic polynucleotide
ggcagctggggatgggcaggctgcagcgtgggcaagacgaggtggctgctgtgactctgccgctgaaccctcaggaagtgatccaggggatgtgtaaggctgtgcccttcgttcaggtgagt

SEQ ID NO: 316 - DNA, 122 bp, Artificial Sequence
  Description of Artificial Sequence Synthetic polynucleotide
ggtcagtcggggcctgcagaccgtgactccgtcacgaaccccaaattcgcttctccccaacgctcgggcctgactgctcaggaggggcttatgtaaccttaacctggtccctccgcacagga

SEQ ID NO: 317 - DNA, 122 bp, Artificial Sequence
  Description of Artificial Sequence Synthetic polynucleotide
tttcttcaaattaaattgctacagcaggaaattactgaactgtggctcttctcctacgtccgccttccctatgtcaattcccatttcccttgctttctccaatagttaggactgtaaattct

SEQ ID NO: 318 - DNA, 122 bp, Artificial Sequence
  Description of Artificial Sequence Synthetic polynucleotide
ggacagatggatggacgctcgcgggcaatgaatgggcgctgcgctcaaccaagacactcgcgcaaagttgtggctccacccaaggcacctgctccgcacactttaagcggcgccctggaggc

SEQ ID NO: 319 - DNA, 122 bp, Artificial Sequence
  Description of Artificial Sequence Synthetic polynucleotide
tatgcgatgatgtttgtttgcccttgacgcacttactcatggatggtacttcttcagcctcgttagacagcctggtgatggaggatgaagaaaccatgtgcttttcattcagttctggactt

SEQ ID NO: 320 - DNA, 122 bp, Artificial Sequence
  Description of Artificial Sequence Synthetic polynucleotide
tgtgtgggacagtcaggtcggcaggagtgcatgagaacggtgtgggcacacgtaagtgcacgatcacacatacaagtgagcttgagagtgtgtattcctgtgcactgtgtgcacacctgtga

SEQ ID NO: 321 - DNA, 122 bp, Artificial Sequence
  Description of Artificial Sequence Synthetic polynucleotide
gcagagtccaattatgtgttttctgataaaagcatatgttcattgaaaacactggaagagcggcatactggaatactggtttatctggtgtatttcgggagtttacagatcacgaaagttgc

SEQ ID NO: 322 - DNA, 122 bp, Artificial Sequence
  Description of Artificial Sequence Synthetic polynucleotide
ccctcccccgccagcctggcgcattgcgggcctcgggctcattgctgagagggggcactgcgcctggcacctctgttaagcaatttaggggctacaacctgagcaagacagatgagcccggc

SEQ ID NO: 323 - DNA, 122 bp, Artificial Sequence
  Description of Artificial Sequence Synthetic polynucleotide
tatgttgagtgaaagaagccagacaaaatcaagtacatatgggatgattccatttatggacgactctagaaaatgcaaactaaaacagatcagtgtttgggctgcggatgagtggagttggg

SEQ ID NO: 324 - DNA, 122 bp, Artificial Sequence
  Description of Artificial Sequence Synthetic polynucleotide
cctacgaagaggtagggcttggcaaggacccacggggcgtgtcctaggactcggtgagggcgtgacctcgggccaggggcggggagagaaccagagggcgaagtgggagggcacaggggaga

SEQ ID NO: 325 - DNA, 122 bp, Artificial Sequence
  Description of Artificial Sequence Synthetic polynucleotide
ccttccggtagctcggtcactagggtcagttttatgactctcagtggaccctaaacagcacgtaatatatgtatttttcaccgccaaatatatcaaacacaataattcaccctccgttccct

SEQ ID NO: 326 - DNA, 122 bp, Artificial Sequence
  Description of Artificial Sequence Synthetic polynucleotide
acccatgagccaattgcagaggcaacagaagaccagtgcaccaaccaggctgggtccctccgccagagggtgtcaccatctaagctgaaagtgtttggggagatcagacattgctgtctggt

SEQ ID NO: 327 - DNA, 122 bp, Artificial Sequence
  Description of Artificial Sequence Synthetic polynucleotide
ggaagctgggctgtgcgtgtatgcgtctaccatgtgggggtgcctgtgagtgtgctggggcgtctgcagtgaaggcctcctgagaccactccacggaaacaccgggaatccctgcagctgag

SEQ ID NO: 328 - DNA, 122 bp, Artificial Sequence
  Description of Artificial Sequence Synthetic polynucleotide
gcgtcgctttcacactcggcggctgcggattgacgcctccgcctgttccccggaggagagcgagtgcaagagaaaaaacacttttattgaaacgatccaaccagcggcggcggagaaaagcg

SEQ ID NO: 329 - DNA, 122 bp, Artificial Sequence
  Description of Artificial Sequence Synthetic polynucleotide
tccggggtttttaccctcggcagtttgatgtcctttgtgtcaaggtctggctgcggaggccgggaaaatgtggcccccgtcagtaagggttgggcagggagcttggcgtggcctggcggatt

SEQ ID NO: 330 - DNA, 122 bp, Artificial Sequence
  Description of Artificial Sequence Synthetic polynucleotide
gcgttacttgcaggatgcaggagtgatgcgatcagagccagccggaaccgagttccgttacgcactacaggactgacctgggcctgacaacccactgccggagttcggatcgcatcactgcc

SEQ ID NO: 331 - DNA, 122 bp, Artificial Sequence
  Description of Artificial Sequence Synthetic polynucleotide
atggttacatgatttccttctgtgcgatcatcagagtcactcgcagacctggatggtgtacggcctacaatgcacctaaaccacctagaggagcctcttgctcgtgggctacaaacctgccc

SEQ ID NO: 332 - DNA, 122 bp, Artificial Sequence
  Description of Artificial Sequence Synthetic polynucleotide
gcggacttgtccggatccgaatagaagcgctgttggatgcggatggggcgccggggttgccgccacaggtgcttcggggctctggtcatgctgtggcggccgcgagagcgactcaacctgct

SEQ ID NO: 333 - DNA, 122 bp, Artificial Sequence
  Description of Artificial Sequence Synthetic polynucleotide
aggctggacatttgctactggtccctgaagttttgcggctgcacccacagacagcaatagcgccacgttccctggaaggcgcacgggacggaagcggaagcagtaacgctggctccggctgc

SEQ ID NO: 334 - DNA, 122 bp, Artificial Sequence
  Description of Artificial Sequence Synthetic polynucleotide
ggagatggcaacagggcaagcgtccagcaatgggtaagcggtggggtcggtgcacgcaggcgtccagcaatgggtaagcggtggggtcggtccacccagggagcgctggtccccctggaagg

SEQ ID NO: 335 - DNA, 122 bp, Artificial Sequence
  Description of Artificial Sequence Synthetic polynucleotide
ggaggtgctgcggtacctaccatggtattcttgtcccggaacgtagtaggtggggttgcccgcaatatgcagggaaatgagcacctcgccctgctccccatccccttccagctccccgtggt

SEQ ID NO: 336 - DNA, 122 bp, Artificial Sequence
  Description of Artificial Sequence Synthetic polynucleotide
gcctctgggagggcaagaccgggaggggtcggcctgtgtcgggggctcctggaaaagcagcgccaccgccacccacctgacgacatggaaggcccaaagcaggcgatctgtgcgaggcccgc

SEQ ID NO: 337 - DNA, 122 bp, Artificial Sequence
  Description of Artificial Sequence Synthetic polynucleotide
ctggcagatgtttgtactgggagattcagatccatccaggcccccactgttaatagcccacgggaaagtccctgcagtctctcagggaagtcattctgtgtagaatctgtaatttcacaggc

SEQ ID NO: 338 - DNA, 122 bp, Artificial Sequence
  Description of Artificial Sequence Synthetic polynucleotide
agctggggctcgcctgttgggagccgcgtccgccggtgttggtgtctgcacttggaaggacgtagggaatgcgttgtccctgctaggtacttttcagtcgcagagttctcttcttcttcttt

SEQ ID NO: 339 - DNA, 122 bp, Artificial Sequence
  Description of Artificial Sequence Synthetic polynucleotide
cttggtgttcagcaccagccgcccccccagccgcatcatcttttctttcaacaacagatgcgcccgtgtttcatctatggatagagctgagccgaagaaagacattgccacagccaacagca

SEQ ID NO: 340 - DNA, 122 bp, Artificial Sequence
  Description of Artificial Sequence Synthetic polynucleotide
tgatagtattttctactgtcctatacacatcaggcaagacttcatggagagcactgaacacgtactcactatgtgcctagcattgttgttaatcactttacatgaattagttcatttaattc

SEQ ID NO: 341 - DNA, 122 bp, Artificial Sequence
  Description of Artificial Sequence Synthetic polynucleotide
caagagcgaccctcgttcttcacacgaggagaagaaatggacacgtgattgaccattaggcgccaccagggccaaactatcttatggaaggaggaaaagaagcacagaaagggcatgaaatt

SEQ ID NO: 342 - DNA, 122 bp, Artificial Sequence
  Description of Artificial Sequence Synthetic polynucleotide
ggggccctggcccgggaccagcgccgcggctataaatgggctgcggcgaggccggcagaacgctgtgacagccacacgccccaaggcctccaagatgagctacacgttggactcgctgggca

SEQ ID NO: 343 - DNA, 122 bp, Artificial Sequence
  Description of Artificial Sequence Synthetic polynucleotide
tcacccccgtcttggggacatcaggtctgtgagcacccataccccagccaggcactgtggcgccccactcgccctcccgcactccctcctagagatgccctcttatatccccggagttcgca

SEQ ID NO: 344 - DNA, 122 bp, Artificial Sequence
  Description of Artificial Sequence Synthetic polynucleotide
agcaagggaagttggatgagaatttgaatccaaagcgtgccatgggaccacaattgcacacgatcaatgagtctcacaaactgaccacggcttatctgaggcagtttagggttgtgcaagag

SEQ ID NO: 345 - DNA, 122 bp, Artificial Sequence
  Description of Artificial Sequence Synthetic polynucleotide
aaaagacgagatgacaagacacagacagcgagcatgtgcctgtgcacatttgggtctgtgcgtctctggatgggggtgagagagaaaataaaagaaggggagtggaggaaagagaatgcccg

SEQ ID NO: 346 - DNA, 122 bp, Artificial Sequence
  Description of Artificial Sequence Synthetic polynucleotide
aggttaaacggcactgaccatgctgagccacagccggtaaagatggcggtggcacactgacgtcacttccgctccgagcctccggccgggtggggctccagggcttgagtttcaggcacgta

SEQ ID NO: 347 - DNA, 122 bp, Artificial Sequence
  Description of Artificial Sequence Synthetic polynucleotide
aagcccacgtgagagggcaggacgcctgagagcttgaggccacacgaggctgtggagcggcgtgactcaaacgtggcgcgcatcagctcgcacacttccaaacctcgcgatagctactggcc

SEQ ID NO: 348 - DNA, 122 bp, Artificial Sequence
  Description of Artificial Sequence Synthetic polynucleotide
gacccaggcgactgacatgttcctctcctctcagctgaaaagctttgctagctctgtctacgcataaagtaaggttaaacacagattttgccccgaagggcattaattagggaccaatttac

SEQ ID NO: 349 - DNA, 122 bp, Artificial Sequence
  Description of Artificial Sequence Synthetic polynucleotide
ctggagggaggaaggtgtggggggacccaggggtcctgtctccaagcctggttgctcttacgcgaaaagttgggacactgaggtgtcacagcttctcttttgaaatggagaggaggtaggag

SEQ ID NO: 350 - DNA, 122 bp, Artificial Sequence
  Description of Artificial Sequence Synthetic polynucleotide
tgccgtggggaaaacctgcctgctgatgagctacgccaacgacgccttcccagaggaatacgtgcccactgtgtttgaccactatgcaggtaagaaaaagtgggaaactctctgcatccaga

SEQ ID NO: 351 - DNA, 122 bp, Artificial Sequence
  Description of Artificial Sequence Synthetic polynucleotide
ctggcagccagtggttcgccggcactgacgactacatctacctcagcctcgtgggctcggcgggctgcagcgagaagcacctgctggacaagcccttctacaacgacttcgagcgtggcgcg

SEQ ID NO: 352 - DNA, 122 bp, Artificial Sequence
  Description of Artificial Sequence Synthetic polynucleotide
tcagtgcgtgttagcgagcagcgccgggagatagctgtcaccgccgcccgctcacagatgcgtagactgaggctcaggtgtcaccacctgaccaaggctagttccgctacaaagctgccgac

SEQ ID NO: 353 - DNA, 122 bp, Artificial Sequence
  Description of Artificial Sequence Synthetic polynucleotide
tgggtggaaaaggaaagggcccattagacgaatctgattcatcttctgtgactaagcacccgcaacagttaggaatttaggcagagctggtgatcctgggacaatagcacttcctaggtaat

SEQ ID NO: 354 - DNA, 122 bp, Artificial Sequence
  Description of Artificial Sequence Synthetic polynucleotide
gcgcgcgtgccgccgccgcgggcactgcgcccgtttgcctgcccctcgtcggggatcgggcgctccctctgagacctgaaagggcacccaagtgccccctgtctgcgaagtccggcgcgggc

SEQ ID NO: 355 - DNA, 122 bp, Artificial Sequence
  Description of Artificial Sequence Synthetic polynucleotide
gacccccggcagggacgtttttctgcaaactcacagcatttgacaaagttacataaacggcgcccggccggccccggcgcccgcccgcccccgccctcactcccggcggcccggagcccacc

SEQ ID NO: 356 - DNA, 122 bp, Artificial Sequence
  Description of Artificial Sequence Synthetic polynucleotide
cgtgcgtggccaggatcacatcgttggggtccatggtggtcttcagcaggcccctgtagacgcggtaatcgccgctagcgtccaggacgcctccagaggccagcgcggtgcggagctgcgcc

SEQ ID NO: 357 - DNA, 122 bp, Artificial Sequence
  Description of Artificial Sequence Synthetic polynucleotide
gagcctcaggggcggagtcttagtgtccagaggggagtcagggcagctggaggtccagggcgggaaccattgaggctgggaccctacgagaaccccctaccccgtgcccttcggcctctctt

SEQ ID NO: 358 - DNA, 122 bp, Artificial Sequence
  Description of Artificial Sequence Synthetic polynucleotide
ggaagcaatccggcccctttttggcagcgagttggcccggtctttggctgcctcagaccgcgttgccctccagcctcgaggcagagagctgcctcggtgccacagctaaataagcccggcgc

SEQ ID NO: 359 - DNA, 122 bp, Artificial Sequence
  Description of Artificial Sequence Synthetic polynucleotide
ggcaagcaggtttggttcctgcccagcaaaggtgagggaggacggaggagactctcccaccgcattcagaactttattcctttatttttgtctcaatcttgtcatagaggagcgcttcactt

SEQ ID NO: 360 - DNA, 122 bp, Artificial Sequence
  Description of Artificial Sequence Synthetic polynucleotide
ccgctgcctagtctgcatctgagtaacatggcggcggcggcggtagccaggctgtggtggcgcgggatcttgggggcctcggcgctgaccaggggtgagcacgggcagccagctgagaccgg

SEQ ID NO: 361 - DNA, 122 bp, Artificial Sequence
  Description of Artificial Sequence Synthetic polynucleotide
caggaacatcacttggtaattaagagatcgccttgcttcagatccttgctctcctagccacgtgactgtgagcaagtgactttgcttctctgtgtctgtttcttcaactataaaataggtat

SEQ ID NO: 362 - DNA, 122 bp, Artificial Sequence
  Description of Artificial Sequence Synthetic polynucleotide
cagacgcttctgaaagggcaaagacgacgccaaagaagacgccggagacctcgaatagggcgcaggtggacatctctgattttcagcagaccagcctgtatgtgtctgaagtctagcaacga

SEQ ID NO: 363 - DNA, 122 bp, Artificial Sequence
  Description of Artificial Sequence Synthetic polynucleotide
gaggagggcgctggtgctcaatgagtgagcccacctggggactaccaggacgaggacgggcgcaggtgaaagtcctgggctcattgccccagcatccaactttcaccctctgtcccctttag

SEQ ID NO: 364 - DNA, 122 bp, Artificial Sequence
  Description of Artificial Sequence Synthetic polynucleotide
aacaaacaaagctaaggttcttaccccacggcttgcactctctcagcagagctgcaggtgcgtggatgattcgttgacacggtcagaattggctgcaggagggaattgaatcgaggttttct

SEQ ID NO: 365 - DNA, 122 bp, Artificial Sequence
  Description of Artificial Sequence Synthetic polynucleotide
accggcgcgagttggaaagtttgcccgagggctggtgcaggcttggagctgggggccgtgcgctgccctgggaatgtgacccggccagcggtgagttggggccggggcagagggcaggggtg

SEQ ID NO: 366 - DNA, 122 bp, Artificial Sequence
  Description of Artificial Sequence Synthetic polynucleotide
cggggcagcccgccccacccctccccccaggctcctccccatccctccctgcccaggccgcgagaatgaccactccacttgcaggcgaagcccctggccgctgtgctgaaggaggtgtgcga

SEQ ID NO: 367 - DNA, 122 bp, Artificial Sequence
  Description of Artificial Sequence Synthetic polynucleotide
gtgtggtgcttccttctgaccttgggcacctccgtcttcagttgcccctcctgtgaaaggcgaaatgtatcgttgggttctttgaggccctttacagctctgacatcctataacattctgta

SEQ ID NO: 368 - DNA, 122 bp, Artificial Sequence
  Description of Artificial Sequence Synthetic polynucleotide
cgggcactatgctgagcaactgcagctcaggtcctgcagagtccccgagagtactttgcacgaagagagctcgagttctgtagtcaggcatatctgacctaccgaacaggtgccctggtcaa

SEQ ID NO: 369 - DNA, 122 bp, Artificial Sequence
  Description of Artificial Sequence Synthetic polynucleotide
tggagcaggaccaacttaccagctcgcggtgctccctagaagctggattcttcgcaggtgcgagcacaccccagatgccagcgtggacccttgagcaactggaagttaaaaacccacgaaaa

SEQ ID NO: 370 - DNA, 122 bp, Artificial Sequence
  Description of Artificial Sequence Synthetic polynucleotide
atcattcattcattcattcattcacccattcactaatcagtaaaatttaagtgtccactacgttccaggacttgcactagactttaaggataacagggtggacaagttccactttggagacc

SEQ ID NO: 371 - DNA, 122 bp, Artificial Sequence
  Description of Artificial Sequence Synthetic polynucleotide
ggaaaagtcattttaagtaaagacaacgagttaatcaggaggcgatgagcccagtccttccgccccgctttcccgcttccagccctcgaacgaaccctcctctaacccccgggaggcaggag

SEQ ID NO: 372 - DNA, 122 bp, Artificial Sequence
  Description of Artificial Sequence Synthetic polynucleotide
tggcttggggtctcagggaaccgaaccgccctccccccagacctgctaccccaggccccacgttggtgcccatttcacaggtgataaaaccgagacccaaagagccggtgtccggcccaagg

SEQ ID NO: 373 - DNA, 122 bp, Artificial Sequence
  Description of Artificial Sequence Synthetic polynucleotide
aaataatcagcagttcctggtggcatgtaaccaagtaaaaaccagttacacagagagccacgaacccccaaggcaagaaagcagaatgtgaaaatgctttatatgggggggtggggaatggt

SEQ ID NO: 374 - DNA, 122 bp, Artificial Sequence
  Description of Artificial Sequence Synthetic polynucleotide
ggcatgggggctgggggccgagatgcccaggtttctgggtgtaaggactcaccatgactccgccagccatcactgcacctgccgtctctccccacttcctctggtggggcaggaagctgagt

SEQ ID NO: 375 - DNA, 122 bp, Artificial Sequence
  Description of Artificial Sequence Synthetic polynucleotide
ttgtgagacactgtttctgagagcagcttttgtggcatcttacagggcagatttctggtacgttctaaaagttgaatttctaactttggctggttgtggcccctgactgttttttttttttt

SEQ ID NO: 376 - DNA, 122 bp, Artificial Sequence
  Description of Artificial Sequence Synthetic polynucleotide
attcctttacttttctataactctgtcatgaccagtttaaaggccccaatgtcatgtcctcgcattaacaaccaaggctacaatgcaagccctgccatgtgcgcttctttacaaaaggtcaa

SEQ ID NO: 377 - DNA, 122 bp, Artificial Sequence
  Description of Artificial Sequence Synthetic polynucleotide
tctgcttacagctgcttccaaattaagcatatctggatggtgtgacactttttgttagtccgagaactgtatgggcatcgcaactgggcctgttccaagatagacttgttgggaccttcaaa

SEQ ID NO: 378 - DNA, 122 bp, Artificial Sequence
  Description of Artificial Sequence Synthetic polynucleotide
cattcttatgcgactgtgtgttcagaatatagctctgatgctaggctggaggtctggacacgggtccaagtccaccgccagctgcttgctagtaacatgacttgtgtaagttatcccagctg

SEQ ID NO: 379 - DNA, 122 bp, Artificial Sequence
  Description of Artificial Sequence Synthetic polynucleotide
agtttgagagaccagggctgctggggcctggtcatgcagggcccggacagggggctgtcccgctgtgaggaagctcttggcttaccctcctctgagcctcagagcttgtgaggttagttcct

SEQ ID NO: 380 - DNA, 122 bp, Artificial Sequence
  Description of Artificial Sequence Synthetic polynucleotide
ccgtctcctcacctgccccacccgtggcctgggtttaaaatccacatacccgtctttccgcggccaaagtgatgctgccaggattggttatgaccccaactgccccgacccccagaagtgca

SEQ ID NO: 381 - DNA, 122 bp, Artificial Sequence
  Description of Artificial Sequence Synthetic polynucleotide
aagaagccctcaccgagagctgtgggaacaagagctgccgggaacaagagctgcgggaagcggctcctacgaattggtggcaggaggcacaaaaacgaaatacctatttttggaatacggaa

SEQ ID NO: 382 - DNA, 122 bp, Artificial Sequence
  Description of Artificial Sequence Synthetic polynucleotide
taaaccagagacttgaattattggcaaatgtccagacaacattcacaatgcttactagcacgctattgccatatgtacctggaaagagcagcataaagaagccatctaatgatattacacac

SEQ ID NO: 383 - DNA, 122 bp, Artificial Sequence
  Description of Artificial Sequence Synthetic polynucleotide
ggtgcctccaggccacgtgggctggcagtcaactcacctgtttctcagaggagtccaggacgcacagaaggtgccggtcactgccctctgccggacccatggaggggtaagggtgtccggcc

SEQ ID NO: 384 - DNA, 122 bp, Artificial Sequence
  Description of Artificial Sequence Synthetic polynucleotide
agccccacctctcccttagggacctccgcccaccctaccctcaagccaggatgcccggagcgtccccggaagtgggtgtggttcaggtgatttaactcattatttaatacgcccgcagggtg

SEQ ID NO: 385 - DNA, 122 bp, Artificial Sequence
  Description of Artificial Sequence Synthetic polynucleotide
cagagtaatttaacccaggattgctgactttttaagagctgagaaagcatagctatggagcgcaaggccccacccagcagggtctaagtattccgtctgcaaaactggcaggccaccaacgg

SEQ ID NO: 386 - DNA, 122 bp, Artificial Sequence
  Description of Artificial Sequence Synthetic polynucleotide
cctggggtgtaagtactgcttgtgggagagccccacaggaaatccagagtattgcgcatgcgtgctgtccagaaggcgcttgaactcggcggcttccgtagcgggagggcgaaagatggcgg

SEQ ID NO: 387 - DNA, 122 bp, Artificial Sequence
  Description of Artificial Sequence Synthetic polynucleotide
gctcggtgcccatggcccactgctgctggaggaacctgtgtctccctttgcagcctgtggcgcgccttccttgcagggtgtgtacactggctgtttgcagagggggtttgtgcatcctagtt

SEQ ID NO: 388 - DNA, 122 bp, Artificial Sequence
  Description of Artificial Sequence Synthetic polynucleotide
gcgcccggagccgggctgcttggttccagtgttgggccacatactgcttgcgtgctaggtcgcccctccgggtggctcagcctcttcccctctctcacaatccctgaatccctctgtccctt

SEQ ID NO: 389 - DNA, 122 bp, Artificial Sequence
  Description of Artificial Sequence Synthetic polynucleotide
cctgaacaccgctctgcagaatcttggtggctaaggtgtccaggagcctctgcagcggaccgccagcctgagaggcgcagagcttgtcgggcaggggcccgcttgtcccactcccctgattt

SEQ ID NO: 390 - DNA, 122 bp, Artificial Sequence
  Description of Artificial Sequence Synthetic polynucleotide
gcccgcccggggctagaggcggccgccgggagggcgcgcggcgccggagacatgtccaggcggaaacagagcaacccccggcagatcaagcgtgagtcaaactttgcccgcggtcccctccg

SEQ ID NO: 391 - DNA, 122 bp, Artificial Sequence
  Description of Artificial Sequence Synthetic polynucleotide
cctcccagtggccacgcgccttctcacgcccctctcccgtgacgtcatgctcctctcgcgcggcatgatgggagaatcctaatgttttccaacagatgctccaagaacagctttcagattaa

SEQ ID NO: 392 - DNA, 122 bp, Artificial Sequence
  Description of Artificial Sequence Synthetic polynucleotide
cacctggtagttgtctagctgctcttcggtgaagatggtctgcttgttccccatggtggccgccgcgccgccgctcgcccgcccgggctccgactcccatcagcggccgccagacccggagc

SEQ ID NO: 393 - DNA, 122 bp, Artificial Sequence
  Description of Artificial Sequence Synthetic polynucleotide
gactccatatgccctagggatgtgttgtgatgaacttttcctactggtactgtttcctcccgcgagggaatgtctagaccagccgcaccttcttgctttgacccttcagaactttggcctgt

SEQ ID NO: 394 - DNA, 122 bp, Artificial Sequence
  Description of Artificial Sequence Synthetic polynucleotide
tctgccgtactgtaactgaaacacaggttcagttgctcactgcttgcagagtccagttaacgagagcgggatctgttataaagaaagtgatttattccaaagcttagcttatgagaagaaat

SEQ ID NO: 395 - DNA, 122 bp, Artificial Sequence
  Description of Artificial Sequence Synthetic polynucleotide
agggaagaaatcaactccgacttctttgcaaaactgaaatctctgtgaaatagccagatgcgcacaccaaataagggtttctaaagagaacccaagttacttttcaattaaaaaaataaaat

SEQ ID NO: 396 - DNA, 122 bp, Artificial Sequence
  Description of Artificial Sequence Synthetic polynucleotide
ataaacccgactcaaaatctgtcttttcctgggcagattgcaaaggattttgcatctccccgttgctgttgctgctgctcacacagtcttgggaaaacgggggaaaatcaaggaaagagagg

SEQ ID NO: 397 - DNA, 122 bp, Artificial Sequence
  Description of Artificial Sequence Synthetic polynucleotide
ccggaggcagcagacaaagactgggcagcaccgggcacgttcccgctcctggcccctccccgggccgcacttccagaatgggagtgaattgcctcccaattaaagaagcaattttttaaaaa

SEQ ID NO: 398 - DNA, 122 bp, Artificial Sequence
  Description of Artificial Sequence Synthetic polynucleotide
gtctttccaaaaggcataggaaatcagcaagtttccaccaaatataccaaaaccctaagacgcgagccagcccaagggtgcaaggttctgcggctgcaggtgatgtgcgtgtgtgcgagtgt

SEQ ID NO: 399 - DNA, 122 bp, Artificial Sequence
  Description of Artificial Sequence Synthetic polynucleotide
ggtgtggttggtgcgcaggtcggcgggtgacgcgcggtctttgcacactgggcaggtgggcgacacctgcacctcccagcagcggctcacgcacccgcggcagaagttgtggccgcagcgca

SEQ ID NO: 400 - DNA, 122 bp, Artificial Sequence
  Description of Artificial Sequence Synthetic polynucleotide
aggacaaatgggtgcagagattcaggctggccaaggctggcacaaggacattcccagtggcgagagcatgagcaagggtcacggatgtgccaggaggggaggcggagagatgcctgggacca

SEQ ID NO: 401 - DNA, 122 bp, Artificial Sequence
  Description of Artificial Sequence Synthetic polynucleotide
tcatctatcaacgtagtaggcactgtcctaggcgctagggattccatgcagagcaaaaaacgtcacagtccatgccttcacatggccttcatggaccaccgcgggtgttctttttcccccga

SEQ ID NO: 402 - DNA, 122 bp, Artificial Sequence
  Description of Artificial Sequence Synthetic polynucleotide
ctctgaaacggacaagatggctgccacctcttcgcgcctcttagtcccacccactcagggcggaggtctgcgtcatgtgaccctccccttcttggctccgcctcctaccgcagtgcttgacg

SEQ ID NO: 403 - DNA, 122 bp, Artificial Sequence
  Description of Artificial Sequence Synthetic polynucleotide
cacttaattcttgcaaatacctctcggtgctgacttcaaggaacttggctggctttgggccgcagaagtgaaaaacacaaagctctccacaatgttcaagttgttttcttcttaatgttacg

SEQ ID NO: 404 - DNA, 122 bp, Artificial Sequence
  Description of Artificial Sequence Synthetic polynucleotide
agcctaacatcaactcttttaattgtcatgacaattctatgagatgggcacttatcgccccgtttcacagacaggggatgcagagggtacagaaaggtacagtggcttcctcggggtcactg

SEQ ID NO: 405 - DNA, 122 bp, Artificial Sequence
  Description of Artificial Sequence Synthetic polynucleotide
ctcggcccacacagcctccgggtggacctgcaggggcctgtttgtgctgtaggcttgacacgtccaggtatctctgtgtgtctgtgtatctcagtgtgagtgtgtgtgtgtgtgcacacttg

SEQ ID NO: 406 - DNA, 122 bp, Artificial Sequence
  Description of Artificial Sequence Synthetic polynucleotide
ggtgcgttgttcgcgggggtgaattgtgaagaaccatcgcggggtccttcctgctgaggccgcggacaccgtgacctcgctgctctgggtctgcagggaaacgtaggaaaaaaagttgtcag

SEQ ID NO: 407 - DNA, 122 bp, Artificial Sequence
  Description of Artificial Sequence Synthetic polynucleotide
ttgcattcaggtagattatttggaagatgatttaaggacgtaccagtgcaggagttgtcgcgggacagtgagaccagggcagtttgacaatcaataaagggtgcatcattggcaagctacct

SEQ ID NO: 408 - DNA, 122 bp, Artificial Sequence
  Description of Artificial Sequence Synthetic polynucleotide
ccaatggggaaaggcagtgtcgggactaagcaatgaatggctcttcaatggccagctgcccgcccaataggataaaagaaaaccccacataatacttccctttgtctccaaaaaaatttata

SEQ ID NO: 409 - DNA, 122 bp, Artificial Sequence
  Description of Artificial Sequence Synthetic polynucleotide
tggagcccgaaggcgccgggcagcctgaaagggagaggtgggtccggaaccacacccaggcgggtagcctggggcatcctcagacggacttcaaaagccgcttcactttcccctggtggcct

SEQ ID NO: 410 - DNA, 122 bp, Artificial Sequence
  Description of Artificial Sequence Synthetic polynucleotide
agtctttcttcttgaaagcattgttgatccaaatccaagtgtcaaggtgcgccccagaaacgctgcttcccagacagtcgtgtctggtcttgcgggaaaggaggaggcgtcccgccaaggaa

SEQ ID NO: 411 - DNA, 122 bp, Artificial Sequence
  Description of Artificial Sequence Synthetic polynucleotide
ccacgaagagcttgatggcgtcgtggtccttcatgggtacggcgggaccggggtttagcccgctcatgccgacgccgctgtccgcggtgctgaaacccaggcgcgggccggggccagcgggc

SEQ ID NO: 412 - DNA, 122 bp, Artificial Sequence
  Description of Artificial Sequence Synthetic polynucleotide
cgcctgtttcccgcctgctctcaggagcgaccgccagggggcgcccgagatggcagggggcgtgggaagcccacatctgcccagcaggtgcgcccaccccgagcaaacagggggccggggcc

SEQ ID NO: 413 - DNA, 122 bp, Artificial Sequence
  Description of Artificial Sequence Synthetic polynucleotide
aaatattactgtttattaccaggcataccccagtaaaataaagaggcaaccaggcgatagcgactatctcaccagccgctgcacctataggacttggagacgtcacgagtcacgcaaccggc

SEQ ID NO: 414 - DNA, 122 bp, Artificial Sequence
  Description of Artificial Sequence Synthetic polynucleotide
agctgccaaacatctggatcaacctgggcactacgaggggttgaatttctaccattatcgcgccttttgatatttttttccagacctcctgctcacatccgtaaagcccactgattctttta

SEQ ID NO: 415 - DNA, 122 bp, Artificial Sequence
  Description of Artificial Sequence Synthetic polynucleotide
aagaaagctcaaaggtaccctgcagacactcaaaacttgagggcacgcaactctcagttacgagtggtggcaatcataatgacagaatgaagtaccagtgcaagaaactggaagcgtgtgga

SEQ ID NO: 416 - DNA, 122 bp, Artificial Sequence
  Description of Artificial Sequence Synthetic polynucleotide
ccatggtgccctggggccctgctacaggtgctcaggtagggaggtagggtgcctgctgtacgctggacctggacctactgggccccaggcaggacatcctttagaccctctgggaggctcca

SEQ ID NO: 417 - DNA, 122 bp, Artificial Sequence
  Description of Artificial Sequence Synthetic polynucleotide
aacacagggtaggacttcaaaacaccagcgtgagcgaggcaggcacacacggactcgcggcggtctgtttgcaacagcgctgggaatgcacattggaaaatcacatcttgcatgctgaaaac

SEQ ID NO: 418 - DNA, 122 bp, Artificial Sequence
  Description of Artificial Sequence Synthetic polynucleotide
cccggttggtgagggagggagtcccaacccagggttatgggtggctggacacacaacacccgacactggacagataagactgacagcagttcagctgcatgtactcacggcctgaggcagga

SEQ ID NO: 419 - DNA, 122 bp, Artificial Sequence
  Description of Artificial Sequence Synthetic polynucleotide
gaaggctcctgggcctttctggctctgggaatgaagcgtggaaaaccctccttaggcgggcgcagtgcttcaagtagccaagctctgacttccgagggaagaaaggaggccatgggcctctg

SEQ ID NO: 420 - DNA, 122 bp, Artificial Sequence
  Description of Artificial Sequence Synthetic polynucleotide
aactcagtcccgtcccttttgttgacaggttgccaggatacatccaggcaacaaagactgcggttcctgttactcagcagcctcaaaaactcacaccagctcctgcaaggaatgtgaatctt

SEQ ID NO: 421 - DNA, 122 bp, Artificial Sequence
  Description of Artificial Sequence Synthetic polynucleotide
ctcgccaggcggcgctgtgcctgggaggactttcccgctcatcgcgggggctgcacgtggcgctgaagccggggtcccacccccaatgtgctcgtcctaccacagccaaggctgggattcca

SEQ ID NO: 422 - DNA, 122 bp, Artificial Sequence
  Description of Artificial Sequence Synthetic polynucleotide
tgtgcggagccattcgctgcgctgaagcagtgcgcatgcgcactggacgcttcttaccagcgtcctgactacaatacccaggacgcacccagcccgccgcctctcggagcccttttcaaacc

SEQ ID NO: 423 - DNA, 122 bp, Artificial Sequence
  Description of Artificial Sequence Synthetic polynucleotide
gcggcggagcggcgggttggggcgtcgcacggtgagaaaggccggggcctgagaacaaaccgccgcggtcgccggggcaacgggacggggcacgtgccccccccgccagagccggaagcggc

SEQ ID NO: 424 - DNA, 122 bp, Artificial Sequence
  Description of Artificial Sequence Synthetic polynucleotide
gtagttgcggggacctgggaggccgggctctttcctccttggcctgccttccgctggctgcgtggggcagccaagaacaaagcctgcgagcttccatcaattgtaaagcaaagcacccttta

SEQ ID NO: 425 - DNA, 122 bp, Artificial Sequence
  Description of Artificial Sequence Synthetic polynucleotide
ggcaggcaggctccatagtgccaggcatctggctggctcagcagcagggggcgatggcatcgtcttcctgcccacctgggagccaatgtttcggctgggcaaggacaagcctcctctgggtc

SEQ ID NO: 426 - DNA, 122 bp, Artificial Sequence
  Description of Artificial Sequence Synthetic polynucleotide
gaaggccctgaccctgctgagcagtgtctttgctgtctgtggcttgggcctcctgggtatcgcggtcagcaccgactactggctgtacctggaggagggtgtgattgtgccccagaaccaga

SEQ ID NO: 427 - DNA, 122 bp, Artificial Sequence
  Description of Artificial Sequence Synthetic polynucleotide
tattagtaaagcgtttactaaattaccgaatcaaaccgaactggcttaggttctcaatagcgtggaaatccactgaaaataaatgaagagggcaaactacaggggctccgcaggttcgggtc

SEQ ID NO: 428 - DNA, 122 bp, Artificial Sequence
  Description of Artificial Sequence Synthetic polynucleotide
caatggctaaggagtatagaaaggatcattatagtgtgtgtctctgtgggtcctatgttacggcaagatgaaacaagcttattaggctctgtcttttaagggcataccagttgaaagagcat

SEQ ID NO: 429 - DNA, 122 bp, Artificial Sequence
  Description of Artificial Sequence Synthetic polynucleotide
acttgcccaacatgagccctggtcttgtctgaccccaaagcccatgggaagtttaggctgcgtggaaggacagcctggtgggctcaggatctgtcccatcacgagttggaacctcagctctg

SEQ ID NO: 430 - DNA, 122 bp, Artificial Sequence
  Description of Artificial Sequence Synthetic polynucleotide
acctaggaagtaagataattttaaaaagagagcactttggcagtggtgaagcaggtgaaacggttgaatacaacacctgtggtttcaaagaaaagttcccacagagcggatacactactcgt

SEQ ID NO: 431 - DNA, 122 bp, Artificial Sequence
  Description of Artificial Sequence Synthetic polynucleotide
ggacggcaaggacgcgtggctggcgacggtttcgcaggggcgcccgttcccctgggggcgcgaagtccccgctccaccgctgccccaactcggctccgaagtgcctttgccgcaagacttgc

SEQ ID NO: 432 - DNA, 122 bp, Artificial Sequence
  Description of Artificial Sequence Synthetic polynucleotide
tgcgccagggcggccacgcaggccaggcagaccacgtggccgcaggacaggttgcgcgggcgccgctgctgccggtggccaaacttctcaaagcacaccttgcactcgagcaggctgatctc

SEQ ID NO: 433 - DNA, 122 bp, Artificial Sequence
  Description of Artificial Sequence Synthetic polynucleotide
tcacatctgtcatctctcaggtcatatccaacacactgggccacccacgcacagggacgacgcgacagccctgtggctccaccgcacaggacagccacgactggcaatcctgtgccggccct

SEQ ID NO: 434 - DNA, 122 bp, Artificial Sequence
  Description of Artificial Sequence Synthetic polynucleotide
tagctatgacacatggcttggaaattaacctttaaccaaacatcttataagtaacgccagcgcagcttcccttgtgaatgtaaagagatccagggctcttggagagggacaagtgagagcca

SEQ ID NO: 435 - DNA, 122 bp, Artificial Sequence
  Description of Artificial Sequence Synthetic polynucleotide
agggggattccaagagagatttttgtaaatgtcaaatagtcgacctcatgctgggcagaacgctgtatttcagtatacagggaagataaagaaagaggtagagaagagattgtcctgttttc

SEQ ID NO: 436 - DNA, 122 bp, Artificial Sequence
  Description of Artificial Sequence Synthetic polynucleotide
gacgaggacaggacctcctggatgcactggaaagtcgaagagacatggtatcagggcaaacgcgttgcagagctgtatttgtgaaagccagaatggagtgccttcttgtctaaaaggtttgg

SEQ ID NO: 437 - DNA, 122 bp, Artificial Sequence
  Description of Artificial Sequence Synthetic polynucleotide
gaggcccagcaggtaagcacttgtggaggccccggtggctgctggttagctcttgaagctcgtccccaccctgcgtgcgttctaaagagccgcgtttctattgcaactgcctgccctgcgct

SEQ ID NO: 438 - DNA, 122 bp, Artificial Sequence
  Description of Artificial Sequence Synthetic polynucleotide
tcagtctccccatatttacaataaaaggggagcgaggtgggatggcgctgaggatccctacgtccgatcctaatctccagctcaggcaggctcggccgccactagcatcctggagcgacaac

SEQ ID NO: 439 - DNA, 122 bp, Artificial Sequence
  Description of Artificial Sequence Synthetic polynucleotide
agctgtaattccattgacagtgaattggagtaatagccctcccccgtctcccaagctctgcgtccagtccacacaaagcccacggcagctgcaggctgagcttgtcctgcttcagatcactc

SEQ ID NO: 440 - DNA, 122 bp, Artificial Sequence
  Description of Artificial Sequence Synthetic polynucleotide
aaacggagactcagcaacggggctgatttgtctgtggacacacagcgaactgtaagtccccgcctccctctgcacccgcgtgcaccagggggctgctgggggtgcggggacgcgggagacct

SEQ ID NO: 441 - DNA, 122 bp, Artificial Sequence
  Description of Artificial Sequence Synthetic polynucleotide
tccttgagcacacaccttctctcaacaaatgacaatacttggcaaactgaactcctcccacgagtcgccctctgctaggaggaattgctggctgctccctgcttattgcattctctcagagc

SEQ ID NO: 442 - DNA, 122 bp, Artificial Sequence
  Description of Artificial Sequence Synthetic polynucleotide
ccggggctgcctggcctcctgggtgcgggaggtgcctccagattggcctggcttctgtgacgctggcccagatcacacaccagagcccttggtgggcagcggcacctgcaagcatactgcag

SEQ ID NO: 443 - DNA, 122 bp, Artificial Sequence
  Description of Artificial Sequence Synthetic polynucleotide
ttccgtgtctcagatggggcctgggtcaagtcctgggagttgatggagcgtttcccaaatcgcaaaaggagaggagctagacttacctccccctcctgggaagtaatgcgcgacaagaattt

SEQ ID NO: 444 - DNA, 122 bp, Artificial Sequence
  Description of Artificial Sequence Synthetic polynucleotide
ataaattaacagtcagatctaggggctcgatcagatttgtgtgtgtgtgtgtgccgtgtgcgcgtgcacagcatgttctttgactaggaggcacacctgctttggttatcttctttttgtaa

SEQ ID NO: 445 - DNA, 122 bp, Artificial Sequence
  Description of Artificial Sequence Synthetic polynucleotide
gaggcctgccccagcctcaggaggaggagcctggcccagtccgttgccaagccgaagcagcggcatttggacaaagcagatcatctgcaggtattatatacatgggcagtgcaaggaggggg

SEQ ID NO: 446 - DNA, 122 bp, Artificial Sequence
  Description of Artificial Sequence Synthetic polynucleotide
tggaggtgctgggcaggggcggcgcccccttccctggccgcggtgcgcccttgcgcccggcgcttgggtcctgcgagatgagggtctagaaatacacagcaccacccgacccccgcatcggg

SEQ ID NO: 447 - DNA, 122 bp, Artificial Sequence
  Description of Artificial Sequence Synthetic polynucleotide
caatattcattttattaggccattgtgagagatctcagctcagcataatgggcaacttcccgtgactctgggccactgggttattctgggacttaactactctgagttttctcactagaaag

SEQ ID NO: 448 - DNA, 122 bp, Artificial Sequence
  Description of Artificial Sequence Synthetic polynucleotide
cttccggcggacttggcctttgcggtgcgagctctgtgctgcaaaagggctcttcgagctcgcgccctggccgcggctgccgccgacccggaaggtcccgaggggggctgcagcctggcctg

SEQ ID NO: 449 - DNA, 122 bp, Artificial Sequence
  Description of Artificial Sequence Synthetic polynucleotide
tttatcaccctttcggtaaatagtggtcccacggctcggcctgcttttggaatgaagctacgcttggtaagttcaactctctttcacagccctctccacagaaagaactctggagttcgttc

SEQ ID NO: 450 - DNA, 122 bp, Artificial Sequence
  Description of Artificial Sequence Synthetic polynucleotide
agagggaactcagcaggacagtgaggtgaccttcgctgtggctgttcctggggactctgccgccacctcttcccctaacgcctccgcgtgtgaatcctctggcaccaccacttgccccatat

SEQ ID NO: 451 - DNA, 122 bp, Artificial Sequence
  Description of Artificial Sequence Synthetic polynucleotide
gctgaccccggggagcgtggactacgagttggcgcccaagtccagaatccgcgcgcaccgcggtaagctgcgccttttgaaaaggctatctgtactccttggaacaaaccaccccgggcaaa

SEQ ID NO: 452 - DNA, 122 bp, Artificial Sequence
  Description of Artificial Sequence Synthetic polynucleotide
tgtgctctggaaaacacatcccatcagagctgaatcacccacatggactgttagctcaggcggggaaacattcaagtcattcaggcccaaggaataatctatagaagtcaaaggcaagagga

SEQ ID NO: 453 - DNA, 122 bp, Artificial Sequence
  Description of Artificial Sequence Synthetic polynucleotide
gagagcgggtagcggggagggccgcccacgacggaggtttctctgtggttacctcagcggcgctcttcgcaatctgaaagttggggcagctgaagagccccaccaccttcacctgcagcggc

SEQ ID NO: 454 - DNA, 122 bp, Artificial Sequence
  Description of Artificial Sequence Synthetic polynucleotide
ctggggcctggggtcacctcccctctctgggccaatcacctgttgagtctggagcactggcggctattcttaggggtttctatatttaaaatggggcctgactggcttgaggtcatctccag

SEQ ID NO: 455 - DNA, 122 bp, Artificial Sequence
  Description of Artificial Sequence Synthetic polynucleotide
gggactattcctagtttatgaggtggttaaggatatcggtggggtgggctggagcggtgtcgggttaggtctgagagaaggcctcgcacaaaacactgtacaaacccgaaaggaagtctgag

SEQ ID NO: 456 - DNA, 122 bp, Artificial Sequence
  Description of Artificial Sequence Synthetic polynucleotide
aaaataaaatcccgccatcctcccccctccccgccccacccccgccaggtttcaacagcacggactccagtccagtgcagtgccgccacaccagagacaacaggtgtttcgggaaaagaccc

SEQ ID NO: 457 - DNA, 122 bp, Artificial Sequence
  Description of Artificial Sequence Synthetic polynucleotide
aggcgccatgtcagcccgggaagtggccgtgctgctgctgtggctgagctgctatggctccgccctttggaggtagagagacgccagtcgcaggcgagcgactaggcggggattacccccgg

SEQ ID NO: 458 - DNA, 122 bp, Artificial Sequence
  Description of Artificial Sequence Synthetic polynucleotide
cccgcacacgtggccctcccgcctccgggccccgcccccttggccgcaactggcaactcccgcctgaagaatagattctctggttcacagccgtctgcaggctcaggaacagatctgggcgg

SEQ ID NO: 459 - DNA, 122 bp, Artificial Sequence
  Description of Artificial Sequence Synthetic polynucleotide
gtcgcgcagccctggcccgagggttcccggggcacggccgctgggcccccggtggaggagcgtttccgccagctgcacctacgaaagcaggtgtcttacaggtaaggaggacgtgggcagag

SEQ ID NO: 460 - DNA, 122 bp, Artificial Sequence
  Description of Artificial Sequence Synthetic polynucleotide
ccacaaagcgaggaagggcaggggctacggagtgggggcaccccgaaagccttgagcccccgagtttgctcggttgagggtgttgggggcacagggatgctggcccccagctccccactgga

SEQ ID NO: 461 - DNA, 122 bp, Artificial Sequence
  Description of Artificial Sequence Synthetic polynucleotide
aatggaaactgctaatttttgaagcagaaggttgacagcttcagtaagatctcaagagagcgagaagactggaatcaggtgaggccataacttcttatctaaacttagtttctggggtggaa

SEQ ID NO: 462 - DNA, 122 bp, Artificial Sequence
  Description of Artificial Sequence Synthetic polynucleotide
gtgggggctgggcagcgtgtttgtcccacctgtgtaaactctgattccagcaacttattccgcatgcgcccagtctaattaaaataaaagtgaatcaaattttgaatggattggtgtttcga

SEQ ID NO: 463 - DNA, 122 bp, Artificial Sequence
  Description of Artificial Sequence Synthetic polynucleotide
gtgtgaattgatgaccaaggcatggcagagcctctctcatctttataatcagttcagcggcggcctccactacagggaactcccagccagtcccgaggcctagggacatccagggagaaacg

SEQ ID NO: 464 - DNA, 122 bp, Artificial Sequence
  Description of Artificial Sequence Synthetic polynucleotide
caggcgcttcccaccagctacagtcggagatttggagcgcttgtgtctgaggctcaatcccgtcaggtgccgcgcaactcagcggcgcattctctttggacccgaggcaccaccatactttc

SEQ ID NO: 465 - DNA, 122 bp, Artificial Sequence
  Description of Artificial Sequence Synthetic polynucleotide
gcctgctccccgtcccacccctccctgagcacgccaccccgccctctccctctctgagagcgagatacccggccagacaccctcacctgcggtgcccagctgcccaggctgaggcaagagaa

SEQ ID NO: 466 - DNA, 122 bp, Artificial Sequence
  Description of Artificial Sequence Synthetic polynucleotide
ctctgcggtggcccgagccccagcggcctcaggtgagcgggcagcatcccgattccctggcggcctagaatggaatcgcaaggtttagagaaattaagggacctgggacttgccaccctggg

SEQ ID NO: 467 - DNA, 122 bp, Artificial Sequence
  Description of Artificial Sequence Synthetic polynucleotide
atacacatttttggccccaacctgcatcgaccaagtcagaaattctgcagtgtgtgttttcgtaagtcctccaggtgactctgatgtactctcagggttcagaaccattgagagagagcagt

SEQ ID NO: 468 - DNA, 122 bp, Artificial Sequence
  Description of Artificial Sequence Synthetic polynucleotide
tgacagccggaggttccagctgcgcgcccacagcccctcggtagcgccgccgactcgtggcgtctataggctgtttctgcgtcactcatgcatggaagaccaatcagagagcgtacttgtct

SEQ ID NO: 469 - DNA, 122 bp, Artificial Sequence
  Description of Artificial Sequence Synthetic polynucleotide
gtgccccctcctctttgctgctgcagtgtctgcgccgggccatttaatgagatttattcacgcacggctcttctcagctttgcgaggggttggcagatccagtgcacagggatttcccacta

SEQ ID NO: 470 - DNA, 122 bp, Artificial Sequence
  Description of Artificial Sequence Synthetic polynucleotide
gcacagctgccctttgaagtacggtctattatatctcttttacagacccagaaactgaggcgcagaagttagggtcagccccaggtcacacagctaacaagagctggcctaggcacccaggg

SEQ ID NO: 471 - DNA, 122 bp, Artificial Sequence
  Description of Artificial Sequence Synthetic polynucleotide
ggggcgtgggtgggtcagcgttccttggggacccgtgaagcctgggcttagggctcacagcgtgggtccccagcacagacaggaggcggacagcttcccgtgaactgcaggggagtcccggg

SEQ ID NO: 472 - DNA, 122 bp, Artificial Sequence
  Description of Artificial Sequence Synthetic polynucleotide
caaactagtgactgttttactgcaggtgaagaaggggcagagatcagaggctctagcaggcgggacaatgcccagggattcatgagccggacaaagctgtatccctccatttccacctgcca

SEQ ID NO: 473 - DNA, 122 bp, Artificial Sequence
  Description of Artificial Sequence Synthetic polynucleotide
ggagcccctgggatgacccatcccaaggtcccagcctaagtctgaggttccagggctggtcgcaggccgtccttgcagccctcgccagagcgttgtctgcacctccgacactaggtggcgcc

SEQ ID NO: 474 - DNA, 122 bp, Artificial Sequence
  Description of Artificial Sequence Synthetic polynucleotide
gagggatggttgtcctcacccctgtgaggcaatatgctgtccattagtatccactgaatgcgtgaaatttttttctaatgggcaaactgaggctcagagaagttcctgtctggctcaaggtt

SEQ ID NO: 475 - DNA, 122 bp, Artificial Sequence
  Description of Artificial Sequence Synthetic polynucleotide
tcccgggcgcggaggatggaaacctggcggtaacctctgcaggtcgtgccactcggtgtgcgcaaggtctccagaggcatcttttcatttttagggggcactttccacgaattcatttgagc

SEQ ID NO: 476 - DNA, 122 bp, Artificial Sequence
  Description of Artificial Sequence Synthetic polynucleotide
gcgcttccagaaggctgcaaatgggaattccagacaaacccacttgggtgaatcccagcacgcgggctgcggcgtagggggagagctcctcacgcggctcagagtgtagcccaggcccgcag

SEQ ID NO: 477 - DNA, 122 bp, Artificial Sequence
  Description of Artificial Sequence Synthetic polynucleotide
gggaaagtctcaaaactgtcaactctgatagaaagctcatgtcagagacctgaagctcagcgatgtagttctgagacatatctaagactttggttttcagcggtaggtcttttggaacatga

SEQ ID NO: 478 - DNA, 122 bp, Artificial Sequence
  Description of Artificial Sequence Synthetic polynucleotide
tctacctagtaacagctgagaaataaggctcgagacaccattggttggttcagcctcactcggccaatcctgggctctaaactgctcagtggaaatcttgggactttttggacacccagaga

SEQ ID NO: 479 - DNA, 122 bp, Artificial Sequence
  Description of Artificial Sequence Synthetic polynucleotide
cagcccacgtgactacaggggcacttgatgggaatcatggcagcatccaggccattgtcccgcttctgggagtggggaaagaacatcgtctgcgtggggaggaactacgcggaccacgtcag

SEQ ID NO: 480 - DNA, 122 bp, Artificial Sequence
  Description of Artificial Sequence Synthetic polynucleotide
aggaggatctctgtaaattgttttcttagggagaaggatagggtgaaggagtagaatcgacgactgtagatttgtgagtagaatcccatttgtagttaaacttgggtaaatgggagaaaggg

SEQ ID NO: 481 - DNA, 122 bp, Artificial Sequence
  Description of Artificial Sequence Synthetic polynucleotide
gcctctctgtggttctgcctggaagacggaaggcaggtggttggctctagtcatccacgacgggctggcacctctccagctgcggccagtctaaccccagggcctgctgggaaatgtagttc

SEQ ID NO: 482 - DNA, 122 bp, Artificial Sequence
  Description of Artificial Sequence Synthetic polynucleotide
gcgcccctggcgtccgggcaggtgccaggtgaggaaagaaatgggggccgctccatgaagcggttcctgccaataaagaaaacgacatccagagaatacccaggcggggaataaaggggtcc

SEQ ID NO: 483 - DNA, 122 bp, Artificial Sequence
  Description of Artificial Sequence Synthetic polynucleotide
cagcacgggcggggggcaggggctggggccgaccgggaggccggtgccaaggatgggggccgcccggctgccccgcgcgtgaggaggccgaggggcgcgccaccccggcccggggcggccgc

SEQ ID NO: 484 - DNA, 122 bp, Artificial Sequence
  Description of Artificial Sequence Synthetic polynucleotide
taatctcttctttggacgtttggcagctccatttcacctccccttaactctgtttgggatcgcttacacaccaaggaagttgggctttgagaattccatcccactggcactgaggagaatat

SEQ ID NO: 485 - DNA, 122 bp, Artificial Sequence
  Description of Artificial Sequence Synthetic polynucleotide
cagcctttccccgggcctggggttcctggactaggctgcgctgcagtgactgtggactggcgtgtggcgggggtcgtggcagcccctgccttacctctaggtgccagccccaggcccgggcc

SEQ ID NO: 486 - DNA, 122 bp, Artificial Sequence
  Description of Artificial Sequence Synthetic polynucleotide
ggctttcccgaatggcgcgcccaggacggctcttgcggctggctgtccaaactgggcccgcgtcctgaagtgaccccagcctgatctcggccagctgcttgtgaccttggcctgtcccagca

SEQ ID NO: 487 - DNA, 122 bp, Artificial Sequence
  Description of Artificial Sequence Synthetic polynucleotide
cctggccggccggctcgctaggcgcggggtctaggccaggctggggctgcttggaggctgcgccctcccctgcccgcggcgccccggcccccgccgtcgagagtggacgcccctctggggta

SEQ ID NO: 488 - DNA, 122 bp, Artificial Sequence
  Description of Artificial Sequence Synthetic polynucleotide
ctctaaaaagtgacattgatgccaactgccagagctggtacccatgccatctgctagtgacgtcacagggcagagagagccatgtgatcctctctcttgggaccttcattctgcactgatca

SEQ ID NO: 489 - DNA, 122 bp, Artificial Sequence
  Description of Artificial Sequence Synthetic polynucleotide
gtttgcactgaaagttgtgttggctcaggagctgcttttccggggatctgcagttgcccccgccacctcctggctgcggttggcaggtccctccctcagcagttcgtcctccgcctgcgccg

SEQ ID NO: 490 - DNA, 122 bp, Artificial Sequence
  Description of Artificial Sequence Synthetic polynucleotide
caaggagggagcaggagcattcgaacgcggaaatcgaggtgctagtccaaactgctcggtcggctttagtcatagctggataatgcccggctcaggtctaccacaagccatacagctgcttt

SEQ ID NO: 491 - DNA, 122 bp, Artificial Sequence
  Description of Artificial Sequence Synthetic polynucleotide
ttgttacgggcgcggtggtgcaggggcaaatcgggactgggatttggtccttacccttaacgtggctctaagaccagaagggaacacctgacttgtgttgacctcttcagttagctgcaggt

SEQ ID NO: 492 - DNA, 122 bp, Artificial Sequence
  Description of Artificial Sequence Synthetic polynucleotide
tgaaaacacagcaagggccccactagctgaaaccaagttgcagagttttgagggtcccaccgccgaccgccggcccgccgcgagccctgccccctgcgcggccacgcccccttgctccccgc

SEQ ID NO: 493 - DNA, 122 bp, Artificial Sequence
  Description of Artificial Sequence Synthetic polynucleotide
taaataaataagggcttttgtttgtttgccggctcctgcacatggctgctgggactcaagcgctcgtgttgtctgcgcctctgtgggactctggggacgggaggcaggggaggcccccgcag

SEQ ID NO: 494 - DNA, 122 bp, Artificial Sequence
  Description of Artificial Sequence Synthetic polynucleotide
gaggctctgaggctgcaacagtctccctcctattgaagctagaacagcaccccgagcctgcgccataagtgcccccagaacttcagcgcccaccatggcgcacaaggccggtgcccagcgcc

SEQ ID NO: 495 - DNA, 122 bp, Artificial Sequence
  Description of Artificial Sequence Synthetic polynucleotide
cttgggcaacgtaggagacctccgtctccacaagtaaaattaattagccggctgtggtggcgcgcacctgtggtcccagctactcaggaggctgaggtaggaggatcacctgagcccgggag

SEQ ID NO: 496 - DNA, 122 bp, Artificial Sequence
  Description of Artificial Sequence Synthetic polynucleotide
gaggctgaggcaggagaatggcgtgaacccgggagaatggtgtgccccgagcctacttcccgcgcagtcctccaagacgcggcctccagcaggggtcgctgcttcgctgccgccctggcctc

SEQ ID NO: 497 - DNA, 122 bp, Artificial Sequence
  Description of Artificial Sequence Synthetic polynucleotide
gccaggaccaaatgcccccggaagcggggagcgacagcagcggagaggaacatgtcctggcgcccccgggcctgcagcctccacactgccccggccagtgtctgatctgggcttgcaagacc

SEQ ID NO: 498 - DNA, 122 bp, Artificial Sequence
  Description of Artificial Sequence Synthetic polynucleotide
gcttggacacttgcagcatgttgcctctgccaactgcctagaatttaagcctgactttgccgccttactgaccccagcaacttaacctgtctgtgccttaattttcttatctacagaatgga

SEQ ID NO: 499 - DNA, 122 bp, Artificial Sequence
  Description of Artificial Sequence Synthetic polynucleotide
aaagttaagtactaagtatgtggtgaacaaataaatccacccttctgaaacacatctaaacgaggtcctgtatttgaaagtgtctggaagattaaaaggcactacaccaaagctgctagcac

SEQ ID NO: 500 - DNA, 122 bp, Artificial Sequence
  Description of Artificial Sequence Synthetic polynucleotide
ggactggtacaggacaggcatctttgaacctatttctgggagttctgaaactactgttctcgtgggccttggcgactgatttgggaaagctgaccctgggttggcctggcttccagccaccg

SEQ ID NO: 501 - DNA, 122 bp, Artificial Sequence
  Description of Artificial Sequence Synthetic polynucleotide
cgacgacgacctcaacagcgtgctggacttcatcctgtccatggggctggatggcctgggcgccgaggccgccccggagccgccgccgccgcccccgccgcctgcgttctattaccccgaac

SEQ ID NO: 502 - DNA, 122 bp, Artificial Sequence
  Description of Artificial Sequence Synthetic polynucleotide
acaaagtgatctggcacagctgcagggtggcattgagtctgaggcttatggtgcagaagccgaagttaaagatgtttctagagcctgaagacttcctcttgagggtgagttgctgcctacaa

SEQ ID NO: 503 - DNA, 122 bp, Artificial Sequence
  Description of Artificial Sequence Synthetic polynucleotide
cgcgggtggaaggtgaaggtcgagggaggtcaggctgcttctgcgtgtcctgacggctggcgtgttctcttgagatgggctcgggctacttggccagcttcaatttaagccacagtgtctcc

SEQ ID NO: 504 - DNA, 122 bp, Artificial Sequence
  Description of Artificial Sequence Synthetic polynucleotide
cccagcccacggcggcccgcgagggacaaaacgcgccgcgcctggttccccgcccacggacgcggtgactttccagaacgtcttaaaggcaacgcactctgactcaaggcccagggaggctg

SEQ ID NO: 505 - DNA, 122 bp, Artificial Sequence
  Description of Artificial Sequence Synthetic polynucleotide
tgtttttgtgggaggccttctgcatggtcccgggaggtcaggcagcccgggagggcctcccggagcagaggctggagtcagtcccaatgccaacagtttcgaaccttgcccgcgggcactgc

SEQ ID NO: 506 - DNA, 122 bp, Artificial Sequence
  Description of Artificial Sequence Synthetic polynucleotide
cctgtcttcagcagcatcgctctggactcagcttccgaggacctgaccagatctggtctgcgtgtatcagctgtatgtgttgggctctggaagctaagaaacgtctgaaaagcactggggtc

SEQ ID NO: 507 - DNA, 122 bp, Artificial Sequence
  Description of Artificial Sequence Synthetic polynucleotide
tgccccggtaactgcctccccaacacctgcctgccttccactgcgaaacctgctctcggacgccctgaccataccgcacacaatactgcaagcctgtgtgggcctgggggtgggatggaccc

SEQ ID NO: 508 - DNA, 122 bp, Artificial Sequence
  Description of Artificial Sequence Synthetic polynucleotide
gatggccctttaagaggcactgtccagctctggttgccatggagacagctggacacagaccgggtagaggcaggcccacagcatgtcctccaaggtttactccacaggtgggaagaggactg

SEQ ID NO: 509 - DNA, 122 bp, Artificial Sequence
  Description of Artificial Sequence Synthetic polynucleotide
ctgggattacaggcgtgagccactgcgcctggcctttgcaaggttttgaggaaagtgaagcgttctgttgaagcagggcttgagttctgttgtaagtgtttcatgaagccctggagacctct

SEQ ID NO: 510 - DNA, 122 bp, Artificial Sequence
  Description of Artificial Sequence Synthetic polynucleotide
tcaggttctggaaccaagacaagtccagggacaaccccaaagctggcctgggctcccgcgcggacagcttttataccctgtacggaaccgcccctgcccaggattgaagtggccccgcctcc

SEQ ID NO: 511 - DNA, 122 bp, Artificial Sequence
  Description of Artificial Sequence Synthetic polynucleotide
aggtggaaatactttcgggcgatggtgggggcctggtgcttcttggactcggaagatgaccgcttggcattctggtacagcaccaccaggcaggccaaggtggccagcagagaccaataggc

SEQ ID NO: 512 - DNA, 122 bp, Artificial Sequence
  Description of Artificial Sequence Synthetic polynucleotide
gaaccagggcccttggcgagagttggggtgggaatcgcgtaagaaaagcaatttctagagcggaaaggtgaccccacattacaaaaagaaatggagtagaaaaataggcttgactattctaa

SEQ ID NO: 513 - DNA, 122 bp, Artificial Sequence
  Description of Artificial Sequence Synthetic polynucleotide
ctatcagcctaacgattaagtcaacatgctaagcagccacacgggggctactaagtgactcgcacgggggaagcaggcagggagacagatgggcaggggagggaatctggggcaatgcacaa